SEQUENCE LISTING (32 sequences)

SEQ ID NO: 1 - RNA, 10936 nt, synthetic construct
  source 1..10936: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatgagggcccctataactctctacggctaacctgaatggactacgacatagtctagtccgccaagatgaaccccaaccagaagatcatcaccatcggcagcatctgcatgacaatcggcaccgccaacctgatcctgcagatcggcaacatcatcagcatctgggtgtcccacagcatccagatcggaaaccagagccagatcgagacatgcaacaagagcgtgatcacctacgagaacaacacctgggtcaaccagaccttcgtgaacatcagcaacaccaacagcgccgccagacagtctgtggcctctgtgaaactggccggcaacagctctctgtgtcctgtgtctggctgggccatctacagcaaggacaactctgtgcggatcggctccaagggcgacgtgttcgtgatcagagagcccttcatcagctgcagccctctggaatgccggacattctttctgacccaaggcgccctgctgaacgacaagcacagcaacggcaccatcaaggacagaagcccctacagaaccctgatgagctgccctatcggcgaggtgccctctccatacaacagcagattcgagtccgtggcttggagcgcctctgcctgtcacgatggcaccaactggctgaccatcggaatcagcggacctgattctggcgctgtggccgtgctgaagtacaatggcatcatcaccgataccatcaagagctggcggaacaagatcctgcggacccaagagtccgagtgcgcctgtgtgaatggcagctgcttcaccatcatgacagacggcccatctgatggccaggccagctacaagatcttccggatcgagaagggcaagatcattaagagcgtggaaatgaaggccccgaactaccactacgaggaatgcagctgttaccccgacagcagcgagatcacctgtgtgtgcagagacaactggcacggcagcaacagaccttgggtgtccttcaaccagaacctggaataccagatgggctacatctgcagcggcgtgttcggcgacaaccccagacctaatgataagaccggcagctgcggccctgtgtctagcaatggtgccaatggcgtgaagggcttcagctttaagtacggcaacggcgtgtggatcggccggacaaagagcatcagcagcagaaagggcttcgagatgatctgggaccccaatggctggaccggcaccgacaacaagttcagcaagaaacaggacatcgtgggcatcaacgagtggagcggctacagcggctctttcgtgcagcaccctgaactgaccggcctgaactgcatcagaccctgcttttgggtcgagctgatccggggcagacccgaggaaaacaccatctggacaagcggcagcagcatcagcttttgcggcgtggactctgacatcgtcggctggtcttggcctgatggcgccgagctgcctttcaccatcgacaagtgatgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 9433 nt, synthetic construct
  source 1..9433: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaa

SEQ ID NO: 3 - RNA, 44 nt, synthetic construct
  source 1..44: mol_type = other RNA
ataggcggcgcatgagagaagcccagaccaattacctacccaaa

SEQ ID NO: 4 - RNA, 61 nt, synthetic construct
  source 1..61: mol_type = other RNA
gggcccctataactctctacggctaacctgaatggactacgacatagtctagtccgccaag

SEQ ID NO: 5 - RNA, 117 nt, synthetic construct
  source 1..117: mol_type = other RNA
atacagcagcaattggcaagctgcttacatagaactcgcggcgattggcatgccgccttaaaatttttattttatttttcttttcttttccgaatcggattttgtttttaatatttc

SEQ ID NO: 6 - RNA, 61 nt, synthetic construct; identical to SEQ ID NO: 4
  source 1..61: mol_type = other RNA

SEQ ID NO: 7 - RNA, 10 nt, synthetic construct
  source 1..10: mol_type = other RNA
atatcgcacc

SEQ ID NO: 8 - RNA, 44 nt, synthetic construct; identical to SEQ ID NO: 3
  source 1..44: mol_type = other RNA

SEQ ID NO: 9 - RNA, 61 nt, synthetic construct; identical to SEQ ID NO: 4
  source 1..61: mol_type = other RNA

SEQ ID NO: 10 - RNA, 117 nt, synthetic construct; identical to SEQ ID NO: 5
  source 1..117: mol_type = other RNA

SEQ ID NO: 11 - RNA, 10 nt, synthetic construct
  source 1..10: mol_type = other RNA
atatcgcacc

SEQ ID NO: 12 - RNA, 44 nt, synthetic construct; identical to SEQ ID NO: 3
  source 1..44: mol_type = other RNA

SEQ ID NO: 13 - RNA, 1605 nt, synthetic construct
  source 1..1605: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 14 - RNA, 2382 nt, synthetic construct
  source 1..2382: mol_type = other RNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 15 - RNA, 1671 nt, synthetic construct
  source 1..1671: mol_type = other RNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 16 - RNA, 1825 nt, synthetic construct
  source 1..1825: mol_type = other RNA
atacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 17 - RNA, 61 nt, synthetic construct; identical to SEQ ID NO: 4
  source 1..61: mol_type = other RNA

SEQ ID NO: 18 - RNA, 1706 nt, synthetic construct
  source 1..1706: mol_type = other RNA
atgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatgagg

SEQ ID NO: 19 - RNA, 59 nt, synthetic construct
  source 1..59: mol_type = other RNA
gcccctataactctctacggctaacctgaatggactacgacatagtctagtccgccaag

SEQ ID NO: 20 - RNA, 1413 nt, synthetic construct
  source 1..1413: mol_type = other RNA
atgaaccccaaccagaagatcatcaccatcggcagcatctgcatgacaatcggcaccgccaacctgatcctgcagatcggcaacatcatcagcatctgggtgtcccacagcatccagatcggaaaccagagccagatcgagacatgcaacaagagcgtgatcacctacgagaacaacacctgggtcaaccagaccttcgtgaacatcagcaacaccaacagcgccgccagacagtctgtggcctctgtgaaactggccggcaacagctctctgtgtcctgtgtctggctgggccatctacagcaaggacaactctgtgcggatcggctccaagggcgacgtgttcgtgatcagagagcccttcatcagctgcagccctctggaatgccggacattctttctgacccaaggcgccctgctgaacgacaagcacagcaacggcaccatcaaggacagaagcccctacagaaccctgatgagctgccctatcggcgaggtgccctctccatacaacagcagattcgagtccgtggcttggagcgcctctgcctgtcacgatggcaccaactggctgaccatcggaatcagcggacctgattctggcgctgtggccgtgctgaagtacaatggcatcatcaccgataccatcaagagctggcggaacaagatcctgcggacccaagagtccgagtgcgcctgtgtgaatggcagctgcttcaccatcatgacagacggcccatctgatggccaggccagctacaagatcttccggatcgagaagggcaagatcattaagagcgtggaaatgaaggccccgaactaccactacgaggaatgcagctgttaccccgacagcagcgagatcacctgtgtgtgcagagacaactggcacggcagcaacagaccttgggtgtccttcaaccagaacctggaataccagatgggctacatctgcagcggcgtgttcggcgacaaccccagacctaatgataagaccggcagctgcggccctgtgtctagcaatggtgccaatggcgtgaagggcttcagctttaagtacggcaacggcgtgtggatcggccggacaaagagcatcagcagcagaaagggcttcgagatgatctgggaccccaatggctggaccggcaccgacaacaagttcagcaagaaacaggacatcgtgggcatcaacgagtggagcggctacagcggctctttcgtgcagcaccctgaactgaccggcctgaactgcatcagaccctgcttttgggtcgagctgatccggggcagacccgaggaaaacaccatctggacaagcggcagcagcatcagcttttgcggcgtggactctgacatcgtcggctggtcttggcctgatggcgccgagctgcctttcaccatcgacaagtgatga

SEQ ID NO: 21 - RNA, 117 nt, synthetic construct; identical to SEQ ID NO: 5
  source 1..117: mol_type = other RNA

SEQ ID NO: 22 - RNA, 79 nt, synthetic construct
  source 1..79: mol_type = other RNA
aaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - RNA, 44 nt, synthetic construct; identical to SEQ ID NO: 3
  source 1..44: mol_type = other RNA

SEQ ID NO: 24 - RNA, 1605 nt, synthetic construct; identical to SEQ ID NO: 13
  source 1..1605: mol_type = other RNA

SEQ ID NO: 25 - RNA, 2382 nt, synthetic construct; identical to SEQ ID NO: 14
  source 1..2382: mol_type = other RNA

SEQ ID NO: 26 - RNA, 1671 nt, synthetic construct; identical to SEQ ID NO: 15
  source 1..1671: mol_type = other RNA

SEQ ID NO: 27 - RNA, 1824 nt, synthetic construct
  source 1..1824: mol_type = other RNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 28 - RNA, 61 nt, synthetic construct; identical to SEQ ID NO: 4
  source 1..61: mol_type = other RNA

SEQ ID NO: 29 - RNA, 10 nt, synthetic construct
  source 1..10: mol_type = other RNA
atatcgcacc

SEQ ID NO: 30 - RNA, 1704 nt, synthetic construct
  source 1..1704: mol_type = other RNA
atgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatga

SEQ ID NO: 31 - RNA, 117 nt, synthetic construct; identical to SEQ ID NO: 5
  source 1..117: mol_type = other RNA

SEQ ID NO: 32 - RNA, 40 nt, synthetic construct
  source 1..40: mol_type = other RNA
aaaaaaaaaaaaaaaaaaaaaaaaaaaaaaaaaaaaaaaa

(19) United States
(12) Patent Application Publication
(10) Pub. No.: US 2024/0009296 A1
(43) Pub. Date: Jan. 11, 2024

(54) SELF-AMPLIFYING RNA ENCODING AN INFLUENZA VIRUS ANTIGEN

(71) Applicant: PFIZER INC., NEW YORK, NY (US)

(72) Inventors: Hui Cai, Ridgewood, NJ (US); Ye Che, Niantic, CT (US); Fernando Martin Diaz, New York, NY (US); Raquel Munoz-Moreno, Cliffside Park, NJ (US); Alicia Solórzano Quijano, New York, NY (US); Chong Wang, East Lyme, CT (US)

(73) Assignee: PFIZER INC., NEW YORK, NY (US)

(21) Appl. No.: 18/347,698

(22) Filed: Jul. 6, 2023

Related U.S. Application Data
(60) Provisional application No. 63/359,857, filed on Jul. 10, 2022.
(60) Provisional application No. 63/484,745, filed on Feb. 13, 2023.
(60) Provisional application No. 63/431,462, filed on Dec. 9, 2022.

(51) Int. Cl. A61K 39/145

(52) U.S. Cl. CPC A61K 39/145; A61K 2039/53

(57) ABSTRACT

Self-amplifying RNA (saRNA) molecules encoding an influenza virus antigen and methods of use thereof are disclosed herein.

### CROSS-REFERENCE TO APPLICATIONS

This application claims the benefit of U.S. provisional application No. 63/359,857, filed Jul. 10, 2022, U.S. provisional application No. 63/431,462, filed Dec. 9, 2022, and U.S. provisional application No. 63/484,745, filed Feb. 13, 2023, each of which is incorporated by reference herein in its entirety.

### FIELD

The invention relates to compositions and methods for the preparation, manufacture and therapeutic use of ribonucleic acid vaccines comprising polynucleotide molecules encoding one or more influenza antigens, such as hemagglutinin antigens.

### BACKGROUND

Influenza viruses are members of the orthomyxoviridae family, and are classified into three types (A, B, and C), based on antigenic differences between their nucleoprotein (NP) and matrix (M) protein.

The genome of influenza A virus includes eight molecules (seven for influenza C virus) of linear, negative polarity, single-stranded RNAs, which encode several polypeptides including: the RNA-directed RNA polymerase proteins (PB2, PB1 and PA) and nucleoprotein (NP), which form the nucleocapsid; the matrix proteins (M1, M2, which is also a surface-exposed protein embedded in the virus membrane); two surface glycoproteins, which project from the lipoprotein envelope: hemagglutinin (HA) and neuraminidase (NA); and nonstructural proteins (NS1 and NS2).

Hemagglutinin is the major envelope glycoprotein of influenza A and B viruses, and hemagglutinin-esterase (HE) of influenza C viruses is a protein homologous to HA.

A challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation.

There is a need for improved compositions, preferably immunogenic compositions, against influenza.

### SUMMARY

The unmet needs for improved compositions, preferably immunogenic compositions, against influenza, among other things, are provided herein. In one aspect, the disclosure relates to a composition comprising a self-amplifying RNA (saRNA) comprising: a 5′ Cap; a 5′ untranslated region (5′ UTR); a coding region for a nonstructural protein derived from an alphavirus; a first subgenomic promoter derived from an alphavirus; a first open reading frame encoding a first gene of interest derived from influenza virus hemagglutinin (HA); a second subgenomic promoter derived from an alphavirus; a second open reading frame encoding a second gene of interest derived from influenza virus; a 3′ untranslated region (3′ UTR); and a 3′ poly A sequence.

In another aspect, the disclosure relates to a composition comprising a self-amplifying RNA (saRNA) comprising: a 5′ Cap; a 5′ untranslated region (5′ UTR); a coding region for a nonstructural protein derived from an alphavirus; a subgenomic promoter derived from an alphavirus; an open reading frame encoding a gene of interest derived from influenza virus; a 3′ untranslated region 3′ UTR); and a 3′ poly A sequence; wherein at least 5% of a total population of a particular nucleotide in the molecule has been replaced with one or more modified or unnatural nucleotides.

In preferred embodiments, the saRNA polynucleotide has a clinical grade purity. In some embodiments, the purity of the RNA polynucleotide is between about 60% and about 100%. In some embodiments, the purified RNA polynucleotide has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or greater as determined by a known method, such as, e.g., capillary electrophoresis. In some embodiments, equal to any one of, at least any one of, at most any one of, or between any two of 35%, 40%, 45%, 50%, 55%, 60%, 65%, 70%, 71%, 72%, 73%, 74%,75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules in the composition are full length RNA transcripts. A “full length” RNA molecule is one that includes a 5′-cap and a poly A tail.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Functional Anti-HA Antibodies Elicited by Immunization of Mice With LNP-Formulated saRNA Encoding Influenza HA and/or NA as Measured by HAI; FIG. 1 depicts 3 weeks post prime and 2 weeks post boost; Female Balb/c mice were immunized IM on Day 0 with 20 ng of the LNP-formulated bicistronic and monocistronic saRNA vaccine preparations, 40 ng total (20 ng each) of the 1:1 mix of saRNA-HA+saRNA-NA, and 200 ng of the modRNA comparator encoding A/Wisconsin/588/2019 (H1N1) HA. Antibody responses against A/Wisconsin/588/2019 were measured by HAI or a 1-Day MNT assay on Day 21 (3 weeks after immunization). HAI titers are reported (Geometric mean with geometric SD).

FIG. 2—Neutralizing Antibodies Elicited by Immunization of Mice With LNP-Formulated saRNA Encoding Influenza HA and/or NA as Measured by 1-Day MNT; FIG. 2 depicts 3 weeks post prime and 2 weeks post boost; Female Balb/c mice were immunized IM on Day 0 with 20 ng of the LNP-formulated bicistronic and monocistronic saRNA vaccine preparations, 40 ng total (20 ng each) of the 1:1 mix of saRNA-HA+saRNA-NA, and 200 ng of the modRNA comparator encoding A/Wisconsin/588/2019 (H1N1) HA. Antibody responses against A/Wisconsin/588/2019 were measured by HAI or a 1-Day MNT assay on Day 21 (3 weeks after immunization). 50% neutralization titers are reported (Geometric mean with geometric SD).

FIG. 3—Neutralizing Antibodies Elicited by Immunization of Mice With LNP-Formulated saRNA Encoding Influenza HA and/or NA as Measured by 3-Day MNT

FIG. 4—Functional Anti-NA Antibodies Elicited by Immunization of Mice With LNP-Formulated saRNA Encoding Influenza HA and/or NA as Measured by NAI; FIG. 4 depicts 3 weeks post prime and 2 weeks post boost; Female Balb/c mice were immunized IM on Day 0 with 20 ng of the LNP-formulated bicistronic and monocistronic saRNA vaccine preparations, 40 ng total (20 ng each) of the 1:1 mix of saRNA-HA+saRNA-NA, and 200 ng of the modRNA comparator encoding A/Wisconsin/588/2019 (H1N1) NA. Antibody responses against A/Wisconsin/588/2019 were measured by NAI on Day 21 (3 weeks after immunization). Geometric mean titers with geometric SD are reported.

FIG. 5—Serum cytokines and chemokines at 24 hours after immunization of Balb/c mice with influenza saRNA-HA vaccine preparations with different amounts of modified nucleosides

FIG. 6—Functional HAI and neutralizing antibodies elicited by immunization of Balb/c mice with LNP-formulated saRNA-HA vaccine preparations with different amounts of modified nucleosides

FIG. 7—Serum cytokines and chemokines at 24 hours after immunization of C57BL6/J mice with influenza saRNA-HA vaccine preparations with different amounts of modified nucleosides

FIG. 8—functional HAI and neutralizing antibodies elicited by immunization of C57NL6/J mice with LNP-formulated saRNA-HA vaccine preparations with different amounts of modified nucleosides; Female Balb/c mice were immunized IM on Day 0 with 20 ng of the LNP-formulated quadrivalent saRNA comprised of 4 bicistronic constructs encoding HA and NA from A/Wisconsin/588/2019 (H1N1), A/Cambodia/e0826360/2020 (H3N2), B/Washington/2/2019 (B/Victoria-lineage), and B/Phuket/3073/2013 (B/Yamagata-lineage), or with 2.4 μg of a licensed, adjuvanted quadrivalent inactivated vaccine (QIV; FluAd). Antibody responses against each vaccine component were measured by HAI or a 1-Day MNT assay on Day 42 (2 weeks after 2nd dose). HAI and 50% neutralization titers are reported (Geometric mean with geometric SD).

FIG. 9—Functional HAI and Neutralizing Antibodies Elicited by Immunization of Mice With LNP-Formulated Quadrivalent Bicistronic saRNA Encoding HA and NA from 4 Seasonal Influenza Strains

FIG. 10—Functional NAI Antibodies Elicited by Immunization of Mice With LNP-Formulated Quadrivalent Bicistronic saRNA Encoding HA and NA from 4 Seasonal Influenza Strains; Female Balb/c mice were immunized IM on Day 0 with 20 ng of the LNP-formulated quadrivalent saRNA comprised of 4 bicistronic constructs encoding HA and NA from A/Wisconsin/588/2019 (H1N1), A/Cambodia/e0826360/2020 (H3N2), B/Washington/2/2019 (B/Victoria-lineage), and B/Phuket/3073/2013 (B/Yamagata-lineage), or with 2.4 μg of a licensed, adjuvanted quadrivalent inactivated vaccine (QIV; FluAd). Antibody responses against each vaccine component were measured by NAI on Day 42 (2 weeks after 2nd dose). NAI titers are reported (Geometric mean with geometric SD) for 3 of the 4 strains. H3N2 NAI titers could not be reported for both the saRNA and QIV due to technical issues with the NAI assay for this strain.

FIG. 11—Geometric Mean Titers and 95% CI: HAI—Vaccine Preparations 1, 2, and Control Groups—Evaluable Immunogenicity Population
Abbreviations: GMT=geometric mean titer; HAI=hemagglutination inhibition; QIV=quadrivalent influenza vaccine, Vax Prep=vaccine preparation.
Note: V1=Day 1 prior to vaccination; V3=1 Week; V4=2 Weeks; V5=4 Weeks.
Note: Dots present individual antibody levels.
Note: Numbers/GMTs within each bar denote the number of participants with valid and determinate assay results for the specified assay at the given sampling time point,
and corresponding geometric mean titers. Average of two samples collected at Day 1 prior to vaccination were used for GMT calculation.
Note: Licensed QIV-15A includes participants in study receiving licensed QIV, whose VRD data were tested at the same time as VRD data of CI groups were tested.
Note: Licensed QIV-18 includes participants in study receiving licensed QIV, whose VRD data were tested at the same time as VRD data of C2-C5 and C7 groups were tested. Note: Placebo includes participants in study receiving placebo as randomized.

FIG. 12—Geometric Mean Titers and 95% CI: HAI—Vaccine Preparations 3, 4, 7, and Control Groups—Evaluable Immunogenicity Population
Abbreviations: GMT=geometric mean titer; HAI=hemagglutination inhibition; QIV=quadrivalent influenza vaccine; Vax Prep=vaccine preparation.
Note: V1=Day 1 prior to vaccination; V3=1 Week; V4=2 Weeks; V5=4 Weeks.
Note: Dots present individual antibody levels.
Note: Numbers/GMT within each bar denote the number of participants with valid and determinate assay results for the specified assay at the given sampling time point, and corresponding geometric mean titers. Average of two samples collected at Day 1 prior to vaccination vaccination were used for GMT calculation.
Note: Licensed Q1V-18 includes participants in study receiving licensed QIV, whose VRD data were tested at the same time as VRD data of C2-C5 and C7 groups were tested.
Note: Placebo includes participants in study receiving placebo as randomized.

FIG. 13—Geometric Mean Titers and 95% CI: HAI—Vaccine Preparations 5, 6, and Control Groups—Evaluable Immunogenicity Population
Abbreviations: GMT=geometric mean titer; HAI=hemagglutination inhibition; QIV=quadrivalent influenza vaccine; Vax Prep=vaccine preparation.
Note: VI=Day 1 prior to vaccination; V3=1 Week, V4=2 Weeks; V5=4 Weeks.
Note: Dots present individual antibody levels.
Note: Numbers/GMTs within each bar denote the number of participants with valid and determinate assay results for the specified assay at the given sampling time point, and corresponding geometric mean titers. Average of two samples collected at Day 1 prior to vaccination were used for GMT calculation.
Note: Licensed QIV-18 includes participants in study receiving licensed QIV, whose VRD data were tested at the same time as VRD data of C2-C5 and C7 groups were tested.
Note: Licensed QIV-15B includes participants in study receiving licensed QIV, whose VRD data were tested at the same time as VRD data of C6 groups were tested.
Note: Placebo includes participants in study receiving placebo as randomized.

FIG. 14—Functional Anti-HA Antibodies Elicited in Mice Following One Dose of HA/NA-Encoding saRNA-LNPs, Quad modRNA, or FluAd Measured by HAI

FIG. 15—Functional Anti-NA Antibodies Elicited in Mice Following One Dose of HA/NA-Encoding saRNA-LNPs or FluAd Measured by NAI

FIG. 16—Virus Neutralizing Antibodies Elicited in Mice Following One Dose of HA/NA-Encoding saRNA-LNPs, Quad modRNA, or FluAd Measured by 1-Day MNT

FIG. 17—Functional Anti-HA Antibodies Elicited in Mice Following Two Doses of HA/NA-Encoding saRNA-LNPs, Quad modRNA, or FluAd Measured by HAI

FIG. 18—Functional Anti-NA Antibodies Elicited in Mice Following Two Doses of HA/NA-Encoding saRNA-LNPs or FluAd Measured by NAI

FIG. 19—Virus Neutralizing Antibodies Elicited in Mice Following Two Doses of HA/NA-Encoding saRNA-LNPs, Quad modRNA, or FluAd Measured by 1-Day MNT

### DETAILED DESCRIPTION

Embodiments of the present disclosure provide compositions that include a self-amplifying RNA (saRNA) polynucleotide encoding an influenza virus antigen. Influenza virus RNA vaccines, as provided herein may be used to induce a balanced immune response, comprising both cellular and humoral immunity.

It is contemplated that any embodiment discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

A. Self-Amplifying RNA (saRNA)

In some embodiments, the RNA molecule, such as the first RNA molecule, is an saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

In some embodiments, the self-amplifying RNA includes at least one or more genes selected from any one of viral replicases, viral proteases, viral helicases and other nonstructural viral proteins. In some embodiments, the self-amplifying RNA may also include 5′- and 3 ‘-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some embodiments, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alphavirus, such as Sinbis virus (SIN), Semliki forest virus and Venezuelan equine encephalitis virus (VEE), one or more genes encoding viral structural proteins, such as capsid and/or envelope glycoproteins, may be omitted.

In some embodiments, a self-amplifying RNA molecule described herein encodes (i) an RNA-dependent RNA polymerase that may transcribe RNA from the self-amplifying RNA molecule and (ii) a polypeptide of interest, e.g., a viral antigen. In some embodiments, the polymerase may be an alphavirus replicase, e.g., including any one of alphavirus protein nsP1, nsP2, nsP3, nsP4, and any combination thereof. In some embodiments, the self-amplifying RNA molecules described herein may include one or more modified nucleotides (e.g., pseudouridine, N6-methyladenosine, 5-methylcytidine, 5-methyluridine). In some embodiments, the self-amplifying RNA molecules does not include a modified nucleotide (e.g., pseudouridine, N6-methyladenosine, 5-methylcytidine, 5-methyluridine).

The saRNA construct may encode at least one non-structural protein (NSP), disposed 5′ or 3′ of the sequence encoding at least one peptide or polypeptide of interest. In some embodiments, the sequence encoding at least one NSP is disposed 5′ of the sequences encoding the peptide or polypeptide of interest. Thus, the sequence encoding at least one NSP may be disposed at the 5′ end of the RNA construct. In some embodiments, at least one non-structural protein encoded by the RNA construct may be the RNA polymerase nsP4. In some embodiments, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some embodiments, the polymerase may be an alphavirus replicase, e.g., comprising one or more of alphavirus proteins nsP1, nsP2, nsP3, and nsP4.

Whereas natural alphavirus genomes encode structural virion proteins in addition to the non-structural replicase polypeptide, in some embodiments, the self-amplifying RNA molecules do not encode alphavirus structural proteins. In some embodiments, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins.

In some embodiments, the self-amplifying RNA molecule may have two open reading frames. The first (5) open reading frame can encode a replicase; the second (3) open reading frame can encode a polypeptide comprising an antigen of interest. In some embodiments the RNA may have additional (e.g., downstream) open reading frames, e.g., to encode further antigens or to encode accessory polypeptides.

In some embodiments, the second RNA or the saRNA molecule further includes (1) an alphavirus 5′ replication recognition sequence, and (2) an alphavirus 3′ replication recognition sequence. In some embodiments, the 5′ sequence of the self-amplifying RNA molecule is selected to ensure compatibility with the encoded replicase.

Optionally, self-amplifying RNA molecules described herein may also be designed to induce production of infectious viral particles that are attenuated or virulent, or to produce viral particles that are capable of a single round of subsequent infection.

In some embodiments, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of semliki forest virus (SFV), sindbis virus (SIN), Venezuelan equine encephalitis virus (VEE), Ross-River virus (RRV), or other viruses belonging to the alphavirus family. In some embodiments, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a sindbis virus, a semliki forest virus, an eastern equine encephalitis virus, a Venezuelan equine encephalitis virus, or mutant variants thereof.

In some embodiments, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-ATCC VR-1251, ATCC VR-622, ATCC VR-1252), Whataroa (ATCC VR-926), and Y-62-33 (ATCC VR-375). In some aspects, one or more of the alphaviruses in the list may be excluded.

In some embodiments, the self-amplifying RNA molecules described herein are larger than other types of RNA (e.g., saRNA). Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some embodiments, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences. In some embodiments, the saRNA molecule may encode one polypeptide of interest or more, such as an antigen or more than one antigen, e.g., two, three, four, five, six, seven, eight, nine, ten, or more polypeptides. Alternatively, or in addition, one saRNA molecule may also encode more than one polypeptide of interest or more, such as an antigen, e.g., a bicistronic, or tricistronic RNA molecule that encodes different or identical antigens.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first RNA molecule to a RNA molecule at the 5′-end or the 3′-end, but also includes insertion of the whole first RNA molecule into any two nucleotides in the second RNA molecule. The first second RNA molecule can be linked to a second RNA molecule by a phosphodiester bond or a linker. The linker can be, e.g., a polynucleotide.

In some embodiments, the self-amplifying RNA described herein may encode one or more polypeptide antigens that include a range of epitopes. In some embodiments, the self-amplifying RNA described herein may encode epitopes capable of eliciting either a helper T-cell response or a cytotoxic T-cell response or both.

In some embodiments, the saRNA molecule is purified, e.g., such as by filtration that may occur via, e.g., ultrafiltration, diafiltration, or, e.g., tangential flow ultrafiltration/diafiltration.

Some embodiments of the disclosure are directed to a composition comprising a self-amplifying RNA molecule comprising a 5′ Cap, a 5′ untranslated region, a coding region comprising a sequence encoding an RNA-dependent RNA polymerase (also referred to as a “replicase”), a subgenomic promoter, such as one derived from an alphavirus, an open reading frame encoding a gene of interest (e.g., an antigen derived from influenza virus), a 3′ untranslated region, and a 3′ poly A sequence. In some embodiments, at least 5% of a total population of a particular nucleotide in the saRNA molecule has been replaced with one or more modified or unnatural nucleotides.

In some embodiments, the saRNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some embodiments, the RNA may include a 5′ cap comprising a 7′-methylguanosine, and the first 1, 2 or 3 5′ ribonucleotides may be methylated at the 2′ position of the ribose.

The efficacy of the product is dependent on expression of the delivered saRNA, which requires a sufficiently intact RNA molecule. RNA integrity is a measure of RNA quality that quantitates intact RNA. The method is also capable of detecting potential degradation products. RNA integrity is preferably determined by capillary gel electrophoresis. The initial specification is set to ensure sufficient RNA integrity in drug product preparations. In some embodiments, the RNA polynucleotide has an integrity of at least about 80%, 85%, 90%, 92%, 94%, 95%, 96%, 97%, 98%, or 99%. In some embodiments, the RNA polynucleotide has an integrity of or greater than about 95%. In some embodiments, the RNA polynucleotide has an integrity of or greater than about 98%. In some embodiments, the RNA polynucleotide has an integrity of or greater than about 99%.

In preferred embodiments, the saRNA polynucleotide has a clinical grade purity. In some embodiments, the purity of the RNA polynucleotide is between about 60% and about 100%. In some embodiments, the purity of the RNA polynucleotide is between about 80% and 99%. In some embodiments, the purity of the RNA polynucleotide is between about 90% and about 99%. In some embodiments, wherein the purified mRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (HPLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor.

In some embodiments, the purified saRNA polynucleotide comprises 5% or less, 4% or less, 3% or less, 2% or less, 1% or less or is substantially free of protein contaminants as determined by capillary electrophoresis. In some embodiments, the purified RNA polynucleotide comprises less than 5%, less than 4%, less than 3%, less than 2%, less than 1%, or is substantially free of salt contaminants determined by high performance liquid chromatography (HPLC). In some embodiments, the purified RNA polynucleotide comprises 5% or less, 4% or less, 3% or less, 2% or less, 1% or less or is substantially free of short abortive transcript contaminants determined by known methods, such as, e.g., high performance liquid chromatography (HPLC). In some embodiments, the purified RNA polynucleotide has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or greater as determined by a known method, such as, e.g., capillary electrophoresis.

### B. Modified Nucleobases

Modified nucleobases which may be incorporated into modified nucleosides and nucleotides and be present in the RNA molecules include, for example, m5C (5-methylcytidine), m5U (5-methyluridine), m6A (N6-methyladenosine), s2U (2-thiouridine), Um (2′-O-methyluridine), mlA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′lm (1,2′-O-dimethylinosine); m3C (3-methylcytidine); Cm (2T-O-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-O-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mlGm (1,2′-0-dimethylguanosine); m′Am (1,2-0-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. In some aspects, one or more of the modified nucleosides in the list may be excluded.

Additional exemplary modified nucleotides include any one of N-1-methylpseudouridine; pseudouridine, N6-methyladenosine, 5-methylcytidine, and 5-methyluridine. In some embodiments, the modified nucleotide is N-1-methylpseudouridine.

In some embodiments, the RNA molecule may include phosphoramidate, phosphorothioate, and/or methylphosphonate linkages.

In some embodiments, the RNA molecule includes a modified nucleotide selected from any one of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, dihydropseudouridine, 5-methoxyuridine, and 2′-O-methyl uridine. In some embodiments, the modified or unnatural nucleotides are selected from the group consisting of 5-methyluridine, N1-methylpseudouridine, 5-methoxyuridine, and 5-methylcytosine.

In some embodiments, at least 10% of a total population of a particular nucleotide in the saRNA molecule has been replaced with one or more modified or unnatural nucleotides. In some embodiments, at least 25% of a total population of a particular nucleotide in the molecule has been replaced with one or more modified or unnatural nucleotides. In some embodiments, at least 50% of a total population of a particular nucleotide in the molecule has been replaced with one or more modified or unnatural nucleotides. In some embodiments, at least 75% of a total population of a particular nucleotide in the molecule has been replaced with one or more modified or unnatural nucleotides. In some embodiments, essentially all of the particular nucleotide population in the molecule has been replaced with one or more modified or unnatural nucleotides.

In some embodiments, at least a portion, or all of a total population of a particular nucleotide in the saRNA molecule has been replaced with two modified or unnatural nucleotides. In some embodiments, the two modified or unnatural nucleotides are provided in a ratio equal to any one of, at least any one of, at most any one of, or between any two of 1:99 to 99:1, including 1:99; 2:98; 3:97; 4:96; 5:95; 6:94; 7:93; 8:92; 9:91; 10:90; 11:89; 12:88; 13:87; 14:86; 15:85; 16:84; 17:83; 18:82, 19:81; 20:80; 21:79; 22:78; 23:77; 24:76; 25:75; 26:74; 27:73; 28:72; 29:71; 31:69; 32:68; 33:67; 34:66; 35:65; 36:64; 37:63; 38:62; 39:61; 40:60; 41:59; 42:58; 43:57; 44:56; 45:55; 46:54; 47:53; 48:52; 49:51; 50:50; 51:49; 52:48; 53:47; 54:46; 55:45; 56:44; 57:43; 58:42; 59:41; 60:40; 61:39; 62:38; 63:37; 64:36; 65:35; 66:34; 67:33; 68:32; 69:31; 70:30; 71:29; 72:28; 73:27; 74:26; 75:25; 76:24; 77:23; 78:22; 79:21; 80:20; 81:19; 82:18; 83:17; 84:16; 85:15; 86:14; 87:13; 88:12; 89:11; 90:10; 91:9; 92:8; 93:7; 94:6; 95:5; 96:4; 97:3; 98:2; and 99:1, or any range derivable therein.

In some embodiments, at least 10% of a total population of a first particular nucleotide in a saRNA molecule as disclosed herein has been replaced with one or more modified or unnatural nucleotides, and at least 10% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, essentially all of a total population of a first particular nucleotide in the molecule has been replaced with one or more modified or unnatural nucleotides, and essentially all of a total population of a second particular nucleotide in the molecule has been replaced with one or more modified or unnatural nucleotides.

In some embodiments, at least 25% of a total population of uridine nucleotides in the saRNA molecule has been replaced with N1-methylpseudouridine. In some embodiments, at least 50% of a total population of uridine nucleotides in the molecule has been replaced with N1-methylpseudouridine. In some embodiments, at least 75% of a total population of uridine nucleotides in the molecule has been replaced with N1-methylpseudouridine. In some embodiments, essentially all uridine nucleotides in the molecule have been replaced with N1-methylpseudouridine. In some embodiments, at least 50% of a total population of uridine nucleotides in the molecule has been replaced with 5-methoxyuridine. In some embodiments, essentially all uridine nucleotides in the molecule have been replaced with 5-methoxyuridine. In some embodiments, at least 50% of a total population of uridine nucleotides in the molecule has been replaced with 5-methyluridine. In some embodiments, essentially all uridine nucleotides in the molecule have been replaced with 5-methyluridine. In some embodiments, at least 50% of a total population of cytosine nucleotides in the molecule has been replaced with 5-methylcytosine. In some embodiments, essentially all cytosine nucleotides in the molecule have been replaced with 5-methylcytosine. In some embodiments, at least 50% of a total population of uridine nucleotides in the molecule has been replaced with 2-thiouridine. In some embodiments, essentially all uridine nucleotides in the molecule have been replaced with 2-thiouridine.

In some embodiments, at least 50% of a total population of uridine nucleotides in the molecule has been replaced with N1-methylpseudouridine and essentially all cytosine nucleotides in the molecule have been replaced with 5-methylcytosine. In some embodiments, at least 50% of a total population of uridine nucleotides in the molecule has been replaced with 5-methoxyuridine and essentially all cytosine nucleotides in the molecule have been replaced with 5-methylcytosine. In some embodiments, at least 50% of a total population of uridine nucleotides in the molecule has been replaced with 5-methyluridine and essentially all cytosine nucleotides in the molecule have been replaced with 5-methylcytosine.

In some embodiments, essentially all uridine nucleotides in the molecule have been replaced with about 50% 5-methoxyuridine and about 50% N1-methylpseudouridine. In some embodiments, essentially all uridine nucleotides in the molecule have been replaced with about 75% 5-methoxyuridine and about 25% N1-methylpseudouridine. In some embodiments, essentially all uridine nucleotides in the molecule have been replaced with about 25% 5-methoxyuridine and about 75% N1-methylpseudouridine.

### C. UTRs

The 5′ untranslated regions (UTR) is a regulatory region of DNA situated at the 5′ end of a protein coding sequence that is transcribed into mRNA but not translated into protein. 5′ UTRs may contain various regulatory elements, e.g., 5′ cap structure, stem-loop structure, and an internal ribosome entry site (IRES), which may play a role in the control of translation initiation. The 3′ UTR, situated downstream of a protein coding sequence, may be involved in regulatory processes including transcript cleavage, stability and polyadenylation, translation, and mRNA localization. In some embodiments, the UTR is derived from an mRNA that is naturally abundant in a specific tissue (e.g., lymphoid tissue), to which the mRNA expression is targeted. In some embodiments, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). According, the UTR sequence may prolong protein synthesis in a tissue-specific manner. In some embodiments, the 5′ UTR and the 3′ UTR sequences are computationally derived. In some embodiments, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some embodiments, the 5′ UTR and the 3′ UTR are derived from an alphavirus. In some embodiments, the 5′ UTR and the 3′ UTR are from a wild-type alphavirus. Examples of alphaviruses are described below.

In some embodiments, the first RNA molecule includes a 5′ UTR and the 3′ UTR derived from a naturally abundant mRNA in a tissue. In some embodiments, the first RNA molecule includes a 5′ UTR and the 3′ UTR derived from an alphavirus. In some embodiments, the second RNA or the saRNA molecule includes a 5′ UTR and the 3′ UTR derived from an alphavirus. In some embodiments, the second RNA or the saRNA molecule includes a 5′ UTR and the 3′ UTR from a wild-type alphavirus. In some embodiments, the RNA molecule includes a 5′ cap.

### D. Open Reading Frame (ORF)

The 5′ and 3′ UTRs may be operably linked to an ORF, which may be a sequence of codons that is capable of being translated into a polypeptide of interest. As stated above, the RNA molecule may include one (monocistronic), two (bicistronic) or more (multicistronic) open reading frames (ORFs).

In some embodiments, the ORF encodes a non-structural viral gene. In some embodiments, the ORF further includes one or more subgenomic promoters. In some embodiments, the RNA molecule includes a subgenomic promoter operably linked to the ORF. In some embodiments, the subgenomic promoter comprises a cis-acting regulatory element. In some embodiments, the cis-acting regulatory element is immediately downstream (5′-3′) of B 2. In some embodiments, the cis-acting regulatory element is immediately downstream (5′-3′) of a guanine that is immediately downstream of B 2. In some embodiments, the cis-acting regulatory element is an AU-rich element. In some embodiments, the AU-rich element is au, auaaaagau, auaaaaagau, auag, auauauauau, auauauau, auauauauauau, augaugaugau, augau, auaaaagaua, or auaaaagaug. In some embodiments, the second RNA or the saRNA molecule may include (i) an ORF encoding a replicase which may transcribe RNA from the second RNA or the saRNA molecule and (ii) an ORF encoding at least one an antigen or polypeptide of interest. The polymerase may be an alphavirus replicase e.g., including any one of the non-structural alphavirus proteins nsP1, nsP2, nsP3 and nsP4, or a combination thereof. In some embodiments, the RNA molecule includes alphavirus nonstructural protein nsP1. In some embodiments, the RNA molecule includes alphavirus nonstructural protein nsP2. In some embodiments, the RNA molecule includes alphavirus nonstructural protein nsP3. In some embodiments, the RNA molecule includes alphavirus nonstructural protein nsP4. In some embodiments, the RNA molecule includes alphavirus nonstructural proteins nsP1, nsP2, and nsP3. In some embodiments, the RNA molecule includes alphavirus nonstructural proteins nsP1, nsP2, nsP3, and nsP4. In some embodiments, the RNA molecule includes any combination of nsP1, nsP2, nsP3, and nsP4. In some embodiments, the RNA molecule does not include nsP4.

In some embodiments, an open reading frame of an RNA (e.g., saRNA) composition is codon-optimized. In some embodiments, the open reading frame which the influenza polypeptide or fragment thereof is encoded is codon-optimized.

### E. Genes Encoding Antigenic Polypeptides

In some embodiments, the antigenic polypeptide encodes a hemagglutinin protein or immunogenic fragment thereof. In some embodiments, the hemagglutinin protein is H1, H2, H3, H4, H5, H6, H7, H8, H9, H10, H11, H12, H13, H14, H15, H16, H17, H18, or an immunogenic fragment thereof. In some embodiments, the hemagglutinin protein does not comprise a head domain. In some embodiments, the hemagglutinin protein comprises a portion of the head domain. In some embodiments, the hemagglutinin protein does not comprise a cytoplasmic domain. In some embodiments, the hemagglutinin protein comprises a portion of the cytoplasmic domain. In some embodiments, the truncated hemagglutinin protein comprises a portion of the transmembrane domain.

Some embodiments provide influenza vaccines comprising one or more RNA polynucleotides having an open reading frame encoding a hemagglutinin protein and a pharmaceutically acceptable carrier or excipient, formulated within a cationic lipid nanoparticle. In some embodiments, the hemagglutinin protein is selected from H1, H7 and H10. In some embodiments, the RNA polynucleotide further encodes neuraminidase (NA) protein. In some embodiments, the hemagglutinin protein is derived from a strain of Influenza A virus or Influenza B virus or combinations thereof. In some embodiments, the Influenza virus is selected from H1N1, H3N2, H7N9, and H10N8.

In some embodiments, the virus is a strain of Influenza A or Influenza B or combinations thereof. In some embodiments, the strain of Influenza A or Influenza B is associated with birds, pigs, horses, dogs, humans, or non-human primates. In some embodiments, the antigenic polypeptide encodes a hemagglutinin protein or fragment thereof. In some embodiments, the hemagglutinin protein is H7 or H10 or a fragment thereof. In some embodiments, the hemagglutinin protein comprises a portion of the head domain (HA1). In some embodiments, the hemagglutinin protein comprises a portion of the cytoplasmic domain. In some embodiments, the truncated hemagglutinin protein. In some embodiments, the protein is a truncated hemagglutinin protein comprises a portion of the transmembrane domain. In some embodiments, the virus is selected from the group consisting of H7N9 and H10N8. Protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length.

In some embodiments, the at least one antigenic polypeptide is one of the defined antigenic subdomains of HA, termed HA1, HA2, or a combination of HA1 and HA2, and at least one antigenic polypeptide selected from neuraminidase (NA), nucleoprotein (NP), matrix protein 1 (M1), matrix protein 2 (M2), non-structural protein 1 (NS1) and non-structural protein 2 (NS2).

In some embodiments, the at least one antigenic polypeptide is HA or derivatives thereof comprising antigenic sequences from HA1 and/or HA2, and at least one antigenic polypeptide selected from HA, NA, NP, M1, M2, NS1 and NS2.

In some embodiments, the at least one antigenic polypeptide is HA or derivatives thereof comprising antigenic sequences from HA1 and/or HA2 and at least two antigenic polypeptides selected from HA, NA, NP, M1, M2, NS1 and NS2.

In some embodiments, a saRNA composition comprises at least one RNA (e.g., saRNA) polynucleotide having an open reading frame encoding an influenza virus protein, or an immunogenic fragment thereof.

In some embodiments, a saRNA composition comprises at least one RNA (e.g., saRNA) polynucleotide having an open reading frame encoding multiple influenza virus proteins, or immunogenic fragments thereof.

In some embodiments, a saRNA composition comprises at least one RNA (e.g., saRNA) polynucleotide having an open reading frame encoding a HA protein, or an immunogenic fragment thereof (e.g., at least one HA1, HA2, or a combination of both).

In some embodiments, a saRNA composition comprises at least one RNA (e.g., saRNA) polynucleotide having an open reading frame encoding a HA protein, or an immunogenic fragment thereof (e.g., at least one HA1, HA2, or a combination of both, of any one of or a combination of any or all of H1, H2, H3, H4, H5, H6, H7, H8, H9, H10, H11, H12, H13, H14, H15, H16, H17, and/or H18) and at least one other RNA (e.g., saRNA) polynucleotide having an open reading frame encoding a protein selected from a HA protein, NP protein, a NA protein, a M1 protein, a M2 protein, a NS1 protein and a NS2 protein obtained from influenza virus.

In some embodiments, a saRNA composition comprises at least one RNA (e.g., saRNA) polynucleotide having an open reading frame encoding a HA protein, or an immunogenic fragment thereof (e.g., at least one any one of or a combination of any or all of H1, H2, H3, H4, H5, H6, H7, H8, H9, H10, H11, H12, H13, H14, H15, H16, H17, and/or H18) and at least two other RNAs (e.g., saRNAs) polynucleotides having two open reading frames encoding two proteins selected from a HA protein, NP protein, a NA protein, a M1 protein, a M2 protein, a NS1 protein and a NS2 protein obtained from influenza virus.

In some embodiments, a saRNA composition comprises at least one RNA (e.g., saRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least three other RNAs (e.g., saRNAs) polynucleotides having three open reading frames encoding three proteins selected from a HA protein, NP protein, a NA protein, a M protein, a M2 protein, a NS1 protein and a NS2 protein obtained from influenza virus.

In some embodiments, a saRNA composition comprises at least one RNA (e.g., saRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least four other RNAs (e.g., saRNAs) polynucleotides having four open reading frames encoding four proteins selected from a HA protein, NP protein, a NA protein, a M1 protein, a M2 protein, a NS1 protein and a NS2 protein obtained from influenza virus.

In some embodiments, a saRNA composition comprises at least one RNA (e.g., saRNA) polynucleotide having an open reading frame encoding a HA protein, or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18) and at least five other RNAs (e.g., saRNAs) polynucleotides having five open reading frames encoding five proteins selected from a HA protein, NP protein, a NA protein, a M1 protein, a M2 protein, a NS1 protein and a NS2 protein obtained from influenza virus.

In some embodiments, a saRNA composition comprises at least one RNA (e.g., saRNA) polynucleotide having an open reading frame encoding a HA protein or an immunogenic fragment thereof (e.g., at least one of any one of or a combination of any or all of H1, H2, H3, H4, H5, H6, H7, H8, H9, H10, H11, H12, H13, H14, H15, H16, H17, and/or H18), a HA protein, NP protein or an immunogenic fragment thereof, a NA protein or an immunogenic fragment thereof, a M1 protein or an immunogenic fragment thereof, a M2 protein or an immunogenic fragment thereof, a NS1 protein or an immunogenic fragment thereof and a NS2 protein or an immunogenic fragment thereof obtained from influenza virus.

In some embodiments, an influenza RNA composition includes an saRNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the influenza antigen. Antigenic fusion proteins, in some embodiments, retain the functional property from each original protein.

### F. 5′ Cap

In some embodiments, the saRNA molecule described herein includes a 5′ cap. In some embodiments, the 5′-cap moiety is a natural 5′-cap.

A “natural 5′-cap” is defined as a cap that includes 7-methylguanosine connected to the 5′ end of an mRNA molecule through a 5′ to 5′ triphosphate linkage. In some embodiments, the 5′-cap moiety is a 5′-cap analog. In some embodiments, the 5′ end of the RNA is capped with a modified ribonucleotide with the structure m7G (5′) ppp (5′) N (cap 0 structure) or a derivative thereof, which may be incorporated during RNA synthesis (e.g., co-transcriptional capping) or may be enzymatically engineered after RNA transcription (e.g., post-transcriptional capping), wherein “N” is any ribonucleotide. In some embodiments, the 5′ end of the RNA molecule is capped with a modified ribonucleotide via an enzymatic reaction after RNA transcription. In some embodiments, capping is performed after purification, e.g., tangential flow filtration, of the RNA molecule. An exemplary enzymatic reaction for capping may include use of Vaccinia Virus Capping Enzyme (VCE) that includes mRNA triphosphatase, guanylyl-transferase, and guanine-7-methytransferase, which catalyzes the construction of N7-monomethylated cap 0 structures. Cap 0 structure can help maintaining the stability and translational efficacy of the RNA molecule. The 5′ cap of the RNA molecule may be further modified by a 2-O-Methyltransferase which results in the generation of a cap 1 structure (m7Gppp [m2′-0] N), which may further increase translation efficacy. In some embodiments, the RNA molecule may be enzymatically capped at the 5′ end using Vaccinia guanylyltransferase, guanosine triphosphate, and S-adenosyl-L-methionine to yield cap 0 structure. An inverted 7-methylguanosine cap is added via a 5′ to 5′ triphosphate bridge. Alternatively, use of a 2′O-methyltransferase with Vaccinia guanylyltransferase yields the cap 1 structure where in addition to the cap 0 structure, the 2′0H group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0) and 7mG(5′)ppp(5′)N1mpNp (cap 1). Cap 0 is a N7-methyl guanosine connected to the 5′ nucleotide through a 5′ to 5′ triphosphate linkage, typically referred to as m7G cap or m7Gppp. In the cell, the cap 0 structure can help provide for efficient translation of the mRNA that carries the cap. An additional methylation on the 2′O position of the initiating nucleotide generates Cap 1, or refers to as m7GpppNm-, wherein Nm denotes any nucleotide with a 2′O methylation. In some embodiments, the 5′ terminal cap includes a cap analog, for example, a 5′ terminal cap may include a guanine analog. Exemplary guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine. In some embodiments, the capping region may include a single cap or a series of nucleotides forming the cap. In this embodiment the capping region may be equal to any one of, at least any one of, at most any one of, or between any two of 1, 2, 3, 4, 5, 6, 7, 8, 9, 10, or at least 2, or 10 or fewer nucleotides in length. In some embodiments, the cap is absent. In some embodiments, the first and second operational regions may be equal to any one of, at least any one of, at most any one of, or between any two of 3 to 40, e.g., 5-30, 10-20, 15, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or at least 4, or 30 or fewer nucleotides in length and may comprise, in addition to a Start and/or Stop codon, one or more signal and/or restriction sequences.

In some embodiments, the 5′ Cap is represented by Formula I:

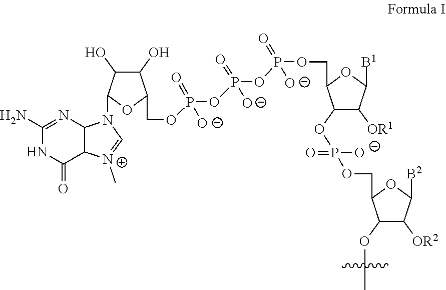

where R¹ and R² are each independently H or Me, and Bland B² are each independently guanine, adenine, or uracil. In some embodiments, B¹ and B² are naturally-occurring bases. In some embodiments, R¹ is methyl and R² is hydrogen. In some embodiments, B¹ is guanine. In some embodiments, B¹ is adenine. In some embodiments, B² is adenine. In some embodiments, B² is uracil. In some embodiments, B² is uracil and at least 5% of a total population of uracil nucleotides in the molecule that are downstream of B² have been replaced with one or more modified or unnatural nucleotides.

In some embodiments, the nucleotide immediately downstream (5′ to 3′ direction) of the Cap comprises guanine. In some embodiments, B¹ is adenine and B² is uracil. In some embodiments, B¹ is adenine, B² is uracil, R¹ is methyl, and R² is hydrogen. In some instances, the saRNA does not comprise a 5′ Cap. In some instances, the 5′ Cap is not represented by Formula I. In some embodiments, the nucleotide immediately downstream (5′ to 3′) of the 5′ Cap comprises guanine, B¹ is adenine, B² is uracil, R¹ is methyl, and R² is hydrogen; this embodiment corresponds to CleanCap AU, and the inclusion of B 2=uracil, while optionally substituting uracil nucleotides downstream of B 2, has been shown to improve saRNA functionality in some embodiments. In some embodiments, the RNA molecule further comprises: (1) an alphavirus 5′ replication recognition sequence, and (2) an alphavirus 3′ replication recognition sequence. In some embodiments, the RNA molecule encodes at least one antigen. In some embodiments, the RNA molecule comprises at least 7000 nucleotides. In some embodiments, the RNA molecule comprises at least 8000 nucleotides. In some embodiments, at least 80% of the total RNA molecules are full length. In some embodiments, the alphavirus is Venezuelan equine encephalitis virus. In some embodiments, the alphavirus is Semliki Forest virus.

In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with N1-methylpseudouridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methoxyuridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methyluridine, and essentially all cytosine nucleotides in the molecule have been replaced with 5-methylcytosine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 50% 5-methoxyuridine and about 50% N1-methylpseudouridine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 75% 5-methoxyuridine and about 25% N1-methylpseudouridine. In some embodiments, the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 25% 5-methoxyuridine and about 75% N1-methylpseudouridine.

In some embodiments, a 5′ terminal cap is 7mG(5′)ppp(5′)NImpNp. In some preferred embodiments, the 5′ cap comprises:

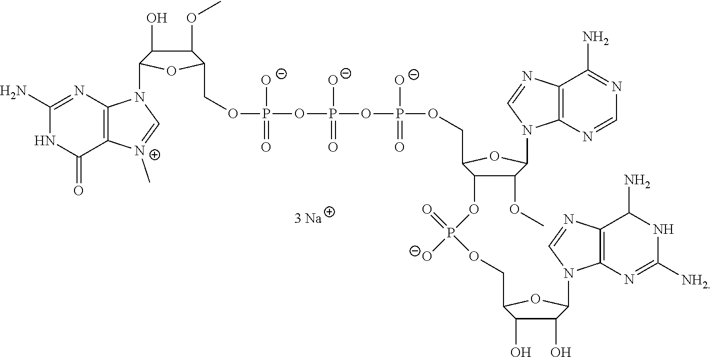 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c3N=C(N)NC4N)C(O)C2O)OC(n2c[n+](C)c3c(=O)[nH]c(N)nc32)C1O]

In some embodiments, the 5′ cap comprises CLEANCAP® Reagent AG (3′ OMe) for co-transcriptional capping of mRNA, m7(3′OMeG)(5′)ppp(5′)(2′OMeA)pG,

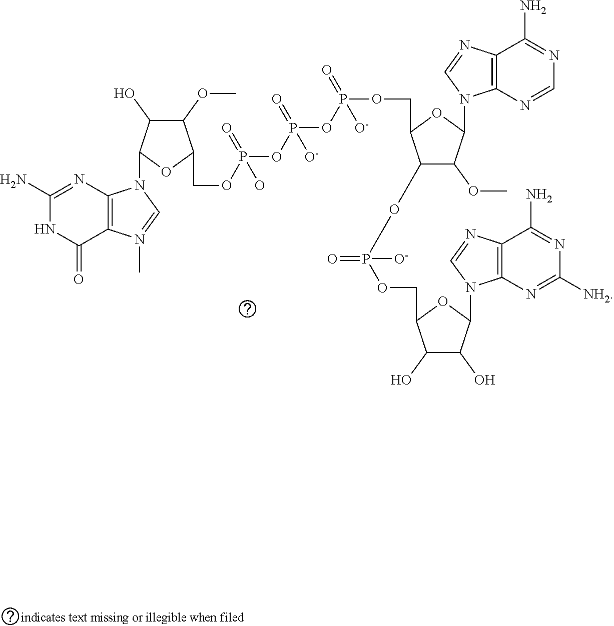

In alternative embodiments, the 5′ cap comprises CLEANCAP® AU for Self-Amplifying mRNA, CLEANCAP® Reagent AU for co-transcriptional capping of mRNA, m7G(5′)ppp(5′)(2′OMeA)pU,

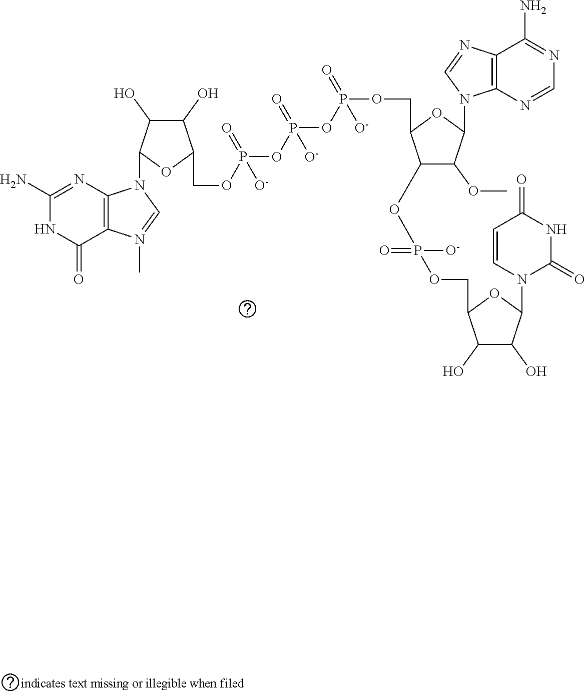

### G. Poly-A Tail

As used herein, “poly A tail” refers to a stretch of consecutive adenine residues, which may be attached to the 3′ end of the RNA molecule. The poly-A tail may increase the half-life of the RNA molecule. Poly-A tails may play key regulatory roles in enhancing translation efficiency and regulating the efficiency of mRNA quality control and degradation. Short sequences or hyperpolyadenylation may signal for RNA degradation. Exemplary designs include a poly-A tails of about 40 adenine residues to about 80 adenine residues. In some embodiments, the RNA molecule further includes an endonuclease recognition site sequence immediately downstream of the poly A tail sequence. In some embodiments, such as for the second RNA or the saRNA molecule, the RNA molecule further includes a poly-A polymerase recognition sequence (e.g., AAUAAA) near its 3′ end. A “full length” RNA molecule is one that includes a 5′-cap and a poly A tail.

In some embodiments, the poly A tail includes 5-400 nucleotides in length. The poly A tail nucleotide length may be equal to any one of, at least any one of, at most any one of, or between any two of 5, 6, 7, 8, 9, 10, 15, 20, 25. 30, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400. In some embodiments, the RNA molecule includes a poly A tail that includes about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some embodiments, the RNA molecule includes a poly A tail includes a sequence of greater than 30 adenosine nucleotides (“As”). In some embodiments, the RNA molecule includes a poly A tail that includes about 40 As. In some embodiments, the RNA molecule includes a poly A tail that includes about 80 As. As used herein, the term “about” refers to a deviation of ±10% of the value(s) to which it is attached. In some embodiments, the 3′ poly-A tail has a stretch of at least 10 consecutive adenosine residues and at most 300 consecutive adenosine residues. In some embodiments, the RNA molecule includes at least 20 consecutive adenosine residues and at most 40 consecutive adenosine residues. In some embodiments, the RNA molecule includes about 40 consecutive adenosine residues. In some embodiments, the RNA molecule includes about 80 consecutive adenosine residues.

### H. Composition

In some instances, the compositions described herein include at least one saRNA as described herein. Some embodiments of the present disclosure provide influenza virus (influenza) vaccines (or compositions or immunogenic compositions) that include at least one saRNA polynucleotide having an open reading frame encoding at least one influenza antigenic polypeptide or an immunogenic fragment thereof (e.g., an immunogenic fragment capable of inducing an immune response to influenza).

In some embodiments, equal to any one of, at least any one of, at most any one of, or between any two of 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules (capped and uncapped) in the composition are capped.

In some embodiments, equal to any one of, at least any one of, at most any one of, or between any two of 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules in the composition are full length RNA transcripts. Purity may be determined as described herein, e.g., via reverse phase HPLC or Bioanalyzer chip-based electrophoresis and measure by, e.g., peak area of full-length RNA molecule relative to total peak. In some embodiments, a fragment analyzer (FA) may be used to quantify and purify the RNA. The fragment analyzer automates capillary electrophoresis and HPLC.

In some embodiments, the composition is substantially free of one or more impurities or contaminants including the linear DNA template and/or reverse complement transcription products and, for instance, includes RNA molecules that are equal to any one of, at least any one of, at most any one of, or between any two of 90%, 91%, 92%, 93%, 94%, 95%, 96%, 97%, 98%, or 99% pure; at least 98% pure, or at least 99% pure.

In some embodiments, the composition comprises an amount of the first RNA molecule that is greater than the amount of the second RNA molecule. In some embodiments, the composition comprises an amount of the first RNA molecule that is at least about 1 to 2 times greater than the amount of the second RNA molecule. In some embodiments, the composition comprises an amount of the first RNA molecule that is at least about 1 to 100 times greater than the amount of the second RNA molecule.

In some embodiments, the composition further includes a pharmaceutically acceptable carrier. In some embodiments, the composition further includes a pharmaceutically acceptable vehicle.

In some embodiments, the composition further includes a lipid-based delivery system, which delivers an RNA molecule to the interior of a cell, where it can then replicate and/or express the encoded polypeptide of interest. The delivery system may have adjuvant effects which enhance the immunogenicity of an encoded antigen. In some embodiments, the composition further includes neutral lipids, cationic lipids, cholesterol, and polyethylene glycol (PEG), and forms nanoparticles that encompass the RNA molecules. In some embodiments, the composition further includes any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion. In some embodiments, the RNA molecule is encapsulated in, bound to or adsorbed on any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion, or a combination thereof.

In some instances, the compositions described herein include at least two RNA molecules: a first saRNA molecule and a second RNA molecule as described herein. To protect against more than one strain of influenza, a combination vaccine composition may be administered that includes RNA (e.g., saRNA) encoding at least one antigenic polypeptide protein (or antigenic portion thereof) of a first influenza virus or organism and further includes a second RNA molecule encoding at least one antigenic polypeptide protein (or antigenic portion thereof) of a second influenza virus or organism. RNA (e.g., saRNA) can be co-formulated, for example, in a single lipid nanoparticle (LNP) or can be formulated in separate LNPs for co-administration.

In some embodiments, the second RNA molecule includes any one of a 5′ cap, a 5′ UTR, an open reading frame, a 3′ UTR, and a poly A sequence, or any combination thereof. In some embodiments, the second RNA molecule includes a 5′ cap moiety. In some embodiments, the second RNA molecule includes a 5′ UTR and a 3′UTR. In some embodiments, the second RNA molecule includes a 5′UTR, an open reading frame, a 3′UTR, and does not further include a 5′ cap. In some embodiments, the second RNA molecule includes a 5′ cap moiety, 5′ UTR, coding region, 3′ UTR, and a 3′ poly A sequence. In some embodiments, the second RNA molecule includes a 5′ cap moiety, 5′ UTR, noncoding region, 3′ UTR, and a 3′ poly A sequence. In some embodiments, the second RNA molecule includes a noncoding region and does not further comprise any one of a 5′ cap moiety, 5′ UTR, 3′ UTR, and a 3′ poly A sequence. In some embodiments, the second RNA molecule includes a 5′ cap moiety, a 5′ untranslated region (5′ UTR), a modified nucleotide, an open reading frame, a 3′ untranslated region (3′ UTR), and a 3′ poly A sequence.

Some aspects of the disclosure are directed to a composition comprising (i) first RNA molecule encoding a gene of interest derived from influenza; and (ii) a second RNA molecule comprising a modified or unnatural nucleotide. In some instances, the first RNA molecule is any one of the saRNA molecules described herein. In some instances, the first RNA molecule comprises a 5′ Cap, a 5′ untranslated region, a coding region for a nonstructural protein comprising a RNA replicase, a subgenomic promoter, an open reading frame encoding a gene of interest, a 3′ untranslated region, and a 3′ poly A sequence. In some instances, at least 5% of a total population of a particular nucleotide in the first RNA molecule has been replaced with one or more modified or unnatural nucleotides. In some instances, the saRNA molecule comprises natural, unmodified nucleotides and does not include a modified or unnatural nucleotide. In some instances, the 5′ Cap is represented by Formula I, where R1 and R2 are each independently H or Me, B¹ and B² are each independently guanine, adenine, or uracil, a 5′ untranslated region, a coding region for a nonstructural protein derived from an alphavirus, a subgenomic promoter, such as one derived from an alphavirus, an open reading frame encoding a gene of interest, a 3′ untranslated region, and a 3′ poly A sequence. In some embodiments, B¹ and B² are naturally-occurring bases. In some embodiments, R1 is methyl and R2 is hydrogen. In some embodiments, B¹ is guanine. In some embodiments, B¹ is adenine. In some embodiments, B² is adenine. In some embodiments, B² is uracil. In some embodiments, the nucleotide immediately downstream (5′ to 3′ direction) of the 5′ Cap comprises guanine.

In some embodiments, B¹ is adenine and B² is uracil. In some embodiments, B¹ is adenine, B² is uracil, R¹ is methyl, and R² is hydrogen. In some embodiments, the nucleotide immediately downstream (5′ to 3′) of the 5′ Cap comprises guanine, B¹ is adenine, B² is uracil, R¹ is methyl, and R² is hydrogen; this embodiment corresponds to CLEANCAP AU (Trilink), and the inclusion of B 2=uracil, while optionally substituting uracil nucleotides downstream of B², which has been shown to provide increased saRNA functionality in some embodiments.

In some embodiments, at least 10% of a total population of a particular nucleotide in the first or second RNA molecule has been replaced with one or more modified or unnatural nucleotides. In some embodiments, at least 25% of a total population of a particular nucleotide in the first or second RNA molecule has been replaced with one or more modified or unnatural nucleotides. In some embodiments, at least 50% of a total population of a particular nucleotide in the first or second RNA molecule has been replaced with one or more modified or unnatural nucleotides. In some embodiments, at least 75% of a total population of a particular nucleotide in the first or second RNA molecule has been replaced with one or more modified or unnatural nucleotides. In some embodiments, essentially all of a particular nucleotide population in the first or second RNA molecule has been replaced with one or more modified or unnatural nucleotides. In some embodiments, the one or more modified or unnatural replacement nucleotides comprise two modified or unnatural nucleotides provided in a ratio ranging from 1:99 to 99:1, or any derivable range therein. In some embodiments, at least 10% of a total population of a first particular nucleotide in the first or second RNA molecule has been replaced with one or more modified or unnatural nucleotides, and at least 10% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 25% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 50% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 10% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 25% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 50% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 25% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 50% of a total population of a first particular nucleotide in the first or second RNA molecule has been replaced with one or more modified or unnatural nucleotides, and at least 75% of a total population of a second particular nucleotide in the first or second RNA molecule has been replaced with one or more modified or unnatural nucleotides. In some embodiments, at least 50% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides. In some embodiments, at least 75% of a total population of a first particular nucleotide in the first or second RNA molecule has been replaced with one or more modified or unnatural nucleotides, and essentially all of a total population of a second particular nucleotide in the first or second RNA molecule has been replaced with one or more modified or unnatural nucleotides.

In some embodiments, at least 25% of a total population of uridine nucleotides in the first RNA molecule has been replaced with N1-methylpseudouridine. In some embodiments, at least 50% of a total population of uridine nucleotides in the first RNA molecule has been replaced with N1-methylpseudouridine. In some embodiments, at least 75% of a total population of uridine nucleotides in the first RNA molecule has been replaced with N1-methylpseudouridine. In some embodiments, essentially all uridine nucleotides in the first RNA molecule have been replaced with N1-methylpseudouridine. In some embodiments, at least 50% of a total population of uridine nucleotides in the first RNA molecule has been replaced with 5-methoxyuridine. In some embodiments, essentially all uridine nucleotides in the molecule have been replaced with 5-methoxyuridine. In some embodiments, at least 50% of a total population of uridine nucleotides in the first RNA molecule has been replaced with 5-methyluridine. In some embodiments, essentially all uridine nucleotides in the first RNA molecule have been replaced with 5-methyluridine. In some embodiments, at least 50% of a total population of cytosine nucleotides in the first RNA molecule has been replaced with 5-methylcytosine. In some embodiments, essentially all cytosine nucleotides in the first RNA molecule have been replaced with 5-methylcytosine. In some embodiments, at least 50% of a total population of uridine nucleotides in the first RNA molecule has been replaced with 2-thiouridine. In some embodiments, essentially all uridine nucleotides in the first RNA molecule have been replaced with 2-thiouridine.

In some embodiments, at least 25% of a total population of uridine nucleotides in the second RNA molecule has been replaced with N1-methylpseudouridine. In some embodiments, at least 50% of a total population of uridine nucleotides in the second RNA molecule has been replaced with N1-methylpseudouridine. In some embodiments, at least 75% of a total population of uridine nucleotides in the second RNA molecule has been replaced with N1-methylpseudouridine. In some embodiments, essentially all uridine nucleotides in the second RNA molecule have been replaced with N1-methylpseudouridine. In some embodiments, at least 50% of a total population of uridine nucleotides in the second RNA molecule has been replaced with 5-methoxyuridine. In some embodiments, essentially all uridine nucleotides in the second RNA molecule have been replaced with 5-methoxyuridine. In some embodiments, at least 50% of a total population of uridine nucleotides in the second RNA molecule has been replaced with 5-methyluridine. In some embodiments, essentially all uridine nucleotides in the second RNA molecule have been replaced with 5-methyluridine. In some embodiments, at least 50% of a total population of cytosine nucleotides in the second RNA molecule has been replaced with 5-methylcytosine. In some embodiments, essentially all cytosine nucleotides in the second RNA molecule have been replaced with 5-methylcytosine. In some embodiments, at least 50% of a total population of uridine nucleotides in the second RNA molecule has been replaced with 2-thiouridine. In some embodiments, essentially all uridine nucleotides in the second RNA molecule have been replaced with 2-thiouridine.

In some embodiments, at least 50% of a total population of uridine nucleotides in the second RNA molecule has been replaced with N1-methylpseudouridine and essentially all cytosine nucleotides in the second RNA molecule have been replaced with 5-methylcytosine. In some embodiments, at least 50% of a total population of uridine nucleotides in the second RNA molecule has been replaced with 5-methoxyuridine and essentially all cytosine nucleotides in the second RNA molecule have been replaced with 5-methylcytosine. In some embodiments, at least 50% of a total population of uridine nucleotides in the second RNA molecule has been replaced with 5-methyluridine and essentially all cytosine nucleotides in the second RNA molecule have been replaced with 5-methylcytosine.

In some embodiments, essentially all uridine nucleotides in the second RNA molecule have been replaced with about 50% 5-methoxyuridine and about 50% N1-methylpseudouridine. In some embodiments, essentially all uridine nucleotides in the second RNA molecule have been replaced with about 75% 5-methoxyuridine and about 25% N1-methylpseudouridine. In some embodiments, essentially all uridine nucleotides in the second RNA molecule have been replaced with about 25% 5-methoxyuridine and about 75% N1-methylpseudouridine.

In some embodiments, essentially all uridine nucleotides in the first RNA molecule have been replaced with N1-methylpseudouridine and at least 50% of a total population of uridine nucleotides in the second RNA molecule has been replaced with N1-methylpseudouridine. In some embodiments, essentially all uridine nucleotides in the first RNA molecule have been replaced with N1-methylpseudouridine and essentially all uridine nucleotides in the second RNA molecule have been replaced with N1-methylpseudouridine. In some embodiments, essentially all uridine nucleotides in the first RNA molecule have been replaced with N1-methylpseudouridine and at least 50% of a total population of uridine nucleotides in the second RNA molecule has been replaced with 5-methoxyuridine. In some embodiments, essentially all uridine nucleotides in the first RNA molecule have been replaced with N1-methylpseudouridine, at least 50% of a total population of uridine nucleotides in the second RNA molecule has been replaced with 5-methyluridine, and essentially all cytosine nucleotides in the second RNA molecule have been replaced with 5-methylcytosine. In some embodiments, essentially all uridine nucleotides in the first RNA molecule have been replaced with N1-methylpseudouridine and essentially all uridine nucleotides in the second RNA molecule have been replaced with about 50% 5-methoxyuridine and about 50% N1-methylpseudouridine.

I. Methods of Use

The saRNA compositions may be utilized to treat and/or prevent an influenza virus of various genotypes, strains, and isolates. Some embodiments provide methods of preventing or treating influenza viral infection comprising administering to a subject any of the saRNA compositions described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the saRNA composition. In some embodiments, the saRNA composition is administered to the subject by intradermal, intramuscular injection, subcutaneous injection, intranasal inoculation, or oral administration.

In some embodiments, the RNA (e.g., saRNA) polynucleotides or portions thereof may encode one or more polypeptides or fragments thereof of an influenza strain as an antigen.

Some aspects of the disclosure are directed to a method of inducing an immune response in a subject, comprising administering to the subject in need thereof an effective amount of a composition as disclosed herein. Some aspects of the disclosure are directed to a method of vaccinating a subject, comprising administering to the subject in need thereof an effective amount of a composition as disclosed herein. Some aspects of the disclosure are directed to a method comprising administering to the subject in need thereof an effective amount of a composition as disclosed herein. In some embodiments, a composition as disclosed herein elicits an immune response comprising an antibody response. In some embodiments, a composition as disclosed herein elicits an immune response comprising a T cell response.

Some embodiments of the present disclosure provide methods of inducing an antigen specific immune response in a subject, comprising administering to the subject any of the RNA (e.g., saRNA) composition as provided herein in an amount effective to produce an antigen-specific immune response. In some embodiments, the RNA (e.g., saRNA) composition is an influenza vaccine. In some embodiments, the RNA (e.g., saRNA) composition is a combination vaccine comprising a combination of influenza vaccines (a broad spectrum influenza vaccine). In some embodiments, an antigen-specific immune response comprises a T cell response or a B cell response. In some embodiments, a method of producing an antigen-specific immune response comprises administering to a subject a single dose (no booster dose) of an influenza RNA (e.g., saRNA) composition of the present disclosure. In some embodiments, a method further comprises administering to the subject a second (booster) dose of an influenza RNA (e.g., saRNA) composition. Additional doses of an influenza RNA (e.g., saRNA) composition may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an influenza RNA (e.g., saRNA) composition is administered to a subject by intradermal injection, intramuscular injection, or by intranasal administration. In some embodiments, an influenza RNA (e.g., saRNA) composition is administered to a subject by intramuscular injection.

Some embodiments, of the present disclosure provide methods of inducing an antigen specific immune response in a subject, including administering to a subject an influenza RNA (e.g., saRNA) composition in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza RNA (e.g., saRNA) compositions of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., saRNA) composition of the present disclosure. In some embodiments, the control is an anti-antigenic polypeptide antibody titer produced in a subject who has been administered a live attenuated or inactivated influenza, or wherein the control is an anti-antigenic polypeptide antibody titer produced in a subject who has been administered a recombinant or purified influenza protein vaccine.

In some embodiments, the RNA (e.g., saRNA) composition is formulated in an effective amount to produce an antigen specific immune response in a subject.

In some embodiments, the effective amount is a total dose of 1 μg to 1000 μg, or 1 μg to 100 μg of saRNA. In some embodiments, the effective amount is a total dose of 30 μg. In some embodiments, the effective amount is a dose of 10 μg administered to the subject a total of two times. In some embodiments, the effective amount is a dose of 10 μg administered to the subject a total of two times. In some embodiments, the effective amount is a dose of 15 μg administered to the subject a total of two times. In some embodiments, the effective amount is a dose of 30 μg administered to the subject a total of two times.

In some embodiments, the method includes administering to the subject a saRNA composition described herein at dosage of between 10 μg/kg and 400 μg/kg is administered to the subject. In some embodiments the dosage of the saRNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the saRNA composition is administered to the subject by intradermal or intramuscular injection. In some embodiments, the saRNA composition is administered to the subject on day zero. In some embodiments, a second dose of the saRNA composition is administered to the subject on day twenty-one.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject has been exposed to influenza (e.g., C. trachomatis); the subject is infected with influenza (e.g., C. trachomatis); or subject is at risk of infection by influenza (e.g., C. trachomatis).

In some embodiments, the subject has been exposed to betacoronavirus (e.g., SARS-CoV-2); the subject is infected with betacoronavirus (e.g., SARS-CoV-2); or subject is at risk of infection by betacoronavirus (e.g., SARS-CoV-2).

In some embodiments, the subject has received at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; the subject has received at least two doses of an immunogenic composition against betacoronavirus (e.g., SARS-CoV-2); the subject is receiving at least one dose of an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine; or the subject is being administered an immunogenic composition against betacoronavirus (e.g., SARS-CoV-2), e.g., selected from any one of COMIRNATY®, the Pfizer-BioNTech COVID-19 vaccine, the Moderna mRNA-1273 COVID-19 vaccine, and the Janssen COVID-19 vaccine at risk of infection by betacoronavirus (e.g., SARS-CoV-2) concomitantly, simultaneously, or within 12-48 hours of any one of the immunogenic compositions against influenza disclosed herein.

In some embodiments, the subject is immunocompromised (has an impaired immune system, e.g., has an immune disorder or autoimmune disorder).

Aspects of the disclosure provide saRNA compositions comprising one or more saRNA polynucleotides having an open reading frame encoding a first antigenic polypeptide, wherein the saRNA polynucleotide is present in the formulation for in vivo administration to a host, which confers an antibody titer superior to the criterion for seroprotection for the first antigen (e.g., HA) for an acceptable percentage of human subjects. In some embodiments, the antibody titer produced by the saRNA composition of the disclosure is a neutralizing antibody titer. In some embodiments the neutralizing antibody titer is greater than a protein vaccine. In other embodiments the neutralizing antibody titer produced by the saRNA composition is greater than an adjuvanted protein vaccine. In yet other embodiments the neutralizing antibody titer produced by the saRNA compositionis 1,000-10,000, 1,200-10,000, 1,400-10,000, 1,500-1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

### J. Nucleic Acids

In certain embodiments, nucleic acid sequences can exist in a variety of instances such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein. Nucleic acids that encode an epitope to which antibodies may bind. Nucleic acids encoding fusion proteins that include these polypeptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

In certain embodiments, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some embodiments, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 12. In some embodiments, the polynucleotide comprises a sequence having at least 70%, 75%, 80%, 85%, 90%, 95%, 96%, 97%, 98%, or 99% or higher sequence identity to SEQ ID NO: 13. In some embodiments, the polynucleotide comprises a sequence having at least 70%, 75%, 80%, 85%, 90%, 95%, 96%, 97%, 98%, or 99% or higher sequence identity to SEQ ID NO: 14. In some embodiments, the polynucleotide comprises a sequence having at least 70%, 75%, 80%, 85%, 90%, 95%, 96%, 97%, 98%, or 99% or higher sequence identity to SEQ ID NO: 15. In some embodiments, the polynucleotide comprises a sequence having at least 70%, 75%, 80%, 85%, 90%, 95%, 96%, 97%, 98%, or 99% or higher sequence identity to SEQ ID NO: 16. In some embodiments, the polynucleotide comprises a sequence having at least 70%, 75%, 80%, 85%, 90%, 95%, 96%, 97%, 98%, or 99% or higher sequence identity to SEQ ID NO: 17. In some embodiments, the polynucleotide comprises a sequence having at least 70%, 75%, 80%, 85%, 90%, 95%, 96%, 97%, 98%, or 99% or higher sequence identity to SEQ ID NO: 18. In some embodiments, the polynucleotide comprises a sequence having at least 70%, 75%, 80%, 85%, 90%, 95%, 96%, 97%, 98%, or 99% or higher sequence identity to SEQ ID NO: 19. In some embodiments, the polynucleotide comprises a sequence having at least 70%, 75%, 80%, 85%, 90%, 95%, 96%, 97%, 98%, or 99% or higher sequence identity to SEQ ID NO: 20. In some embodiments, the polynucleotide comprises a sequence having at least 70%, 75%, 80%, 85%, 90%, 95%, 96%, 97%, 98%, or 99% or higher sequence identity to SEQ ID NO: 21. In some embodiments, the polynucleotide comprises a sequence having at least 70%, 75%, 80%, 85%, 90%, 95%, 96%, 97%, 98%, or 99% or higher sequence identity to SEQ ID NO: 22.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 12; a polynucleotide sequence having at least 70%, 75%, 80%, 85%, 90%, 95%, 96%, 97%, 98%, or 99% or higher sequence identity to SEQ ID NO: 13; a polynucleotide sequence having at least 70%, 75%, 80%, 85%, 90%, 95%, 96%, 97%, 98%, or 99% or higher sequence identity to SEQ ID NO: 14; a polynucleotide sequence having at least 70%, 75%, 80%, 85%, 90%, 95%, 96%, 97%, 98%, or 99% or higher sequence identity to SEQ ID NO: 15; a polynucleotide sequence having at least 70%, 75%, 80%, 85%, 90%, 95%, 96%, 97%, 98%, or 99% or higher sequence identity to SEQ ID NO: 16; a polynucleotide sequence having at least 70%, 75%, 80%, 85%, 90%, 95%, 96%, 97%, 98%, or 99% or higher sequence identity to SEQ ID NO: 17; a polynucleotide sequence encoding a polypeptide selected from HA, NA, NP, M1, M2, NS1 and NS2; a polynucleotide sequence having at least 70%, 75%, 80%, 85%, 90%, 95%, 96%, 97%, 98%, or 99% or higher sequence identity to SEQ ID NO: 19; a polynucleotide sequence encoding a polypeptide selected from HA, NA, NP, M1, M2, NS1 and NS2; a polynucleotide sequence having at least 70%, 75%, 80%, 85%, 90%, 95%, 96%, 97%, 98%, or 99% or higher sequence identity to SEQ ID NO: 21; and a poly A tail comprising at least 20 consecutive adenines.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 12; a polynucleotide sequence having at least 70%, 75%, 80%, 85%, 90%, 95%, 96%, 97%, 98%, or 99% or higher sequence identity to SEQ ID NO: 13; a polynucleotide sequence having at least 70%, 75%, 80%, 85%, 90%, 95%, 96%, 97%, 98%, or 99% or higher sequence identity to SEQ ID NO: 14; a polynucleotide sequence having at least 70%, 75%, 80%, 85%, 90%, 95%, 96%, 97%, 98%, or 99% or higher sequence identity to SEQ ID NO: 15; a polynucleotide sequence having at least 70%, 75%, 80%, 85%, 90%, 95%, 96%, 97%, 98%, or 99% or higher sequence identity to SEQ ID NO: 16; a polynucleotide sequence having at least 70%, 75%, 80%, 85%, 90%, 95%, 96%, 97%, 98%, or 99% or higher sequence identity to SEQ ID NO: 17; a polynucleotide sequence having at least 70%, 75%, 80%, 85%, 90%, 95%, 96%, 97%, 98%, or 99% or higher sequence identity to SEQ ID NO: 18; a polynucleotide sequence having at least 70%, 75%, 80%, 85%, 90%, 95%, 96%, 97%, 98%, or 99% or higher sequence identity to SEQ ID NO: 19; a polynucleotide sequence having at least 70%, 75%, 80%, 85%, 90%, 95%, 96%, 97%, 98%, or 99% or higher sequence identity to SEQ ID NO: 20; a polynucleotide sequence having at least 70%, 75%, 80%, 85%, 90%, 95%, 96%, 97%, 98%, or 99% or higher sequence identity to SEQ ID NO: 21; and a poly A tail comprising at least 20 consecutive adenines.

In some embodiments, the polynucleotide comprises a 5′ UTR sequence having SEQ ID NO: 12; a polynucleotide sequence having SEQ ID NO: 13; a polynucleotide sequence having SEQ ID NO: 14; a sequence having SEQ ID NO: 15; a polynucleotide sequence having SEQ ID NO: 16; a polynucleotide sequence having SEQ ID NO: 17; a polynucleotide sequence encoding a polypeptide selected from HA, NA, NP, M1, M2, NS1 and NS2; a polynucleotide sequence having SEQ ID NO: 19; a polynucleotide sequence encoding a polypeptide selected from HA, NA, NP, M1, M2, NS1 and NS2; a polynucleotide sequence having SEQ ID NO: 21; and a poly A tail comprising at least 20 consecutive adenines.

In some embodiments, the polynucleotide comprises a 5′ UTR sequence having SEQ ID NO: 12; a polynucleotide sequence having SEQ ID NO: 13; a polynucleotide sequence having SEQ ID NO: 14; a sequence having SEQ ID NO: 15; a polynucleotide sequence having SEQ ID NO: 16; a polynucleotide sequence having SEQ ID NO: 17; a polynucleotide sequence having at least 70%, 75%, 80%, 85%, 90%, 95%, 96%, 97%, 98%, or 99% or higher sequence identity to SEQ ID NO: 18; a polynucleotide sequence having SEQ ID NO: 19; a polynucleotide sequence having at least 70%, 75%, 80%, 85%, 90%, 95%, 96%, 97%, 98%, or 99% or higher sequence identity to SEQ ID NO: 20; a polynucleotide sequence having SEQ ID NO: 21; and a poly A tail comprising at least 20 consecutive adenines.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 23. In some embodiments, the polynucleotide comprises a sequence having at least 70%, 75%, 80%, 85%, 90%, 95%, 96%, 97%, 98%, or 99% or higher sequence identity to SEQ ID NO: 24. In some embodiments, the polynucleotide comprises a sequence having at least 70%, 75%, 80%, 85%, 90%, 95%, 96%, 97%, 98%, or 99% or higher sequence identity to SEQ ID NO: 25. In some embodiments, the polynucleotide comprises a sequence having at least 70%, 75%, 80%, 85%, 90%, 95%, 96%, 97%, 98%, or 99% or higher sequence identity to SEQ ID NO: 26. In some embodiments, the polynucleotide comprises a sequence having at least 70%, 75%, 80%, 85%, 90%, 95%, 96%, 97%, 98%, or 99% or higher sequence identity to SEQ ID NO: 27. In some embodiments, the polynucleotide comprises a sequence having at least 70%, 75%, 80%, 85%, 90%, 95%, 96%, 97%, 98%, or 99% or higher sequence identity to SEQ ID NO: 28. In some embodiments, the polynucleotide comprises a sequence having at least 70%, 75%, 80%, 85%, 90%, 95%, 96%, 97%, 98%, or 99% or higher sequence identity to SEQ ID NO: 29. In some embodiments, the polynucleotide comprises a sequence having at least 70%, 75%, 80%, 85%, 90%, 95%, 96%, 97%, 98%, or 99% or higher sequence identity to SEQ ID NO: 30. In some embodiments, the polynucleotide comprises a sequence having at least 70%, 75%, 80%, 85%, 90%, 95%, 96%, 97%, 98%, or 99% or higher sequence identity to SEQ ID NO: 31. In some embodiments, the polynucleotide comprises a sequence having at least 70%, 75%, 80%, 85%, 90%, 95%, 96%, 97%, 98%, or 99% or higher sequence identity to SEQ ID NO: 32.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 23; a polynucleotide sequence having at least 70%, 75%, 80%, 85%, 90%, 95%, 96%, 97%, 98%, or 99% or higher sequence identity to SEQ ID NO: 24; a sequence having at least 70%, 75%, 80%, 85%, 90%, 95%, 96%, 97%, 98%, or 99% or higher sequence identity to SEQ ID NO: 25; a polynucleotide sequence having at least 70%, 75%, 80%, 85%, 90%, 95%, 96%, 97%, 98%, or 99% or higher sequence identity to SEQ ID NO: 26; a polynucleotide sequence having at least 70%, 75%, 80%, 85%, 90%, 95%, 96%, 97%, 98%, or 99% or higher sequence identity to SEQ ID NO: 27; a polynucleotide sequence having at least 70%, 75%, 80%, 85%, 90%, 95%, 96%, 97%, 98%, or 99% or higher sequence identity to SEQ ID NO: 28; a polynucleotide sequence having at least 70%, 75%, 80%, 85%, 90%, 95%, 96%, 97%, 98%, or 99% or higher sequence identity to SEQ ID NO: 29; a polynucleotide sequence encoding a polypeptide selected from HA, NA, NP, M1, M2, NS1 and NS2; a polynucleotide sequence having at least 70%, 75%, 80%, 85%, 90%, 95%, 96%, 97%, 98%, or 99% or higher sequence identity to SEQ ID NO: 31; and a poly A tail comprising at least 20 consecutive adenines.

In some embodiments, the polynucleotide comprises a 5′ UTR sequence having at least 70%, 75%, 80%, 85%, 90%, 95%, 96%, 97%, 98%, or 99% or higher sequence identity to SEQ ID NO: 23; a polynucleotide sequence having at least 70%, 75%, 80%, 85%, 90%, 95%, 96%, 97%, 98%, or 99% or higher sequence identity to SEQ ID NO: 24; a sequence having at least 70%, 75%, 80%, 85%, 90%, 95%, 96%, 97%, 98%, or 99% or higher sequence identity to SEQ ID NO: 25; a polynucleotide sequence having at least 70%, 75%, 80%, 85%, 90%, 95%, 96%, 97%, 98%, or 99% or higher sequence identity to SEQ ID NO: 26; a polynucleotide sequence having at least 70%, 75%, 80%, 85%, 90%, 95%, 96%, 97%, 98%, or 99% or higher sequence identity to SEQ ID NO: 27; a polynucleotide sequence having at least 70%, 75%, 80%, 85%, 90%, 95%, 96%, 97%, 98%, or 99% or higher sequence identity to SEQ ID NO: 28; a polynucleotide sequence having at least 70%, 75%, 80%, 85%, 90%, 95%, 96%, 97%, 98%, or 99% or higher sequence identity to SEQ ID NO: 29; a polynucleotide sequence having at least 70%, 75%, 80%, 85%, 90%, 95%, 96%, 97%, 98%, or 99% or higher sequence identity to SEQ ID NO: 30; a polynucleotide sequence having at least 70%, 75%, 80%, 85%, 90%, 95%, 96%, 97%, 98%, or 99% or higher sequence identity to SEQ ID NO: 31; and a poly A tail comprising at least 20 consecutive adenines.

In some embodiments, the polynucleotide comprises a 5′ UTR sequence SEQ ID NO: SEQ ID NO: 23; a polynucleotide sequence having SEQ ID NO: 24; a polynucleotide sequence having SEQ ID NO: 25; a polynucleotide sequence having SEQ ID NO: 26; a polynucleotide sequence having SEQ ID NO: 27; a polynucleotide sequence having SEQ ID NO: 28; a polynucleotide sequence having SEQ ID NO: 29; a polynucleotide sequence encoding a polypeptide selected from HA, NA, NP, M1, M2, NS1 and NS2; a polynucleotide sequence having SEQ ID NO: 31; and a poly A tail comprising at least 20 consecutive adenines.

In some embodiments, the polynucleotide comprises a 5′ UTR sequence SEQ ID NO: SEQ ID NO: 23; a polynucleotide sequence having SEQ ID NO: 24; a polynucleotide sequence having SEQ ID NO: 25; a polynucleotide sequence having SEQ ID NO: 26; a polynucleotide sequence having SEQ ID NO: 27; a polynucleotide sequence having SEQ ID NO: 28; a polynucleotide sequence having SEQ ID NO: 29; a polynucleotide sequence having at least 70%, 75%, 80%, 85%, 90%, 95%, 96%, 97%, 98%, or 99% or higher sequence identity to SEQ ID NO: a polynucleotide sequence having SEQ ID NO: 31; and a poly A tail comprising at least 20 consecutive adenines. The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### K. Lipid Delivery

In some embodiments, the saRNA composition comprises lipids. The lipids and saRNA may together form nanoparticles. The lipids may encapsulate the mRNA in the form of a lipid nanoparticle (LNP) to aid cell entry and stability of the RNA/lipid nanoparticles.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

An LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), derivatives thereof, and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, Tris buffer, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.05, 0.06, 0.07, 0.08, 0.09, 0.10, 0.11, 0.12, 0.13, 0.14, 0.15, 0.16, 0.17, 0.18, 0.19, 0.20, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

An LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol phospholipid, about 30 mol % to about 40 mol % structural lipid, and about 0 mol % to about 10 mol % of PEG lipid. In a particular embodiment, the lipid component includes about 50 mol said cationic lipid, about 10 mol % phospholipid, about 38.5 mol % structural lipid, and about 1.5 mol % of PEG lipid. In another embodiment, the lipid component includes about 40 mol said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipid is a compound of Formula (I):

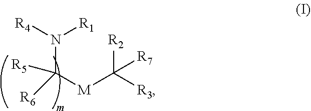

or their N-oxides, or salts or isomers thereof, wherein: Ri is selected from the group consisting of C5-30 alkyl, C5-20 alkenyl, —R*YR″, —YR″, and —R″M′R′; R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, —CHAR, —CQ(R)2, and unsubstituted C₁-6 alkyl, where Q is selected from a carbocycle, heterocycle, —OR, -0(CH2)nN(R)2, —C(0)OR, —OC(0)R, —CX3, —CX2H, —CXH2, —CN, —N(R)2, —C(0)N(R)2, —N(R)C(0)R, —N(R)S(0)2R, —N(R)C(0)N(R)2, —N(R)C(S)N(R)2, —N(R)Re, N(R)S(0)2R8, -0(CH2)nOR, —N(R)C(═NR9)N(R)2, —N(R)C(═CHR9)N(R)2, -0C(0)N(R)2J —N(R)C(0)0R, —N(0R)C(0)R, —N(0R)S(0)2R, —N(0R)C(0)0R, —N(0R)C(0)N(R)₂, —N(0R)C(S)N(R)2, —N(OR)C(═NR9)N(R)2, —N(OR)C(═CHR9)N(R)2, —C(═NR9)N(R)2, —C(═NR9)R, —C(0)N(R)0R, and —C(R)N(R)2C(0)0R, and each n is independently selected from 1, 2, 3, 4, and 5; each R5 is independently selected from the group consisting of C1-3 alkyl, C2-3 alkenyl, and H; each R^{e} is independently selected from the group consisting of C1-3 alkyl, C2-3 alkenyl, and H; M and M′ are independently selected from —C(0)0-, -0C(0)-, -0C(0)-M″-C(0)0-, —C(0)N(R′)—, —N(R′)C(0)-, —C(O)—, —C(S)—, —C(S)S—, —SC(S)—, —CH(OH)—, —P(0)(0R′)0-, —S(0)₂—, —S—S—, an aryl group, and a heteroaryl group, in which M″ is a bond, C1-13 alkyl or C2-13 alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO2, Ci-6 alkyl, —OR, —S(0)₂R, —S(0)₂N(R)2, C2-6 alkenyl, C3-6 carbocycle and heterocycle; each R is independently selected from the group consisting of C1-3 alkyl, C2-3 alkenyl, and H; each R′ is independently selected from the group consisting of Ci-is alkyl, C2-is alkenyl, —R*YR″, —YR″, and H; each R″ is independently selected from the group consisting of C3-15 alkyl and C3-15 alkenyl; each R* is independently selected from the group consisting of Ci-i2 alkyl and C2-i2 alkenyl; each Y is independently a C3-6 carbocycle; each X is independently selected from the group consisting of F, Cl, Br, and I; and m is selected from 5, 6, 7, 8, 9, 10, 11, 12, and 13; and wherein when R4 is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2. In some embodiments, the ionizable lipid is:

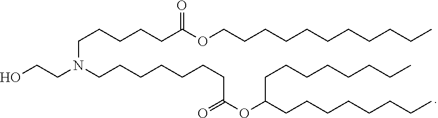 [SMILES: CCCCCCCCCCCOC(=O)CCCCCN(CCO)CCCCCCCC(=O)OC(CCCCCCCC)CCCCCCCC]

In some embodiments, the compounds have the following structure (I):

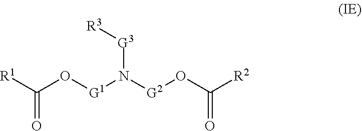

or a pharmaceutically acceptable salt, tautomer, prodrug or stereoisomer thereof, wherein: one of L1 or L2 is —O(C═O)—, —(C═O)O—, —C(═O)—, —O—, —S(O)x, —S—S—, —C(═O)S—, SC(═O)—, —NRaC(═O)—, —C(═O)NRa—, NRaC(═O)NRa—, —OC(═O)NRa- or —NRaC(═O)O—, and the other of L1 or L2 is —O(C═O)—, —(C═O)O—, —C(═O)—, —O—, —S(O)x, —S—S—, C(═O)S—, SC(═O)—, —NRaC(═O)—, —C(═O)NRa—, NRaC(═O)NRa—, —OC(═O)NRa— or —NRaC(═O)O— or a direct bond; G1 and G2 are each independently unsubstituted C₁-C12 alkylene or C1-C12 alkenylene; G3 is C₁-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, —C(═O)OR4, —OC(═O)R4 or —NR5C(═O)R4; R4 is C₁-C12 alkyl; R5 is H or C1-C6 alkyl; and x is 0, 1 or 2. In a preferred embodiment, the ionizable lipid is:

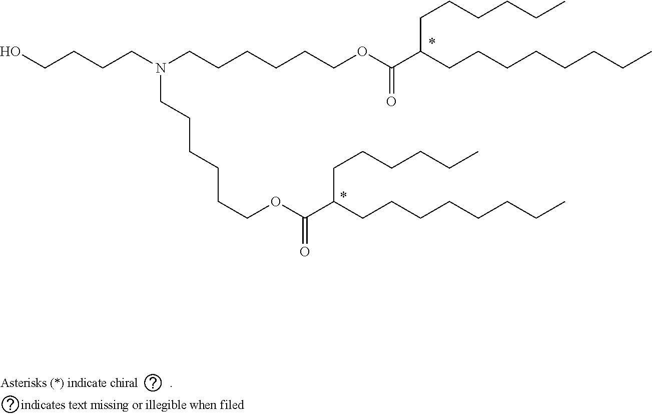 [SMILES: CCCCCCCCC(CCCCCC)C(=O)OCCCCCCN(CCCCO)CCCCCCOC(=O)C(CCCCCC)CCCCCCCC]

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG) -modified lipids. Non-limiting examples of PEG lipids include PEG-modified phosphatidylethanolamine and phosphatidic acid, PEG-ceramide conjugates (e.g., PEG-CerCl4 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

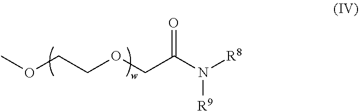

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

### L. Formulation

In one aspect, the disclosure relates to an immunogenic composition including: (i) a first ribonucleic acid (RNA) polynucleotide having an open reading frame encoding a first antigen, said antigen including at least one influenza virus antigenic polypeptide or an immunogenic fragment thereof, and (ii) a second RNA polynucleotide having an open reading frame encoding a second antigen, said second antigen including at least one influenza virus antigenic polypeptide or an immunogenic fragment thereof, wherein the first and second RNA polynucleotides are formulated in a lipid nanoparticle (LNP). In some embodiments, the first and second antigens include hemagglutinin (HA), or an immunogenic fragment or variant thereof. In some embodiments, the first antigen includes an HA from a different subtype of influenza virus to the influenza virus antigenic polypeptide or an immunogenic fragment thereof of the second antigen. In some embodiments, the composition further includes (iii) a third antigen including at least one influenza virus antigenic polypeptide or an immunogenic fragment thereof, wherein the third antigen is from influenza virus but is from a different strain of influenza virus to both the first and second antigens. In some embodiments, the first, second and third RNA polynucleotides are formulated in a lipid nanoparticle.

In some embodiments, the composition further includes (iv) a fourth RNA polynucleotide having an open reading frame encoding a fourth antigen, said antigen including at least one influenza virus antigenic polypeptide or an immunogenic fragment thereof, wherein the fourth antigen is from influenza virus but is from a different strain of influenza virus to the first, second and third antigens. In some embodiments, the first, second, third, and fourth RNA polynucleotides are formulated in a lipid nanoparticle.

In some embodiments, the RNA polynucleotides are mixed in desired ratios in a single vessel and are subsequently formulated into lipid nanoparticles. In some embodiments, the initial input of different RNA polynucleotides at a known ratio to be formulated in a single LNP process results in LNPs encapsulating the different RNA polynucleotides in about the same ratio as the input ratio. Such embodiments may be referred to herein as “pre-mix”. Accordingly, in some embodiments, first and second RNA polynucleotides are formulated in a single lipid nanoparticle. In some embodiments, the first, second, third, and fourth RNA polynucleotides are formulated in a single LNP. In some embodiments, the first, second, third, fourth, and fifth RNA polynucleotides are formulated in a single LNP. In some embodiments, the first, second, third, fourth, fifth, and sixth RNA polynucleotides are formulated in a single LNP. In some embodiments, the first, second, third, fourth, fifth, sixth, and seventh RNA polynucleotides are formulated in a single LNP. In some embodiments, the first, second, third, fourth, fifth, sixth, seventh, and eighth RNA polynucleotides are formulated in a single LNP.

In some embodiments, the molar ratio of the first RNA polynucleotide to the second RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the second RNA polynucleotide is greater than 1:1.

In some embodiments, the molar ratio of the first RNA polynucleotide to the third RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the third RNA polynucleotide is greater than 1.1.

In some embodiments, the molar ratio of the first RNA polynucleotide to the fourth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fourth RNA polynucleotide is greater than 1:1. In some embodiments, the molar ratio of the first RNA polynucleotide to the fifth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the fifth RNA polynucleotide is greater than 1:1. In some embodiments, the molar ratio of the first RNA polynucleotide to the sixth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the sixth RNA polynucleotide is greater than 1:1. In some embodiments, the molar ratio of the first RNA polynucleotide to the seventh RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the seventh RNA polynucleotide is greater than 1:1. In some embodiments, the molar ratio of the first RNA polynucleotide to the eighth RNA polynucleotide in the mix of RNA polynucleotides prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first RNA polynucleotide to the eighth RNA polynucleotide is greater than 1:1.

In alternative embodiments, each RNA polynucleotide encoding a particular antigen is formulated in an individual LNP, such that each LNP encapsulates an RNA polynucleotide encoding identical antigens. Such embodiments may be referred to herein as “post-mix”. Accordingly, in some embodiments, the first RNA polynucleotide is formulated in a first LNP; the second RNA polynucleotide is formulated in a second LNP; the third RNA polynucleotide is formulated in a third LNP; the fourth RNA polynucleotide is formulated in a fourth LNP; the fifth RNA polynucleotide is formulated in a fifth LNP; the sixth RNA polynucleotide is formulated in a sixth LNP; the seventh RNA polynucleotide is formulated in a seventh LNP; and the eighth RNA polynucleotide is formulated in an eighth LNP.

In some embodiments, the molar ratio of the first LNP to the second LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the second LNP is greater than 1:1.

In some embodiments, the molar ratio of the first LNP to the third LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the third LNP is greater than 1:1.

In some embodiments, the molar ratio of the first LNP to the fourth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fourth LNP is greater than 1:1. In some embodiments, the molar ratio of the first LNP to the fifth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the fifth LNP is greater than 1:1. In some embodiments, the molar ratio of the first LNP to the sixth LNP in the mix of LNPs prior to formulation into LNPs is about 1:50, about 1:25, about 1:10 about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the sixth LNP is greater than 1:1. In some embodiments, the molar ratio of the first LNP to the seventh LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the seventh LNP is greater than 1:1. In some embodiments, the molar ratio of the first LNP to the eighth LNP in the mix of LNPs prior to formulation into LNPs is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In some embodiments, the molar ratio of the first LNP to the eighth LNP is greater than 1:1.

In some embodiments, the relative amount of RNA encoding an antigen of a type B influenza virus may be increased as compared to RNA encoding an influenza type A virus (e.g., an immune response that comprises higher neutralization titers against an influenza type B virus (e.g., higher neutralization titers as compared to a composition comprising equal amounts of RNA encoding an influenza type A antigen and RNA encoding an influenza type B antigen (e.g., as determined by a pseudovirus neutralization assay described herein))). The present disclosure also provides exemplary doses of RNA that can produce strong immune responses against both types of influenza viruses (e.g., neutralizing titers and/or seroconversion rates that are at clinically relevalent levels (e.g., (i) neutralizing titers that are comparable or superior to those previously shown to prevent influenza symptoms, and/or (ii) neutralizing titers and/or seroconversion rates that are comparable or superior to those induced by a relevant comparator (e.g., a commercially approved influenza vaccine or an influenza RNA vaccine))). In some embodiments, a composition comprising a greater amount of RNA encoding influenza B antigens as compared to RNA encoding influenza A antigens produces an immune response against each of an influenza type B virus and influenza type A virus that is comparable or superior to that induced by a non-RNA influenza vaccine (e.g., an approved vaccine) and/or an RNA vaccine comprising equal amounts of RNA encoding influenza A antigens and RNA encoding influenza B antigens.

In some embodiments, the concentration of RNA in a pharmaceutical RNA preparation is about 0.1-0.2 mg/ml. In some embodiments, the concentration of RNA in a pharmaceutical RNA preparation is about 0.1 mg/ml. In some embodiments, the concentration of RNA in a pharmaceutical RNA preparation is about 0.12 mg/ml. In some embodiments, the concentration of RNA in a pharmaceutical RNA preparation is about 0.14 mg/ml. In some embodiments, the concentration of RNA in a pharmaceutical RNA preparation is about 0.16 mg/ml. In some embodiments, the concentration of RNA in a pharmaceutical RNA preparation is about 0.18 mg/ml. In some embodiments about 30 ug of RNA is administered by administering about 200 uL of RNA preparation. In some embodiments, the RNA in a pharmaceutical RNA preparation is diluted prior to administration (e.g., diluted to a concentration of about 0.05 mg/ml). In some embodiments, administration volumes are between about 200 μl and about 300 μl. In some embodiments, the RNA in a pharmaceutical RNA preparation is formulated in about 10 mM Tris buffer, and about 10% sucrose. In some embodiments, a pharmaceutical RNA preparation comprises RNA in a concentration of about 0.1 mg/ml, and is formulated in about 10 mM Tris buffer, and about 10% sucrose. In some embodiments, a pharmaceutical RNA preparation comprises RNA in a concentration of about 0.12 mg/ml, and is formulated in about 10 mM Tris buffer, and about 10% sucrose. In some embodiments, a pharmaceutical RNA preparation comprises RNA in a concentration of about 0.14 mg/ml, and is formulated in about 10 mM Tris buffer, and about 10% sucrose. In some embodiments, a pharmaceutical RNA preparation comprises RNA in a concentration of about 0.16 mg/ml, and is formulated in about 10 mM Tris buffer, and about 10% sucrose. In some embodiments, a pharmaceutical RNA preparation comprises RNA in a concentration of about 0.18 mg/ml, and is formulated in about 10 mM Tris buffer, and about 10% sucrose. Such a formulation can be diluted as needed prior to administration to administer different doses of RNA while keeping total injection volume relatively constant. For example, a dose of RNA of about 10 μg can be administered by diluting such a pharmaceutical RNA preparation by about 1:1 and administering about 200 μl of diluted pharmaceutical RNA preparation.

In some embodiments, a vaccine is formulated in a vial (e.g., a glass vial). In some embodiments, a glass vial is sealed with a bromobutyl elastomeric stopper and an aluminum seal with flip-off plastic cap.

In some embodiments, a composition comprises an RNA encoding an antigen (e.g., an HA protein) of an influenza virus that is recommended by a relevant health authority for inclusion in a seasonally-adapted vaccine (e.g., a cell-based, recombinant, or live attenuated virus). In some embodiments a composition comprises a plurality of RNAs, encoding antigens (e.g., HA proteins) of each influenza virus recommended by a relevant health authority for inclusion in a seasonally-adapted vaccine (e.g., a cell-based, recombinant, or live attenuated virus). In some embodiments, the influenza virus is an influenza A, influenza B, or influenza C virus. In some embodiments, the influenza A virus is an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, or H10N8 virus. In some embodiments, the influenza A virus is an H1N1, H3N2, H5N1, or H5N8 virus. In some embodiments, the influenza A virus is an H1N1 virus (e.g., A/Wisconsin/588/2019 or A/Sydney/5/2021). In some embodiments the influenza A virus is an H3N2 virus. In some embodiments the H3N2 virus is A/Cambodia/e0826360/2020 or A/Darwin/6/2021. In some embodiments, the influenza B virus is of a B/Yamagata or B/Victoria lineage. In some embodiments, the B/Victoria lineage influenza virus is B/Washington/02/2019. In some embodiments, the B/Victoria lineage virus is B/Austria/1359417/2021. In some embodiments, the B/Yamagata lineage influenza virus is B/Phuket/3073/2013.

In some embodiments, a composition described herein comprises a multivalent influenza vaccine. In some embodiments, a multivalent influenza vaccine comprises 2 to 50 RNA distinct molecules (e.g., 2 to 40, 2 to 30, or 2 to 20 RNA molecules), each of which, in some embodiments, may encode a different antigenic polypeptide (or a different version of a particular antigenic polypeptide) associated with influenza, e.g., as described in Arevalo, Claudia P., et al. “A multivalent nucleoside-modified mRNA vaccine against all known influenza virus subtypes.” Science 378.6622 (2022): 899-904. In some embodiments, a composition described herein comprises a trivalent influenza vaccine. In some embodiments, a trivalent influenza vaccine comprises RNAs encoding an antigenic polypeptide associated with two type A viruses and one type B virus that are predicted to be prevalent in a relevant jurisdiction. In some embodiments, a composition described herein comprises a tetravalent influenza vaccine. In some embodiments, a tetravalent influenza vaccine comprises RNAs encoding an antigenic polypeptide associated with two type A viruses and two type B viruses that are predicted to be prevalent in a relevant jurisdiction. In some embodiments, a composition described herein comprises an octavalent influenza vaccine. In some embodiments, an octavalent influenza vaccine comprises RNAs encoding two antigenic polypeptides associated with each of two type A viruses and two type B viruses that are predicted to be prevalent in a relevant jurisdiction (e.g., an HA protein and an NA protein associated with each virus, or immunogenic fragments thereof). In some embodiments, a composition disclosed herein comprises a tetravalent influenza vaccine comprising an RNA comprising a nucleotide sequence encoding an HA protein associated with an H1N1 virus (e.g., A/Wisconsin/588/2019), an RNA comprising a nucleotide sequence encoding an HA protein associated with an H3N2 virus (e.g., A/Cambodia/e0826360/2020), an RNA comprising a nucleotide sequence encoding an HA protein associated with a B/Victoria lineage influenza virus (e.g., B/Washington/02/2019), and an HA protein associated with a BNamagata lineage influenza virus (e.g., B/Phuket/3073/2013).

In some embodiments, a composition comprises a tetravalent influenza vaccine comprises RNA encoding an antigenic polypeptide associated with two type A viruses and two type B viruses that are predicted to be prevalent in a relevant jurisdiction. In some embodiments, a tetravalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, the tetravalent influenza vaccine comprises RNA associated with influenza types that are predicted to be prevalent in a relevant jurisdiction (e.g., HA polypeptides associated with the H1N1, H3N2, B/Victoria, and BNamagata influenza viruses that are predicted to be prevalent in a relevant jurisdiction).

In some embodiments, a composition comprises an octavalent influenza vaccine comprises RNA encoding an antigenic polypeptide associated with two type A viruses and two type B viruses that are predicted to be prevalent in a relevant jurisdiction. In some embodiments, an octavalent influenza vaccine comprises RNA encoding an antigenic polypeptide derived from HA from an influenza type A virus, RNA encoding an antigenic polypeptide derived from HA from an influenza type A virus, RNA encoding an antigenic polypeptide derived from HA from an influenza type B virus, RNA encoding an antigenic polypeptide derived from HA from an influenza type B virus, RNA encoding an antigenic polypeptide derived from one antigenic polypeptide selected from NA, NP, M1, M2, NS1 and NS2 from an influenza type A virus, RNA encoding an antigenic polypeptide derived from one antigenic polypeptide selected from NA, NP, M1, M2, NS1 and NS2 from an influenza type A virus, RNA encoding an antigenic polypeptide derived from one antigenic polypeptide selected from NA, NP, M1, M2, NS1 and NS2 from an influenza type B virus, and RNA encoding an antigenic polypeptide derived from one antigenic polypeptide selected from NA, NP, M1, M2, NS1 and NS2 from an influenza type B virus. In some embodiments, an octavalent influenza vaccine comprises RNA encoding an antigenic polypeptide derived from HA from an influenza type A virus, RNA encoding an antigenic polypeptide derived from HA from an influenza type A virus, RNA encoding an antigenic polypeptide derived from HA from an influenza type B virus, RNA encoding an antigenic polypeptide derived from HA from an influenza type B virus, RNA encoding an antigenic polypeptide derived from NA from an influenza type A virus, RNA encoding an antigenic polypeptide derived from NA from an influenza type A virus, RNA encoding an antigenic polypeptide derived from NA from an influenza type B virus, and RNA encoding an antigenic polypeptide derived from NA from an influenza type B virus. In some embodiments, an octavalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, an octavalent influenza vaccine comprises RNA associated with influenza types that are predicted to be prevalent in a relevant jurisdiction (e.g., HA polypeptides associated with the H1N1, H3N2, B/Victoria, and B/Yamagata influenza viruses that are predicted to be prevalent in a relevant jurisdiction).

In some embodiments, each of the RNAs in a composition disclosed herein encodes an antigenic polypeptide associated with an infectious agent that is predicted to be prevalent in a relevant jurisdiction. Such compositions can reduce the number of vaccinations needed.

In some embodiments, a nucleic acid containing particle comprises two or more RNA molecules, each comprising a nucleotide sequence encoding an antigen (e.g., an HA protein) associated with a different influenza virus. In some embodiments, a nucleic acid containing particle comprises three or more RNA molecules, each comprising a nucleotide sequence encoding an antigen (e.g., an HA protein) associated with a different influenza virus. In some embodiments, a nucleic acid containing particle comprises four or more RNA molecules, each comprising a nucleotide sequence encoding an antigen (e.g., an HA protein) associated with a different influenza virus. In some embodiments, a nucleic acid containing particle comprises an RNA molecule comprising a nucleotide sequence encoding an antigenic polypeptide associated with an H1N1 influenza virus, an RNA molecule comprising a nucleotide sequence encoding an antigenic polypeptide associated with an H3N2 influenza virus, an RNA molecule comprising a nucleotide sequence encoding an antigenic polypeptide associated with a B/Victoria lineage influenza virus, and an RNA molecule comprising a nucleotide sequence encoding an antigenic polypeptide associated with a BNamagata influenza virus. In some embodiments, each RNA in a composition comprising a nucleotide sequence encoding an antigenic polypeptide associated with an influenza virus is formulated in the same nucleic acid containing particle. In some embodiments, each RNA in a composition comprising a nucleotide sequence encoding an antigenic polypeptide associated with an influenza virus is formulated in separate nucleic acid containing particles.

In some embodiments, a nucleic acid containing particle (e.g., in some embodiments an LNP as described herein) comprising two or more RNA molecules, comprises each RNA molecule in the same amount (i.e., at a 1:1 ratio).

In some embodiments, a nucleic acid containing particle (e.g., in some embodiments an LNP as described herein) comprising two or more RNA molecules, comprises a different amount of each RNA molecule. For example, in some embodiments, a nucleic acid containing particle comprises a first RNA molecule and a second RNA molecule, where the first RNA molecule is present in an amount that is 0.01 to 100 times that of the second RNA molecule (e.g., wherein the amount of the first RNA molecule is 0.01 to 50, 0.01 to 4, 0.01 to 30, 0.01 to 25, 0.01 to 20, to 15, 0.01 to 10, 0.01 to 9, 0.01 to 8, 0.01 to 7, 0.01 to 6, 0.01 to 5, 0.01 to 4, 0.01 to 3, to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times higher than the second RNA molecule). In some embodiments, a nucleic acid containing particle comprises a first RNA molecule and a second RNA molecule, wherein the concentration of the first RNA molecule is 1 to 10 times that of the second RNA molecule. In some embodiments, a nucleic acid containing particle comprises a first RNA molecule and a second RNA molecule, wherein the concentration of the first RNA molecule is 1 to 5 times that of the second RNA molecule. In some embodiments, a nucleic acid containing particle comprises a first RNA molecule and a second RNA molecule, wherein the concentration of the first RNA molecule is 1 to 3 times that of the second RNA molecule. In some embodiments, a nucleic acid containing particle comprises a first RNA molecule and a second RNA molecule, wherein the concentration of the first RNA molecule is 2 times that of the second RNA molecule. In some embodiments, a nucleic acid containing particle comprises a first RNA molecule and a second RNA molecule, wherein the concentration of the first RNA molecule is 3 times that of the second RNA molecule.

In some embodiments, a nucleic acid containing particle (e.g., in some embodiments an LNP as described herein) comprising three RNA molecules, comprises each RNA molecule in the same amount (i.e., at a 1:1:1 ratio).

In some embodiments, a nucleic acid containing particle (e.g., in some embodiments an LNP as described herein) comprising three RNA molecules, comprises a different amount of each RNA molecule. For example, in some embodiments, the ratio of first RNA molecule: second RNA molecule: third RNA molecule is 1:0.01-100:0.01-100 (e.g., 1:0.01-50:0.01-50; 1:0.01-40:0.01-40; 1:0.01-30:0.01-25; 1:0.01-25:0.01-25; 1:0.01-20:0.01-20; 1:0.01-15:0.01-15; 1:0.01-10:0.01-9; 1:0.01-9:0.01-9; 1:0.01-8:0.01-8; 1:0.01-7:0.01-7; 1:0.01-6:0.01-6; 1:0.01-5; 1:0.01-4:0.01-4; 1:0.01-3:0.01-3; 1:0.01-2:0.01-2; or 1:0.01-1.5:0.01-1.5). In some embodiments, the ratio of first RNA molecule: second RNA molecule: third RNA molecule is 1:1:3. In some embodiments, the ratio of first RNA molecule: second RNA molecule: third RNA molecule is 1:3:3.

The term “dose” as used herein refers in general to a “dose amount” which relates to the amount of RNA administered per administration, i.e., per dosing. In some embodiments, administration of an immunogenic composition or vaccine of the present disclosure may be performed by single administration or boosted by multiple administrations. In some embodiments, a regimen described herein includes at least one dose. In some embodiments, a regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a provided regimen consists of two doses. In some embodiments, a regimen comprises three doses.

In one embodiment, the disclosure envisions administration of a single dose. In one embodiment, the disclosure envisions administration of a priming dose followed by one or more booster doses. The booster dose or the first booster dose may be administered 7 to 28 days or 14 to 24 days following administration of the priming dose. In some embodiments, a first booster dose may be administered 1 week to 3 months (e.g., 1 week, 2 weeks, 3 weeks, 4 weeks, 5 weeks, 6 weeks, 7 weeks, 8 weeks, 9 weeks, 10 weeks, 11 weeks, 12 weeks) following administration of a priming dose. In some embodiments, a subsequent booster dose may be administered at least 1 week or longer, including, e.g., at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or longer, following a preceding booster dose. In some embodiments, subsequent booster doses may be administered about 5-9 weeks or 6-8 weeks apart. In some embodiments, at least one subsequent booster dose (e.g., after a first booster dose) may be administered at least 3 months or longer, including, e.g., at least 4 months, at least months, at least 6 months, at least 7 months, at least 8 months, at least 9 months, at least 10 months, or longer, following a preceding dose.

In some embodiments, a dose comprises a total amount of RNA of 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, a dose comprises a total amount of RNA (e.g., modRNA) of up to about 100 μg. In some embodiments, a dose comprises 0.1 μg to 100 μg of one or more first RNAs and 0.1 μg to 100 μg of one or more second RNAs, wherein the one or more first RNAs each comprise a nucleotide sequence encoding an antigenic polypeptide associated with a first infectious agent (e.g., a coronavirus), and the one or more second RNAs each comprise a nucleotide sequence encoding an antigenic polypeptide associated with a second infectious agent (e.g., influenza). In some embodiments, a dose comprises 3 to 60 μg of one or more first RNAs and 3 to 90 μg of one or more second RNAs. In some embodiments, a dose comprises 3 to 60 μg of one or more first RNAs and 3 to 90 μg of one or more second RNAs, wherein the dose comprises up to 100 μg of RNA total. In some embodiments, a dose comprises 3 to 30 μg of one or more first RNAs and 3 to 60 μg of one or more second RNAs, wherein the dose comprises up to 100 μg of RNA total. In some embodiments, a dose comprises 3 μg of one or more first RNAs and 3 μg of one or more second RNAs. In some embodiments, a dose comprises 3 μg of one or more first RNAs and 6 μg of one or more second RNAs. In some embodiments, a dose comprises 10 μg of one or more first RNAs and 10 μg of one or more second RNAs. In some embodiments, a dose comprises 10 μg of one or more first RNAs and 20 μg of one or more second RNAs. In some embodiments, a dose comprises 30 μg of one or more first RNAs and 30 μg of one or more second RNAs. In some embodiments, a dose comprises 30 μg of one or more first RNAs and 60 μg of one or more second RNAs. In some embodiments, a dose comprises 60 μg of one or more first RNAs and 30 μg of one or more second RNAs.

In some embodiments, a subsequent dose given to an individual (e.g., as part of a primary regimen or booster regimen) can have the same amount of RNA as previously given to the individual. In some embodiments, a subsequent dose given to an individual (e.g., as part of a primary regimen or booster regimen) can differ in the amount of RNA, as compared to the amount previously given to the individual. For example, in some embodiments, a subsequent dose can be higher or lower than the prior dose, for example, based on consideration of various factors, including, e.g., immunogenicity and/or reactogenicity induced by the prior dose, prevalence of the disease, etc. In some embodiments, a subsequent dose can be higher than a prior dose by at least 30%, at least 40%, at least 50%, at least 60%, at least 70%, at least 80%, at least 90%, or higher. In some embodiments, a subsequent dose can be higher than a prior dose by at least 1.5-fold, at least 2-fold, at least 2.5 fold, at least 3-fold, or higher. In some embodiments, a subsequent dose can be higher than a prior dose by at least 30%, at least 40%, at least 50%, at least 60%, at least 70%, at least 80%, at least 90%, or higher. In some embodiments, a subsequent dose can be lower than a prior dose by at least 10%, at least 20%, at least 30%, at least 40%, at least 50%, at least 60%, at least 70% or lower. In some embodiments, an amount the RNA described herein from 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 70 μg, about 80 μg, about 90 μg, or about 100 μg may be administered per dose (e.g., in a given dose).

In some embodiments, an amount of the RNA described herein of 60 μg or lower, 55 μg or lower, 50 μg or lower, 45 μg or lower, 40 μg or lower, 35 μg or lower, 30 μg or lower, 25 μg or lower, 20 μg or lower, 15 μg or lower, 10 μg or lower, 5 μg or lower, 3 μg or lower, 2.5 μg or lower, or 1 μg or lower may be administered per dose (e.g., in a given dose).

In some embodiments, an amount of the RNA described herein of at least 0.25 μg, at least 0.5 μg, at least 1 μg, at least 2 μg, at least 3 μg, at least 4 μg, at least 5 μg, at least 10 μg, at least 15 μg, at least 20 μg, at least 25 μg, at least 30 μg, at least 40 μg, at least 50 μg, or at least 60 μg may be administered per dose (e.g., in a given dose). In some embodiments, an amount of the RNA described herein of at least 3 μg may be administered in at least one of given doses. In some embodiments, an amount of the RNA described herein of at least 10 ug may be administered in at least one of given doses. In some embodiments, an amount of the RNA described herein of at least 15 ug may be administered in at least one of given doses. In some embodiments, an amount of the RNA described herein of at least 20 ug may be administered in at least one of given doses. In some embodiments, an amount of the RNA described herein of at least 25 ug may be administered in at least one of given doses. In some embodiments, an amount of the RNA described herein of at least 30 ug may be administered in at least one of given doses. In some embodiments, an amount of the RNA described herein of at least 50 ug may be administered in at least one of given doses. In some embodiments, an amount of the RNA described herein of at least 60 ug may be administered in at least one of given doses. In some embodiments, combinations of aforementioned amounts may be administered in a regimen comprising two or more doses (e.g., a prior dose and a subsequent dose can be of different amounts as described herein). In some embodiments, combinations of aforementioned amounts may be administered in a primary regimen and a booster regimen (e.g., different doses can be given in a primary regimen and a booster regimen).

In some embodiments, an amount of an RNA described herein of 0.25 μg to 60 μg, 0.5 μg to 55 μg, 1 μg to 50 μg, 5 μg to 40 μg, or 10 μg to 30 μg may be administered per dose. In some embodiments, an amount of the RNA described herein of 3 μg to 30 μg may be administered in at least one of given doses. In some embodiments, an amount of the RNA described herein of 3 μg to 20 μg may be administered in at least one of given doses. In some embodiments, an amount of the RNA described herein of 3 μg to 15 μg may be administered in at least one of given doses. In some embodiments, an amount of the RNA described herein of 3 μg to 10 μg may be administered in at least one of given doses. In some embodiments, an amount of the RNA described herein of 10 μg to 30 μg may be administered in at least one of given doses. In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart. In some embodiments, doses may be about 7 to about 28 days apart. In some embodiments, doses may be about 14 to about 24 days. In some embodiments, doses may be about 21 to about 42 days.

In some embodiments, a vaccination regimen comprises a first dose and a second dose. In some embodiments, a first dose and a second dose are administered by at least 21 days apart. In some embodiments, a first dose and a second dose are administered by at least 28 days apart.

In some embodiments, a vaccination regimen comprises a first dose and a second dose, wherein the amount of RNA administered in the first dose is the same as the amount of RNA administered in the second dose. In some embodiments, a vaccination regimen comprises a first dose and a second dose wherein the amount of RNA administered in the first dose differs from that administered in the second dose.

In some embodiments, a vaccination regimen comprises a first dose and a second dose, wherein the amount of RNA administered in the first dose is less than that administered in the second dose. In some embodiments, the amount of RNA administered in the first dose is 10%-90% of the second dose. In some embodiments, the amount of RNA administered in the first dose is 10%-50% of the second dose. In some embodiments, the amount of RNA administered in the first dose is 10%-20% of the second dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart.

In some embodiments, a first dose comprises less than about 30 ug of RNA and a second dose comprises at least about 30 ug of RNA. In some embodiments, a first dose comprises about 1 to less than about 30 ug of RNA (e.g., about 0.1, about 1, about 3, about 5, about 10, about 15, about 20, about 25, or less than about 30 ug of RNA) and a second dose comprises about 30 to about 100 ug of RNA (e.g., about 30, about 40, about 50, or about 60 ug of RNA). In some embodiments, a first dose comprises about 1 to about 20 ug of RNA, about 1 to about 10 ug of RNA, or about 1 to about 5 ug of RNA and a second dose comprises about 30 to about 60 ug of RNA.

In some embodiments, a first dose comprises about 1 to about 10 ug of RNA (e.g., about 1, about 2, about 3, about 4, about 5, about 6, about 7, about 8, about 9, or about 10 ug of RNA) and a second dose comprises about 30 to about 60 ug of RNA (e.g., about 30, about 35, about about 45, about 50, about 55, or about 60 ug of RNA).

In some embodiments, a first dose comprises about 1 ug of RNA and a second dose comprises about 30 ug of RNA. In some embodiments, a first dose comprises about 3 ug of RNA and a second dose comprises about 30 ug of RNA. In some embodiments, a first dose comprises about 5 ug of RNA and a second dose comprises about 30 ug of RNA. In some embodiments, a first dose comprises about 10 ug of RNA and a second dose comprises about 30 ug of RNA. In some embodiments, a first dose comprises about 15 ug of RNA and a second dose comprises about 30 ug of RNA.

In some embodiments, a first dose comprises about 1 ug of RNA and a second dose comprises about 60 ug of RNA. In some embodiments, a first dose comprises about 3 ug of RNA and a second dose comprises about 60 ug of RNA. In some embodiments, a first dose comprises about 5 ug of RNA and a second dose comprises about 60 ug of RNA. In some embodiments, a first dose comprises about 6 ug of RNA and a second dose comprises about 60 ug of RNA. In some embodiments, a first dose comprises about 10 ug of RNA and a second dose comprises about 60 ug of RNA. In some embodiments, a first dose comprises about 15 ug of RNA and a second dose comprises about 60 ug of RNA. In some embodiments, a first dose comprises about 20 ug of RNA and a second dose comprises about 60 ug of RNA. In some embodiments, a first dose comprises about 25 ug of RNA and a second dose comprises about 60 ug of RNA.

In some embodiments, a first dose comprises about 30 ug of RNA and a second dose comprises about 60 ug of RNA.

In some embodiments, a first dose comprises less than about 10 ug of RNA and a second dose comprises at least about 10 ug of RNA. In some embodiments, a first dose comprises about 0.1 to less than about 10 ug of RNA (e.g., about 0.1, about 0.5, about 1, about 2, about 3, about 4, about 5, about 6, about 7, about 8, or less than about 10 ug of RNA) and a second dose comprises about 10 to about 30 ug of RNA (e.g., about 10, about 15, about 20, about 25, or about 30 ug of RNA). In some embodiments, a first dose comprises about 0.1 to about 10 ug of RNA, about 1 to about 5 ug of RNA, or about 0.1 to about 3 ug of RNA and a second dose comprises about 10 to about 30 ug of RNA.

In some embodiments, a first dose comprises about 0.1 to about 5 ug of RNA (e.g., about 0.1, about 0.5, about 1, about 2, about 3, about 4, about 5 ug of RNA) and a second dose comprises about 10 to about 20 ug of RNA (e.g., about 10, about 12, about 14, about 16, about 18, about 20 ug of RNA).

In some embodiments, a first dose comprises about 0.1 ug of RNA and a second dose comprises about 10 ug of RNA. In some embodiments, a first dose comprises about 0.3 ug of RNA and a second dose comprises about 10 ug of RNA. In some embodiments, a first dose comprises about 1 ug of RNA and a second dose comprises about 10 ug of RNA. In some embodiments, a first dose comprises about 3 ug of RNA and a second dose comprises about 10 ug of RNA.

In some embodiments, a first dose comprises less than about 3 ug of RNA and a second dose comprises at least about 3 ug of RNA. In some embodiments, a first dose comprises about 0.1 to less than about 3 ug of RNA (e.g., about 0.1, about 0.2, about 0.3, about 0.5, about 0.6, about 0.7, about 0.8, about 0.9, about 1.0, about 1.5, about 2.0, or about 2.5 ug of RNA) and a second dose comprises about 3 to about 10 ug of RNA (e.g., about 3, about 4, about 5, about 6, or about 7, about 8, about 9, or about 10 ug of RNA). In some embodiments, a first dose comprises about 0.1 to about 3 ug of RNA, about 0.1 to about 1 ug of RNA, or about 0.1 to about 0.5 ug of RNA and a second dose comprises about 3 to about 10 ug of RNA.

In some embodiments, a first dose comprises about 0.1 to about 1.0 ug of RNA (e.g., about 0.1, about 0.2, about 0.3, about 0.4, about 0.5, about 0.6, about 0.7, about 0.8, about 0.9, or about 1.0 ug of RNA) and a second dose comprises about 1 to about 3 ug of RNA (e.g., about 1.0, about 1.5, about 2.0, about 2.5, or about 3.0 ug of RNA).

In some embodiments, a first dose comprises about 0.1 ug of RNA and a second dose comprises about 3 ug of RNA. In some embodiments, a first dose comprises about 0.3 ug of RNA and a second dose comprises about 3 ug of RNA. In some embodiments, a first dose comprises about 0.5 ug of RNA and a second dose comprises about 3 ug of RNA. In some embodiments, a first dose comprises about 1 ug of RNA and a second dose comprises about 3 ug of RNA.

In some embodiments, a vaccination regimen comprises a first dose and a second dose, wherein the amount of RNA administered in the first dose is greater than that administered in the second dose. In some embodiments, the amount of RNA administered in the second dose is 10%-90% of the first dose. In some embodiments, the amount of RNA administered in the second dose is 10%-50% of the first dose. In some embodiments, the amount of RNA administered in the second dose is 10%-20% of the first dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart In some embodiments, a first dose comprises at least about 30 ug of RNA and a second dose comprises less than about 30 ug of RNA. In some embodiments, a first dose comprises about 30 to about 100 ug of RNA (e.g., about 30, about 40, about 50, or about 60 ug of RNA) and a second dose comprises about 1 to about 30 ug of RNA (e.g., about 0.1, about 1, about 3, about about 10, about 15, about 20, about 25, or about 30 ug of RNA). In some embodiments, a second dose comprises about 1 to about 20 ug of RNA, about 1 to about 10 ug of RNA, or about 1 to 5 ug of RNA. In some embodiments, a first dose comprises about 30 to about 60 ug of RNA and a second dose comprises about 1 to about 20 ug of RNA, about 1 to about 10 ug of RNA, or about 0.1 to about 3 ug of RNA.

In some embodiments, a first dose comprises about 30 to about 60 ug of RNA (e.g., about 30, about 35, about 40, about 45, about 50, about 55, or about 60 ug of RNA) and a second dose comprises about 1 to about 10 ug of RNA (e.g., about 1, about 2, about 3, about 4, about 5, about 6, about 7, about 8, about 9, or about 10 ug of RNA).

In some embodiments, a first dose comprises about 30 ug of RNA and a second dose comprises about 1 ug of RNA. In some embodiments, a first dose comprises about 30 ug of RNA and a second dose comprises about 3 ug of RNA. In some embodiments, a first dose comprises about 30 ug of RNA and a second dose comprises about 5 ug of RNA. In some embodiments, a first dose comprises about 30 ug of RNA and a second dose comprises about ug of RNA. In some embodiments, a first dose comprises about 30 ug of RNA and a second dose comprises about 15 ug of RNA.

In some embodiments, a first dose comprises about 60 ug of RNA and a second dose comprises about 1 ug of RNA. In some embodiments, a first dose comprises about 60 ug of RNA and a second dose comprises about 3 ug of RNA. In some embodiments, a first dose comprises about 60 ug of RNA and a second dose comprises about 5 ug of RNA. In some embodiments, a first dose comprises about 60 ug of RNA and a second dose comprises about 6 ug of RNA. In some embodiments, a first dose comprises about 60 ug of RNA and a second dose comprises about 10 ug of RNA. In some embodiments, a first dose comprises about 60 ug of RNA and a second dose comprises about 15 ug of RNA. In some embodiments, a first dose comprises about 60 ug of RNA and a second dose comprises about 20 ug of RNA. In some embodiments, a first dose comprises about 60 ug of RNA and a second dose comprises about 25 ug of RNA. In some embodiments, a first dose comprises about 60 ug of RNA and a second dose comprises about 30 ug of RNA.

In some embodiments, a first dose comprises at least about 10 ug of RNA and a second dose comprises less than about 10 ug of RNA. In some embodiments, a first dose comprises about to about 30 ug of RNA (e.g., about 10, about 15, about 20, about 25, or about 30 ug of RNA) and a second dose comprises about 0.1 to less than about 10 ug of RNA (e.g., about 0.1, about 0.5, about 1, about 2, about 3, about 4, about 5, about 6, about 7, about 8, or less than about 10 ug of RNA). In some embodiments, a first dose comprises about 10 to about 30 ug of RNA, or about 0.1 to about 3 ug of RNA and a second dose comprises about 1 to about 10 ug of RNA, or about 1 to about 5 ug of RNA.

In some embodiments, a first dose comprises about 10 to about 20 ug of RNA (e.g., about 10, about 12, about 14, about 16, about 18, about 20 ug of RNA) and a second dose comprises about 0.1 to about 5 ug of RNA (e.g., about 0.1, about 0.5, about 1, about 2, about 3, about 4, or about 5 ug of RNA).

In some embodiments, a first dose comprises about 10 ug of RNA and a second dose comprises about 0.1 ug of RNA. In some embodiments, a first dose comprises about 10 ug of RNA and a second dose comprises about 0.3 ug of RNA. In some embodiments, a first dose comprises about 10 ug of RNA and a second dose comprises about 1 ug of RNA. In some embodiments, a first dose comprises about 10 ug of RNA and a second dose comprises about 3 ug of RNA.

In some embodiments, a first dose comprises at least about 3 ug of RNA and a second dose comprises less than about 3 ug of RNA. In some embodiments, a first dose comprises about 3 to about 10 ug of RNA (e.g., about 3, about 4, about 5, about 6, or about 7, about 8, about 9, or about 10 ug of RNA) and a second dose comprises 0.1 to less than about 3 ug of RNA (e.g., about 0.1, about 0.2, about 0.3, about 0.5, about 0.6, about 0.7, about 0.8, about 0.9, about 1.0, about 1.5 about 2.0, or about 2.5 ug of RNA). In some embodiments, a first dose comprises about 3 to about 10 ug of RNA and a second dose comprises about 0.1 to about 3 ug of RNA, about 0.1 to about 1 ug of RNA, or about 0.1 to about 0.5 ug of RNA.

In some embodiments, a first dose comprises about 1 to about 3 ug of RNA (e.g., about 1, about 1.5, about 2.0, about 2.5, or about 3.0 ug of RNA) and a second dose comprises about to 0.3 ug of RNA (e.g., about 0.1, about 0.2, about 0.3, about 0.4, about 0.5, about 0.6, about 0.7, about 0.8, about 0.9, or about 1.0 ug of RNA).

In some embodiments, a first dose comprises about 3 ug of RNA and a second dose comprises about 0.1 ug of RNA. In some embodiments, a first dose comprises about 3 ug of RNA and a second dose comprises about 0.3 ug of RNA. In some embodiments, a first dose comprises about 3 ug of RNA and a second dose comprises about 0.6 ug of RNA. In some embodiments, a first dose comprises about 3 ug of RNA and a second dose comprises about 1 ug of RNA. In some embodiments, a vaccination regimen comprises at least two doses, including, e.g., at least three doses, at least four doses or more. In some embodiments, a vaccination regimen comprises three doses. In some embodiments, the time interval between the first dose and the second dose can be the same as the time interval between the second dose and the third dose. In some embodiments, the time interval between the first dose and the second dose can be longer than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by at least 1 month (including, e.g., at least 2 months, at least 3 months, at least 4 months, at least 5 months, at least 6 months, at least 7 months, at least 8 months, at least 9 months, at least 10 months, at least 11 months, at least 12 months, or longer).

In some embodiments, a last dose of a primary regimen and a first dose of a booster regimen are given at least 2 months, at least 3 months, at least 4 months, at least 5 months, at least 6 months, at least 7 months, at least 8 months, at least 9 months, at least 10 months, at least 11 months, at least 12 months, or more apart. In some embodiments, a primary regimen may comprises two doses. In some embodiments, a primary regimen may comprises three doses. In some embodiments, a first dose and a second dose (and/or other subsequent dose) may be administered by intramuscular injection. In some embodiments, a first dose and a second dose (and/or other subsequent dose) may be administered in the deltoid muscle. In some embodiments, a first dose and a second dose (and/or other subsequent dose) may be administered in the same arm.

In some embodiments, an mRNA composition described herein is administered (e.g., by intramuscular injection) as a series of two doses (e.g., 0.3 mL each) 21 days apart. In some embodiments, an mRNA composition described herein is administered (e.g., by intramuscular injection) as a series of two doses (e.g., 0.2 mL each) 21 days apart. In some embodiments, an mRNA composition described herein is administered (e.g., by intramuscular injection) as a series of three doses (e.g., 0.3 mL or lower including, e.g., 0.2 mL), wherein doses are given at least 3 weeks apart. In some embodiments, the first and second doses may be administered 3 weeks apart, while the second and third doses may be administered at a longer time interval than that between the first and the second doses, e.g., at least 4 weeks apart or longer (including, at least 5 weeks, at least 6 weeks, at least 7 weeks, at least 8 weeks, at least 9 weeks, or longer). In some embodiments, each dose is about 60 ug. In some embodiments, each dose is about 50 ug. In some embodiments, each dose is about 30 ug. In some embodiments, each dose is about 25 ug. In some embodiments, each dose is about 20 ug. In some embodiments, each dose is about 15 ug. In some embodiments, each dose is about 10 ug. In some embodiments, each dose is about 3 ug.

In some embodiments, at least one dose given in a vaccination regimen (e.g., a primary vaccination regimen and/or a booster vaccination regimen) is about 60 ug. In some embodiments, at least one dose given in a vaccination regimen (e.g., a primary vaccination regimen and/or a booster vaccination regimen) is about 50 ug. In some embodiments, at least one dose given in a vaccination regimen (e.g., a primary vaccination regimen and/or a booster vaccination regimen) is about 30 ug. In some embodiments, at least one dose given in a vaccination regimen (e.g., a primary vaccination regimen and/or a booster vaccination regimen) is about 25 ug. In some embodiments, at least one dose given in a vaccination regimen (e.g., a primary vaccination regimen and/or a booster vaccination regimen) is about 20 ug. In some embodiments, at least one dose given in a vaccination regimen (e.g., a primary vaccination regimen and/or a booster vaccination regimen) is about 15 ug. In some embodiments, at least one dose given in a vaccination regimen (e.g., a primary vaccination regimen and/or a booster vaccination regimen) is about 10 ug. In some embodiments, at least one dose given in a vaccination regimen (e.g., a primary vaccination regimen and/or a booster vaccination regimen) is about 3 ug.

In one embodiment, an amount of the RNA described herein of about 60 μg is administered per dose. In one embodiment, an amount of the RNA described herein of about 50 μg is administered per dose. In one embodiment, an amount of the RNA described herein of about 30 μg is administered per dose. In one embodiment, an amount of the RNA described herein of about 25 μg is administered per dose. In one embodiment, an amount of the RNA described herein of about 20 μg is administered per dose. In one embodiment, an amount of the RNA described herein of about 15 μg is administered per dose. In one embodiment, an amount of the RNA described herein of about 10 μg is administered per dose. In one embodiment, an amount of the RNA described herein of about 5 μg is administered per dose. In one embodiment, an amount of the RNA described herein of about 3 μg is administered per dose. In one embodiment, at least two of such doses are administered. For example, a second dose may be administered about 21 days following administration of the first dose.

In some embodiments, the efficacy of the RNA vaccine described herein (e.g., administered in two doses, wherein a second dose may be administered about 21 days following administration of the first dose, and administered, for example, in an amount of about 30 μg per dose) is at least 70%, at least 80%, at least 90, or at least 95% beginning 7 days after administration of the second dose (e.g., beginning 28 days after administration of the first dose if a second dose is administered 21 days following administration of the first dose). In some embodiments, such efficacy is observed in populations of age of at least 50, at least 55, at least 60, at least 65, at least 70, or older. In some embodiments, the efficacy of the RNA vaccine described herein (e.g., administered in two doses, wherein a second dose may be administered about 21 days following administration of the first dose, and administered, for example, in an amount of about 30 μg per dose) beginning 7 days after administration of the second dose (e.g., beginning 28 days after administration of the first dose if a second dose is administered 21 days following administration of the first dose) in populations of age of at least 65, such as 65 to 80, 65 to 75, or 65 to 70, is at least 90%, at least 91%, at least 92%, at least 93%, at least 94%, or at least 95%. Such efficacy may be observed over time periods of up to 1 month, 2 months, 3 months, 6 months or even longer.

In one embodiment, vaccine efficacy is defined as the percent reduction in the number of subjects with evidence of infection (vaccinated subjects vs. non-vaccinated subjects).

In one embodiment, methods and agents described herein are administered to a paediatric population. In various embodiments, the paediatric population comprises or consists of subjects under 18 years, e.g., 5 to less than 18 years of age, 12 to less than 18 years of age, 16 to less than 18 years of age, 12 to less than 16 years of age, 5 to less than 12 years of age, or 6 months to less than 12 years of age. In various embodiments, the paediatric population comprises or consists of subjects under 5 years, e.g., 2 to less than 5 years of age, 12 to less than 24 months of age, 7 to less than 12 months of age, or less than 6 months of age. In some such embodiments, an mRNA composition described herein is administered to subjects of less than 2 years old, for example, 6 months to less than 2 years old. In some such embodiments, an mRNA composition described herein is administered to subjects of less than 6 months old, for example, 1 month to less than 4 months old. In some embodiments, a dosing regimen (e.g., doses and/or dosing schedule) for a paediatric population may vary for different age groups. For example, in some embodiments, a subject 6 months through 4 years of age may be administered according to a primary regimen comprising at least three doses, in which the initial two doses are administered at least 3 weeks (including, e.g., at least 4 weeks, at least 5 weeks, at least 6 weeks, or longer) apart followed by a third dose administered at least 8 weeks (including, e.g., at least 9 weeks, at least 10 weeks, at least 11 weeks, at least 12 weeks, or longer) after the second dose. In some such embodiments, at least one dose administered is 3 ug RNA described herein. In some embodiments, a subject 5 years of age and older may be administered according to a primary regimen comprising at least two doses, in which the two doses are administered at least 3 weeks (including, e.g., at least 3 weeks, at least 4 weeks, at least 5 weeks, at least 6 weeks, or longer) apart. In some such embodiments, at least one dose administered is 10 ug RNA described herein. In some embodiments, a subject 5 years of age and older who are immunocompromised (e.g., in some embodiments subjects who have undergone solid organ transplantation, or who are diagnosed with conditions that are considered to have an equivalent of immunocompromise) may be administered according to a primary regimen comprising at least three doses, in which the initial two doses are administered at least 3 weeks (including, e.g., at least 3 weeks, at least 4 weeks, at least 5 weeks, at least 6 weeks, or longer) apart, followed by a third dose administered at least 4 weeks (including, e.g., at least 5 weeks, at least 6 weeks, at least 7 weeks, at least 8 weeks, at least 9 weeks, at least weeks, at least 11 weeks, at least 12 weeks, or longer) after the second dose.

In some embodiments, an mRNA composition described herein is administered to subjects of age 12 or older and each dose is about 30 ug. In some embodiments, an mRNA composition described herein is administered to subjects of age 12 or older (including, e.g., age 18 or older) and each dose is higher than 30 ug, including, e.g., 35 ug, 40 ug, 45 ug, 50 ug, 55 ug, 60 ug, 65 ug, 70 ug, or higher. In some such embodiments, an mRNA composition described herein is administered to subjects of age 12 or older and each dose is about 60 ug. In some such embodiments, an mRNA composition described herein is administered to subjects of age 12 or older and each dose is about 50 ug. In one embodiment, the paediatric population comprises or consists of subjects 12 to less than 18 years of age including subjects 16 to less than 18 years of age and/or subjects 12 to less than 16 years of age. In this embodiment, treatments may comprise 2 vaccinations 21 days apart, wherein, in one embodiment, the vaccine is administered in an amount of 30 μg RNA per dose, e.g., by intramuscular administration. In some embodiments, higher doses are administered to older pediatric patients and adults, e.g., to patients 12 years or older, compared to younger children or infants, e.g. 2 to less than 5 years old, 6 months to less than 2 years old, or less than 6 months old. In some embodiments, higher doses are administered to children who are 2 to less than 5 years old, as compared to toddlers and/or infants, e.g., who are 6 months to less than 2 years old, or less than 6 months old.

In one embodiment, the paediatric population comprises or consists of subjects 5 to less than 18 years of age including subjects 12 to less than 18 years of age and/or subjects 5 to less than 12 years of age. In this embodiment, treatments may comprise 2 vaccinations 21 days apart, wherein, in various embodiments, the vaccine is administered in an amount of 10 μg, 20 μg, or 30 μg RNA per dose, e.g., by intramuscular administration. In some such embodiments, an mRNA composition described herein is administered to subjects of age 5 to 11 and each dose is about 10 ug.

In one embodiment, the paediatric population comprises or consists of subjects less than 5 years of age including subjects 2 to less than 5 years of age, subjects 12 to less than 24 months of age, subjects 7 to less than 12 months of age, subjects 6 to less than 12 months of age and/or subjects less than 6 months of age. In this embodiment, treatments may comprise 2 vaccinations, e.g., 21 to 42 days apart, e.g., 21 days apart, wherein, in various embodiments, the vaccine is administered in an amount of 3 μg, 10 μg, 20 μg, or 30 μg RNA per dose, e.g., by intramuscular administration. In some such embodiments, an mRNA composition described herein is administered to subjects of age 2 to less than 5 and each dose is about 3 ug. In some such embodiments, an mRNA composition described herein is administered to subjects of about 6 months to less than about 5 years and each dose is about 3 ug.

In some embodiments, an mRNA composition described herein is administered to subjects of age 12 or older and at least one dose given in a vaccination regimen (e.g., a primary vaccination regimen and/or a booster vaccination regimen) is about 60 ug. In some embodiments, an mRNA composition described herein is administered to subjects of age 12 or older and at least one dose given in a vaccination regimen (e.g., a primary vaccination regimen and/or a booster vaccination regimen) is about 30 ug. In some embodiments, an mRNA composition described herein is administered to subjects of age 12 or older and at least one dose given in a vaccination regimen (e.g., a primary vaccination regimen and/or a booster vaccination regimen) is about 15 ug. In some embodiments, an mRNA composition described herein is administered to subjects of age 5 to less than 12 years of age and at least one dose given in a vaccination regimen (e.g., a primary vaccination regimen and/or a booster vaccination regimen) is about 10 ug. In some embodiments, an mRNA composition described herein is administered to subjects of age 2 to less than 5 and at least one dose given in a vaccination regimen (e.g., a primary vaccination regimen and/or a booster vaccination regimen) is about 3 ug. In some embodiments, an mRNA composition described herein is administered to subjects of 6 months to less than age 2 and at least one dose given in a vaccination regimen (e.g., a primary vaccination regimen and/or a booster vaccination regimen) is about 3 ug or lower, including, e.g., 2 ug, 1 ug, or lower). In some embodiments, an mRNA composition described herein is administered to infants of less than 6 months and at least one dose given in a vaccination regimen (e.g., a primary vaccination regimen and/or a booster vaccination regimen) is about 3 ug or lower, including, e.g., 2 ug, 1 ug, 0.5 ug, or lower).

In some embodiments, a dose administered to subjects in need thereof may comprise administration of a single mRNA composition described herein.

In some embodiments, a dose administered to subjects in need thereof may comprise administration of at least two or more (including, e.g., at least three or more) different drug products/formulations. For example, in some embodiments, at least two or more different drug products/formulations may comprise at least two different mRNA compositions described herein (e.g., in some embodiments each comprising a different RNA construct).

In some embodiments, a subject is administered two or more RNAs (e.g., as part of either a primary regimen or a booster regimen), wherein the two or more RNAs are administered on the same day or same visit. In some embodiments, the two or more RNAs are administered in separate compositions, e.g., by administering each RNA to a separate part of the subject (e.g., by intramuscular administration to different arms of the subject or to different sites of the same arm of the subject). In some embodiments, the two or more RNAs are mixed prior to administration (e.g., mixed immediately prior to administration, e.g., by the administering practitioner). In some embodiments, the two or more RNAs are formulated together (e.g., by (a) mixing separate populations of LNPs, each population comprising a different RNA; or (b) by mixing two or more RNAs prior to LNP formulation, so that each LNP comprises two or more RNAs).

In some embodiments, a subject is administered or a composition comprises one or more first RNAs and one or more second RNAs, each in the same amount (i.e., at a 1:1 ratio). In some embodiments, a subject is administered or a composition comprises one or more first RNAs and one or more second RNAs, each in a different amount. For example, in some embodiments, a subject is administered or a composition comprises one or more first RNAs in an amount that is 0.01 to 100 times that of one or more second RNAs (e.g., wherein the amount of the one or more first RNAs is 0.01 to 50, 0.01 to 4, 0.01 to 30, 0.01 to 25, 0.01 to 20, to 15, 0.01 to 10, 0.01 to 9, 0.01 to 8, 0.01 to 7, 0.01 to 6, 0.01 to 5, 0.01 to 4, 0.01 to 3, to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times that of the one or more second RNAs).

In some embodiments, a subject is administered or a composition comprises one or more first RNAs and one or more second RNAs, wherein the concentration of the one or more first RNAs is 1 to 10 times that of the one or more second RNAs. In some embodiments, a subject is administered or a composition comprises one or more first RNAs and one or more second RNAs, wherein the amount of the one or more first RNAs is 1 to 5 times that of the one or more second RNAs. In some embodiments, a subject is administered or a composition comprises one or more first RNAs and one or more second RNAs, wherein the concentration of the one or more first RNAs is 1 to 3 times that of the one or more second RNAs. In some embodiments, a subject is administered or a composition comprises one or more first RNAs and one or more second RNAs, wherein the amount of the one or more first RNAs is 2 times that of the one or more second RNAs. In some embodiments, a subject is administered or a composition comprises one or more first RNAs and one or more second RNAs, wherein the concentration of the one or more first RNAs is 3 times that of the one or more second RNAs.

In some embodiments, a subject is administered or a composition comprises two first RNAs, each encoding an antigen derived from an influenza strain or variant, wherein the amount of each RNA is not the same. For example, in some embodiments, the ratio of the two first RNAs is 1:0.01-100 (e.g., 1:0.01-50; 1:0.01-40; 1:0.01-30; 1:0.01-25; 1:0.01-20; 1:0.01-15; 1:1: 0.01-9; 1:0.01-8; 1:0.01-7; 1:0.01-6; 1:0.01-5; 1:0.01-4; 1:0.01-3; 1:0.01-2; 1:1:0.1-10, 1:0.1-5, 1:0.1-3, 1:2-10, 1:2-5, or 1: 2-3). In some embodiments, a subject is administered or a composition comprises two first RNAs at a ratio of 1:3. In some embodiments, a subject is administered or a composition comprises two first RNAs at a ratio of 1:2.

For example, in some embodiments, the ratio of the three first RNAs is 1:0.01-100:0.01-100 (e.g., 1:0.01-50:0.01-50; 1:0.01-40:0.01-40; 1:0.01-30:0.01-30; 1:0.01-25:0.01-25; 1:0.01-20:0.01-20; 1:0.01-15:0.01-15; 1:0.01-10:0.01-10; 1:0.01-9:0.01-9; 1:0.01-8: 0.01-8; 1:0.01-7; 1:0.01-6:0.01-6; 1:0.01-5:0.01-5; 1:0.01-4:0.01-4; 1:0.01-3:0.01-3; 1:0.01-2:0.01-2; 1:0.01-1.5:0.01-1.5; 1:0.1-10:0.1-10, 1:0.1-5:0.1-5, 1:0.1-3:0.1-3, 1:2-10: 2-10, 1:2-5: 2-5, or 1: 2-3:2-3). In some embodiments, a subject is administered or a composition comprises three first RNAs at a ratio of 1:1:3. In some embodiments, a subject is administered or a composition comprises three first RNAs at a ratio of 1:3:3.

In some embodiments, a subject is administered or a composition comprises two or more second RNAs, one or more of which encode an HA protein of a Type A influenza virus, and one or more of which encode an HA protein of a Type B influenza virus. In some embodiments, the one or more second RNAs that encode an HA protein of a Type A influenza virus and the one or more second RNAs that encode an HA protein of a Type B influenza virus are present or are administered in the same amount (i.e., at a ratio of 1:1). In some embodiments, the one more second RNAs that encode an HA protein of a Type A influenza virus and the one or more second RNAs that encode an HA protein of a Type B influenza virus are administered in different amounts (e.g., in a ratio of between 1:10 and 10:1, or in a ratio of 1:2, 1:3, 1:4, 1:5, 2:1, 3:1, 4:1, or 5:1 (total RNA encoding an A antigen:total RNA encoding a B antigen).

In some embodiments, a subject is administered or a composition comprises two second RNAs, each encoding an HA protein of a different influenza virus type (e.g., a second RNA encoding an HA protein of a Type A influenza virus and a second RNA encoding an HA protein of a Type B influenza virus). In some embodiments, the second RNAs are administered or are present in the same amount (i.e., at a 1:1 ratio). In some embodiments, the second RNAs are administered or are present in different amounts (e.g., in a ratio of between 1:10 and 10:1, or in a ratio of 1:2, 1:3, 1:4, 1:5, 2:1, 3:1, 4:1, or 5:1 (A:B)).

In some embodiments, a subject is administered or a composition comprises three second RNAs, each encoding an HA protein of a different influenza virus subtype (e.g., an HA protein of an A/Wisconsin (H1N1) virus, an A/Darwin (H3N2) virus, and a B/Austria (Victoria) virus). In some embodiments, a subject is administered or a composition comprises each of the three second RNAs in the same amount (i.e., at a 1:1:1 ratio). In some embodiments, a subject is administered or a composition comprises a different amount of one or more of the three second RNAs (e.g., in a ratio of between 1:1:2 and 1:1:10 (e.g., in a ratio of 1:1:2, 1:1:3, 1:1:4, or 1:1:5), or in a ratio of between 2:2:1 and 2:2:10, (e.g., in a ratio of 2:2:1, 3:3:1, 4:4:1, or 5:5:1). In some embodiments, a subject is administered or a composition comprises three second RNAs, two of which encode HA proteins of different influenza type A virus, and one of which encodes an HA protein of an influenza type B virus. In some such embodiments, the second RNA encoding an HA protein of an influenza type B virus is present or is administered in a higher amount as compared to either second RNA encoding an HA protein from a type A virus (e.g., in some embodiments, the ratios of the two second RNAs encoding HA proteins from type A influenza viruses relative to the second RNA encoding an HA protein from a type B influenza virus is 1:1:1-10, 1:1:2, 1:1:3, 1:1:4, or 1:1:5 (A:A:B)). In some embodiments, a subject is administered or a composition comprises three second RNAs, two encoding an HA protein of an influenza type A virus and one encoding an HA protein of an influenza type B virus, wherein the ratio of the three second RNAs 1:1:4 (A:A:B). In some embodiments, the two second RNAs encoding an HA protein of an influenza type A viruses are each present or are each administered in a higher amount as compared to the second RNA encoding an HA protein from a type B virus (e.g., in some embodiments, the ratios of the two second RNAs encoding HA proteins from type A influenza viruses relative to the second RNA encoding an HA protein from a type B influenza virus is 1-10:1-10:1, 2:2:1, 3:3:1, 4:4:1, or 5:5:1 (A:A:B)).

In some embodiments, a subject is administered or a composition comprises four second RNAs, each encoding an HA protein of a different influenza virus subtype. In some such embodiments, the four second RNAs comprise two second RNAs encoding HA proteins of different influenza type A viruses and two second RNAs encoding HA proteins of different influenza type B virus (e.g., an HA protein of an H1N1 virus, an HA protein of an H3N2 virus, an HA protein of a B/Victoria lineage virus, and an HA protein of a BNamagata lineage virus). In some embodiments, each of the two second RNAs encoding an HA protein of an influenza type A virus and each of the two second RNAs encoding an HA protein of an influenza type B virus are present in the same amount (i.e., the ratio of the four second RNAs is 1:1:1:1). In some embodiments, the two second RNAs encoding an HA protein of an influenza type B virus are each administered or are each present in a higher amount as compared to either second RNA encoding an HA protein from a type A virus (e.g., in some embodiments, the ratios of the two second RNAs encoding HA proteins from type A influenza viruses relative to the two second RNAs encoding an HA protein from a type B influenza virus is 1:1:2-10:2-10, 1:1:2-5:2-5, 1:1:2:2, 1:1:3:3, 1:1:4:4, 1:1:5:5, 1:1:6:6, 1:1:7:7, 1:1:8:8, 1:1:9:9, 1:1:10:10 (A:A:B:B)). In some embodiments, a subject is administered or a composition comprises four second RNAs, two encoding an HA protein of an influenza type A virus and two encoding an HA protein of an influenza type B virus, wherein the ratio of the four second RNAs 1:1:5:5 (A:A:B:B). In some embodiments, the two second RNAs encoding an HA protein of an influenza type A virus are each administered or are each present in a higher amount as compared to either second RNA encoding an HA protein from a type B virus (e.g., in some embodiments, the ratios of the two second RNAs encoding HA proteins from type A influenza viruses relative to the two second RNAs encoding an HA protein from a type B influenza virus is 2-10:2-10:1:1, 2-5:2-5:1:1, 2:2:1:1, 3:3:1:1, 4:4:1:1, 5:5:1:1, 6:6:1:1, 7:7:1:1, 8:8:1:1, 9:9:1:1, 10:10:1:1 (A:A:B:B)).

In some embodiments, a composition comprises or a subject is administered four second RNAs, comprising three second RNAs that encode HA proteins of different influenza type A viruses and one second RNA encoding an HA protein of an influenza type B virus (e.g., A/Wisconsin (H1N1), A/Darwin (H3N2), A/Cambodia (H3N2), and B/Austria (Victoria)). In some such embodiments, each of the four second RNAs is administered or is present in the same amount (i.e., at a 1:1:1:1 ratio). In some embodiments, the amount of second RNA encoding an HA protein of an influenza type B virus is higher than any one of the second RNAs encoding an HA protein of an influenza type A virus (e.g., in some embodiments, the ratio of second RNAs is 1:1:1:1-10, 1:1:1:1-5, 1:1:1:2, 1:1:1:3, 1:1:1:4, or 1:1:1:5 (A:A:A:B)). In some embodiments, the ratio of second RNAs administered or in a composition is 1:1:1:5 (A:A:A:B).

In some embodiments, the amount of each of the second RNAs encoding an HA protein of an influenza type A virus is higher than that of the second RNA encoding an HA protein of an influenza type B virus (e.g., in some embodiments, the ratio of second RNAs is 1-10:1-10:1-1-5:1-5:1-5:1, 2:2:2:1, 3:3:3:1, 4:4:4:1, or 5:5:5:1 (A:A:A:B)).

In some embodiments, a subject is administered or a composition comprises one or more second RNAs encoding an HA protein of an influenza virus (e.g., two second RNAs, three second RNAs, or four second RNAs, each encoding an HA protein of a different influenza virus) in a total amount of 0.1 to 100 μg (e.g., 1 to 90 μg, 3 to 90 μg, 1 to 60 μg, 3 to 60 μg, 5 to 60 μg, to 60 μg, 30 to 60 μg, 3 to 30 μg). In some embodiments, a subject is administered or a composition comprises one or more second RNAs encoding an HA protein of an influenza virus in a total amount of 3 μg, 5 μg, 6 μg, 10 μg, 15 μg, 20 μg, 25 μg, 30 μg, 45 μg, 60 μg, 75 μg, or 90 μg.

In some embodiments, a subject is administered or a composition comprises three or four second RNAs, each encoding an HA antigen of a different influenza strain, in one of the amounts listed in the below Table C (each “Influenza Component” corresponding to a second RNA encoding an HA antige (e.g., a second RNA as described herein).

In some embodiments, a composition described herein is characterized in that it produces influenza neutralizing antibody titers that are within at least two fold of those produced by a reference vaccine for each influenza virus that it encodes antigens of (e.g., wherein the reference vaccine is a quadrivalent influenza RNA vaccine administered alone, or an approved (non-RNA) influenza vaccine).

In some embodiments, the influenza vaccine is an alphainfluenza virus, a betainfluenza virus, a gammainfluenza virus or a deltainfluenza virus vaccine. In some embodiments the vaccine is an Influenza A virus, an Influenza B virus, an Influenza C virus, or an Influenza D virus vaccine. In some embodiments, the influenza A virus vaccine comprises a hemagglutinin selected from H1, H2, H3, H4, H5, H6, H7, H8, H9, H10, H11, H12, H13, H14, H15, H16, H17, and H18, or an immunogenic fragment or variant of the same, or a nucleic acid (e.g., RNA) encoding any one of the same. In some embodiments the influenza A vaccine comprises or encodes a neuraminidase (NA) selected from N1, N2, N3, N4, N5, N6, N7, N8, N9, N10, and N11, or an immunogenic fragment or variant of the same, or a nucleic acid (e.g., RNA) encoding any one of the same. In some embodiments, the influenza vaccine comprises at least one Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or variant thereof, or a nucleic acid (e.g., RNA) encoding any of one of the same.

### EXAMPLES

### Example 1: Description of Manufacturing Process

A description of the manufacturing process and process controls for the influenza saRNA vaccine drug substances are provided in this section. The manufacturing process includes RNA synthesis via in vitro transcription (IVT) step and a purification step by ultrafiltration/diafiltration (UFDF-1). The RNA is then enzymatically capped and purified by chromatography and a final UFDF-2, followed by final filtration and dispense.

The process has been scaled-up to 1.5 L starting IVT volume scale for manufacturing of clinical material. There were no significant changes from the nonclinical toxicology/development process other than those necessary for process scale-up to 1.5 L.

### RT-ddPCR (Identity of Encoded RNA Sequence)

Identity of the influenza saRNA is confirmed if the tested sample is positive for replicase sequence (confirms self-amplification of RNA construct) and the sequence of interest (confirms encoded flu sequence) upon performing one-step reverse transcription (RT)-ddPCR assay of the RNA in the sample. Digital droplet polymerase chain reaction (ddPCR) technology is a digital form of polymerase chain reaction (PCR) that uses a water-oil emulsion system to quantify target nucleic acids. The RNA sample is diluted to a final theoretical input concentration which is within the linear range of the ddPCR assay. A reaction mixture containing reverse transcriptase, DNA polymerase and sequence specific primers and probes is partitioned into droplets and the PCR reaction is carried out in each partition individually. Results are calculated by counting amplified target sequence (positive droplets, as measured by fluorescence amplitude above background) and the number of partitions in which there is no amplification (negative droplets). Identity is confirmed if positive droplet counts are above an established threshold, after the positive and negative controls have been examined and determined to be valid and acceptable.

### Reversed-Phase HPLC (Presence of Pseudo-Uridine)

Presence of pseudo-Uridine is determined by Reversed-Phase High Performance Liquid Chromatography (RP-HPLC) after complete digestion of the mRNA. The resulting individual nucleosides have a characteristic elution pattern including resolution of uridine and pseudo-uridine. The presence of pseudo-uridine is confirmed by comparison with a uridine and pseudo-uridine reference as well as a limit standard.

### Capillary Gel Electrophoresis (RNA Integrity)

RNA integrity is determined by capillary gel electrophoresis (CGE) based on the differential migration of RNA of different molecular weights in an applied electric field. RNA is subjected to a denaturant that unfolds the RNA and dissociates non-covalent complexes. When subjected to an electric field, the denatured RNA species migrate through the gel matrix, as a function of length and size, toward the anode. An intercalating dye binds to RNA and associated fragments during migration allowing for fluorescence detection. The intact RNA is separated from any fragmented species allowing for the quantitation of RNA integrity by determining the relative percent time corrected area for the intact (main) peak.

qPCR (Residual DNA Template)

The level of residual DNA template is determined by quantitative polymerase chain reaction (qPCR) using fluorescence technology. A qPCR master mix containing target specific primers and fluorescent qPCR quantitation reagent is added to all the sample wells. Samples are prepared in a series of dilutions and are analyzed by qPCR in real-time. The measured fluorescence signal is proportional to the amount of PCR product. The quantitation of DNA is performed during the exponential phase of the reaction at a cycle threshold (Ct) where amplification of a target sequence is first detected above the established signal threshold. This Ct point is dependent on the amount of DNA originally present in the sample. The concentration of DNA in the test sample is interpolated from the linear regression of the standard curve, taking into account the dilution factor. The results are reported in ng DNA/mg RNA.

Batch details and batch analysis summary data are provided in Table 1 for 1 batch of regulatory toxicology material and 1 GMP batch of drug substance to be used in clinical trials.

TABLE 1
S.4.4.6-2. Batch Results for Influenza saRNA Vaccine TC83-delkozak-HA-SGP-NA-80A
Drug Substance

| | | | | Nonclinical | Clinical Drug | |
|---|---|---|---|---|---|---|
| | | | | Toxicology | Substance | |
| Quality | Analytical | Acceptance | | (Batch 00714477- | (Batch | |
| Attribute | Procedure | Criteria | | 0135) | 22V541F101) | |
| Appearance | Clarity | ≤6 | NTU | Clear | 2 | NTU |

| (Clarity) | | | | | | | |
|---|---|---|---|---|---|---|---|
| Appearance | Coloration | Not more | | Colorless solution | | ≤B9 | |
| (Coloration) | | intensely colored | | | | | |
| | | than level 7 of | | | | | |
| | | the brown (B) | | | | | |
| | | color standard. | | | | | |
| pH | Potentiometry | 7.0 ± 0.5 | | 6.7 | | 7.0 | |
| Content (RNA | UV | 1.25 ± 0.25 | | 1.81 | mg/mL | 1.22 | mg/ml |
| concentration) | spectroscopy | mg/ml | | | | | |
| ddPCR | Identity of | Identity | | Positive^{a} | | Confirmed | |
| | Encoded RNA | confirmed | | | | | |
| | Sequence | | | | | | |
| RNA integrity | Capillary gel | ≥60% | | 79% | | 85% | |
| | electrophoresis | | | | | | |
| Residual DNA | qPCR | ≤990 ng | | 31 | ng/mg | NMT 1 ng/mg | |
| template | | DNA/mg RNA | | | | | |
| Endotoxin | Endotoxin | ≤12.5 | EU/mL | <0.06 | EU/mg | 0.2 | EU/mL |

| | (LAL) | | | | |
|---|---|---|---|---|---|
| Bioburden | Bioburden | ≤1 CFU/10 mL | ≤10 | CFU/mL | 0 CFU/10 mL |

^{a}Identity was determined via reverse transcription, quantitative polymerase chain reaction
Specifications only apply to clinical supplies
Abbreviations: NTU = nephelometric turbidity units; NT = not tested; TBP = to be provided in the IND amendment; ddPCR = digital droplet polymerase chain reaction; qPCR = quantitative polymerase chain reaction; LAL = limulus amebocyte lysate; NMT = not more than; EU = Endotoxin unit; CFU = Colony forming unit

### Example 2: S. 4.1 Description and Composition of the Drug Product

The PF-07867246 (Construct 6 (TC83-delkozak-HA-SGP-NA-80A)(SEQ ID NO: 1)) drug product is a preservative-free, sterile dispersion of liquid nanoparticles (LNP) in aqueous cryoprotectant buffer for intramuscular administration. The drug product is formulated at 0.06 mg/mL RNA in 10 mM Tris buffer, 10% sucrose, and optionally 20 mM Glutamic Acid, pH 7.4. The drug product is supplied in a 2 mL glass vial sealed with a chlorobutyl elastomeric stopper and an aluminum seal with flip-off plastic cap (nominal volume of 0.5 mL). The composition of the drug product, including unit formula, amounts per vial, function and quality standard applicable to each component, is given in Table 2.

TABLE 2
P.1-4. Composition of PF-07867246 (TC83-delkozak-HA-SGP-NA-80A) Drug Product

| | | | | | Nominal |
|---|---|---|---|---|---|
| | | | | | Amount or |
| | | | | Filled | Net |
| | | | Unit | Amount | Quantity |
| Name of | Grade/Quality | | Formula | (Total | (Net |
| Ingredient | Standard | Function | (mg/mL) | mg/vial)ª | mg/vial) |
| PF-07867246 | In-house | Active | 0.06 | 0.042 | 0.030 |
| drug substance | specification | ingredient | | | |
| (TC83-delkozak- | | | | | |
| HA-SGP-NA-80A) | | | | | |
| ALC-0315^{b} | In-house | Functional lipid | 0.86 | 0.60 | 0.43 |
| | specification | | | | |
| ALC-0159^{c} | In-house | Functional lipid | 0.11 | 0.08 | 0.06 |
| | specification | | | | |
| DSPC^{d} | In-house | Structural lipid | 0.19 | 0.13 | 0.10 |
| | specification | | | | |
| Cholesterol | Ph. Eur., NF | Structural lipid | 0.37 | 0.26 | 0.19 |
| Sucrose | Ph. Eur., NF | Cryoprotectant | 100 | 70 | 50 |
| Tromethamine^{e} | Ph. Eur., USP | Buffer | 0.20 | 0.14 | 0.10 |
| (Tris base) | | component | | | |
| Tris | | | | | 0.66 |
| (hydroxymethyl) | In-house | Buffer | 1.32 | 0.92 | |
| aminomethane | specification | component | | | |
| hydrochloride | | | | | |
| (Tris HCl) | | | | | |
| Glutamic Acid | FCC, Ph. Eur., | Buffer | 2.94 | 2.06 | 1.47 |
| | JP | component | | | |
| Water for | Ph. Eur., USP, | Solvent | q.s.^{f} to 1.00 | q.s.^{f} to 0.70 | q.s.^{f} to 0.50 |
| Injection | JP | | mL | mL | mL |

^{a}Filled amount includes overfill. This is calculated by multiplying unit formula (mg/mL) by actual fill volume (0.7 mL).
^{b}ALC-0315 = ((4-hydroxybutyl)azanediyl)bis(hexane-6,1-diyl)bis(2-hexyldecanoate)
^{c}ALC-0159 = 2-[(polyethylene glycol)-2000]-N,N-ditetradecylacetamide
^{d}DSPC = 1,2-Distearoyl-sn-glycero-3-phosphocholine
^{e}Also known as Trometamol
^{f}q.s. is an abbreviation for quantum satis meaning as much as is sufficient.
Sodium Hydroxide (Ph. Eur., NF) is used for buffer pH adjustment

TABLE 3
3.6. TC83-delkozak-HA-SGP-NA-80A (A/Wisconsin/588/2019)--VV00050 (Construct 6)
Nucleotide sequence 5′→3′
Sequence length: 10936 nucleotides; 3113 A; 2761 C; 2796 G; 2266 U
A = Adenine; C = Cytosine; G = Guanine; U = Uridine

| | | | | Expected | SEQ ID |
|---|---|---|---|---|---|
| Element | Origin | Start | End | Function | NO: |
| Cap | In vitro (VCE) | 1 | 1 | Provides | |
| | | | | cap1 RNA | |
| | | | | transcripts | |
| 5′ UTR | Venezuelan equine | 2 | 45 | 5′ UTR of | 12 |
| | encephalitis virus | | | RNA | |
| Nsp1 | Venezuelan equine | 46 | 1650 | Non- | 13 |
| | encephalitis virus | | | structural | |
| | (TC83) | | | polyprotein | |
| | | | | subunit of | |
| | | | | the viral RNA | |
| | | | | replicase | |
| Nsp2 | Venezuelan equine | 1651 | 4032 | Non- | 14 |
| | encephalitis virus | | | structural | |
| | (TC83) | | | polyprotein | |
| | | | | subunit of | |
| | | | | the viral RNA | |
| | | | | replicase | |
| Nsp3 | Venezuelan equine | 4033 | 5703 | Non- | 15 |
| | encephalitis virus | | | structural | |
| | (TC83) | | | polyprotein | |
| | | | | subunit of | |
| | | | | the viral RNA | |
| | | | | replicase | |
| Nsp4 | Venezuelan equine | 5704 | 7527 | Non- | 16 |
| | encephalitis virus | | | structural | |
| | (TC83) | | | polyprotein | |
| | | | | subunit of | |
| | | | | the viral RNA | |
| | | | | replicase | |
| VEEV | Venezuelan equine | 7502 | 7562 | Drives | 17 |
| subgenomic | encephalitis virus | | | transcription | |
| promoter | | | | of the flu | |
| | | | | gene | |
| HA_Wisconsin | A/Wisconsin/588/2019 | 7563 | 9268 | Mediates | 18 |
| | H1N1 | | | absorption of | |
| | | | | virus to | |
| | | | | cellular | |
| | | | | receptors | |
| | | | | and aids | |
| | | | | subsequent | |
| | | | | uncoating of | |
| | | | | virus | |
| VEEV | Venezuelan equine | 9269 | 9327 | Drives | 19 |
| subgenomic | encephalitis virus | | | transcription | |
| promoter | | | | of the flu | |
| | | | | gene | |
| NA | neuraminidase | 9328 | 10740 | | 20 |
| 3′ UTR | Venezuelan equine | 10741 | 10857 | 3′ UTR of | 21 |
| | encephalitis virus | | | RNA | |
| polyA (40A) | Artificial | 10858 | 10936 | A poly(A)-tail | 22 |
| | | | | measuring | |
| | | | | 40 | |
| | | | | nucleotides | |
| | | | | in length | |
| | | | | designed to | |
| | | | | enhance | |
| | | | | RNA stability | |

| | |
|---|---|
| Cap-GAUAGGCGGC GCAUGAGAGA AGCCCAGACC AAUUACCUAC CCAAAAUGGA GAAAGUUCAC | 60 |
| GUUGACAUCG AGGAAGACAG CCCAUUCCUC AGAGCUUUGC AGCGGAGCUU CCCGCAGUUU | 120 |
| GAGGUAGAAG CCAAGCAGGU CACUGAUAAU GACCAUGCUA AUGCCAGAGC GUUUUCGCAU | 180 |
| CUGGCUUCAA AACUGAUCGA AACGGAGGUG GACCCAUCCG ACACGAUCCU UGACAUUGGA | 240 |
| AGUGCGCCCG CCCGCAGAAU GUAUUCUAAG CACAAGUAUC AUUGUAUCUG UCCGAUGAGA | 300 |
| UGUGCGGAAG AUCCGGACAG AUUGUAUAAG UAUGCAACUA AGCUGAAGAA AAACUGUAAG | 360 |
| GAAAUAACUG AUAAGGAAUU GGACAAGAAA AUGAAGGAGC UCGCCGCCGU CAUGAGCGAC | 420 |
| CCUGACCUGG AAACUGAGAC UAUGUGCCUC CACGACGACG AGUCGUGUCG CUACGAAGGG | 480 |
| CAAGUCGCUG UUUACCAGGA UGUAUACGCG GUUGACGGAC CGACAAGUCU CUAUCACCAA | 540 |
| GCCAAUAAGG GAGUUAGAGU CGCCUACUGG AUAGGCUUUG ACACCACCCC UUUUAUGUUU | 600 |
| AAGAACUUGG CUGGAGCAUA UCCAUCAUAC UCUACCAACU GGGCCGACGA AACCGUGUUA | 660 |
| ACGGCUCGUA ACAUAGGCCU AUGCAGCUCU GACGUUAUGG AGCGGUCACG UAGAGGGAUG | 720 |
| UCCAUUCUUA GAAAGAAGUA UUUGAAACCA UCCAACAAUG UUCUAUUCUC UGUUGGCUCG | 780 |
| ACCAUCUACC ACGAGAAGAG GGACUUACUG AGGAGCUGGC ACCUGCCGUC UGUAUUUCAC | 840 |
| UUACGUGGCA AGCAAAAUUA CACAUGUCGG UGUGAGACUA UAGUUAGUUG CGACGGGUAC | 900 |
| GUCGUUAAAA GAAUAGCUAU CAGUCCAGGC CUGUAUGGGA AGCCUUCAGG CUAUGCUGCU | 960 |
| ACGAUGCACC GCGAGGGAUU CUUGUGCUGC AAAGUGACAG ACACAUUGAA CGGGGAGAGG | 1020 |
| GUCUCUUUUC CCGUGUGCAC GUAUGUGCCA GCUACAUUGU GUGACCAAAU GACUGGCAUA | 1080 |
| CUGGCAACAG AUGUCAGUGC GGACGACGCG CAAAAACUGC UGGUUGGGCU CAACCAGCGU | 1140 |
| AUAGUCGUCA ACGGUCGCAC CCAGAGAAAC ACCAAUACCA UGAAAAAUUA CCUUUUGCCC | 1200 |
| GUAGUGGCCC AGGCAUUUGC UAGGUGGGCA AAGGAAUAUA AGGAAGAUCA AGAAGAUGAA | 1260 |
| AGGCCACUAG GACUACGAGA UAGACAGUUA GUCAUGGGGU GUUGUUGGGC UUUUAGAAGG | 1320 |
| CACAAGAUAA CAUCUAUUUA UAAGCGCCCG GAUACCCAAA CCAUCAUCAA AGUGAACAGC | 1380 |
| GAUUUCCACU CAUUCGUGCU GCCCAGGAUA GGCAGUAACA CAUUGGAGAU CGGGCUGAGA | 1440 |
| ACAAGAAUCA GGAAAAUGUU AGAGGAGCAC AAGGAGCCGU CACCUCUCAU UACCGCCGAG | 1500 |
| GACGUACAAG AAGCUAAGUG CGCAGCCGAU GAGGCUAAGG AGGUGCGUGA AGCCGAGGAG | 1560 |
| UUGCGCGCAG CUCUACCACC UUUGGCAGCU GAUGUUGAGG AGCCCACUCU GGAAGCCGAU | 1620 |
| GUCGACUUGA UGUUACAAGA GGCUGGGGCC GGCUCAGUGG AGACACCUCG UGGCUUGAUA | 1680 |
| AAGGUUACCA GCUACGAUGG CGAGGACAAG AUCGGCUCUU ACGCUGUGCU UUCUCCGCAG | 1740 |
| GCUGUACUCA AGAGUGAAAA AUUAUCUUGC AUCCACCCUC UCGCUGAACA AGUCAUAGUG | 1800 |
| AUAACACACU CUGGCCGAAA AGGGCGUUAU GCCGUGGAAC CAUACCAUGG UAAAGUAGUG | 1860 |
| GUGCCAGAGG GACAUGCAAU ACCCGUCCAG GACUUUCAAG CUCUGAGUGA AAGUGCCACC | 1920 |
| AUUGUGUACA ACGAACGUGA GUUCGUAAAC AGGUACCUGC ACCAUAUUGC CACACAUGGA | 1980 |
| GGAGCGCUGA ACACUGAUGA AGAAUAUUAC AAAACUGUCA AGCCCAGCGA GCACGACGGC | 2040 |
| GAAUACCUGU ACGACAUCGA CAGGAAACAG UGCGUCAAGA AAGAACUAGU CACUGGGCUA | 2100 |
| GGGCUCACAG GCGAGCUGGU GGAUCCUCCC UUCCAUGAAU UCGCCUACGA GAGUCUGAGA | 2160 |
| ACACGACCAG CCGCUCCUUA CCAAGUACCA ACCAUAGGGG UGUAUGGCGU GCCAGGAUCA | 2220 |
| GGCAAGUCUG GCAUCAUUAA AAGCGCAGUC ACCAAAAAAG AUCUAGUGGU GAGCGCCAAG | 2280 |
| AAAGAAAACU GUGCAGAAAU UAUAAGGGAC GUCAAGAAAA UGAAAGGGCU GGACGUCAAU | 2340 |
| GCCAGAACUG UGGACUCAGU GCUCUUGAAU GGAUGCAAAC ACCCCGUAGA GACCCUGUAU | 2400 |
| AUUGACGAAG CUUUUGCUUG UCAUGCAGGU ACUCUCAGAG CGCUCAUAGC CAUUAUAAGA | 2460 |
| CCUAAAAAGG CAGUGCUCUG CGGGGAUCCC AAACAGUGCG GUUUUUUUAA CAUGAUGUGC | 2520 |
| CUGAAAGUGC AUUUUAACCA CGAGAUUUGC ACACAAGUCU UCCACAAAAG CAUCUCUCGC | 2580 |
| CGUUGCACUA AAUCUGUGAC UUCGGUCGUC UCAACCUUGU UUUACGACAA AAAAAUGAGA | 2640 |
| ACGACGAAUC CGAAAGAGAC UAAGAUUGUG AUUGACACUA CCGGCAGUAC CAAACCUAAG | 2700 |
| CAGGACGAUC UCAUUCUCAC UUGUUUCAGA GGGUGGGUGA AGCAGUUGCA AAUAGAUUAC | 2760 |
| AAAGGCAACG AAAUAAUGAC GGCAGCUGCC UCUCAAGGGC UGACCCGUAA AGGUGUGUAU | 2820 |
| GCCGUUCGGU ACAAGGUGAA UGAAAAUCCU CUGUACGCAC CCACCUCAGA ACAUGUGAAC | 2880 |
| GUCCUACUGA CCCGCACGGA GGACCGCAUC GUGUGGAAAA CACUAGCCGG CGACCCAUGG | 2940 |
| AUAAAAACAC UGACUGCCAA GUACCCUGGG AAUUUCACUG CCACGAUAGA GGAGUGGCAA | 3000 |
| GCAGAGCAUG AUGCCAUCAU GAGGCACAUC UUGGAGAGAC CGGACCCUAC CGACGUCUUC | 3060 |
| CAGAAUAAGG CAAACGUGUG UUGGGCCAAG GCUUUAGUGC CGGUGCUGAA GACCGCUGGC | 3120 |
| AUAGACAUGA CCACUGAACA AUGGAACACU GUGGAUUAUU UUGAAACGGA CAAAGCUCAC | 3180 |
| UCAGCAGAGA UAGUAUUGAA CCAACUAUGC GUGAGGUUCU UUGGACUCGA UCUGGACUCC | 3240 |
| GGUCUAUUUU CUGCACCCAC UGUUCCGUUA UCCAUUAGGA AUAAUCACUG GGAUAACUCC | 3300 |
| CCGUCGCCUA ACAUGUACGG GCUGAAUAAA GAAGUGGUCC GUCAGCUCUC UCGCAGGUAC | 3360 |
| CCACAACUGC CUCGGGCAGU UGCCACUGGA AGAGUCUAUG ACAUGAACAC UGGUACACUG | 3420 |
| CGCAAUUAUG AUCCGCGCAU AAACCUAGUA CCUGUAAACA GAAGACUGCC UCAUGCUUUA | 3480 |
| GUCCUCCACC AUAAUGAACA CCCACAGAGU GACUUUUCUU CAUUCGUCAG CAAAUUGAAG | 3540 |
| GGCAGAACUG UCCUGGUGGU CGGGGAAAAG UUGUCCGUCC CAGGCAAAAU GGUUGACUGG | 3600 |
| UUGUCAGACC GGCCUGAGGC UACCUUCAGA GCUCGGCUGG AUUUAGGCAU CCCAGGUGAU | 3660 |
| GUGCCCAAAU AUGACAUAAU AUUUGUUAAU GUGAGGACCC CAUAUAAAUA CCAUCACUAU | 3720 |
| CAGCAGUGUG AAGACCAUGC CAUUAAGCUU AGCAUGUUGA CCAAGAAAGC UUGUCUGCAU | 3780 |
| CUGAAUCCCG GCGGAACCUG UGUCAGCAUA GGUUAUGGUU ACGCUGACAG GGCCAGCGAA | 3840 |
| AGCAUCAUUG GUGCUAUAGC GCGGCAGUUC AAGUUUUCCC GGGUAUGCAA ACCGAAAUCC | 3900 |
| UCACUUGAAG AGACGGAAGU UCUGUUUGUA UUCAUUGGGU ACGAUCGCAA GGCCCGUACG | 3960 |
| CACAAUCCUU ACAAGCUUUC AUCAACCUUG ACCAACAUUU AUACAGGUUC CAGACUCCAC | 4020 |
| GAAGCCGGAU GUGCACCCUC AUAUCAUGUG GUGCGAGGGG AUAUUGCCAC GGCCACCGAA | 4080 |
| GGAGUGAUUA UAAAUGCUGC UAACAGCAAA GGACAACCUG GCGGAGGGGU GUGCGGAGCG | 4140 |
| CUGUAUAAGA AAUUCCCGGA AAGCUUCGAU UUACAGCCGA UCGAAGUAGG AAAAGCGCGA | 4200 |
| CUGGUCAAAG GUGCAGCUAA ACAUAUCAUU CAUGCCGUAG GACCAAACUU CAACAAAGUU | 4260 |
| UCGGAGGUUG AAGGUGACAA ACAGUUGGCA GAGGCUUAUG AGUCCAUCGC UAAGAUUGUC | 4320 |
| AACGAUAACA AUUACAAGUC AGUAGCGAUU CCACUGUUGU CCACCGGCAU CUUUUCCGGG | 4380 |
| AACAAAGAUC GACUAACCCA AUCAUUGAAC CAUUUGCUGA CAGCUUUAGA CACCACUGAU | 4440 |
| GCAGAUGUAG CCAUAUACUG CAGGGACAAG AAAUGGGAAA UGACUCUCAA GGAAGCAGUG | 4500 |
| GCUAGGAGAG AAGCAGUGGA GGAGAUAUGC AUAUCCGACG ACUCUUCAGU GACAGAACCU | 4560 |
| GAUGCAGAGC UGGUGAGGGU GCAUCCGAAG AGUUCUUUGG CUGGAAGGAA GGGCUACAGC | 4620 |
| ACAAGCGAUG GCAAAACUUU CUCAUAUUUG GAAGGGACCA AGUUUCACCA GGCGGCCAAG | 4680 |
| GAUAUAGCAG AAAUUAAUGC CAUGUGGCCC GUUGCAACGG AGGCCAAUGA GCAGGUAUGC | 4740 |
| AUGUAUAUCC UCGGAGAAAG CAUGAGCAGU AUUAGGUCGA AAUGCCCCGU CGAAGAGUCG | 4800 |
| GAAGCCUCCA CACCACCUAG CACGCUGCCU UGCUUGUGCA UCCAUGCCAU GACUCCAGAA | 4860 |
| AGAGUACAGC GCCUAAAAGC CUCACGUCCA GAACAAAUUA CUGUGUGCUC AUCCUUUCCA | 4920 |
| UUGCCGAAGU AUAGAAUCAC UGGUGUGCAG AAGAUCCAAU GCUCCCAGCC UAUAUUGUUC | 4980 |
| UCACCGAAAG UGCCUGCGUA UAUUCAUCCA AGGAAGUAUC UCGUGGAAAC ACCACCGGUA | 5040 |
| GACGAGACUC CGGAGCCAUC GGCAGAGAAC CAAUCCACAG AGGGGACACC UGAACAACCA | 5100 |
| CCACUUAUAA CCGAGGAUGA GACCAGGACU AGAACGCCUG AGCCGAUCAU CAUCGAAGAG | 5160 |
| GAAGAAGAGG AUAGCAUAAG UUUGCUGUCA GAUGGCCCGA CCCACCAGGU GCUGCAAGUC | 5220 |
| GAGGCAGACA UUCACGGGCC GCCCUCUGUA UCUAGCUCAU CCUGGUCCAU UCCUCAUGCA | 5280 |
| UCCGACUUUG AUGUGGACAG UUUAUCCAUA CUUGACACCC UGGAGGGAGC UAGCGUGACC | 5340 |
| AGCGGGGCAA CGUCAGCCGA GACUAACUCU UACUUCGCAA AGAGUAUGGA GUUUCUGGCG | 5400 |
| CGACCGGUGC CUGCGCCUCG AACAGUAUUC AGGAACCCUC CACAUCCCGC UCCGCGCACA | 5460 |
| AGAACACCGU CACUUGCACC CAGCAGGGCC UGCUCGAGAA CCAGCCUAGU UUCCACCCCG | 5520 |
| CCAGGCGUGA AUAGGGUGAU CACUAGAGAG GAGCUCGAGG CGCUUACCCC GUCACGCACU | 5580 |
| CCUAGCAGGU CGGUCUCGAG AACCAGCCUG GUCUCCAACC CGCCAGGCGU AAAUAGGGUG | 5640 |
| AUUACAAGAG AGGAGUUUGA GGCGUUCGUA GCACAACAAC AAUGACGGUU UGAUGCGGGU | 5700 |
| GCAUACAUCU UUUCCUCCGA CACCGGUCAA GGGCAUUUAC AACAAAAAUC AGUAAGGCAA | 5760 |
| ACGGUGCUAU CCGAAGUGGU GUUGGAGAGG ACCGAAUUGG AGAUUUCGUA UGCCCCGCGC | 5820 |
| CUCGACCAAG AAAAAGAAGA AUUACUACGC AAGAAAUUAC AGUUAAAUCC CACACCUGCU | 5880 |
| AACAGAAGCA GAUACCAGUC CAGGAAGGUG GAGAACAUGA AAGCCAUAAC AGCUAGACGU | 5940 |
| AUUCUGCAAG GCCUAGGGCA UUAUUUGAAG GCAGAAGGAA AAGUGGAGUG CUACCGAACC | 6000 |
| CUGCAUCCUG UUCCUUUGUA UUCAUCUAGU GUGAACCGUG CCUUUUCAAG CCCCAAGGUC | 6060 |
| GCAGUGGAAG CCUGUAACGC CAUGUUGAAA GAGAACUUUC CGACUGUGGC UUCUUACUGU | 6120 |
| AUUAUUCCAG AGUACGAUGC CUAUUUGGAC AUGGUUGACG GAGCUUCAUG CUGCUUAGAC | 6180 |
| ACUGCCAGUU UUUGCCCUGC AAAGCUGCGC AGCUUUCCAA AGAAACACUC CUAUUUGGAA | 6240 |
| CCCACAAUAC GAUCGGCAGU GCCUUCAGCG AUCCAGAACA CGCUCCAGAA CGUCCUGGCA | 6300 |
| GCUGCCACAA AAAGAAAUUG CAAUGUCACG CAAAUGAGAG AAUUGCCCGU AUUGGAUUCG | 6360 |
| GCGGCCUUUA AUGUGGAAUG CUUCAAGAAA UAUGCGUGUA AUAAUGAAUA UUGGGAAACG | 6420 |
| UUUAAAGAAA ACCCCAUCAG GCUUACUGAA GAAAACGUGG UAAAUUACAU UACCAAAUUA | 6480 |
| AAAGGACCAA AAGCUGCUGC UCUUUUUGCG AAGACACAUA AUUUGAAUAU GUUGCAGGAC | 6540 |
| AUACCAAUGG ACAGGUUUGU AAUGGACUUA AAGAGAGACG UGAAAGUGAC UCCAGGAACA | 6600 |
| AAACAUACUG AAGAACGGCC CAAGGUACAG GUGAUCCAGG CUGCCGAUCC GCUAGCAACA | 6660 |
| GCGUAUCUGU GCGGAAUCCA CCGAGAGCUG GUUAGGAGAU UAAAUGCGGU CCUGCUUCCG | 6720 |
| AACAUUCAUA CACUGUUUGA UAUGUCGGCU GAAGACUUUG ACGCUAUUAU AGCCGAGCAC | 6780 |
| UUCCAGCCUG GGGAUUGUGU UCUGGAAACU GACAUCGCGU CGUUUGAUAA AAGUGAGGAC | 6840 |
| GACGCCAUGG CUCUGACCGC GUUAAUGAUU CUGGAAGACU UAGGUGUGGA CGCAGAGCUG | 6900 |
| UUGACGCUGA UUGAGGCGGC UUUCGGCGAA AUUUCAUCAA UACAUUUGCC CACUAAAACU | 6960 |
| AAAUUUAAAU UCGGAGCCAU GAUGAAAUCU GGAAUGUUCC UCACACUGUU UGUGAACACA | 7020 |
| GUCAUUAACA UUGUAAUCGC AAGCAGAGUG UUGAGAGAAC GGCUAACCGG AUCACCAUGU | 7080 |
| GCAGCAUUCA UUGGAGAUGA CAAUAUCGUG AAAGGAGUCA AAUCGGACAA AUUAAUGGCA | 7140 |
| GACAGGUGCG CCACCUGGUU GAAUAUGGAA GUCAAGAUUA UAGAUGCUGU GGUGGGCGAG | 7200 |
| AAAGCGCCUU AUUUCUGUGG AGGGUUUAUU UUGUGUGACU CCGUGACCGG CACAGCGUGC | 7260 |
| CGUGUGGCAG ACCCCCUAAA AAGGCUGUUU AAGCUUGGCA AACCUCUGGC AGCAGACGAU | 7320 |
| GAACAUGAUG AUGACAGGAG AAGGGCAUUG CAUGAAGAGU CAACACGCUG GAACCGAGUG | 7380 |
| GGUAUUCUUU CAGAGCUGUG CAAGGCAGUA GAAUCAAGGU AUGAAACCGU AGGAACUUCC | 7440 |
| AUCAUAGUUA UGGCCAUGAC UACUCUAGCU AGCAGUGUUA AAUCAUUCAG CUACCUGAGA | 7500 |
| GGGGCCCCUA UAACUCUCUA CGGCUAACCU GAAUGGACUA CGACAUAGUC UAGUCCGCCA | 7560 |
| AGAUGAAGGC CAUCCUGGUG GUCAUGCUGU ACACCUUCAC CACCGCCAAC GCCGACACAC | 7620 |
| UGUGUAUCGG CUACCACGCC AACAACAGCA CCGACACCGU GGAUACCGUG CUGGAAAAGA | 7680 |
| ACGUGACCGU GACACACAGC GUGAACCUGC UGGAAGAUAA GCACAACGGC AAGCUGUGCA | 7740 |
| AGCUGAGAGG CGUGGCACCU CUGCACCUGG GCAAGUGUAA UAUCGCCGGC UGGAUCCUGG | 7800 |
| GCAACCCUGA GUGUGAAAGC CUGAGCACCG CCAGAUCCUG GUCCUACAUC GUGGAAACCA | 7860 |
| GCAACAGCGA CAACGGCACA UGCUACCCCG GCGACUUCAU CAACUACGAG GAACUGCGGG | 7920 |
| AACAGCUGAG CAGCGUGUCC AGCUUCGAGA GAUUCGAGAU CUUCCCCAAG ACCAGCAGCU | 7980 |
| GGCCCAACCA CGACUCUGAC AAUGGCGUGA CAGCCGCCUG UCCUCAUGCC GGCGCUAAGA | 8040 |
| GCUUCUACAA GAACCUGAUC UGGCUGGUCA AGAAGGGCAA GAGCUACCCC AAGAUCAACC | 8100 |
| AGACCUACAU CAACGACAAG GGCAAAGAGG UGCUGGUCCU CUGGGGCAUC CACCAUCCUC | 8160 |
| CAACAAUCGC CGAUCAGCAG AGCCUGUACC AGAACGCCGA UGCCUAUGUG UUCGUGGGCA | 8220 |
| CCAGCCGGUA CAGCAAGAAG UUCAAGCCCG AGAUCGCCAC CAGGCCUAAA GUGCGGGAUC | 8280 |
| AAGAGGGCAG AAUGAACUAC UACUGGACCC UGGUGGAACC CGGCGACAAG AUCACAUUUG | 8340 |
| AGGCCACCGG CAAUCUGGUG GCCCCUAGAU ACGCCUUCAC CAUGGAAAGA GAUGCCGGCA | 8400 |
| GCGGCAUCAU CAUCAGCGAU ACCCCUGUGC ACGACUGCAA CACCACCUGU CAGACACCUG | 8460 |
| AGGGCGCCAU CAAUACCAGC CUGCCUUUCC AGAACGUGCA CCCCAUCACC AUCGGCAAGU | 8520 |
| GCCCCAAAUA CGUGAAGUCC ACCAAGCUGA GGCUGGCCAC AGGCCUGAGA AAUGUGCCCU | 8580 |
| CCAUCCAGAG CAGAGGCCUG UUUGGAGCCA UUGCCGGCUU UAUCGAAGGC GGCUGGACAG | 8640 |
| GCAUGGUGGA CGGAUGGUAC GGAUACCACC ACCAGAACGA GCAAGGCUCU GGCUAUGCCG | 8700 |
| CCGACCUGAA GUCUACCCAG AAUGCCAUCG AUAAGAUCAC CAACAAAGUG AACAGCGUGA | 8760 |
| UCGAGAAGAU GAACACCCAG UUCACCGCCG UGGGAAAAGA GUUCAACCAC CUGGAAAAGC | 8820 |
| GCAUCGAGAA CCUGAACAAG AAGGUGGACG ACGGCUUCCU GGACAUCUGG ACCUACAAUG | 8880 |
| CCGAACUGCU GGUGCUGCUG GAGAACGAGA GAACCCUGGA CUACCACGAC AGCAACGUGA | 8940 |
| AGAACCUGUA CGAGAAAGUG CGCAACCAGC UGAAGAACAA CGCCAAAGAG AUCGGCAACG | 9000 |
| GCUGCUUCGA GUUCUACCAC AAGUGCGACA AUACCUGCAU GGAAAGCGUG AAGAAUGGCA | 9060 |
| CCUACGACUA CCCUAAGUAC AGCGAGGAAG CCAAGCUGAA CCGCGAGAAG AUCGACGGCG | 9120 |
| UGAAGCUGGA UAGCACCCGG AUCUACCAGA UUCUGGCCAU CUACAGCACC GUGGCCUCUA | 9180 |
| GCCUGGUGCU GGUGGUUUCU CUGGGCGCUA UCAGCUUCUG GAUGUGCAGC AAUGGCAGCC | 9240 |
| UGCAGUGCCG GAUCUGCAUC UGAUGAGGGC CCCUAUAACU CUCUACGGCU AACCUGAAUG | 9300 |
| GACUACGACA UAGUCUAGUC CGCCAAGAUG AACCCCAACC AGAAGAUCAU CACCAUCGGC | 9360 |
| AGCAUCUGCA UGACAAUCGG CACCGCCAAC CUGAUCCUGC AGAUCGGCAA CAUCAUCAGC | 9420 |
| AUCUGGGUGU CCCACAGCAU CCAGAUCGGA AACCAGAGCC AGAUCGAGAC AUGCAACAAG | 9480 |
| AGCGUGAUCA CCUACGAGAA CAACACCUGG GUCAACCAGA CCUUCGUGAA CAUCAGCAAC | 9540 |
| ACCAACAGCG CCGCCAGACA GUCUGUGGCC UCUGUGAAAC UGGCCGGCAA CAGCUCUCUG | 9600 |
| UGUCCUGUGU CUGGCUGGGC CAUCUACAGC AAGGACAACU CUGUGCGGAU CGGCUCCAAG | 9660 |
| GGCGACGUGU UCGUGAUCAG AGAGCCCUUC AUCAGCUGCA GCCCUCUGGA AUGCCGGACA | 9720 |
| UUCUUUCUGA CCCAAGGCGC CCUGCUGAAC GACAAGCACA GCAACGGCAC CAUCAAGGAC | 9780 |
| AGAAGCCCCU ACAGAACCCU GAUGAGCUGC CCUAUCGGCG AGGUGCCCUC UCCAUACAAC | 9840 |
| AGCAGAUUCG AGUCCGUGGC UUGGAGCGCC UCUGCCUGUC ACGAUGGCAC CAACUGGCUG | 9900 |
| ACCAUCGGAA UCAGCGGACC UGAUUCUGGC GCUGUGGCCG UGCUGAAGUA CAAUGGCAUC | 9960 |
| AUCACCGAUA CCAUCAAGAG CUGGCGGAAC AAGAUCCUGC GGACCCAAGA GUCCGAGUGC | 10020 |
| GCCUGUGUGA AUGGCAGCUG CUUCACCAUC AUGACAGACG GCCCAUCUGA UGGCCAGGCC | 10080 |
| AGCUACAAGA UCUUCCGGAU CGAGAAGGGC AAGAUCAUUA AGAGCGUGGA AAUGAAGGCC | 10140 |
| CCGAACUACC ACUACGAGGA AUGCAGCUGU UACCCCGACA GCAGCGAGAU CACCUGUGUG | 10200 |
| UGCAGAGACA ACUGGCACGG CAGCAACAGA CCUUGGGUGU CCUUCAACCA GAACCUGGAA | 10260 |
| UACCAGAUGG GCUACAUCUG CAGCGGCGUG UUCGGCGACA ACCCCAGACC UAAUGAUAAG | 10320 |
| ACCGGCAGCU GCGGCCCUGU GUCUAGCAAU GGUGCCAAUG GCGUGAAGGG CUUCAGCUUU | 10380 |
| AAGUACGGCA ACGGCGUGUG GAUCGGCCGG ACAAAGAGCA UCAGCAGCAG AAAGGGCUUC | 10440 |
| GAGAUGAUCU GGGACCCCAA UGGCUGGACC GGCACCGACA ACAAGUUCAG CAAGAAACAG | 10500 |
| GACAUCGUGG GCAUCAACGA GUGGAGCGGC UACAGCGGCU CUUUCGUGCA GCACCCUGAA | 10560 |
| CUGACCGGCC UGAACUGCAU CAGACCCUGC UUUUGGGUCG AGCUGAUCCG GGGCAGACCC | 10620 |
| GAGGAAAACA CCAUCUGGAC AAGCGGCAGC AGCAUCAGCU UUUGCGGCGU GGACUCUGAC | 10680 |
| AUCGUCGGCU GGUCUUGGCC UGAUGGCGCC GAGCUGCCUU UCACCAUCGA CAAGUGAUGA | 10740 |
| AUACAGCAGC AAUUGGCAAG CUGCUUACAU AGAACUCGCG GCGAUUGGCA UGCCGCCUUA | 10800 |
| AAAUUUUUAU UUUAUUUUUC UUUUCUUUUC CGAAUCGGAU UUUGUUUUUA AUAUUUCAAA | 10860 |
| AAAAAAAAAA AAAAAAAAAA AAAAAAAAAA AAAAAAAAAA AAAAAAAAAA AAAAAAAAAA | 10920 |
| AAAAAAAAAA AAAAAA (SEQ ID NO: 1) | 10936 |

### Example 3: S.4.1. Description and Composition of the Drug Product

The PF-07871987 (construct 7 TC83-HA-40A 50U-50pU (SEQ ID NO: 2)) drug product is a preservative-free, sterile dispersion of liquid nanoparticles (LNP) in aqueous cryoprotectant buffer for intramuscular administration. The drug product is formulated at 0.06 mg/mL RNA in 10 mM Tris buffer, 10% sucrose, and optionally 20 mM glutamic acid, pH 7.4.

The drug product is supplied in a 2 mL glass vial sealed with a chlorobutyl elastomeric stopper and an aluminum seal with flip-off plastic cap (nominal volume of 0.5 mL).

The composition of the drug product, including unit formula, amounts per vial, function and quality standard applicable to each component, is given in Table 4.

TABLE 4
P.1-5. Composition of PF-07871987 Drug Product (TC83-HA-40A 50U-50pU)

| | | | | Filled | |
|---|---|---|---|---|---|
| | | | Unit | Amount | Unit |
| Name of | Grade/Quality | | Formula | (Total | Formulation |
| Ingredient | Standard | Function | (mg/ml) | mg/vial)^{a} | (mg/vial) |
| PF-07871987 | In-house | Active | 0.06 | 0.042 | 0.030 |
| drug substance | specification | ingredient | | | |
| (TC83-HA-40A | | | | | |
| 50U-50pU) | | | | | |
| ALC-0315^{b} | In-house | Functional lipid | 0.86 | 0.60 | 0.43 |
| | specification | | | | |
| ALC-0159^{c} | In-house | Functional lipid | 0.11 | 0.08 | 0.06 |
| | specification | | | | |
| DSPC^{d} | In-house | Structural lipid | 0.19 | 0.13 | 0.10 |
| | specification | | | | |
| Cholesterol | Ph. Eur., NF | Structural lipid | 0.37 | 0.26 | 0.19 |
| Sucrose | Ph. Eur., NF | Cryoprotectant | 100 | 70 | 50 |
| Tromethamine^{e} | Ph. Eur., USP | Buffer | 0.20 | 0.14 | 0.10 |
| (Tris base) | | component | | | |
| Tris | | | | | |
| (hydroxymethyl) | In-house | Buffer | 1.32 | 0.92 | 0.66 |
| aminomethane | specification | component | | | |
| hydrochloride | | | | | |
| (Tris HCl) | | | | | |
| Glutamic Acid | FCC, Ph. Eur., | Buffer | 2.94 | 2.06 | 1.47 |
| | JP | component | | | |
| Water for | Ph. Eur., USP, | Solvent | q.s.^{f} to 1.00 | q.s.^{f} to 0.70 | q.s.^{f} to 0.50 |
| Injection | JP | | mL | mL | mL |

g. Filled amount includes overfill. This is calculated by multiplying unit formula (mg/mL) by actual fillvolume (0.7 mL).
h. ALC-0315 = ((4-hydroxybutyl)azanediyl)bis(hexane-6,1-diyl)bis(2-hexyldecanoate)
i. ALC-0159 = 2-[(polyethylene glycol)-2000]-N,N-ditetradecylacetamide
j. DSPC = 1,2-Distearoyl-sn-glycero-3-phosphocholine
k. Also known as Trometamol
l. q.s. is an abbreviation for quantum satis meaning as much as is sufficient.
Sodium Hydroxide (Ph. Eur., NF) is used for buffer pH adjustment

TABLE 5
S.4.4.6-4. Batch Results for Influenza saRNA Vaccine TC83-HA-40A 50U-50pU
Drug Substance

| | | | | Nonclinical | Clinical Drug | |
|---|---|---|---|---|---|---|
| | | | | Toxicology | Substance | |
| Quality | Analytical | Acceptance | | (Batch | (Batch | |
| Attribute | Procedure | Criteria | | 00710958-0369) | 22V543F101) | |
| Appearance | Clarity | ≤6 | NTU | Clear | 0 | NTU |

| (Clarity) | | | | | | |
|---|---|---|---|---|---|---|
| Appearance | Coloration | Not more | Colorless solution | | ≤B9 | |
| (Coloration) | | intensely colored | | | | |
| | | than level 7 of | | | | |
| | | the brown (B) | | | | |
| | | color standard. | | | | |
| pH | Potentiometry | 7.0 ± 0.5 | 7.1 | | 6.9 | |
| Content (RNA | UV | 1.25 ± 0.25 | 2.00 | mg/mL | 1.27 | mg/ml |

| concentration) | spectroscopy | mg/mL | | | | |
|---|---|---|---|---|---|---|
| ddPCR | Identity of | Identity | | Positivea | | Confirmed |
| | Encoded RNA | confirmed | | | | |
| | Sequence | | | | | |
| Presence of | RP-HPLC | Identity | | Confirmedb | | Confirmed |
| pseudoU | | confirmed | | | | |
| RNA integrity | Capillary gel | ≥60% | | 88% | | 87% |
| | electrophoresis | | | | | |
| Residual DNA | qPCR | ≤990 ng | | 37 | ng/mg | NMT 1 ng/mg |
| template | | DNA/mg RNA | | | | |
| Endotoxin | Endotoxin | ≤12.5 | EU/mL | <0.025 | EU/mg | NMT 0.2 EU/mL |

| | (LAL) | | | | |
|---|---|---|---|---|---|
| Bioburden | Bioburden | ≤1 CFU/10 mL | ≤10 | CFU/mL | 0 CFU/10 mL |

^{a}Identity was determined via reverse transcription, quantitative polymerase chain reaction
^{b}Nonclinical toxicology reported an approximate percentage of pseudoU. Its presence can be inferred to be “confirmed” from the reported result of it being present.
Specifications only apply to clinical supplies
Abbreviations: NTU = nephelometric turbidity units; NT = not tested; TBP = to be provided in the IND amendment; ddPCR = digital droplet polymerase chain reaction; qPCR = quantitative polymerase chain reaction; LAL = limulus amebocyte lysate; NMT = not more than; EU = Endotoxin unit; CFU = Colony forming unit

TABLE 6
Sequence 3.1. TC83-HA-40-50U-50pU (A/Wisconsin/588/2019) pKT177 with
50%U + 50% pseudo
Construct 7
Nucleotide sequence 5′→3′
Sequence length: 9433 nucleotides; 2703 A; 2327 C; 2396 G; 2007 U
A = Adenine; C = Cytosine; G = Guanine; (*)U = Uridine or N1-Methylpseudouridine
(*) The RNA derived from this construct is composed of 50% Uridine and 50% N1
Methylpseudouridine.

| | | | | | SEQ |
|---|---|---|---|---|---|
| Element | Origin | Start | End | Expected Function | ID NO: |
| Cap | In vitro (VCE) | 1 | 1 | Provides cap1 RNA | |
| | | | | transcripts | |
| 5′ UTR | Venezuelan equine | 2 | 45 | 5′ UTR of RNA | 23 |
| | encephalitis virus | | | | |
| Nsp1 | Venezuelan equine | 46 | 1650 | Non-structural | 24 |
| | encephalitis virus | | | polyprotein subunit of | |
| | (TC83) | | | the viral RNA | |
| | | | | replicase | |
| Nsp2 | Venezuelan equine | 1651 | 4032 | Non-structural | 25 |
| | encephalitis virus | | | polyprotein subunit of | |
| | (TC83) | | | the viral RNA | |
| | | | | replicase | |
| Nsp3 | Venezuelan equine | 4033 | 5703 | Non-structural | 26 |
| | encephalitis virus | | | polyprotein subunit of | |
| | (TC83) | | | the viral RNA | |
| | | | | replicase | |
| Nsp4 | Venezuelan equine | 5704 | 7527 | Non-structural | 27 |
| | encephalitis virus | | | polyprotein subunit of | |
| | (TC83) | | | the viral RNA | |
| | | | | replicase | |
| VEEV | Venezuelan equine | 7502 | 7562 | Drives transcription of | 28 |
| subgenomic | encephalitis virus | | | the flu gene | |
| promoter | | | | | |
| Kozak | Eukaryotic origin | 7563 | 7572 | Protein translation | 29 |
| Sequence | | | | initiation site | |
| HA_Wisconsin | A/Wisconsin/588/2019 | 7573 | 9276 | Mediates absorption | 30 |
| | H1N1 | | | of virus to cellular | |
| | | | | receptors and aids | |
| | | | | subsequent uncoating | |
| | | | | of virus | |
| 3′ UTR | Venezuelan equine | 9277 | 9393 | 3′ UTR of RNA | 31 |
| | encephalitis virus | | | | |
| polyA (40A) | Artificial | 9394 | 9433 | A poly(A)-tail | 32 |
| | | | | measuring 40 | |
| | | | | nucleotides in length | |
| | | | | designed to enhance | |
| | | | | RNA stability | |

| | |
|---|---|
| Cap-GAUAGGCGGC GCAUGAGAGA AGCCCAGACC AAUUACCUAC CCAAAAUGGA GAAAGUUCAC | 60 |
| GUUGACAUCG AGGAAGACAG CCCAUUCCUC AGAGCUUUGC AGCGGAGCUU CCCGCAGUUU | 120 |
| GAGGUAGAAG CCAAGCAGGU CACUGAUAAU GACCAUGCUA AUGCCAGAGC GUUUUCGCAU | 180 |
| CUGGCUUCAA AACUGAUCGA AACGGAGGUG GACCCAUCCG ACACGAUCCU UGACAUUGGA | 240 |
| AGUGCGCCCG CCCGCAGAAU GUAUUCUAAG CACAAGUAUC AUUGUAUCUG UCCGAUGAGA | 300 |
| UGUGCGGAAG AUCCGGACAG AUUGUAUAAG UAUGCAACUA AGCUGAAGAA AAACUGUAAG | 360 |
| GAAAUAACUG AUAAGGAAUU GGACAAGAAA AUGAAGGAGC UCGCCGCCGU CAUGAGCGAC | 420 |
| CCUGACCUGG AAACUGAGAC UAUGUGCCUC CACGACGACG AGUCGUGUCG CUACGAAGGG | 480 |
| CAAGUCGCUG UUUACCAGGA UGUAUACGCG GUUGACGGAC CGACAAGUCU CUAUCACCAA | 540 |
| GCCAAUAAGG GAGUUAGAGU CGCCUACUGG AUAGGCUUUG ACACCACCCC UUUUAUGUUU | 600 |
| AAGAACUUGG CUGGAGCAUA UCCAUCAUAC UCUACCAACU GGGCCGACGA AACCGUGUUA | 660 |
| ACGGCUCGUA ACAUAGGCCU AUGCAGCUCU GACGUUAUGG AGCGGUCACG UAGAGGGAUG | 720 |
| UCCAUUCUUA GAAAGAAGUA UUUGAAACCA UCCAACAAUG UUCUAUUCUC UGUUGGCUCG | 780 |
| ACCAUCUACC ACGAGAAGAG GGACUUACUG AGGAGCUGGC ACCUGCCGUC UGUAUUUCAC | 840 |
| UUACGUGGCA AGCAAAAUUA CACAUGUCGG UGUGAGACUA UAGUUAGUUG CGACGGGUAC | 900 |
| GUCGUUAAAA GAAUAGCUAU CAGUCCAGGC CUGUAUGGGA AGCCUUCAGG CUAUGCUGCU | 960 |
| ACGAUGCACC GCGAGGGAUU CUUGUGCUGC AAAGUGACAG ACACAUUGAA CGGGGAGAGG | 1020 |
| GUCUCUUUUC CCGUGUGCAC GUAUGUGCCA GCUACAUUGU GUGACCAAAU GACUGGCAUA | 1080 |
| CUGGCAACAG AUGUCAGUGC GGACGACGCG CAAAAACUGC UGGUUGGGCU CAACCAGCGU | 1140 |
| AUAGUCGUCA ACGGUCGCAC CCAGAGAAAC ACCAAUACCA UGAAAAAUUA CCUUUUGCCC | 1200 |
| GUAGUGGCCC AGGCAUUUGC UAGGUGGGCA AAGGAAUAUA AGGAAGAUCA AGAAGAUGAA | 1260 |
| AGGCCACUAG GACUACGAGA UAGACAGUUA GUCAUGGGGU GUUGUUGGGC UUUUAGAAGG | 1320 |
| CACAAGAUAA CAUCUAUUUA UAAGCGCCCG GAUACCCAAA CCAUCAUCAA AGUGAACAGC | 1380 |
| GAUUUCCACU CAUUCGUGCU GCCCAGGAUA GGCAGUAACA CAUUGGAGAU CGGGCUGAGA | 1440 |
| ACAAGAAUCA GGAAAAUGUU AGAGGAGCAC AAGGAGCCGU CACCUCUCAU UACCGCCGAG | 1500 |
| GACGUACAAG AAGCUAAGUG CGCAGCCGAU GAGGCUAAGG AGGUGCGUGA AGCCGAGGAG | 1560 |
| UUGCGCGCAG CUCUACCACC UUUGGCAGCU GAUGUUGAGG AGCCCACUCU GGAAGCCGAU | 1620 |
| GUCGACUUGA UGUUACAAGA GGCUGGGGCC GGCUCAGUGG AGACACCUCG UGGCUUGAUA | 1680 |
| AAGGUUACCA GCUACGAUGG CGAGGACAAG AUCGGCUCUU ACGCUGUGCU UUCUCCGCAG | 1740 |
| GCUGUACUCA AGAGUGAAAA AUUAUCUUGC AUCCACCCUC UCGCUGAACA AGUCAUAGUG | 1800 |
| AUAACACACU CUGGCCGAAA AGGGCGUUAU GCCGUGGAAC CAUACCAUGG UAAAGUAGUG | 1860 |
| GUGCCAGAGG GACAUGCAAU ACCCGUCCAG GACUUUCAAG CUCUGAGUGA AAGUGCCACC | 1920 |
| AUUGUGUACA ACGAACGUGA GUUCGUAAAC AGGUACCUGC ACCAUAUUGC CACACAUGGA | 1980 |
| GGAGCGCUGA ACACUGAUGA AGAAUAUUAC AAAACUGUCA AGCCCAGCGA GCACGACGGC | 2040 |
| GAAUACCUGU ACGACAUCGA CAGGAAACAG UGCGUCAAGA AAGAACUAGU CACUGGGCUA | 2100 |
| GGGCUCACAG GCGAGCUGGU GGAUCCUCCC UUCCAUGAAU UCGCCUACGA GAGUCUGAGA | 2160 |
| ACACGACCAG CCGCUCCUUA CCAAGUACCA ACCAUAGGGG UGUAUGGCGU GCCAGGAUCA | 2220 |
| GGCAAGUCUG GCAUCAUUAA AAGCGCAGUC ACCAAAAAAG AUCUAGUGGU GAGCGCCAAG | 2280 |
| AAAGAAAACU GUGCAGAAAU UAUAAGGGAC GUCAAGAAAA UGAAAGGGCU GGACGUCAAU | 2340 |
| GCCAGAACUG UGGACUCAGU GCUCUUGAAU GGAUGCAAAC ACCCCGUAGA GACCCUGUAU | 2400 |
| AUUGACGAAG CUUUUGCUUG UCAUGCAGGU ACUCUCAGAG CGCUCAUAGC CAUUAUAAGA | 2460 |
| CCUAAAAAGG CAGUGCUCUG CGGGGAUCCC AAACAGUGCG GUUUUUUUAA CAUGAUGUGC | 2520 |
| CUGAAAGUGC AUUUUAACCA CGAGAUUUGC ACACAAGUCU UCCACAAAAG CAUCUCUCGC | 2580 |
| CGUUGCACUA AAUCUGUGAC UUCGGUCGUC UCAACCUUGU UUUACGACAA AAAAAUGAGA | 2640 |
| ACGACGAAUC CGAAAGAGAC UAAGAUUGUG AUUGACACUA CCGGCAGUAC CAAACCUAAG | 2700 |
| CAGGACGAUC UCAUUCUCAC UUGUUUCAGA GGGUGGGUGA AGCAGUUGCA AAUAGAUUAC | 2760 |
| AAAGGCAACG AAAUAAUGAC GGCAGCUGCC UCUCAAGGGC UGACCCGUAA AGGUGUGUAU | 2820 |
| GCCGUUCGGU ACAAGGUGAA UGAAAAUCCU CUGUACGCAC CCACCUCAGA ACAUGUGAAC | 2880 |
| GUCCUACUGA CCCGCACGGA GGACCGCAUC GUGUGGAAAA CACUAGCCGG CGACCCAUGG | 2940 |
| AUAAAAACAC UGACUGCCAA GUACCCUGGG AAUUUCACUG CCACGAUAGA GGAGUGGCAA | 3000 |
| GCAGAGCAUG AUGCCAUCAU GAGGCACAUC UUGGAGAGAC CGGACCCUAC CGACGUCUUC | 3060 |
| CAGAAUAAGG CAAACGUGUG UUGGGCCAAG GCUUUAGUGC CGGUGCUGAA GACCGCUGGC | 3120 |
| AUAGACAUGA CCACUGAACA AUGGAACACU GUGGAUUAUU UUGAAACGGA CAAAGCUCAC | 3180 |
| UCAGCAGAGA UAGUAUUGAA CCAACUAUGC GUGAGGUUCU UUGGACUCGA UCUGGACUCC | 3240 |
| GGUCUAUUUU CUGCACCCAC UGUUCCGUUA UCCAUUAGGA AUAAUCACUG GGAUAACUCC | 3300 |
| CCGUCGCCUA ACAUGUACGG GCUGAAUAAA GAAGUGGUCC GUCAGCUCUC UCGCAGGUAC | 3360 |
| CCACAACUGC CUCGGGCAGU UGCCACUGGA AGAGUCUAUG ACAUGAACAC UGGUACACUG | 3420 |
| CGCAAUUAUG AUCCGCGCAU AAACCUAGUA CCUGUAAACA GAAGACUGCC UCAUGCUUUA | 3480 |
| GUCCUCCACC AUAAUGAACA CCCACAGAGU GACUUUUCUU CAUUCGUCAG CAAAUUGAAG | 3540 |
| GGCAGAACUG UCCUGGUGGU CGGGGAAAAG UUGUCCGUCC CAGGCAAAAU GGUUGACUGG | 3600 |
| UUGUCAGACC GGCCUGAGGC UACCUUCAGA GCUCGGCUGG AUUUAGGCAU CCCAGGUGAU | 3660 |
| GUGCCCAAAU AUGACAUAAU AUUUGUUAAU GUGAGGACCC CAUAUAAAUA CCAUCACUAU | 3720 |
| CAGCAGUGUG AAGACCAUGC CAUUAAGCUU AGCAUGUUGA CCAAGAAAGC UUGUCUGCAU | 3780 |
| CUGAAUCCCG GCGGAACCUG UGUCAGCAUA GGUUAUGGUU ACGCUGACAG GGCCAGCGAA | 3840 |
| AGCAUCAUUG GUGCUAUAGC GCGGCAGUUC AAGUUUUCCC GGGUAUGCAA ACCGAAAUCC | 3900 |
| UCACUUGAAG AGACGGAAGU UCUGUUUGUA UUCAUUGGGU ACGAUCGCAA GGCCCGUACG | 3960 |
| CACAAUCCUU ACAAGCUUUC AUCAACCUUG ACCAACAUUU AUACAGGUUC CAGACUCCAC | 4020 |
| GAAGCCGGAU GUGCACCCUC AUAUCAUGUG GUGCGAGGGG AUAUUGCCAC GGCCACCGAA | 4080 |
| GGAGUGAUUA UAAAUGCUGC UAACAGCAAA GGACAACCUG GCGGAGGGGU GUGCGGAGCG | 4140 |
| CUGUAUAAGA AAUUCCCGGA AAGCUUCGAU UUACAGCCGA UCGAAGUAGG AAAAGCGCGA | 4200 |
| CUGGUCAAAG GUGCAGCUAA ACAUAUCAUU CAUGCCGUAG GACCAAACUU CAACAAAGUU | 4260 |
| UCGGAGGUUG AAGGUGACAA ACAGUUGGCA GAGGCUUAUG AGUCCAUCGC UAAGAUUGUC | 4320 |
| AACGAUAACA AUUACAAGUC AGUAGCGAUU CCACUGUUGU CCACCGGCAU CUUUUCCGGG | 4380 |
| AACAAAGAUC GACUAACCCA AUCAUUGAAC CAUUUGCUGA CAGCUUUAGA CACCACUGAU | 4440 |
| GCAGAUGUAG CCAUAUACUG CAGGGACAAG AAAUGGGAAA UGACUCUCAA GGAAGCAGUG | 4500 |
| GCUAGGAGAG AAGCAGUGGA GGAGAUAUGC AUAUCCGACG ACUCUUCAGU GACAGAACCU | 4560 |
| GAUGCAGAGC UGGUGAGGGU GCAUCCGAAG AGUUCUUUGG CUGGAAGGAA GGGCUACAGC | 4620 |
| ACAAGCGAUG GCAAAACUUU CUCAUAUUUG GAAGGGACCA AGUUUCACCA GGCGGCCAAG | 4680 |
| GAUAUAGCAG AAAUUAAUGC CAUGUGGCCC GUUGCAACGG AGGCCAAUGA GCAGGUAUGC | 4740 |
| AUGUAUAUCC UCGGAGAAAG CAUGAGCAGU AUUAGGUCGA AAUGCCCCGU CGAAGAGUCG | 4800 |
| GAAGCCUCCA CACCACCUAG CACGCUGCCU UGCUUGUGCA UCCAUGCCAU GACUCCAGAA | 4860 |
| AGAGUACAGC GCCUAAAAGC CUCACGUCCA GAACAAAUUA CUGUGUGCUC AUCCUUUCCA | 4920 |
| UUGCCGAAGU AUAGAAUCAC UGGUGUGCAG AAGAUCCAAU GCUCCCAGCC UAUAUUGUUC | 4980 |
| UCACCGAAAG UGCCUGCGUA UAUUCAUCCA AGGAAGUAUC UCGUGGAAAC ACCACCGGUA | 5040 |
| GACGAGACUC CGGAGCCAUC GGCAGAGAAC CAAUCCACAG AGGGGACACC UGAACAACCA | 5100 |
| CCACUUAUAA CCGAGGAUGA GACCAGGACU AGAACGCCUG AGCCGAUCAU CAUCGAAGAG | 5160 |
| GAAGAAGAGG AUAGCAUAAG UUUGCUGUCA GAUGGCCCGA CCCACCAGGU GCUGCAAGUC | 5220 |
| GAGGCAGACA UUCACGGGCC GCCCUCUGUA UCUAGCUCAU CCUGGUCCAU UCCUCAUGCA | 5280 |
| UCCGACUUUG AUGUGGACAG UUUAUCCAUA CUUGACACCC UGGAGGGAGC UAGCGUGACC | 5340 |
| AGCGGGGCAA CGUCAGCCGA GACUAACUCU UACUUCGCAA AGAGUAUGGA GUUUCUGGCG | 5400 |
| CGACCGGUGC CUGCGCCUCG AACAGUAUUC AGGAACCCUC CACAUCCCGC UCCGCGCACA | 5460 |
| AGAACACCGU CACUUGCACC CAGCAGGGCC UGCUCGAGAA CCAGCCUAGU UUCCACCCCG | 5520 |
| CCAGGCGUGA AUAGGGUGAU CACUAGAGAG GAGCUCGAGG CGCUUACCCC GUCACGCACU | 5580 |
| CCUAGCAGGU CGGUCUCGAG AACCAGCCUG GUCUCCAACC CGCCAGGCGU AAAUAGGGUG | 5640 |
| AUUACAAGAG AGGAGUUUGA GGCGUUCGUA GCACAACAAC AAUGACGGUU UGAUGCGGGU | 5700 |
| GCAUACAUCU UUUCCUCCGA CACCGGUCAA GGGCAUUUAC AACAAAAAUC AGUAAGGCAA | 5760 |
| ACGGUGCUAU CCGAAGUGGU GUUGGAGAGG ACCGAAUUGG AGAUUUCGUA UGCCCCGCGC | 5820 |
| CUCGACCAAG AAAAAGAAGA AUUACUACGC AAGAAAUUAC AGUUAAAUCC CACACCUGCU | 5880 |
| AACAGAAGCA GAUACCAGUC CAGGAAGGUG GAGAACAUGA AAGCCAUAAC AGCUAGACGU | 5940 |
| AUUCUGCAAG GCCUAGGGCA UUAUUUGAAG GCAGAAGGAA AAGUGGAGUG CUACCGAACC | 6000 |
| CUGCAUCCUG UUCCUUUGUA UUCAUCUAGU GUGAACCGUG CCUUUUCAAG CCCCAAGGUC | 6060 |
| GCAGUGGAAG CCUGUAACGC CAUGUUGAAA GAGAACUUUC CGACUGUGGC UUCUUACUGU | 6120 |
| AUUAUUCCAG AGUACGAUGC CUAUUUGGAC AUGGUUGACG GAGCUUCAUG CUGCUUAGAC | 6180 |
| ACUGCCAGUU UUUGCCCUGC AAAGCUGCGC AGCUUUCCAA AGAAACACUC CUAUUUGGAA | 6240 |
| CCCACAAUAC GAUCGGCAGU GCCUUCAGCG AUCCAGAACA CGCUCCAGAA CGUCCUGGCA | 6300 |
| GCUGCCACAA AAAGAAAUUG CAAUGUCACG CAAAUGAGAG AAUUGCCCGU AUUGGAUUCG | 6360 |
| GCGGCCUUUA AUGUGGAAUG CUUCAAGAAA UAUGCGUGUA AUAAUGAAUA UUGGGAAACG | 6420 |
| UUUAAAGAAA ACCCCAUCAG GCUUACUGAA GAAAACGUGG UAAAUUACAU UACCAAAUUA | 6480 |
| AAAGGACCAA AAGCUGCUGC UCUUUUUGCG AAGACACAUA AUUUGAAUAU GUUGCAGGAC | 6540 |
| AUACCAAUGG ACAGGUUUGU AAUGGACUUA AAGAGAGACG UGAAAGUGAC UCCAGGAACA | 6600 |
| AAACAUACUG AAGAACGGCC CAAGGUACAG GUGAUCCAGG CUGCCGAUCC GCUAGCAACA | 6660 |
| GCGUAUCUGU GCGGAAUCCA CCGAGAGCUG GUUAGGAGAU UAAAUGCGGU CCUGCUUCCG | 6720 |
| AACAUUCAUA CACUGUUUGA UAUGUCGGCU GAAGACUUUG ACGCUAUUAU AGCCGAGCAC | 6780 |
| UUCCAGCCUG GGGAUUGUGU UCUGGAAACU GACAUCGCGU CGUUUGAUAA AAGUGAGGAC | 6840 |
| GACGCCAUGG CUCUGACCGC GUUAAUGAUU CUGGAAGACU UAGGUGUGGA CGCAGAGCUG | 6900 |
| UUGACGCUGA UUGAGGCGGC UUUCGGCGAA AUUUCAUCAA UACAUUUGCC CACUAAAACU | 6960 |
| AAAUUUAAAU UCGGAGCCAU GAUGAAAUCU GGAAUGUUCC UCACACUGUU UGUGAACACA | 7020 |
| GUCAUUAACA UUGUAAUCGC AAGCAGAGUG UUGAGAGAAC GGCUAACCGG AUCACCAUGU | 7080 |
| GCAGCAUUCA UUGGAGAUGA CAAUAUCGUG AAAGGAGUCA AAUCGGACAA AUUAAUGGCA | 7140 |
| GACAGGUGCG CCACCUGGUU GAAUAUGGAA GUCAAGAUUA UAGAUGCUGU GGUGGGCGAG | 7200 |
| AAAGCGCCUU AUUUCUGUGG AGGGUUUAUU UUGUGUGACU CCGUGACCGG CACAGCGUGC | 7260 |
| CGUGUGGCAG ACCCCCUAAA AAGGCUGUUU AAGCUUGGCA AACCUCUGGC AGCAGACGAU | 7320 |
| GAACAUGAUG AUGACAGGAG AAGGGCAUUG CAUGAAGAGU CAACACGCUG GAACCGAGUG | 7380 |
| GGUAUUCUUU CAGAGCUGUG CAAGGCAGUA GAAUCAAGGU AUGAAACCGU AGGAACUUCC | 7440 |
| AUCAUAGUUA UGGCCAUGAC UACUCUAGCU AGCAGUGUUA AAUCAUUCAG CUACCUGAGA | 7500 |
| GGGGCCCCUA UAACUCUCUA CGGCUAACCU GAAUGGACUA CGACAUAGUC UAGUCCGCCA | 7560 |
| AGAUAUCGCA CCAUGAAGGC CAUCCUGGUG GUCAUGCUGU ACACCUUCAC CACCGCCAAC | 7620 |
| GCCGACACAC UGUGUAUCGG CUACCACGCC AACAACAGCA CCGACACCGU GGAUACCGUG | 7680 |
| CUGGAAAAGA ACGUGACCGU GACACACAGC GUGAACCUGC UGGAAGAUAA GCACAACGGC | 7740 |
| AAGCUGUGCA AGCUGAGAGG CGUGGCACCU CUGCACCUGG GCAAGUGUAA UAUCGCCGGC | 7800 |
| UGGAUCCUGG GCAACCCUGA GUGUGAAAGC CUGAGCACCG CCAGAUCCUG GUCCUACAUC | 7860 |
| GUGGAAACCA GCAACAGCGA CAACGGCACA UGCUACCCCG GCGACUUCAU CAACUACGAG | 7920 |
| GAACUGCGGG AACAGCUGAG CAGCGUGUCC AGCUUCGAGA GAUUCGAGAU CUUCCCCAAG | 7980 |
| ACCAGCAGCU GGCCCAACCA CGACUCUGAC AAUGGCGUGA CAGCCGCCUG UCCUCAUGCC | 8040 |
| GGCGCUAAGA GCUUCUACAA GAACCUGAUC UGGCUGGUCA AGAAGGGCAA GAGCUACCCC | 8100 |
| AAGAUCAACC AGACCUACAU CAACGACAAG GGCAAAGAGG UGCUGGUCCU CUGGGGCAUC | 8160 |
| CACCAUCCUC CAACAAUCGC CGAUCAGCAG AGCCUGUACC AGAACGCCGA UGCCUAUGUG | 8220 |
| UUCGUGGGCA CCAGCCGGUA CAGCAAGAAG UUCAAGCCCG AGAUCGCCAC CAGGCCUAAA | 8280 |
| GUGCGGGAUC AAGAGGGCAG AAUGAACUAC UACUGGACCC UGGUGGAACC CGGCGACAAG | 8340 |
| AUCACAUUUG AGGCCACCGG CAAUCUGGUG GCCCCUAGAU ACGCCUUCAC CAUGGAAAGA | 8400 |
| GAUGCCGGCA GCGGCAUCAU CAUCAGCGAU ACCCCUGUGC ACGACUGCAA CACCACCUGU | 8460 |
| CAGACACCUG AGGGCGCCAU CAAUACCAGC CUGCCUUUCC AGAACGUGCA CCCCAUCACC | 8520 |
| AUCGGCAAGU GCCCCAAAUA CGUGAAGUCC ACCAAGCUGA GGCUGGCCAC AGGCCUGAGA | 8580 |
| AAUGUGCCCU CCAUCCAGAG CAGAGGCCUG UUUGGAGCCA UUGCCGGCUU UAUCGAAGGC | 8640 |
| GGCUGGACAG GCAUGGUGGA CGGAUGGUAC GGAUACCACC ACCAGAACGA GCAAGGCUCU | 8700 |
| GGCUAUGCCG CCGACCUGAA GUCUACCCAG AAUGCCAUCG AUAAGAUCAC CAACAAAGUG | 8760 |
| AACAGCGUGA UCGAGAAGAU GAACACCCAG UUCACCGCCG UGGGAAAAGA GUUCAACCAC | 8820 |
| CUGGAAAAGC GCAUCGAGAA CCUGAACAAG AAGGUGGACG ACGGCUUCCU GGACAUCUGG | 8880 |
| ACCUACAAUG CCGAACUGCU GGUGCUGCUG GAGAACGAGA GAACCCUGGA CUACCACGAC | 8940 |
| AGCAACGUGA AGAACCUGUA CGAGAAAGUG CGCAACCAGC UGAAGAACAA CGCCAAAGAG | 9000 |
| AUCGGCAACG GCUGCUUCGA GUUCUACCAC AAGUGCGACA AUACCUGCAU GGAAAGCGUG | 9060 |
| AAGAAUGGCA CCUACGACUA CCCUAAGUAC AGCGAGGAAG CCAAGCUGAA CCGCGAGAAG | 9120 |
| AUCGACGGCG UGAAGCUGGA UAGCACCCGG AUCUACCAGA UUCUGGCCAU CUACAGCACC | 9180 |
| GUGGCCUCUA GCCUGGUGCU GGUGGUUUCU CUGGGCGCUA UCAGCUUCUG GAUGUGCAGC | 9240 |
| AAUGGCAGCC UGCAGUGCCG GAUCUGCAUC UGAUGAAUAC AGCAGCAAUU GGCAAGCUGC | 9300 |
| UUACAUAGAA CUCGCGGCGA UUGGCAUGCC GCCUUAAAAU UUUUAUUUUA UUUUUCUUUU | 9360 |
| CUUUUCCGAA UCGGAUUUUG UUUUUAAUAU UUCAAAAAAA AAAAAAAAAA AAAAAAAAAA | 9420 |
| AAAAAAAAAA AAA (SEQ ID NO: 2) | 9433 |

### Example 4: S.4.4.1 Construct 7: Tc83-Ha-40a 50U-50Pu (Pf-07871987)

Batch details and batch analysis summary data are provided in Table 7 for 1 batch of regulatory toxicology material and 1 GMP batch of drug substance to be used in clinical trials.

TABLE 7
S.4.4.6-7. Batch Results for Influenza saRNA Vaccine TC83-HA-40A 50U-50pU
Drug Substance

| | | | | Nonclinical | | Clinical Drug |
|---|---|---|---|---|---|---|
| | | | | Toxicology | | Substance |
| Quality | Analytical | Acceptance | | (Batch 00710958- | | (Batch |
| Attribute | Procedure | Criteria | | 0369) | | 22V543F101) |
| Appearance | Clarity | ≤6 | NTU | Clear | | 0 NTU |
| (Clarity) | | | | | | |
| Appearance | Coloration | Not more | | Colorless solution | | ≤B9 |
| (Coloration) | | intensely colored | | | | |
| | | than level 7 of | | | | |
| | | the brown (B) | | | | |
| | | color standard. | | | | |
| pH | Potentiometry | 7.0 ± 0.5 | | 7.1 | | 6.9 |
| Content (RNA | UV | 1.25 ± 0.25 | | 2.00 | mg/mL | 1.27 mg/mL |
| concentration) | spectroscopy | mg/mL | | | | |
| ddPCR | Identity of | Identity | | Positive^{a} | | Confirmed |
| | Encoded RNA | confirmed | | | | |
| | Sequence | | | | | |
| Presence of | RP-HPLC | Identity | | Confirmed^{b} | | Confirmed |
| pseudoU | | confirmed | | | | |
| RNA integrity | Capillary gel | ≥60% | | 88% | | 87% |
| | electrophoresis | | | | | |
| Residual DNA | qPCR | ≤990 ng | | 37 | ng/mg | NMT 1 ng/mg |
| template | | DNA/mg RNA | | | | |
| Endotoxin | Endotoxin | ≤12.5 | EU/mL | <0.025 | EU/mg | NMT 0.2 EU/mL |

| | (LAL) | | | | |
|---|---|---|---|---|---|
| Bioburden | Bioburden | ≤1 CFU/10 mL | ≤10 | CFU/mL | 0 CFU/10 mL |

^{a}Identity was determined via reverse transcription, quantitative polymerase chain reaction
^{b}Nonclinical toxicology reported an approximate percentage of pseudoU. Its presence can be inferred to be ″confirmed″ from the reported result of it being present.
Specifications only apply to clinical supplies
Abbreviations: NTU = nephelometric turbidity units; NT = not tested; TBP = to be provided in the IND amendment; ddPCR = digital droplet polymerase chain reaction; qPCR = quantitative polymerase chain reaction; LAL = limulus amebocyte lysate; NMT = not more than; EU = Endotoxin unit; CFU = Colony forming unit

### Example 5: Assays

### Hemagglutination Inhibition Assay

The primary serological assay used to measure vaccine-induced immune responses to influenza is the hemagglutinin inhibition assay (HAI). The HAI quantitatively measures functional antibodies in serum that prevent HA-mediated agglutination of red blood cells in reactions containing receptor-destroying enzyme pretreated serum samples, influenza virus and red blood cells derived from turkey or guinea pig. The HAI titer is the reciprocal of the highest serum dilution resulting in loss of HA activity, visualized as a teardrop shape when the microtiter plate is tilted. Titers from multiple determinations per sample are reported as geometric mean titers (GMT). A HAI titer of 1:40 is generally accepted as protective in humans.

### Influenza Microneutralization Assay

The influenza virus microneutralization assay (MNT) quantitatively measures functional antibodies in serum that neutralize influenza virus activity, preventing productive infection of a host cell monolayer. A neutralization reaction occurs when influenza virus is incubated with serum samples; this reaction mixture is then applied to a monolayer of Madin-Darby Canine Kidney (MDCK) cells to measure the extent of neutralization. MNT titers are reported as the reciprocal of the dilution that results in 50% or 90% reduction in infection when compared to a no serum control. The 1-Day MNT measures anti-HA neutralizing antibodies and the 3-Day MNT measures both anti-HA and anti-NA neutralizing antibodies.

### Neuraminidase Inhibition Assay

The neuraminidase inhibition assay (NAI) quantitatively measures functional antibodies in serum that prevent NA-mediated cleavage of sialic acid cleavage in an enzyme-linked lectin assay. Briefly, antibody-containing serum is incubated with influenza virus, and the mixture is transferred to a fetuin lectin-coated plate. Cleavage of sialic acid from the fetuin is monitored through a colorimetric reaction following binding of horseradish peroxidase-conjugated peanut agglutinin to exposed galactose moieties and addition of substrate. The NAI titer is the reciprocal of the highest serum dilution resulting in reduction of NA activity by 50% compared to a no-serum control. Titers from multiple determinations per sample are reported as geometric mean titers (GMT).

### Example 6: Evaluation of Flu Bicistronic HA-NA saRNA Vaccine Designs in Mice

This study was conducted to compare the immunogenicity of bicistronic saRNA vaccine candidates encoding influenza hemaglutinin (HA) and neuraminidase (NA) to determine the optimal bicistronic HA-NA saRNA vaccine design. The saRNA vectors used in this study were all based on the TC-83 backbone, however the study was designed to evaluate immunogenicity with or without an exogenous kozak sequence upstream of the first gene-of-interest and the effect of poly A tail length (40A or 80A).

The key bicistronic design elements that were evaluated in this study include the regulatory element used to drive expression of the second gene-of-interest (subgenomic promoter (SGP) vs. internal ribosomal entry site (IRES)) and the order of antigen placement on the vector (HA-NA or NA-HA).

Intramuscular immunization of Balb/c mice with LNP-formulated saRNA vaccines encoding the A/Wisconsin/588/2019 (H1N1) HA and/or NA antigen induced functional and neutralizing antibody responses.

Overall, similar titers were elicited with all bicistronic saRNA vaccines tested, and these titers were also similar to the saRNA vaccine comprised of individually-formulated saRNA-HA+saRNA-NA. These results confirmed that the bicistronic saRNA approach elicited titers and is feasible.

The saRNA vectors used in this study were all based on the TC-83 backbone, however the study was designed to evaluate immunogenicity with or without an exogenous kozak sequence upstream of the first gene-of-interest and the effect of poly A tail length (40A or 80A). The key bicistronic design elements that were evaluated in this study include the regulatory element required to drive expression of the second gene-of-interest, and the order of antigen placement on the vector (HA-NA or NA-HA). The regulatory elements selected for comparison were the native VEEV subgenomic promoter (SGP; 61 nucleotides) and the internal ribosomal entry site (IRES, 587 nucleotides) derived from an Encephalomyocarditis Virus. As comparators, modRNA vaccines encoding influenza HA or NA were also included in the study. Mice were immunized with saRNA or modRNA LNP formulations on Days 0 and 28, and sera were collected 21 days post prime and 14 days post boost. Neutralizing and functional antibodies were measured on Days 21 and 42 to determine immunogenicity.

This study was designed with 15 groups as shown in Table 8 each containing a total of female mice (strain of mice: BALB/c). The mRNA drug products were evaluated at 0.05 mL dose volume

TABLE 8
Study Design

| | | | | Dose | | |
|---|---|---|---|---|---|---|
| | | | Dose | Vol/ | Vax | Bleed |
| Gp# | Mice | Self-amplifying RNA DP Description | (μg) | Route | (Day) | (Day) |
| 1 | 10 | Saline | — | 50 μl/ | 0, 28 | 21, 42 |
| | | | | IM | | |
| 2 | 10 | TC83-Kan-Wisc-HA-IRES-NA (kozak, 40As)- | 0.02 | 50 μl/ | 0, 28 | 21, 42 |
| | | set 1 | | IM | | |
| 3 | 10 | TC83-Kan-Wisc-HA-SGP-NA (kozak, 40As)- | 0.02 | 50 μl/ | 0, 28 | 21, 42 |
| | | set 1 | | IM | | |
| 4 | 10 | TC83-Kan-Wisc-NA-SGP-HA (kozak, 40As)- | 0.02 | 50 μl/ | 0, 28 | 21, 42 |
| | | set 1 | | IM | | |
| 5 | 10 | TC83-Wisc-HA-IRES-NA (no kozak, 40As)- | 0.02 | 50 μl/ | 0, 28 | 21, 42 |
| | | set 2 | | IM | | |
| 6 | 10 | TC83-Wisc-HA-SGP-NA (no kozak, 40As)- | 0.02 | 50 μl/ | 0, 28 | 21, 42 |
| | | set 2 | | IM | | |
| 7 | 10 | TC83-Wisc-NA-SGP-HA (no kozak, 40As)- | 0.02 | 50 μl/ | 0, 28 | 21, 42 |
| | | set 2 | | IM | | |
| 8 | 10 | TC83-Wisc-HA-IRES-NA (no kozak, 80As)- | 0.02 | 50 μl/ | 0, 28 | 21, 42 |
| | | set 3 | | IM | | |
| 9 | 10 | TC83-Wisc-HA-SGP-NA (no kozak, 80As)- | 0.02 | 50 μl/ | 0, 28 | 21, 42 |
| | | set 3 | | IM | | |
| 10 | 10 | TC83-Wisc-NA-SGP-HA (no kozak, 80As)- | 0.02 | 50 μl/ | 0, 28 | 21, 42 |
| | | set 3 | | IM | | |
| 11 | 10 | saRNA A/Wisc. H1 (RMM59)-kozak 40As | 0.02 | 50 μl/ | 0, 28 | 21, 42 |
| | | | | IM | | |
| 12 | 10 | saRNA A/Wisc. N1 (RMM65)-kozak 40As | 0.02 | 50 μl/ | 0, 28 | 21, 42 |
| | | | | IM | | |
| 13 | 10 | saRNA A/WVisc. H1 (RMM59) + saRNA | 0.04 | 50 μl/ | 0, 28 | 21, 42 |
| | | A/Wisc. N1 (RMM65)-kozak 40As | | IM | | |
| | | (RMM59 + RMM65) post-mix | | | | |
| 14 | 10 | modRNA H1 (RMM71) | 0.2 | 50 μl/ | 0, 28 | 21, 42 |
| | | | | IM | | |
| 15 | 10 | modRNA N1 (RMM72) | 0.2 | 50 μl/ | 0, 28 | 21, 42 |
| | | | | IM | | |

TABLE 9
Study Schedule

| Day | Procedure |
|---|---|
| 0 | Vax #1 |
| 21 | Bleed |
| 28 | Vax #2 |
| 42 | Terminal bleed |

TABLE 10
Test articles and diluents for the study (provided for prime and boost)

| | | | | Gps to | |
|---|---|---|---|---|---|
| Item | | | Formulation Matrix | be used | *Vials#/ |
| # | Test Articles/Diluent | DP Lot # | and information | for | Storage |
| 1 | Saline | B2011082 | 0.9 % NaCl in water | 1 and | 2 bottles |
| | | | | diluent | RT |
| | | | | for grps | |
| | | | | 2-15 | |
| 2 | TC83-Kan-Wisc-HA- | 00715982- | 0.066 mg/ml of saRNA | 2 | 3 × 0.3 mL |
| | IRES-NA (kozak, | 0018-F1 | LNP in 10 mM Tris/10% | | vials |
| | 40As) - set 1 | | Surose/20 mM Glutamic | | −80° C. |
| | | | Acid, pH 7.4 | | |
| 3 | TC83-Kan-Wisc-HA- | 00715982- | 0.065 mg/ml of saRNA | 3 | 3 × 0.3 mL |
| | SGP-NA (kozak, | 0018-F2 | LNP in 10 mM Tris/10% | | vials |
| | 40As) - set 1 | | Surose/20 mM Glutamic | | −80°° C. |
| | | | Acid, pH 7.4 | | |
| 4 | TC83-Kan-Wisc-NA- | 00715982- | 0.068 mg/ml of saRNA | 4 | 3 × 0.3 mL |
| | SGP-HA (kozak, | 0018-F3 | LNP in 10 mM Tris/10% | | vials |
| | 40As) - set 1 | | Surose/20 mM Glutamic | | −80°° C. |
| | | | Acid, pH 7.4 | | |
| 5 | TC83-Wisc-HA-IRES- | 00715725- | 0.077 mg/ml of saRNA | 5 | 3 × 0.3 mL |
| | NA (no kozak, 40As) - | 0035-F2 | LNP in 10 mM Tris/10% | | vials |
| | set 2 | | Surose/20 mM Glutamic | | −80° C. |
| | | | Acid, pH 7.4 | | |
| 6 | TC83-Wisc-HA-SGP- | 00715725- | 0.074 mg/ml of saRNA | 6 | 3 × 0.3 mL |
| | NA (no kozak, 40As) - | 0035-F4 | LNP in 10 mM Tris/10% | | vials |
| | set 2 | | Surose/20 mM Glutamic | | −80° C. |
| | | | Acid, pH 7.4 | | |
| 7 | TC83-Wisc-NA-SGP- | 00715725- | 0.069 mg/ml of saRNA | 7 | 3 × 0.3 mL |
| | HA (no kozak, 40As) - | 0035-F6 | LNP in 10 mM Tris/10% | | vials |
| | set 2 | | Surose/20 mM Glutamic | | −80° C. |
| | | | Acid, pH 7.4 | | |
| 8 | TC83-Wisc-HA-IRES- | 00715725- | 0.079 mg/ml of saRNA | 8 | 3 × 0.3 mL |
| | NA (no kozak, 80As) - | 0035-F1 | LNP in 10 mM Tris/10% | | vials |
| | set 3 | | Surose/20 mM Glutamic | | −80°° C. |
| | | | Acid, pH 7.4 | | |
| 9 | TC83-Wisc-HA-SGP- | 00715725- | 0.069 mg/ml of saRNA | 9 | 3 × 0.3 mL |
| | NA (no kozak, 80As) - | 0035-F3 | LNP in 10 mM Tris/10% | | vials |
| | set 3 | | Surose/20 mM Glutamic | | −80° C. |
| | | | Acid, pH 7.4 | | |
| 10 | TC83-Wisc-NA-SGP- | 00715725- | 0.069 mg/ml of saRNA | 10 | 3 × 0.3 mL |
| | HA (no kozak, 80As) - | 0035-F5 | LNP in 10 mM Tris/10% | | vials |
| | set 3 | | Surose/20 mM Glutamic | | −80°° C. |
| | | | Acid, pH 7.4 | | |
| 11 | saRNA A/Wisc. H1 | 00715982- | 0.061 mg/ml of saRNA | 11 and | 3 × 0.3 mL |
| | (RMM59) - kozak | 0018-F4 | LNP in 10 mM Tris/10% | 13 | vials |
| | 40As | | Surose/20 mM Glutamic | | −80° C. |
| | | | Acid, pH 7.4 | | |
| 12 | saRNA A/Wisc. N1 | 00715982- | 0.062 mg/ml of saRNA | 12 and | 3 × 0.3 mL |
| | (RMM65) - kozak | 0018-F5 | LNP in 10 mM Tris/10% | 13 | vials |
| | 40As | | Surose/20 mM Glutamic | | −80° C. |
| | | | Acid, pH 7.4 | | |
| 13 | modRNA H1 | 00715725- | 0.093 mg/mL of | 14 | 3 × 0.3 mL |
| | (RMM71) | 0032-F10 | modRNA LNP in 10 mM | | vials |
| | | | Tris/300 mM Surose, pH | | −80° C. |
| | | | 7.4 | | |
| 14 | modRNA N1 | 000715982- | 0.099 mg/ml of | 15 | 3 × 0.3 mL |
| | (RMM72) | 0018-F6 | modRNA LNP in 10 mM | | vials |
| | | | Tris/300 mM Surose, pH | | −80° C. |
| | | | 7.4 | | |

M. Analytical Test Results of Test Articles

TABLE 11
Drug Product Materials and Analytical Results

| | Concentration | | Size | | Integrity | Cap % | IVE EC50 | Endotoxin |
|---|---|---|---|---|---|---|---|---|
| DP Lot # | (μg/mL) | Encapsulation | (nm) | PDI | by FA | (DS) | (ng) | (EU/mL) |
| 00715982- | 66 | 97% | 70 | 0.07 | 72% | 100% | HA: 43.3 | 0.12 |
| 0018-F1 | | | | | | | NA: 67.8 | |
| 00715982- | 65 | 98% | 69 | 0.06 | 64% | 100% | HA: 37.6 | 0.14 |
| 0018-F2 | | | | | | | NA: 47.5 | |
| 00715982- | 68 | 98% | 67 | 0.06 | 78% | 100% | HA: 25.1 | 0.12 |
| 0018-F3 | | | | | | | NA: 110.9 | |
| 00715982- | 61 | 97% | 70 | 0.05 | 78% | 100% | HA: 22.6 | 0.58 |
| 0018-F4 | | | | | | | | |
| 00715982- | 62 | ≥98% | 66 | 0.07 | 79% | 100% | NA: 31.5 | <0.12 |
| 0018-F5 | | | | | | | | |
| 00715982- | 99 | 97% | 70 | 0.10 | 92% | 80% | NA: 51.5 | 0.22 |
| 0018-F6 | | | | | | | | |
| 00715725- | 79 | 95% | 74 | 0.06 | 78% | 99% | HA: 11.4 | <0.05 |
| 0035-F1 | | | | | | | NA: 13.1 | |
| 00715725- | 77 | 96% | 72 | 0.12 | 77% | 99% | HA: 32.8 | <0.07 |
| 0035-F2 | | | | | | | NA: 61.5 | |
| 00715725- | 69 | 96% | 73 | 0.08 | 75% | 98% | HA: 13.6 | <0.05 |
| 0035-F3 | | | | | | | NA: 22.7 | |
| 00715725- | 74 | 95% | 74 | 0.12 | 79% | 99% | HA: 15.9 | <0.05 |
| 0035-F4 | | | | | | | NA: 33.0 | |
| 00715725- | 69 | 96% | 69 | 0.11 | 74% | 99% | HA: 28.8 | 0.06 |
| 0035-F5 | | | | | | | NA: 36.4 | |
| 00715725- | 69 | 96% | 69 | 0.14 | 80% | 99% | HA: 40.9 | 0.07 |
| 0035-F6 | | | | | | | NA: 55.0 | |
| 00715725- | 93 | 94% | 65 | 0.09 | 87% | 79% | HA: 43.5 | <0.3 |
| 0032-F10 | | | | | | | | |

### 2. Results and Discussion

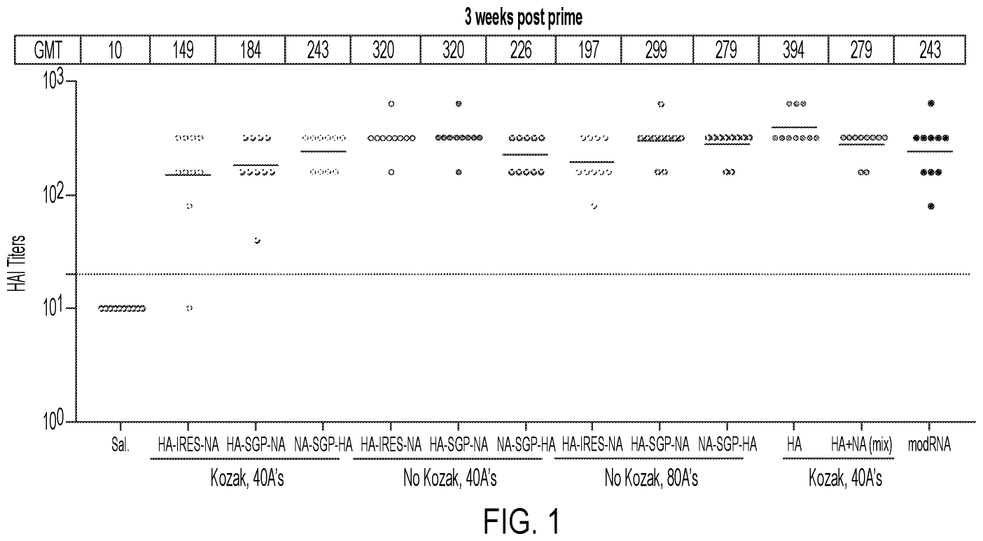

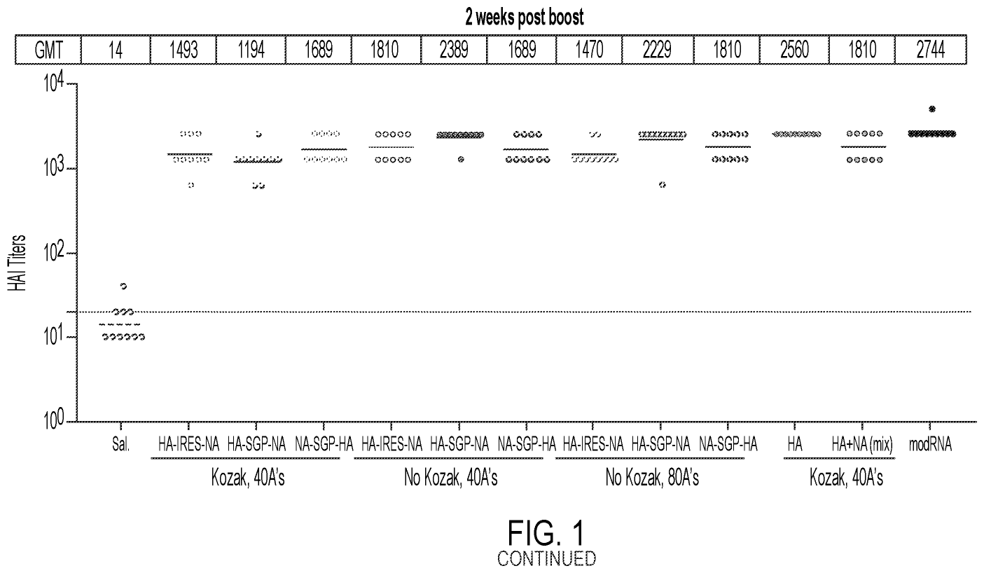

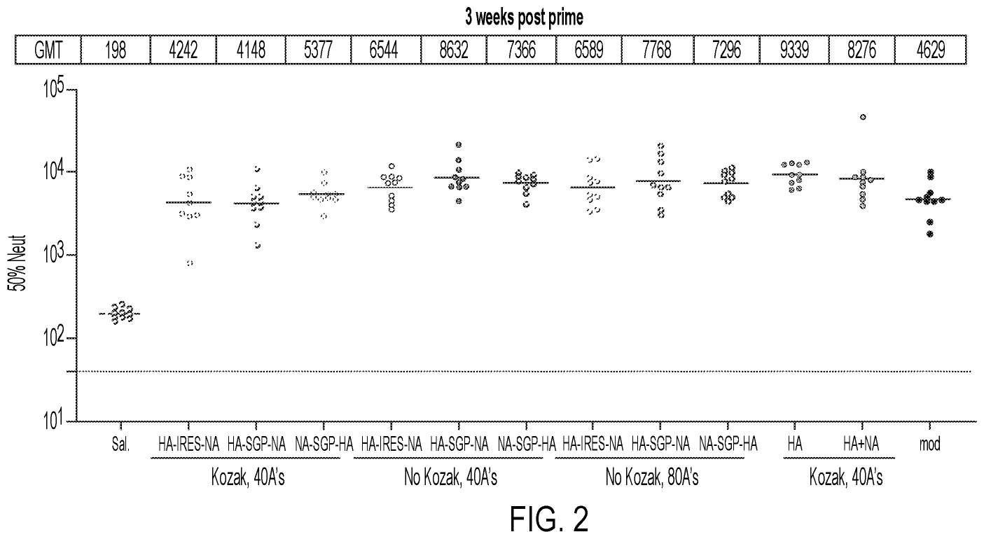

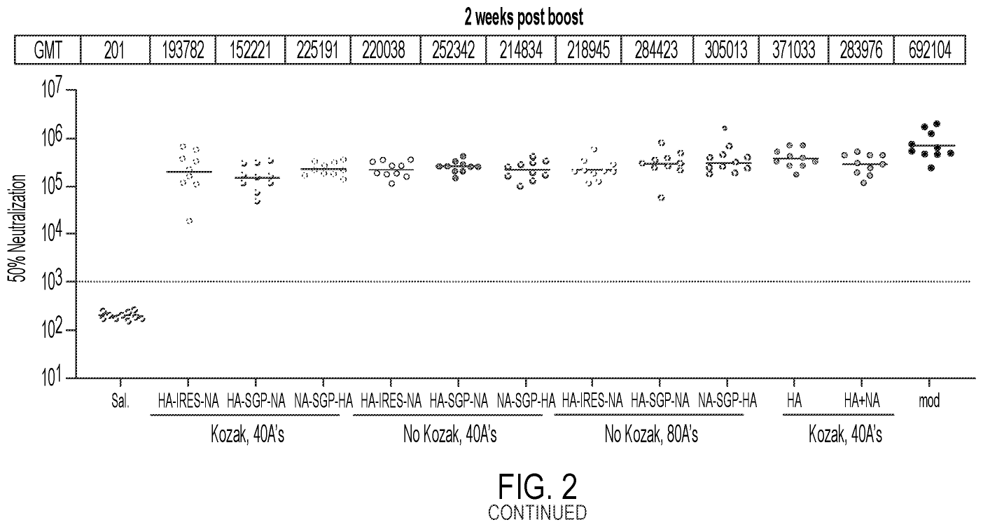

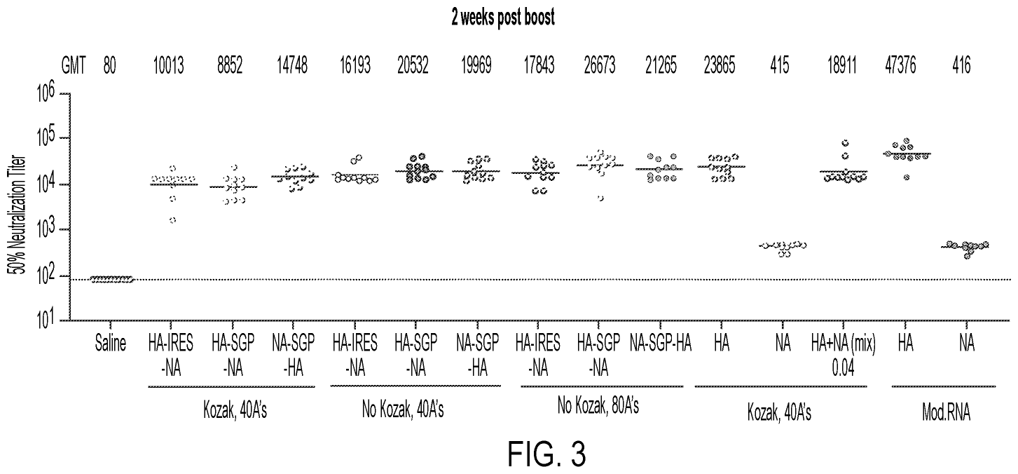

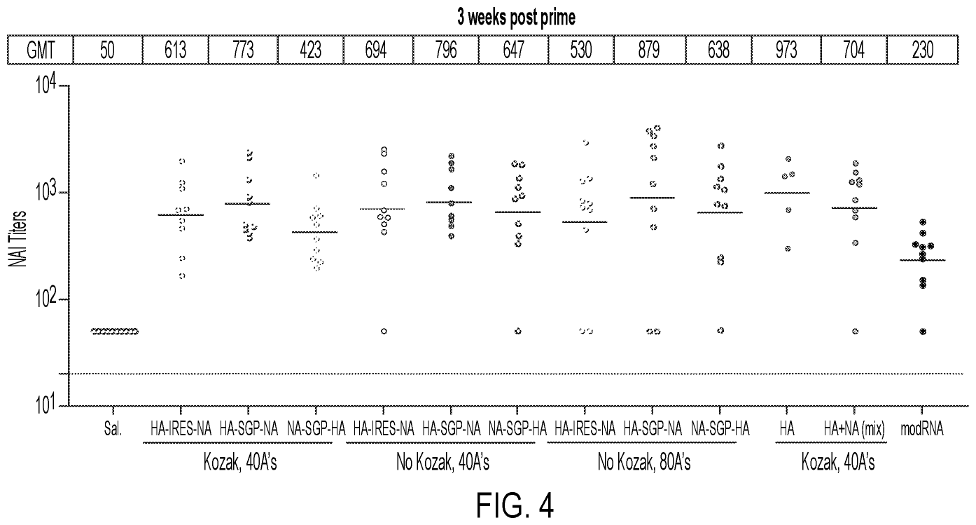

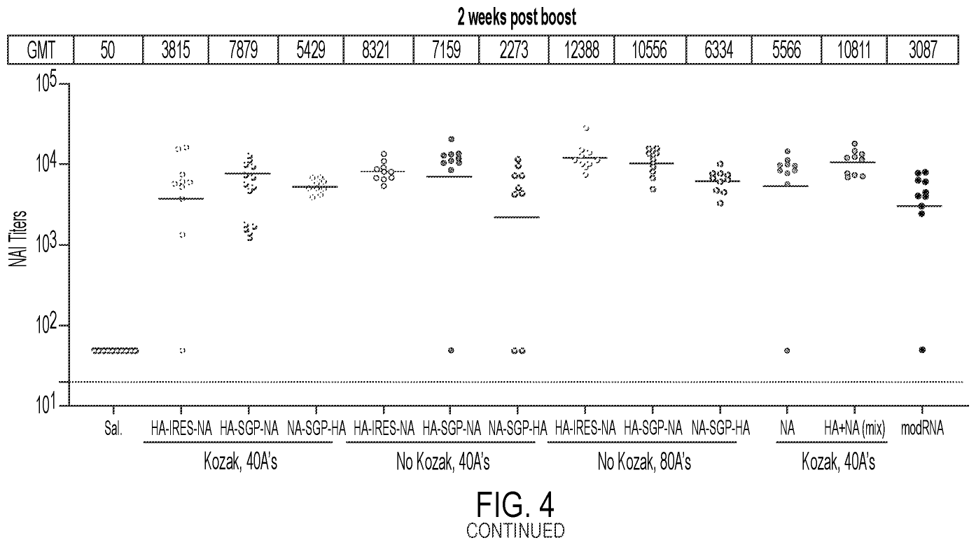

Intramuscular immunization of Balb/c mice with LNP-formulated saRNA vaccines encoding the A/Wisconsin/588/2019 (H1N1) HA and/or NA antigen induced functional and neutralizing antibody responses as measured by HAI, 1-Day MNT, 3-Day MNT, and NAI (FIG. 1, FIG. 2, FIG. 3, and FIG. 4, respectively) with a clear boosting effect two weeks after the second immunization. The 3-Day MNT results on Day 42 showed that NA contributed minimally to neutralization as compared to HA in this assay. Overall, similar titers were achieved with all bicistronic saRNA vaccines designs evaluated, and these titers were also similar to the saRNA vaccine comprised of individually-formulated HA and NA monocistronic saRNAs (HA/NA Post-Mix) and modRNA. Titers for saRNA vaccines expressing two antigens were similar or just slightly lower than the saRNA-HA or -NA only controls. These data confirmed that the bicistronic saRNA approach is feasible.

Neither the deletion of Kozak sequence, polyA tail length, regulatory element used to drive the second gene-of-interest (IRES vs. SGP) nor the order of antigen placement (HA-NA or NA-HA) had a clear impact on titers elicited.

With respect to FIG. 1, female Balb/c mice were immunized IM on Days 0 and 28 with a different LNP-formulated influenza saRNA vaccine constructs and with the influenza modRNA comparator encoding A/Wisconsin/588/2019 (H1N1) HA and/or NA. The HA/NA post-mix preparation was comprised of a 1:1 mixture of individually-formulated saRNA-HA plus saRNA-NA. Functional antibody responses against A/Wisconsin/588/2019 were measured by HAI on Day 21 (3 weeks post prime) and on Day 42 (2 weeks post boost).

With respect to FIG. 2, female Balb/c mice were immunized IM on Days 0 and 28 with a different LNP-formulated influenza saRNA vaccine constructs and with the influenza modRNA comparator encoding A/Wisconsin/588/2019 (H1N1) HA and/or NA. The HA/NA post-mix preparation was comprised of a 1:1 mixture of individually-formulated saRNA-HA plus saRNA-NA. Functional antibody responses against A/Wisconsin/588/2019 were measured by a 1-Day MNT assay on Day 21 (3 weeks post prime) and on Day 42 (2 weeks post boost). 50% neutralizing titers are reported.

With respect to FIG. 3, female Balb/c mice were immunized IM on Days 0 and 28 with a different LNP-formulated influenza saRNA vaccine constructs and with the influenza modRNA comparator encoding A/Wisconsin/588/2019 (H1N1) HA and/or NA. The HA/NA post-mix preparation was comprised of a 1:1 mixture of individually-formulated saRNA-HA plus saRNA-NA. Functional antibody responses against A/Wisconsin/588/2019 were measured by a 3-Day MNT assay on Day 42 (2 weeks post boost). 50% neutralizing titers are reported.

With respect to FIG. 4, female Balb/c mice were immunized IM on Days 0 and 28 with a different LNP-formulated influenza saRNA vaccine constructs and with the influenza modRNA comparator encoding A/Wisconsin/588/2019 (H1N1) HA and/or NA. The HA/NA post-mix preparation was comprised of a 1:1 mixture of individually-formulated saRNA-HA plus saRNA-NA. Functional antibody responses against A/Wisconsin/588/2019 were measured by NAI on Day 21 (3 weeks post prime) and on Day 42 (2 weeks post boost).

### 3. Conclusion

The seasonal influenza saRNA vaccine is intended to express 4 different HA and 4 different NA proteins to match the dominant circulating influenza strains each season. This could potentially be achieved using 8 individual saRNA components or 4 bicistronic saRNA components. To that end, the feasibility of a bicistronic saRNA approach was evaluated in a mouse immunogenicity study. A bicistronic saRNA vaccine candidate encoding both HA and NA antigens (TC83-delkozak-HA-SGP-NA-80A) was compared to a monocistronic saRNA-HA or saRNA-NA control encoding a single antigen (TC83-HA-40A or TC83-NA-40A), and also to a 1:1 mix of individually formulated saRNA-HA+saRNA-NA components. ModRNA vaccine candidates encoding the same A/Wisconsin/588/2019 (H1N1) HA or NA antigens were also included as additional comparators. Balb/c mice were immunized IM on Day 0 with 20 ng of the bicistronic and monocistronic saRNA vaccine preparations, 40 ng total (20 ng each) of the 1:1 mix of saRNA-HA+saRNA-NA, and 200 ng of the modRNA comparators. All saRNA LNPs were in the 10 mM Tris/10% Sucrose+20 mM Glutamic Acid, pH 7.4 matrix that is selected for clinical use. On Day 21 (3 weeks after primary immunization), anti-HA antibody responses were induced as measured by HAI and MNT (FIG. 1 and FIG. 2) and anti-NA antibody responses were also induced as measured by NAI (FIG. 4). The bicistronic saRNA vaccine candidate achieved similar titers as the saRNA vaccine comprised of a 1:1 mix of individually-formulated monocistronic saRNA HA+saRNA-NA. Titers for saRNA vaccine formulations expressing two antigens were similar to or just slightly lower than the titers produced by saRNA controls expressing only a single antigen. These results confirmed that the bicistronic saRNA approach is feasible and indicated that the saRNA vaccines provided dose sparing compared to modRNA in Balb/c mice based on antibody titers after a single immunization.

Overall, the bicistronic saRNA construct evaluated produced functional and neutralizing antibody titers that were similar to the levels induced by an saRNA vaccine comprised of individually-formulated HA and NA monocistronic saRNAs. Titers for saRNA vaccines expressing two antigens were similar or just slightly lower than the saRNA-HA or -NA only controls. These preliminary results confirmed that the bicistronic saRNA approach is feasible, irrespective of the regulatory element used to drive the second gene-of-interest (IRES vs. SGP), the order of antigen placement (HA-NA or NA-HA), deletion of kozak sequence, and polyA tail length (40A vs. 80A).

### Example 7: Immunogenicity of an SaRNA Influenza Vaccine Containing Modified Nucleosides

To determine if incorporation of modified bases can produce a more tolerable and potent saRNA vaccine, saRNA preparations expressing the influenza HA were generated by replacing uridine with varying amounts of N1-methylpseudouridine from 0% to 100%. The impact of increasing the percentage of modified bases on in vitro antigen expression was dependent on cell type. In particular, in an immune-competent human cell line such as HeLa, saRNA containing 25-75% N1-methylpseudouridine resulted in higher in vitro antigen expression than the unmodified (0%) saRNA control. However, saRNA with 100% base modification consistently produced low levels of antigen regardless of cell type, likely due to an impairment in replicase function. Increasing the percentage of modified nucleosides incorporated into saRNA also correlated with a reduction in the level of activation of different PRRs or RNA sensors, such as TLR3, TLR7, and RIG-1 in reporter cell lines (data not shown).

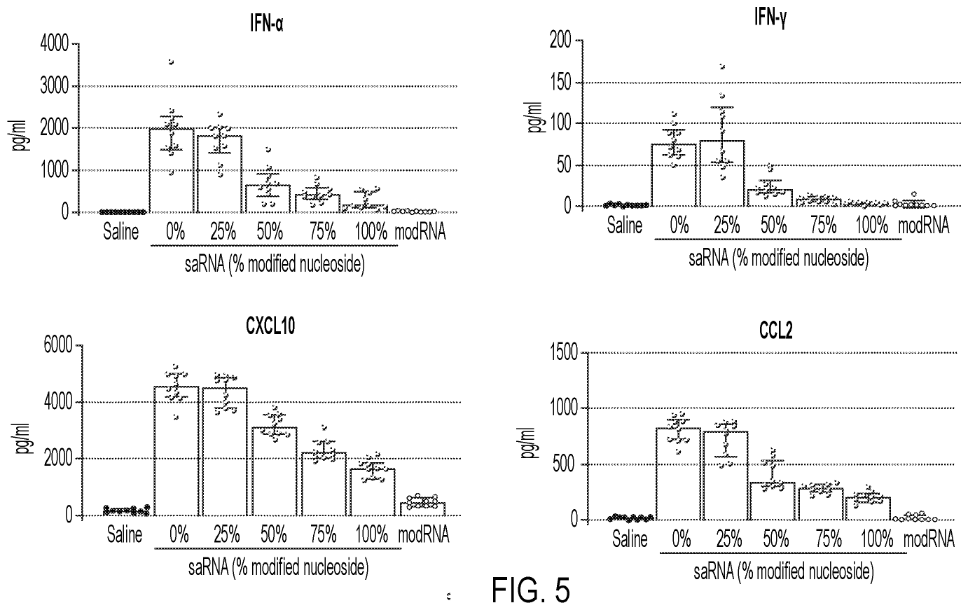

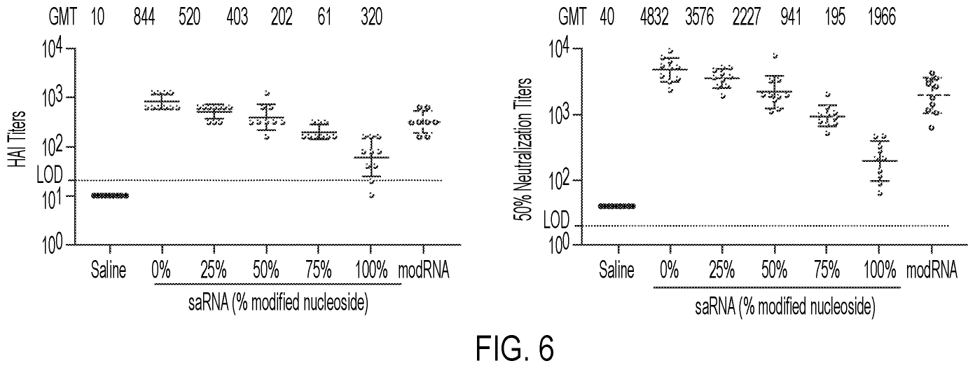

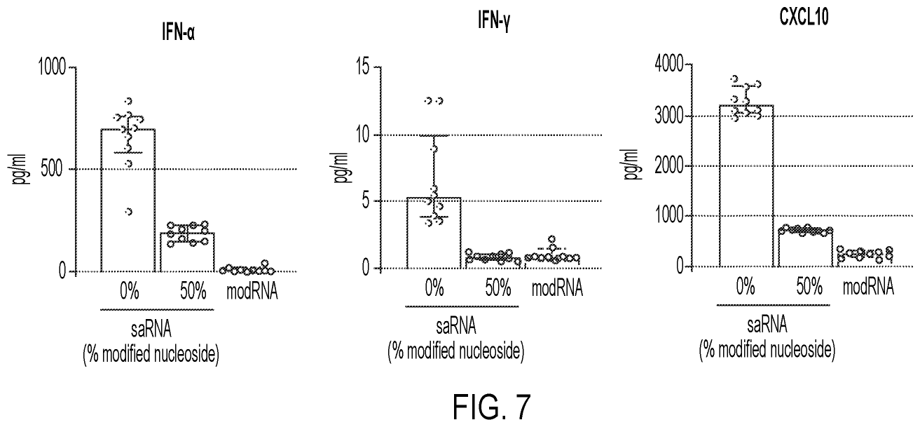

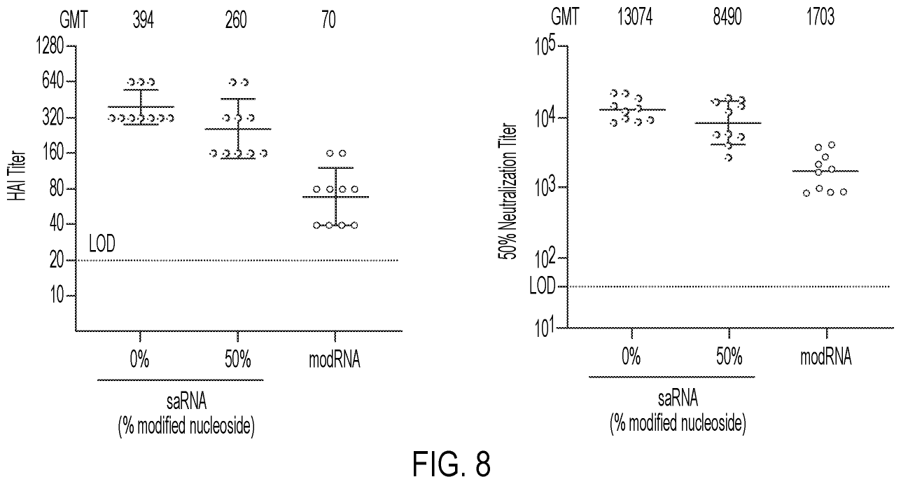

To assess immunogenicity, Balb/c mice were immunized IM on Day 0 with 200 ng of saRNA vaccine preparations containing different amounts of N1-methylpseudouridine. Incorporating higher amounts of modified nucleosides correlated with less innate immune activation, as measured by cytokine and chemokine secretion in the serum on Day 1 post vaccination (FIG. 5), which may improve tolerability of the vaccine. However, higher amounts of modified nucleosides in saRNA also correlated with a reduction in neutralizing antibody titers at 3 weeks after vaccination (FIG. 6), which could potentially reflect the impact on replicase activity. These results were confirmed in a different C57BL6/J mouse species (FIG. 7 and FIG. 8). Based on the mice data, an saRNA construct with 50% incorporation of modified nucleosides substantially reduced secretion of cytokines and chemokines compared to the unmodified control, with a more modest decrease in antibody titers to levels that were similar to or higher than the modRNA-HA benchmark. Overall, the data suggest that partial incorporation of modified bases can be tolerated by saRNA to reduce early innate immune stimulation while still eliciting strong adaptive humoral responses.

Usage of 50% modified bases may partially impact replicase function but the enhanced tolerability could potentially result in an improved saRNA vaccine in humans.

With respect to FIG. 5, female Balb/c mice were immunized IM on Day 0 with 200 ng of LNP-formulated influenza saRNA vaccine preparations containing different amounts (0% to 100%) of N1-methylpseudouridine or with the influenza modRNA comparator encoding A/Wisconsin/588/2019 (H1N1) HA. Serum cytokines and chemokines were measured 24 hours after the first immunization using a Mouse Anti-Virus Response panel LEGENDplex assay. Data reported as median with inter quartile range.

With respect to FIG. 6, female Balb/c mice were immunized IM on Day 0 with 200 of LNP-formulated influenza saRNA vaccine preparations containing different amounts (0% to 100%) of N1-methylpseudouridine or the modRNA comparator encoding A/Wisconsin/588/2019 (H1N1) HA. Antibody responses against A/Wisconsin/588/2019 were measured by HAI or a 1-Day MNT assay on Day 21 (3 weeks after immunization). HAI and 50% neutralization titers are reported (Geometric mean with geometric SD).

With respect to FIG. 7, Female C57BL6/J mice were immunized IM on Day 0 with 200 ng of LNP-formulated influenza saRNA vaccine preparations containing different amounts (0% or 50%) of N1-methylpseudouridine or with the influenza modRNA comparator encoding A/Wisconsin/588/2019 (H1N1) HA. Serum cytokines and chemokines were measured 24 hours after the first immunization using a Mouse Anti-Virus Response panel LEGENDplex assay. Data reported as median with inter quartile range.

With respect to FIG. 8, Female C57BL6/J mice were immunized IM on Day 0 with 200 of LNP-formulated influenza saRNA vaccine preparations containing different amounts (0% or 50%) of N1-methylpseudouridine or the modRNA comparator encoding A/Wisconsin/588/2019 (H1N1) HA. Antibody responses against A/Wisconsin/588/2019 were measured by HAI or a 1-Day MNT assay on Day 21 (3 weeks after immunization). HAI and 50% neutralization titers are reported (Geometric mean with geometric SD).

### Example 8: Immunogenicity of a Quadrivalent Bicistronic SARNA Influenza Vaccine Encoding HA and Na from Four Seasonal Influenza Strains

The primary pharmacology of the influenza saRNA vaccine was evaluated in nonclinical studies in vitro and in vivo. In vitro and in vivo studies demonstrated that the influenza saRNA vaccine encodes influenza HA and/or NA proteins that induce strong functional and neutralizing antibody responses and robust CD4+ and CD8+ T cell responses, and allow significantly smaller doses compared to modRNA in mice. Replication of the saRNA also results in innate immune activation, which may potentiate the adaptive immune response to the expressed antigen(s). Efficient in vitro expression of the HA and NA glycoproteins from influenza saRNA vaccines was demonstrated in cultured cells. Mouse, rat, and ferret immunogenicity studies demonstrated that different influenza saRNA vaccine preparations elicited strong functional and neutralizing antibody responses and T-cell responses. Innate immune activation, as measured by release of serum cytokines and chemokines 24 hours post immunization, were also demonstrated in mice and rats. Immunogenicity studies in mice, benchmarked against influenza modRNA vaccines, also support the use of a bicistronic influenza saRNA construct expressing two separate influenza antigens (HA and NA) from the same saRNA vector. Immunogenicity studies in mice also show that saRNA can tolerate partial incorporation of modified nucleosides to reduce early innate immune stimulation while still eliciting strong adaptive humoral responses. Lastly, immunogenicity studies in mice also support the combination of four bicistronic influenza saRNA constructs, each encoding a different HA and NA, to target four seasonal influenza strains.

Influenza saRNA vaccine candidates selected for initial POC testing contain the full-length, codon-optimized coding sequence for the HA or NA glycoprotein from the A/Wisconsin/588/2019 (H1N1) cell-based virus strain recommended for use in the 2021 2022 and 2022-2023 Northern Hemisphere and the 2022 Southern Hemisphere influenza seasons.

TABLE 12
saRNA Preparations

| | | | | Poly A |
|---|---|---|---|---|
| | RNA | Vector | | tail |
| RNA Construct | number | Backbone | Vaccine Antigen(s) | length |
| 1. TC83-HA-40A- | PF- | TC-83 | HA from | 40 |
| Wisconsin | 07852352 | | A/Wisconsin/588/ | |
| | | | 2019 | |
| 2. TC83-HA-80A- | PF- | TC-83 | HA from | 80 |
| Wisconsin | 07836391 | | A/Wisconsin/588/ | |
| | | | 2019 | |
| 3. TC83-Mit2 | PF- | TC-83- | HA from | 40 |
| aBc1-HA-40A- | 07836394 | Mit2 | A/Wisconsin/588/ | |
| Wisconsin | | aBc1 | 2019 | |
| 4. TRD-HA-40A- | PF- | TRD | HA from | 40 |
| Wisconsin | 07836395 | | A/Wisconsin/588/ | |
| | | | 2019 | |
| 5. TC83-NA-80A- | PF- | TC-83 | NA from | 80 |
| Wisconsin | 07836396 | | A/Wisconsin/588/ | |
| | | | 2019 | |
| 6. TC83-delkozak- | PF- | TC-83 | HA and NA from | 80 |
| HA-SGP-NA-80ª- | 07867246 | | A/Wisconsin/588/ | |
| Wisconsin | | | 2019 | |
| 7. TC83-HA-40A- | PF- | TC-83 | HA from | 40 |
| 50U-50pU- | 07871987 | | A/Wisconsin/588/ | |
| Wisconsin | | | 2019 | |
| 8. TC83-delkozak- | PF- | TC-83 | HA and NA from | 80 |
| HA-SGP-NA- | 07914705 | | B/Austria/1359417/ | |
| 80A-Austria | | | 2021 | |
| 9. TC83-delkozak- | PF- | TC-83 | HA and NA from | 80 |
| HA-SGP-NA- | 07915048 | | A/Wisconsin/588/ | |
| 80A-Quadrivalent | | | 2019, | |
| | | | A/Darwin/6/2021, | |
| | | | B/Austria/1359417/ | |
| | | | 2021, and | |
| | | | B/Phuket/3073/2013 | |

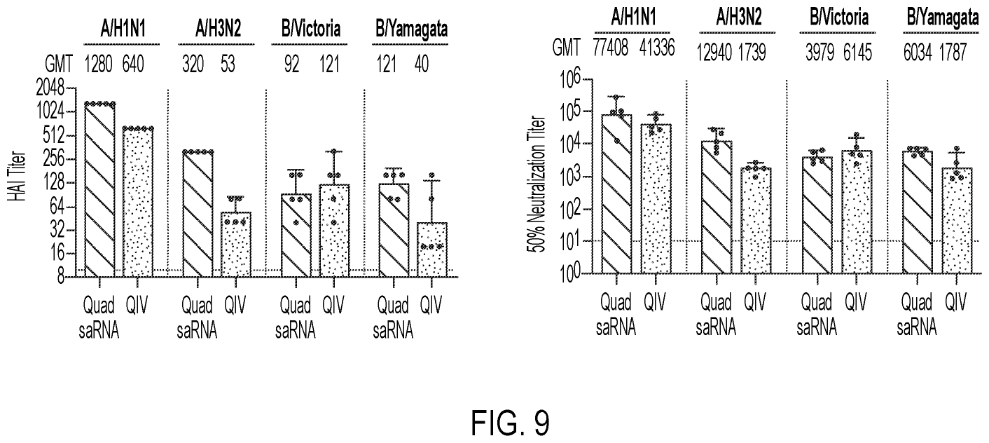

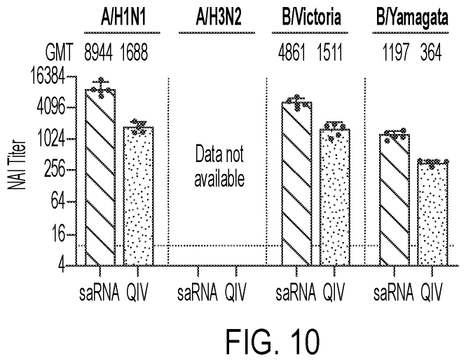

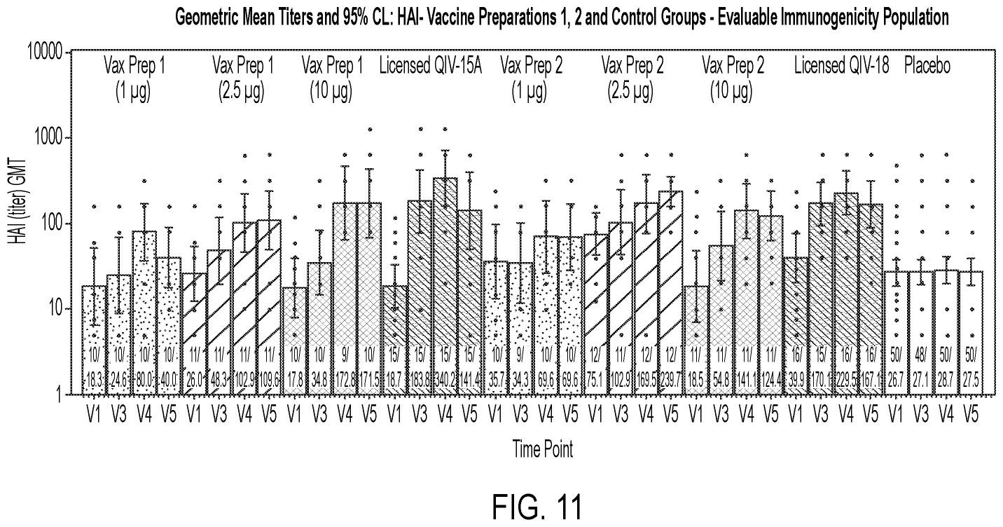

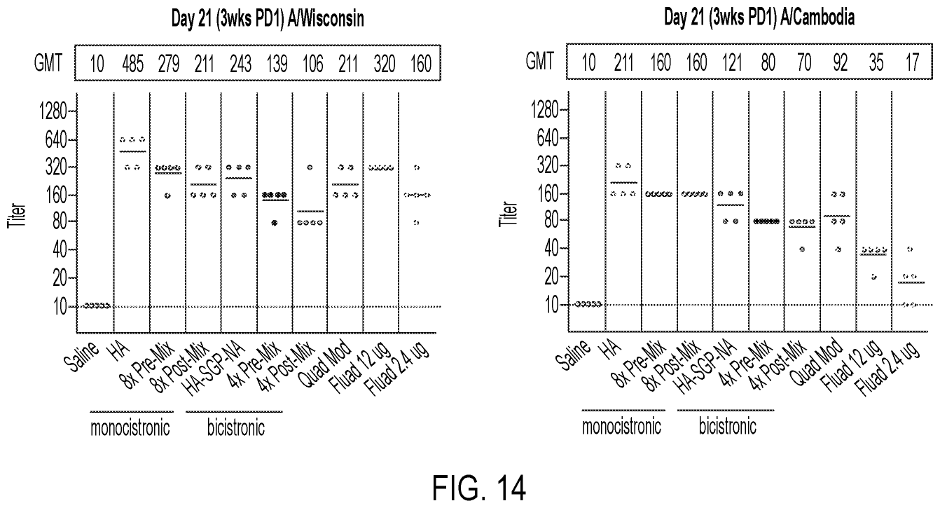

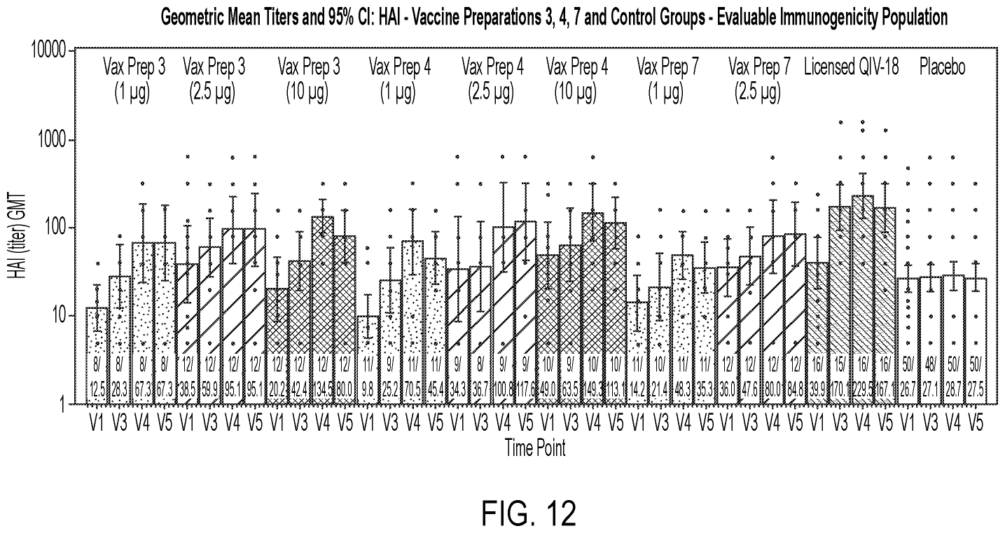

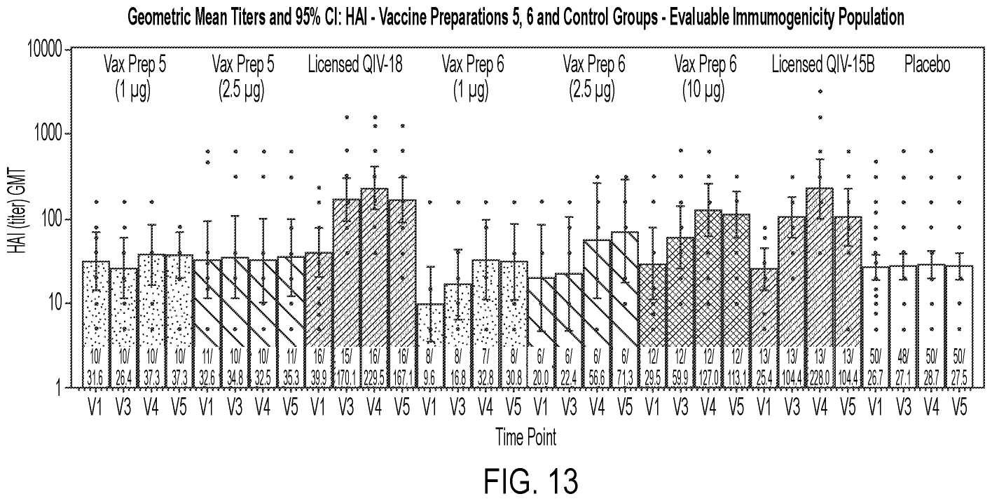

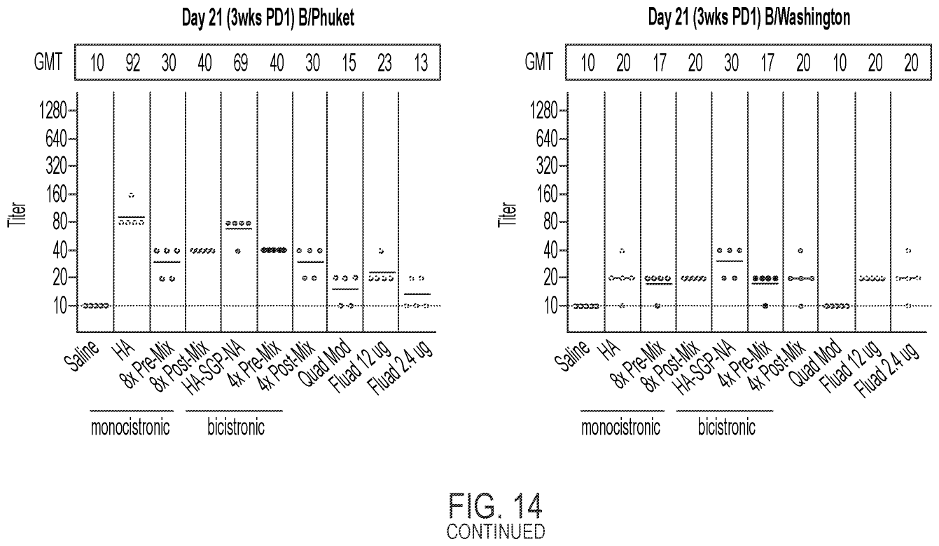

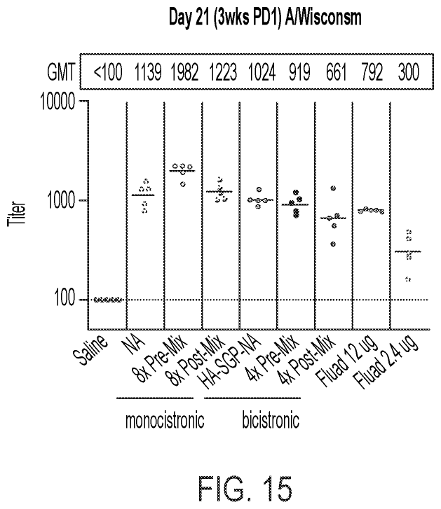

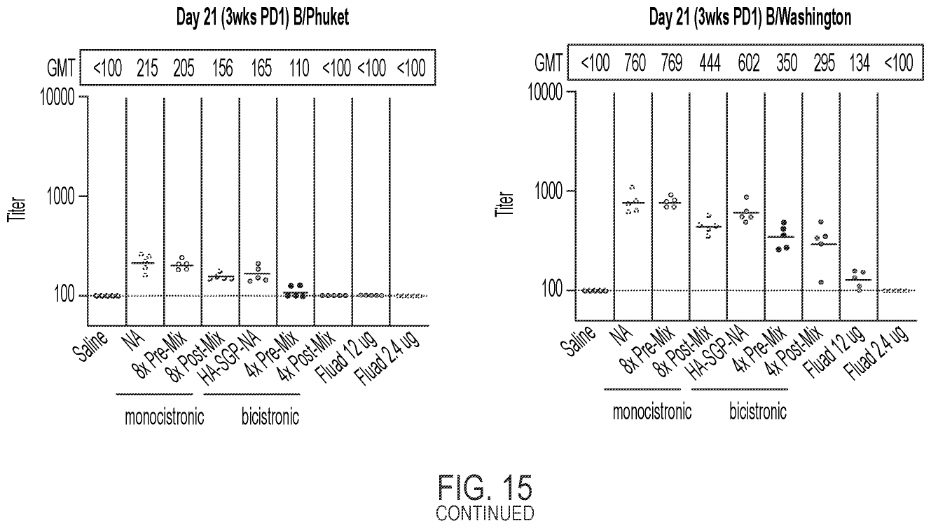

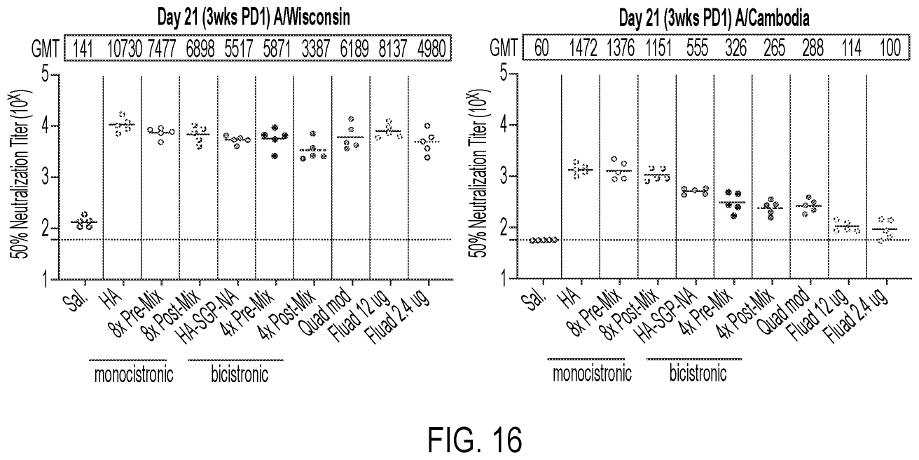

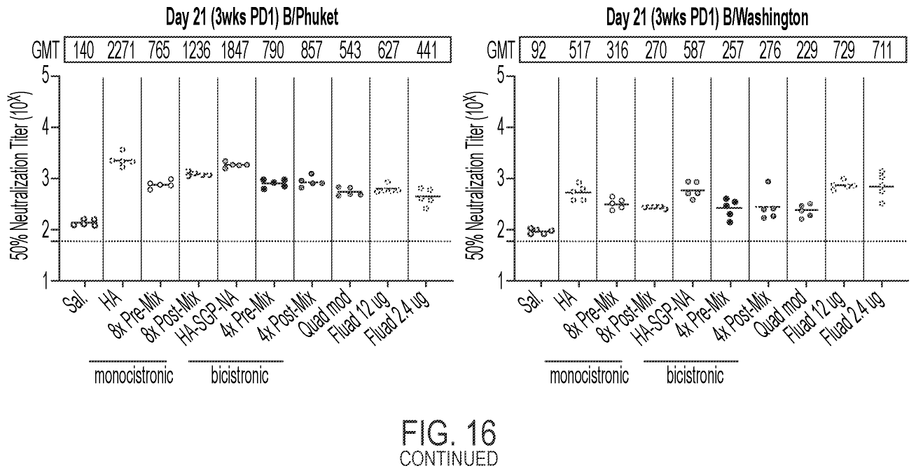

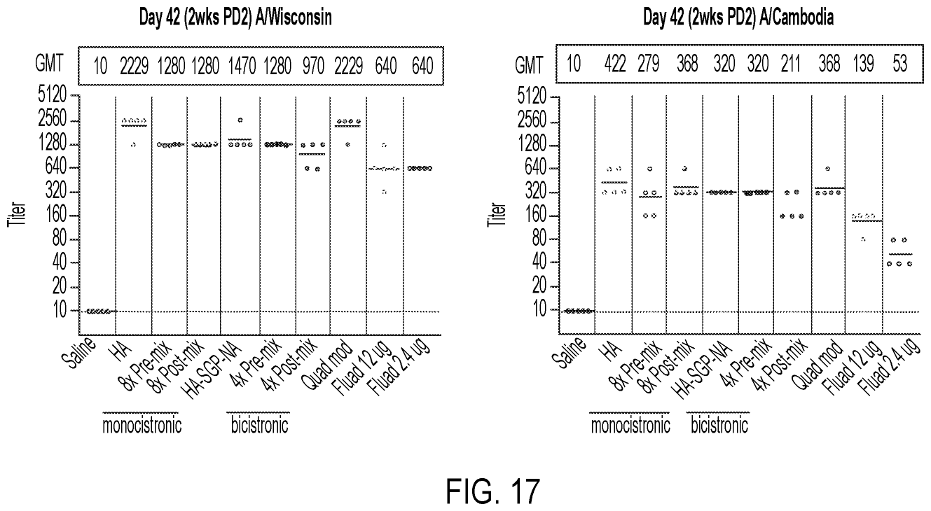

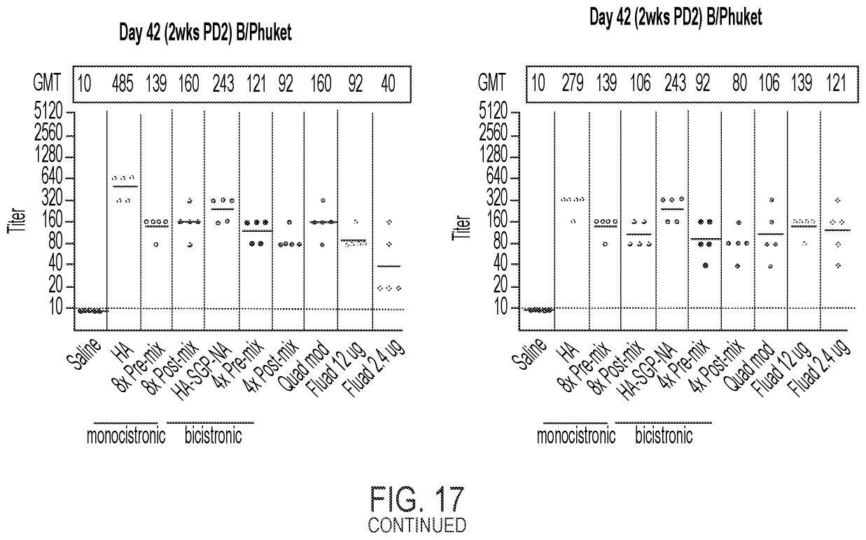

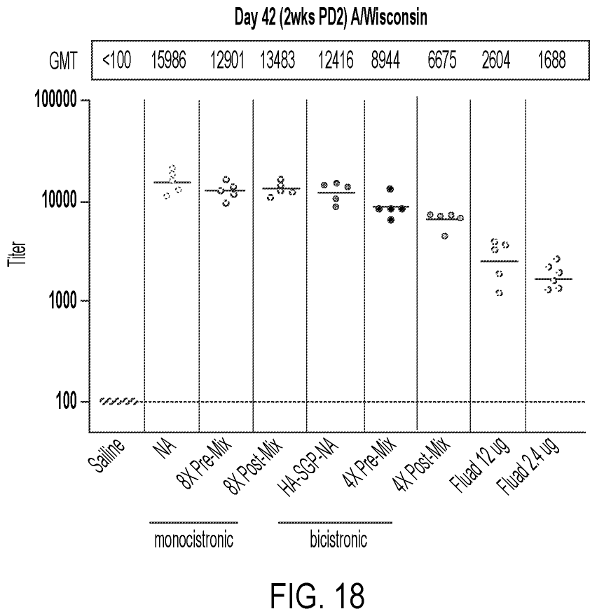

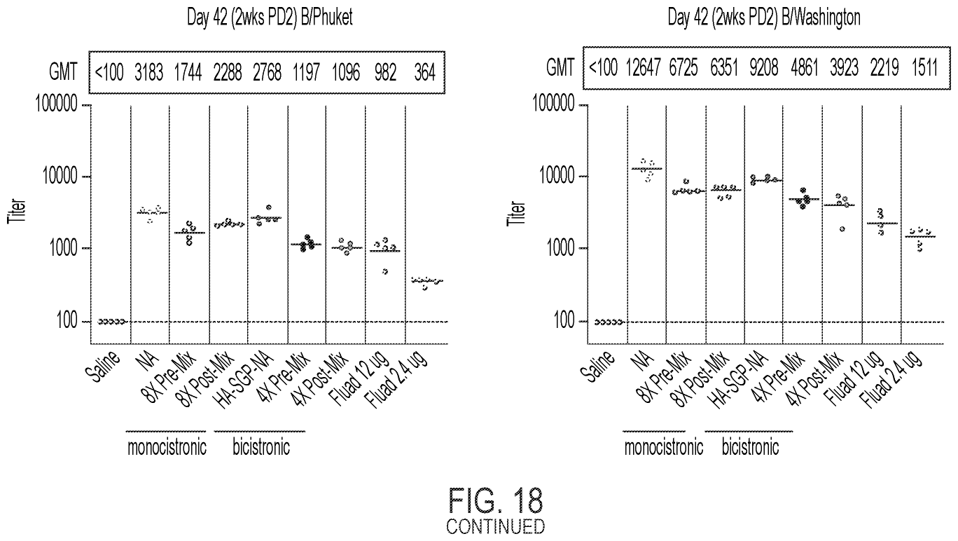

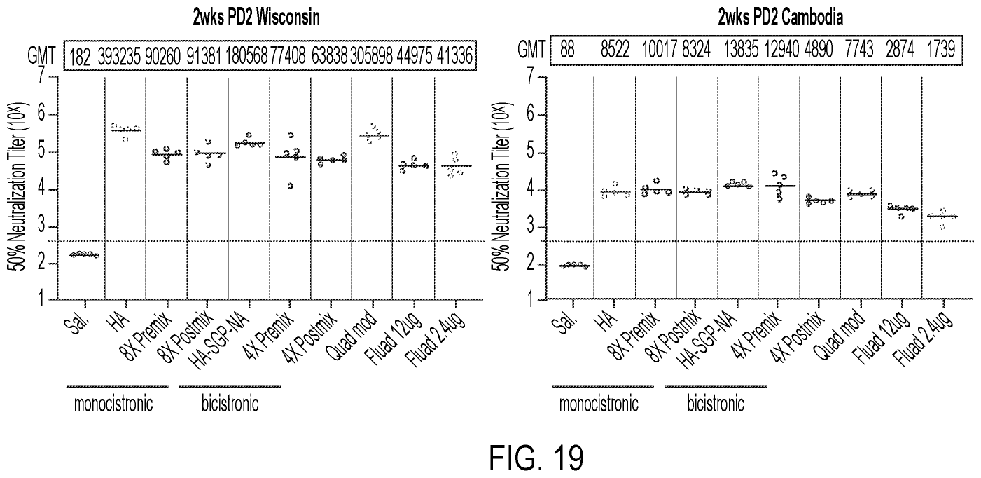

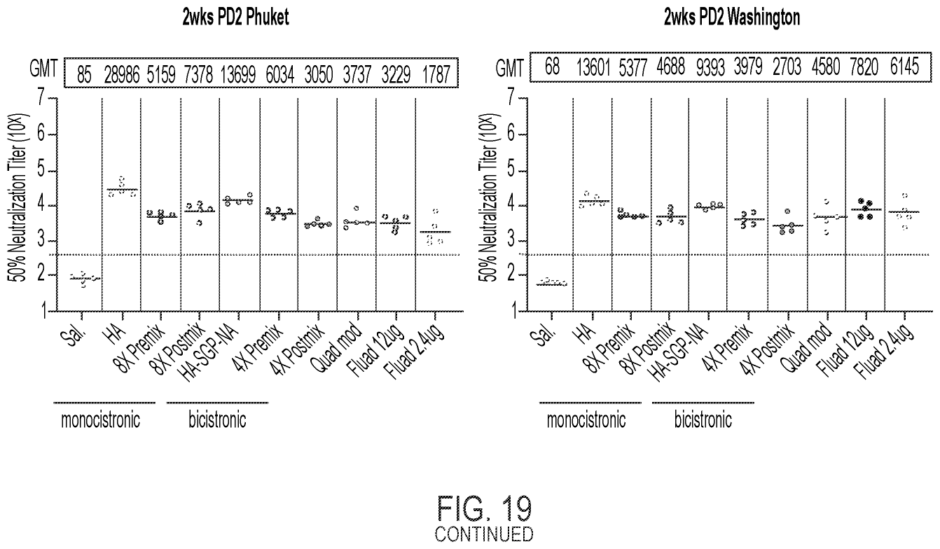

A seasonal influenza saRNA vaccine expressing 4 different HA and 4 different NA proteins to match the dominant circulating influenza strains each season can be achieved using 4 bicistronic saRNA components. The feasibility of a quadrivalent bicistronic saRNA approach was evaluated in a mouse immunogenicity study, benchmarked against the Northern Hemisphere 2021-22 licensed adjuvanted seasonal quadrivalent influenza vaccine (QIV; FluAd). BALB/c mice were immunized IM on Days 0 and 28 with 0.8 μg total dose of a quadrivalent saRNA vaccine (0.2 μg per component) or 2.4 μg of the licensed QIV comparator. On Day 42 (2 weeks after the second immunization), anti-HA antibody responses against each of the four components were induced as measured by HAI and MNT (FIG. 9) and anti-NA antibody responses were also induced as measured by NAI (FIG. 10). The quadrivalent bicistronic saRNA vaccine candidate achieved similar or higher HA and NA titers as the QIV comparator.

With respect to FIG. 9, female Balb/c mice were immunized IM on Day 0 with 20 ng of the LNP-formulated quadrivalent saRNA comprised of 4 bicistronic constructs encoding HA and NA from A/Wisconsin/588/2019 (H1N1), A/Cambodia/e0826360/2020 (H3N2), B/Washington/2/2019 (B/Victoria-lineage), and B/Phuket/3073/2013 (B/Yamagata-lineage), or with 2.4 μg of a licensed, adjuvanted quadrivalent inactivated vaccine (QIV; FluAd). Antibody responses against each vaccine component were measured by HAI or a 1-Day MNT assay on Day 42 (2 weeks after 2nd dose). HAI and 50% neutralization titers are reported (Geometric mean with geometric SD).

With respect to FIG. 10, Female Balb/c mice were immunized IM on Day 0 with 20 ng of the LNP-formulated quadrivalent saRNA comprised of 4 bicistronic constructs encoding HA and NA from A/Wisconsin/588/2019 (H1N1), A/Cambodia/e0826360/2020 (H3N2), B/Washington/2/2019 (B/Victoria-lineage), and B/Phuket/3073/2013 (B/Yamagata-lineage), or with 2.4 μg of a licensed, adjuvanted quadrivalent inactivated vaccine (QIV; FluAd). Antibody responses against each vaccine component were measured by NAI on Day 42 (2 weeks after 2nd dose). NAI titers are reported (Geometric mean with geometric SD) for 3 of the 4 strains. H3N2 NAI titers could not be reported for both the saRNA and QIV due to technical issues with the NAI assay for this strain.

### Example 9: Effects in Humans

C4861001 is an ongoing Phase 1 FIH study assessing safety, tolerability, and immunogenicity of PF-07845104. As of the data cut-off date of 15 Nov. 2022, 253 participants were randomized and 248 participants received the vaccination. A total of 5 participants were not vaccinated (1 participant from vaccine preparation 4 in 2.5 μg group, 2 participants from vaccine preparation 5 in 2.5 μg group, 1 participant from vaccine preparation 5 in 10 μg group, and 1 participant from vaccine preparation 6 in 2.5 μg group). A licensed QIV was used as a comparator.

### Safety and Efficacy—Study C4861001

C4861001 is an ongoing FIH Phase 1, randomized, placebo-controlled, observer-blinded, sponsor-unblinded, dose-finding, and vaccine composition/formulation selection study in healthy adults. This study evaluates the safety, tolerability, and immunogenicity of a single dose of different monocistronic and ultimately bicistronic saRNA vaccine preparations against influenza. Participants 18 through 49 years of age are randomized 4:1 to receive either an saRNA vaccine preparation or placebo, respectively. An additional group of participants are independently enrolled to receive licensed QIV as a control.

Table 12 provides the details of the saRNA vaccine preparation.

TABLE 13
Vaccine Preparation Details

| | | | Poly A |
|---|---|---|---|
| | Vector | Vaccine | Tail |
| Vaccine Preparation | Backbone | Antigen(s) | Length |
| 1. PF-07852352: Influenza | TC83 | HA (monocistronic) | 40 |
| saRNA (TC83-HA-40A) | | | |
| 2. PF-07836391: Influenza | TC83 | HA (monocistronic) | 80 |
| saRNA (TC83-HA-80A) | | | |
| 3. PF-07836394: Influenza | TC83-MIT2 | HA (monocistronic) | 40 |
| saRNA (TC83-MIT2 aBc1- | aBc1 | | |
| HA-40A) | | | |
| 4. PF-07836395: Influenza | TRD | HA (monocistronic) | 40 |
| saRNA (TRD-HA-40A) | | | |
| 5. PF-07836396: Influenza | TC83 | NA (monocistronic) | 80 |
| saRNA (TC83-NA-80A) | | | |
| 6. PF-07867246: Influenza | TC83 | HA and NA | 80 |
| saRNA (TC83-delkozak- | | (bicistronic) | |
| HA-SGP-NA-80A) | | | |
| (bicistronic) | | | |
| 7. PF-07871987: Influenza | TC83 | HA (monocistronic) | 40 |
| saRNA (TC83-HA-40A- | | | |
| 50U-50pU) | | | |

Abbreviations: 50 pU = 50% N1-methylpseudouridine; 50 U = 50% uridine; HA = hemagglutinin; NA = neuraminidase; poly A = polyadenylated; SGP = subgenomic promoter; TRD = Trinidad donkey strain.
Note:
This list is not exhaustive, and preparations may be added or omitted.

### Vaccine Preparations 1, 2 and Control Groups—Randomized Participants

A total of 36 participants each were randomized into Vaccine Preparation 1 and Vaccine Preparation 2 groups, 63 participants into placebo group and 33 participants into control groups.

### Vaccine Preparation 1

In this group, 11 participants received 1 μg, 13 participants received 2.5 μg, and 12 participants received 10 μg of vaccination. One participant was randomized to 2.5 μg but was erroneously dosed with 1 μg and has therefore been included in the 1 μg group for the purposes of safety analysis.

Five participants (1 participant from 1 μg group and 2 participants each from 2.5 μg and 10 μg groups) withdrew from the study after vaccination

### Vaccine Preparation 2

In this group, 12 participants each received 1 μg, 2.5 μg, and 10 μg of vaccination. Oneparticipant from 1 μg group lost to follow up after vaccination.

### Vaccine Preparations 3, 4, 7 and Control Groups—Randomized Participants

A total of 36 participants each were randomized into Vaccine Preparation 3, Vaccine Preparation 4, and Vaccine Preparation 7 groups, 63 participants into placebo. One participant from Vaccine Preparation 4 2.5-μg group was not vaccinated.

### Vaccine Preparation 3

In this group, 12 participants each received 1 μg, 2.5 μg, and 10 μg of vaccination. Two participants from 1 μg group withdrew from the study after vaccination.

### Vaccine Preparation 4

In this group, 12 participants each received 1 μg and 10 μg of vaccination and 11 participants received 2.5 μg of the vaccination.

Two participants (1 participant each from 1 μg and 10 μg groups) withdrew from the study after vaccination.

### Vaccine Preparation 7

In this group, 12 participants each received 1 μg, 2.5 μg, and 10 μg of vaccination.

### Vaccine Preparations 5, 6 and Control Groups—Randomized Participants

A total of 38 participants were randomized into Vaccine Preparation 5, 35 participants into Vaccine Preparation 6, 63 participants into placebo, and 33 participants into control groups. Three participants from Vaccine Preparation 5 (2 participants from 2.5 μg group and 1 participant from 10 μg group) and 1 participant from Vaccination Preparation 6 2.5-μg were not vaccinated.

### Vaccine Preparation 5

In this group, 12 participants each received 1 μg and 2.5 μg of vaccination and 11 participants received 10 μg of vaccination. Two participants from 2.5 μg group and 1 participant from 10 μg group were not vaccinated.

Two participants from 1 μg group withdrew from the study after vaccination.

### Vaccine Preparation 6

In this group, 11 participants each received 1 μg and 2.5 μg of vaccination, and 12 participants received 10 μg of vaccination. One participant from 2.5 μg group was not vaccinated.

Five participants (1 participant from 1 μg group and 4 participants from 2.5 lig group) withdrew from the study after vaccination.

### Immunogenicity

A total of 253 participants were randomized to receive the vaccination of which 196 participants were evaluable for immunogenicity.

A dose-dependent increase in HAI GMTs was observed 4 weeks following administration of vaccine. HAI GMTs at Day 1 (prior to vaccination), and at 1, 2, and 4 weeks following administration of vaccine are shown in FIG. 11, FIG. 12, and FIG. 13.

### Vaccination Preparation 1 and 2

Proportion of participants achieving seroconversion 4 weeks following 10 μg is higher than the proportion of participants achieving seroconversion following 1 μg and 2.5 lig (Table 14).

### Vaccination Preparation 3, 4, and 7

In Vaccination Preparation 3, proportion of participants achieving seroconversion 4 weeks following 10 μg is higher than the proportion of participants achieving seroconversion following 1 μg and 2.5 μg (Table 15).

TABLE 14
Proportion of Participants Achieving HAI Seroconversion for A/Wisconsin/588/2019
(H1N1) Strain After Vaccination - Vaccine Preparations 1, 2 and
Control Groups - Evaluable Immunogenicity Population

| | Vaccine Group (as Randomized) | | | | | | | | |
|---|---|---|---|---|---|---|---|---|---|
| | Vax Prep 1 | | | | Vax Prep 2 | | | | |
| | 1 | 2.5 | 10 | Licensed | 1 | 2.5 | 10 | Licensed | |
| | μg | μg | μg | QIV-15A^{a} | μg | μg | μg | QIV-18^{b} | Placebo^{c} |
| | N^{e} | N^{e} | N^{e} | N^{e} | N^{e} | N^{e} | N^{e} | N^{e} | N^{e} |
| | n^{f} | n^{f} | n^{f} | n^{f} | n^{f} | n^{f} | n_{f} | n^{f} | n^{f} |
| Sampling | (%) | (%) | (%) | (%) | (%) | (%) | (%) | (%) | (%) |
| Time | (95% | (95% | (95% | (95% | (95% | (95% | (95% | (95% | (95% |
| Point^{d} | CI^{g}) | CI^{g}) | CI^{g}) | CI^{g}) | CI^{g}) | CI^{g}) | CI^{g}) | CI^{g}) | CI^{g}) |
| 1 Week | 10 | 11 | 10 | 15 | 9 | 11 | 11 | 15 | 48 |
| | 1 | 2 | 1 | 10 | 0 | 2 | 3 | 7 | 1 |
| | (10.0) | (18.2) | (10.0) | (66.7) | (0.0) | (18.2) | (27.3) | (46.7) | (2.1) |
| | (0.3, | (2.3, | (0.3, | (38.4, | (0.0, | (2.3, | (6.0, | (21.3, | (0.1, |
| | 44.5) | 51.8) | 44.5) | 88.2) | 33.6) | 51.8) | 61.0) | 73.4) | 11.1) |
| 2 Weeks | 10 | 11 | 9 | 15 | 10 | 12 | 11 | 16 | 50 |
| | 4 | 6 | 8 | 12 | 1 | 7 | 7 | 9 | 2 |
| | (40.0) | (54.5) | (88.9) | (80.0) | (10.0) | (58.3) | (63.6) | (56.3) | (4.0) |
| | (12.2, | (23.4, | (51.8, | (51.9, | (0.3, | (27.7, | (30.8, | (29.9, | (0.5, |
| | 73.8) | 83.3) | 99.7) | 95.7) | 44.5) | 84.8) | 89.1) | 80.2) | 13.7) |
| 4 Weeks | 10 | 11 | 10 | 15 | 10 | 12 | 11 | 16 | 50 |
| | 2 | 5 | 8 | 10 | 0 | 7 | 8 | 6 | 3 |
| | (20.0) | (45.5) | (80.0) | (66.7) | (0.0) | (58.3) | (72.7) | (37.5) | (6.0) |
| | (2.5, | (16.7, | (44.4, | (38.4, | (0.0, | (27.7, | (39.0, | (15.2, | (1.3, |
| | 55.6) | 76.6) | 97.5) | 88.2) | 30.8) | 84.8) | 94.0) | 64.6) | 16.5) |

^{a}Includes participants in study receiving licensed QIV, whose VRD data were tested at the same time as VRD data of C1 groups were tested.
^{b}Includes participants in study receiving licensed QIV, whose VRD data were tested at the same time as VRD data of C2-C5 and C7 groups were tested.
^{c}Includes participants in study receiving placebo as randomized.
^{d}Protocol-specified timing for blood sample collection.
^{e}N = number of participants with valid and determinate assay results for the specified assay both before vaccination and at the specified sampling time point.
^{f}n = Number of participants with valid and determinate assay results for the specified assay achieving HAI seroconversion at the specified sampling time point.
^{g}Exact 2-sided CI based on the Clopper and Pearson method.
Note:
HAI refers to assay name of FLU_HAI_H1N1_WIS19 tested at Baseline, 1 Week, 2 Weeks post vaccination, and FLU_HAI_H1N1_II_WIS19 tested at Baseline, 4 Weeks post vaccination.
Note:
Seroconversion is defined as an HAI titer <1:10 prior to vaccination and ≥1:40 at the time point of interest, or an HAI titer of ≥1:10 prior to vaccination with a 4-fold rise at the time point of interest.

TABLE 15
Proportion of Participants Achieving HAI Seroconversion for A/Wisconsin/588/2019 (H1N1) Strain After
Vaccination - Vaccine Preparations 3, 4, 7 and Control Groups - Evaluable Immunogenicity Population

| | Vaccine Group (as Randomized) | | | | | | | | | |
|---|---|---|---|---|---|---|---|---|---|---|
| | Vax Prep 3 | | | Vax Prep 4 | | | Vax Prep 7 | | | |
| | 1 | 2.5 | 10 | 1 | 2.5 | 10 | 1 | 2.5 | Licensed | |
| | μg | μg | μg | μg | μg | μg | μg | μg | QIV-18^{a} | Placebo^{b} |
| | N^{d} | N^{d} | N^{d} | N^{d} | N^{d} | N^{d} | N^{d} | N^{d} | N^{d} | N^{d} |
| | n^{e} | n^{e} | n^{e} | n^{e} | n^{e} | n^{e} | n^{e} | n^{e} | n^{e} | n^{e} |
| Sampling | (%) | (%) | (%) | (%) | (%) | (%) | (%) | (%) | (%) | (%) |
| Time | (95% | (95% | (95% | (95% | (95% | (95% | (95% | (95% | (95% | (95% |
| Point^{c} | CI^{f}) | CI^{f}) | CI^{f}) | CI^{f}) | CI^{f}) | CI^{f}) | CI^{f}) | CI^{f}) | CI^{f}) | CI^{f}) |
| 1 Week | 8 | 12 | 12 | 9 | 8 | 9 | 10 | 12 | 15 | 48 |
| | 4 | 2 | 3 | 3 | 1 | 1 | 1 | 0 | 7 | 1 |
| | (50.0) | (16.7) | (25.0) | (33.3) | (12.5) | (11.1) | (10.0) | (0.0) | (46.7) | (2.1) |
| | (15.7, | (2.1, | (5.5, | (7.5, | (0.3, | (0.3, | (0.3, | (0.0, | (21.3, | (0.1, |
| | 84.3) | 48.4) | 57.2) | 70.1) | 52.7) | 48.2) | 44.5) | 26.5) | 73.4) | 11.1) |
| 2 Weeks | 8 | 12 | 12 | 11 | 9 | 10 | 11 | 12 | 16 | 50 |
| | 6 | 5 | 8 | 9 | 2 | 3 | 4 | 5 | 9 | 2 |
| | (75.0) | (41.7) | (66.7) | (81.8) | (22.2) | (30.0) | (36.4) | (41.7) | (56.3) | (4.0) |
| | (34.9, | (15.2, | (34.9, | (48.2, | (2.8, | (6.7, | (10.9, | (15.2, | (29.9, | (0.5, |
| | 96.8) | 72.3) | 90.1) | 97.7) | 60.0) | 65.2) | 69.2) | 72.3) | 80.2) | 13.7) |
| 4 Weeks | 8 | 12 | 12 | 11 | 9 | 10 | 11 | 12 | 16 | 50 |
| | 6 | 5 | 7 | 7 | 2 | 4 | 4 | 4 | 6 | 3 |
| | (75.0) | (41.7) | (58.3) | (63.6) | (22.2) | (40.0) | (36.4) | (33.3) | (37.5) | (6.0) |
| | (34.9, | (15.2 | (27.7, | (30.8, | (2.8, | (12.2, | (10.9, | (9.9, | (15.2, | (1.3, |
| | 96.8) | 72.3) | 84.8) | 89.1) | 60.0) | 73.8) | 69.2) | 65.1) | 64.6) | 16.5) |

^{a}Includes participants in study receiving licensed QIV, whose VRD data were tested at the same time as VRD data of C2-C5 and C7 groups were tested.
^{b}Includes participants in study receiving placebo as randomized.
^{c}Protocol-specified timing for blood sample collection.
^{d}N = number of participants with valid and determinate assay results for the specified assay both before vaccination and at the specified sampling time point.
^{e}n = Number of participants with valid and determinate assay results for the specified assay achieving HAI seroconversion at the specified sampling time point.
^{f}Exact 2-sided CI based on the Clopper and Pearson method.
Note:
HAI refers to assay name of FLU_HAI_H1N1_WIS19 tested at Baseline, 1 Week, 2 Weeks post vaccination, and FLU_HAI_H1N1_II_WIS19 tested at Baseline, 4 Weeks post vaccination.
Note:
Seroconversion is defined as an HAI titer <1:10 prior to vaccination and ≥1:40 at the time point of interest, or an HAI titer of ≥1:10 prior to vaccination with a 4-fold rise at the time point of interest.

TABLE 16
Proportion of Participants Achieving HAI Seroconversion for A/Wisconsin/588/2019
(H1N1) Strain After Vaccination - Vaccine Preparations 5, 6 and
Control Groups - Evaluable Immunogenicity Population

| | Vaccine Group (as Randomized) | | | | | | | |
|---|---|---|---|---|---|---|---|---|
| | Vax Prep 5 | | | Vax Prep 6 | | | | |
| | 1 | 2.5 | Licensed | 1 | 2.5 | 10 | Licensed | |
| | μg | μg | QIV-18^{a} | μg | μg | μg | QIV-15B^{b} | Placebo^{c} |
| | N^{e} | N^{f} | N^{f} | N^{f} | N^{f} | N^{f} | N^{f} | N^{f} |
| | n^{f} | n^{g} | n^{g} | n^{g} | n^{g} | n^{g} | n^{g} | n^{g} |
| Sampling | (%) | (%) | (%) | (%) | (%) | (%) | (%) | (%) |
| Time | (95% | (95% | (95% | (95% | (95% | (95% | (95% | (95% |
| Point^{d} | CI^{g}) | CI^{h}) | CI^{h}) | CI^{h}) | CI^{h}) | CI^{h}) | CI^{h}) | CI^{h}) |
| 1 Week | 10 | 10 | 15 | 8 | 6 | 12 | 13 | 48 |
| | 0 (0.0) | 0 (0.0) | 7 (46.7) | 0 (0.0) | 0 (0.0) | 1 (8.3) | 5 (38.5) | 1 (2.1) |
| | (0.0, | (0.0, | (21.3, | (0.0, | (0.0, | (0.2, | (13.9, | (0.1, |
| | 30.8) | 30.8) | 73.4) | 36.9) | 45.9) | 38.5) | 68.4) | 11.1) |
| 2 Weeks | 10 | 10 | 16 | 7 | 6 | 12 | 13 | 50 |
| | 0 | 0 | 9 | 2 | 1 | 5 | 7 | 2 |
| | (0.0) | (0.0) | (56.3) | (28.6) | (16.7) | (41.7) | (53.8) | (4.0) |
| | (0.0, | (0.0, | (29.9, | (3.7, | (0.4, | (15.2, | (25.1, | (0.5, |
| | 30.8) | 30.8) | 80.2) | 71.0) | 64.1) | 72.3) | 80.8) | 13.7) |
| 4 Weeks | 10 | 11 | 16 | 8 | 6 | 12 | 13 | 50 |
| | 0 | 0 | 6 | 3 | 2 | 6 | 5 | 3 |
| | (0.0) | (0.0) | (37.5) | (37.5) | (33.3) | (50.0) | (38.5) | (6.0) |
| | (0.0, | (0.0, | (15.2, | (8.5, | (4.3, | (21.1, | (13.9, | (1.3, |
| | 30.8) | 28.5) | 64.6) | 75.5) | 77.7) | 78.9) | 68.4) | 16.5) |

^{a}Includes participants in study receiving licensed QIV, whose VRD data were tested at the same time as VRD data of C2-C5 and C7 groups were tested.
^{b}Includes participants in study receiving licensed QIV, whose VRD data were tested at the same time as VRD data of C6 groups were tested.
^{c}Includes participants in study receiving placebo as randomized.
^{d}Protocol-specified timing for blood sample collection.
^{e}N = number of participants with valid and determinate assay results for the specified assay both before vaccination and at the specified sampling time point.
^{f}n = Number of participants with valid and determinate assay results for the specified assay achieving HAI seroconversion at the specified sampling time point.
^{g}Exact 2-sided CI based on the Clopper and Pearson method.
Note:
HAI refers to assay name of FLU_HAI_H1N1_WIS19 tested at Baseline, 1 Week, 2 Weeks post vaccination, and FLU_HAI_H1N1_II_WIS19 tested at Baseline, 4 Weeks post vaccination.
Note:
Seroconversion is defined as an HAI titer <1:10 prior to vaccination and ≥1:40 at the time point of interest, or an HAI titer of ≥1:10 prior to vaccination with a 4-fold rise at the time point of interest.

In Vaccination Preparation 4, proportion of participants achieving seroconversion 4 weeks following 1 μg is higher than the proportion of participants achieving seroconversion following 2.5 μg and 10 μg (Table 15).

In Vaccination Preparation 7, proportion of participants achieving seroconversion 4 weeks following 1 μg is equal to the proportion of participants achieving seroconversion following 2.5 μg (Table 15).

### Vaccination Preparation 5 and 6

In Vaccination Preparation 5, there were no participants achieved seroconversion after 4 weeks. In Vaccination Preparation 6, proportion of participants achieving seroconversion 4 weeks following 10 μg is higher than the proportion of participants achieving seroconversion following 1 μg and 2.5 μg (Table 16).

### Example 10: Immunogenicity of Influenza Octavalent HA/NA SaRNA Vaccines in Mice

This study was performed to test the immunogenicity of octavalent saRNA-LNP vaccines encoding HA and NA antigens from four influenza virus strains in a mouse model. The saRNA constructs used in this study encoded for the hemagglutinin (HA) and/or neuraminidase (NA) proteins from influenza virus strains A/Wisconsin/588/2019, A/Cambodia/e0926360/2020, B/Phuket/3073/2013, or B/Washington/02/2019. Compared to the licensed comparator FluAd, octavalent saRNA-LNP vaccines elicited comparable or higher levels of functional anti-HA, anti-NA, and virus neutralizing antibodies.

Octavalent vaccine formulations composed of either monocistronic or bicistronic saRNA constructs had comparable immunogenicity in mice after two doses. Multivalent saRNA vaccines mixed either prior to (“pre-mix”) or following formulation (“post-mix”) with LNPs had comparable immunogenicity in mice. Octavalent saRNA-LNP vaccines did result in modest interference for some virus strains, in particular for the immunogenicity of influenza B virus strains, compared to mice vaccinated with monocistronic single antigen controls. Overall, these data provide support for the continued evaluation of multivalent saRNA vaccines encoding influenza virus antigens.

The primary objective of this study was to evaluate the immunogenicity of octavalent saRNA vaccines encoding the hemagglutinin (HA) or neuraminidase (NA) proteins from influenza viruses with monocistronic and bicistronic saRNA vaccine controls in mice. The octavalent vaccines were comprised of either eight monocistronic HA or NA saRNAs or four bicistronic HA-NA saRNA constructs. Octavalent saRNA vaccines were also compared to a licensed quadrivalent inactivated influenza virus vaccine comparator (containing 8 HA/NA antigens from 4 strains) and a quadrivalent modified RNA (modRNA)-LNP vaccine encoding 4 HA proteins. A secondary objective of this study was to compare the immunogenicity of octavalent vaccine constructs combined either pre- or post-formulation with lipid nanoparticles (LNPs) in the mouse model.

This study tested saRNA (TC83-delkozak-80A) constructs that encoded for the HA or NA alone (monocistronic) or both HA and NA proteins (bicistronic) from H1N1 A/Wisconsin/588/2019, H3N2 A/Cambodia/e0926360/2020, B/Yam B/Phuket/3073/2013, or B/Vic B/Washington/02/2019. Octavalent formulations, comprising either 8 monocistronic (TC83-delkozak-HA-80A or TC83-delkozak-NA-80A) or 4 bicistronic (TC83-delkozak-HA-SGP-NA-80A) saRNAs formulated in LNPs were tested in mice. Octavalent saRNA vaccines were either mixed prior to formulation in LNPs (premixed) or mixed after formulation of each saRNA construct in LNPs (postmixed). Two alternative quadrivalent vaccine comparators were included in this study: a nucleoside-modified RNA (modRNA)-LNP vaccine encoding 4 HA proteins, and FluAd, a licensed comparator composed of 4 inactivated influenza viruses. Monocistronic and bicistronic saRNA-LNP vaccines for each strain were also included as controls to evaluate any potential interference in antibody responses observed in the octavalent vaccine formulations.

This study was designed with 20 groups as shown in Table 17, each containing a total of 10 female mice (strain of mice: Balb/c). The study schedule assays used in the study are documented below.

TABLE 17
Study Design

| | | | | | Dose | | |
|---|---|---|---|---|---|---|---|
| | | | | Dose | Vol/ | Vax | Bleed |
| Gp# | Mice | RNA DP Description | DP Lot | (μg) | Route | (Day) | (Day) |
| 1 | 10 | Saline | B2012021 | — | 50 μl/IM | 0, 28 | 21, 42 |
| 2 | 10 | RMM234 A/Wisconsin | 00715982-0040-G2 | 0.2 | 50 μl/IM | 0, 28 | 21, 42 |
| | | (H1N1) saRNA NA | | | | | |
| | | monocistronic | | | | | |
| 3 | 10 | RMM235 A/Wisconsin | 00715982-0040-G3- | 0.2 | 50 μl/IM | 0, 28 | 21, 42 |
| | | (H1N1) saRNA HA | repeat | | | | |
| | | monocistronic | | | | | |
| 4 | 10 | RMM223 A/Cambodia | 00715982-0040-G4 | 0.2 | 50 μl/IM | 0, 28 | 21, 42 |
| | | (H3N2) saRNA HA | | | | | |
| | | monocistronic | | | | | |
| 5 | 10 | RMM226 A/Cambodia | 00715982-0040-G5 | 0.2 | 50 μl/IM | 0, 28 | 21, 42 |
| | | (H3N2) saRNA NA | | | | | |
| | | monocistronic | | | | | |
| 6 | 10 | RMM224 B/Phuket (By) | 00715982-0040-G6 | 0.2 | 50 μl/IM | 0, 28 | 21, 42 |
| | | saRNA HA monocistronic | | | | | |
| 7 | 10 | RMM227 B/Phuket (By) | 00715982-0040-G7 | 0.2 | 50 μl/IM | 0, 28 | 21, 42 |
| | | saRNA NA monocistronic | | | | | |
| 8 | 10 | RMM225 B/Washington | 00715982-0040-G8 | 0.2 | 50 μl/IM | 0, 28 | 21, 42 |
| | | (Bv) saRNA HA | | | | | |
| | | monocistronic | | | | | |
| 9 | 10 | RMM228 B/Washington | 00715982-0040-G9 | 0.2 | 50 μl/IM | 0, 28 | 21, 42 |
| | | (Bv) saRNA NA | | | | | |
| | | monocistronic | | | | | |
| 10 | 10 | 8x saRNA monocistronics | 00715982-0040-G10- | 1.6 | 50 μl/IM | 0, 28 | 21, 42 |
| | | premixed & coformulate | repeat | | | | |
| 11 | 10 | 8x saRNA monocistronics | Materials from | 1.6 | 50 μl/IM | 0, 28 | 21, 42 |
| | | postmixed | groups 2-9 | | | | |
| 12 | 10 | TC83-delkozak-HA-SGP- | 00715982-0040-G12 | 0.2 | 50 μl/IM | 0, 28 | 21, 42 |
| | | NA-80A Wisconsin | | | | | |
| | | (bicistronic) | | | | | |
| 13 | 10 | TC83-delkozak-HA-SGP- | 00715982-0040-G13 | 0.2 | 50 μl/IM | 0, 28 | 21, 42 |
| | | NA-80A Cambodia | | | | | |
| | | (bicistronic) | | | | | |
| 14 | 10 | TC83-delkozak-HA-SGP- | 00715982-0040-G14 | 0.2 | 50 μl/IM | 0, 28 | 21, 42 |
| | | NA-80A Phuket | | | | | |
| | | (bicistronic) | | | | | |
| 15 | 10 | TC83-delkozak-HA-SGP- | 00715982-0040-G15 | 0.2 | 50 μl/IM | 0, 28 | 21, 42 |
| | | NA-80A Washington | | | | | |
| | | (bicistronic) | | | | | |
| 16 | 10 | 4x saRNA bicistronics | 00715982-0040-G16 | 0.8 | 50 μl/IM | 0, 28 | 21, 42 |
| | | premixed & coformulate | | | | | |
| 17 | 10 | 4x saRNA bicistronics | Material from | 0.8 | 50 μl/IM | 0, 28 | 21, 42 |
| | | postmixed | groups 12-15 | | | | |
| 18 | 10 | Quadrivalent modRNA | 00709594-0593 | 0.887 | 50 μl/IM | 0, 28 | 21, 42 |
| | | (4x modRNA-HA RMM71, | | | | | |
| | | 81, 90, 91 premixed) | | | | | |
| 19 | 10 | Licensed Flu Vaccine | 312855 | 12 | 100 μl/IM | 0, 28 | 21, 42 |
| | | Comparator (FluAd-QIV) | | | (50 μl/site) | | |
| 20 | 10 | Licensed Flu Vaccine | 312855 | 2.4 | 20 μl/IM | 0, 28 | 21, 42 |
| | | Comparator (FluAd-QIV) | | | | | |

Assays for the Study:

HAI (groups 1,3,4,6,8,10-20);

NAI (groups 1,2,5,7,9,10-17, 19, 20);

1D neut

D21, 42

One 0.3 mL syringe was filled to 0.05 mL, and vaccine was administered via the intramuscular route for each animal. Procedure was repeated on day 28 for the booster vaccination.

TABLE 18
Test Articles and Diluent for the Study

| Item | | Formulation Matrix | Gps to | Vials#/ |
|---|---|---|---|---|
| # | Test articles/Diluent | and information | be used | Storage |
| 1 | Saline | 0.9% NaCl in water | 1 and as | RT |
| | | | diluent for | 2 bottles |
| | | | groups | |
| | | | 2-18 | |
| 2 | LNP RMM234 | 0.083 mg/mL of saRNA LNP in 10 mM | 2 and 11 | 7 × 0.3 mL |
| | A/Wisconsin (H1N1) | Tris 10% Sucrose 20 mM Glutamic acid, | | vials - |
| | saRNA NA | pH 7.4 | | 80° C. |
| | monocistronic | | | (1) prime, |
| | Lot# 00715982-0040- | | | (1) boost, |
| | G2 | | | (3) for gps |
| | | | | 11, 2 extra |
| 3 | LNP RMM235 | 0.06 mg/ml of saRNA LNP in | 3 and 11 | 7 × 0.3 mL |
| | A/Wisconsin (H1N1) | 10 mM Tris 10% Sucrose 20 mM Glutamic | | vials - |
| | saRNA HA | acid, pH 7.4 | | 80° C. |
| | monocistronic | | | (1) prime, |
| | Lot# 00715982-0040- | | | (1) boost, |
| | G3-repeat | | | (3) for gps |
| | | | | 11, 2 extra |
| 4 | RMM223 A/Cambodia | 0.065 mg/ml of saRNA LNP in 10 mM | 4 and 11 | 7 × 0.3 mL |
| | (H3N2) saRNA HA | Tris 10% Sucrose 20 mM Glutamic acid, | | vials - |
| | monocistronic | pH 7.4 | | 80° C. |
| | | | | (1) prime, |
| | | | | (1) boost, |
| | | | | (3) for gps |
| | | | | 11, 2 extra |
| 5 | RMM226 A/Cambodia | 0.084 mg/ml of saRNA LNP in 10 mM | 5 and 11 | 7 × 0.3 mL |
| | (H3N2) saRNA NA | Tris 10% Sucrose 20 mM Glutamic acid, | | vials - |
| | monocistronic | pH 7.4 | | 80° C. |
| | | | | (1) prime, |
| | | | | (1) boost, |
| | | | | (3) for gps |
| | | | | 11, 2 extra |
| 6 | RMM224 B/Phuket | 0.077 mg/ml of saRNA LNP in 10 mM | 6 and 11 | 7 × 0.3 mL |
| | (By) saRNA HA | Tris 10% Sucrose 20 mM Glutamic acid, | | vials - |
| | monocistronic | pH 7.4 | | 80° C. |
| | | | | (1) prime, |
| | | | | (1) boost, |
| | | | | (3) for gps |
| | | | | 11, 2 extra |
| 7 | RMM227 B/Phuket | 0.075 mg/mL of saRNA LNP in 10 mM | 7 and 11 | 7 × 0.3 mL |
| | (By) saRNA NA | Tris 10% Sucrose 20 mM Glutamic acid, | | vials - |
| | monocistronic | pH 7.4 | | 80° C. |
| | | | | (1) prime, |
| | | | | (1) boost, |
| | | | | (3) for gps |
| | | | | 11, 2 extra |
| 8 | RMM225 | 0.086 mg/ml of saRNA LNP in 10 mM | 8 and 11 | 7 × 0.3 mL |
| | B/Washington (Bv) | Tris 10% Sucrose 20 mM Glutamic acid, | | vials - |
| | saRNA HA | pH 7.4 | | 80° C. |
| | monocistronic | | | (1) prime, |
| | | | | (1) boost, |
| | | | | (3) for gps |
| | | | | 11, 2 extra |
| 9 | RMM228 | 0.065 mg/ml of saRNA LNP in 10 mM | 9 and 11 | 7 × 0.3 mL |
| | B/Washington (Bv) | Tris 10% Sucrose 20 mM Glutamic acid, | | vials - |
| | saRNA NA | pH 7.4 | | 80° C. |
| | monocistronic | | | (1) prime, |
| | | | | (1) boost, |
| | | | | (3) for gps |
| | | | | 11, 2 extra |
| 10 | 8× saRNA | 0.066 mg/ml of saRNA LNP in 10 mM | 10 | 10 × 0.3 |
| | monocistronics | Tris 10% Sucrose 20 mM Glutamic acid, | | mL vials - |
| | premixed & | pH 7.4 | | 80° C. |
| | coformulate | | | (3) prime, |
| | | | | (3) boost, |
| | | | | 4 extra |
| 11 | TC83-delkozak-HA- | 0.081 mg/mL of saRNA LNP in 10 mM | 12 and 17 | 7 × 0.3 mL |
| | SGP-NA-80A | Tris 10% Sucrose 20 mM Glutamic acid, | | vials - |
| | Wisconsin (bicistronic) | pH 7.4 | | 80° C. |
| | | | | (1) prime, |
| | | | | (1) boost, |
| | | | | (3) for gps |
| | | | | 17, 2 extra |
| 12 | TC83-delkozak-HA- | 0.070 mg/mL of saRNA LNP in 10 mM | 13 and 17 | 7 × 0.3 mL |
| | SGP-NA-80A | Tris 10% Sucrose 20 mM Glutamic acid, | | vials - |
| | Cambodia (bicistronic) | pH 7.4 | | 80° C. |
| | | | | (1) prime, |
| | | | | (1) boost, |
| | | | | (3) for gps |
| | | | | 17, 2 extra |
| 13 | TC83-delkozak-HA- | 0.065 mg/ml of saRNA LNP in 10 mM | 14 and 17 | 7 × 0.3 mL |
| | SGP-NA-80A Phuket | Tris 10% Sucrose 20 mM Glutamic acid, | | vials - |
| | (bicistronic) | pH 7.4 | | 80° C. |
| | | | | (1) prime, |
| | | | | (1) boost, |
| | | | | (3) for gps |
| | | | | 17, 2 extra |
| 14 | TC83-delkozak-HA- | 0.066 mg/ml of saRNA LNP in 10 mM | 15 and 17 | 7 × 0.3 mL |
| | SGP-NA-80A | Tris 10% Sucrose 20 mM Glutamic acid, | | vials - |
| | Washington | pH 7.4 | | 80° C. |
| | (bicistronic) | | | (1) prime, |
| | | | | (1) boost, |
| | | | | (3) for gps |
| | | | | 17, 2 extra |
| 15 | 4× saRNA bicistronics | 0.060 mg/ml of saRNA LNP in 10 mM | 16 | 4 × 0.3 mL |
| | premixed & | Tris 10% Sucrose 20 mM Glutamic acid, | | vials - |
| | coformulate | pH 7.4 | | 80° C. |
| | | | | (2) prime, |
| | | | | (2) boost, |
| | | | | 2 extra |
| 16 | Quadrivalent modRNA | 0.111 mg/ml of modRNA LNP in 10 mM | 18 | 4 × 0.3 mL |
| | (4× modRNA-HA | Tris 300 mM Sucrose, pH 7.4 | | vials - |
| | RMM71, 81, 90, 91 | | | 80° C. |
| | premixed) | | | (1) prime, |
| | | | | (1) boost, |
| | | | | 2 extra |
| 17 | Quad FluAd (NH21-22) - | A/Victoria/2570/2019 IVR-215, | 19 and 20 | 6 × 0.5 mL |
| | 60 ug/0.5 mL (15 ug | A/Cambodia/e0826360/2020 IVR-224, | | syringe |
| | per strain) | B/Victoria/705/2018 BVR-11, | | 2-8° C. (for |
| | | B/Phuket/3073/2013 BVR-1B | | prime and |
| | | | | boost) |

TABLE 19
Drug Product Materials and Analytical Results

| | | | | | | | | T0 IVE % | | |
|---|---|---|---|---|---|---|---|---|---|---|
| | RNA | | Encapsulation | Size | | Integrity | Cap % | Cells | IVE | Endotoxin |
| Group # | description | Concentration | (%) | (nm) | PDI | by FA(%) | (*DS) | Positive | EC50 | (EU/mL) |
| 2, 11 | RMM234 | 83 μg/mL | 94% | 61 | 0.15 | Main: | 100% | NA: 84% | NA: 20 ng | <0.05 |
| | A/Wisconsin | | | | | 86%, | | (125 ng) | | EU/mL |
| | (H1N1) | | | | | LMS: | | | | |
| | saRNA | | | | | 6% | | | | |
| | NA | | | | | | | | | |
| | monocistrlonic | | | | | | | | | |
| 3, 11 | RMM235 | 60 μg/mL | 94% | 61 | 0.16 | Main: | 100% | HA: 79% | HA: 20 ng | <0.05 |
| | A/Wisconsin | | | | | 75%, | | (125 ng) | | EU/mL |
| | (H1N1) | | | | | LMS: | | | | |
| | saRNA | | | | | 8% | | | | |
| | HA | | | | | | | | | |
| | monocistronic | | | | | | | | | |
| 4, 11 | RMM223 | 65 μg/mL | 93% | 62 | 0.14 | Main: | 98% | HA: 86% | HA: 24 ng | <0.05 |
| | A/Cambodia | | | | | 84%, | | (125 ng) | | EU/mL |
| | (H3N2) | | | | | LMS: | | | | |
| | saRNA | | | | | 6% | | | | |
| | HA | | | | | | | | | |
| | monocistronic | | | | | | | | | |
| 5, 11 | RMM226 | 84 μg/mL | 95% | 62 | 0.16 | Main: | 99% | NA: 72% | NA: 61 ng | 0.07 |
| | A/Cambodia | | | | | 86%, | | (125 ng) | | EU/mL |
| | (H3N2) | | | | | LMS: | | | | |
| | saRNA | | | | | 5% | | | | |
| | NA | | | | | | | | | |
| | monocistronic | | | | | | | | | |
| 6, 11 | RMM224 | 77 μg/mL | 94% | 62 | 0.15 | Main: | 98% | HA: 87% | HA: 12 ng | 0.18 |
| | B/Phuket | | | | | 88%, | | (125 ng) | | EU/mL |
| | (By) | | | | | LMS: | | | | |
| | saRNA | | | | | 5% | | | | |
| | HA | | | | | | | | | |
| | monocistronic | | | | | | | | | |
| 7, 11 | RMM 227 | 75 μg/mL | 97% | 61 | 0.13 | Main: | 99% | NA: 71% | NA: 24 ng | <0.05 |
| | B/Phuket | | | | | 87%, | | (125 ng) | | EU/mL |
| | (By) | | | | | LMS: | | | | |
| | saRNA | | | | | 5% | | | | |
| | NA | | | | | | | | | |
| | monocistronic | | | | | | | | | |
| 8, 11 | RMM225 | 86 μg/mL | 97% | 63 | 0.16 | Main: | 98% | HA: 88% | HA: 17 ng | <0.05 |
| | B/Washington | | | | | 85%, | | (125 ng) | | EU/mL |
| | (Bv) | | | | | LMS: | | | | |
| | saRNA | | | | | 6% | | | | |
| | HA | | | | | | | | | |
| | monocistonic | | | | | | | | | |
| 9, 11 | RMM228 | 65 μg/mL | 97% | 61 | 0.15 | Main: | 99% | NA: 75% | NA: 18 ng | 0.07 |
| | B/Washington | | | | | 84%, | | (125 ng) | | EU/mL |
| | (Bv) | | | | | LMS: | | | | |
| | saRNA | | | | | 7% | | | | |
| | NA | | | | | | | | | |
| | monocistronic | | | | | | | | | |
| 10 | 8x | 72 μg/mL | 96% | 58 | 0.12 | Main: | N/A | A/Wisc | A/Wisc | <0.05 |
| | saRNA | | | | | 79%, | | HA: 55% | HA: 64 ng | EU/mL |
| | monocistronics | | | | | LMS: | | (125 ng) | NA: 42 ng | |
| | premixed & | | | | | 7% | | NA: 58% | A/Cam | |
| | coformulate | | | | | | | (125 ng) | HA: 18 ng | |
| | | | | | | | | A/Cam | NA: >125 ng | |
| | | | | | | | | HA: 62% | B/Phu | |
| | | | | | | | | (63 ng) | HA: 30 ng | |
| | | | | | | | | NA: 29% | NA: 22 ng | |
| | | | | | | | | (63 ng) | B/Wash | |
| | | | | | | | | B/Phu | HA: 8 ng | |
| | | | | | | | | HA: 54% | NA: 7 ng | |
| | | | | | | | | (63 ng) | | |
| | | | | | | | | NA: 58% | | |
| | | | | | | | | (63 ng) | | |
| | | | | | | | | B/Wash | | |
| | | | | | | | | HA: 69% | | |
| | | | | | | | | (63 ng) | | |
| | | | | | | | | NA: 62% | | |
| | | | | | | | | (63 ng) | | |
| 12, 17 | TC83- | 81 μg/mL | 98% | 59 | 0.14 | Main: | 100% | HA: 91% | HA: 26 ng | <0.05 |
| | delkozak- | | | | | 80%, | | (125 ng) | NA: 25 ng | EU/mL |
| | HA-SGP- | | | | | LMS: | | NA: 93% | | |
| | NA-80A | | | | | 5% | | (125 ng) | | |
| | Wisconsin | | | | | | | | | |
| | (bicistronic) | | | | | | | | | |
| 13, 17 | TC83- | 70 μg/mL | 98% | 63 | 0.16 | Main: | 100% | HA: 89% | HA: 31 ng | <0.05 |
| | delkozak- | | | | | 85%, | | (125 ng) | NA: 40 ng | EU/mL |
| | HA-SGP- | | | | | LMS: | | NA: 87% | | |
| | NA-80A | | | | | 6% | | (125 ng) | | |
| | Cambodia | | | | | | | | | |
| | (bicistronic) | | | | | | | | | |
| 14, 17 | TC83- | 65 μg/mL | 98% | 60 | 0.17 | Main: | 100% | HA: 92% | HA: 18 ng | <0.07 |
| | delkozak- | | | | | 85%, | | (125 ng) | NA: 17 ng | EU/mL |
| | HA-SGP- | | | | | LMS: | | NA: 93% | | |
| | NA-80A | | | | | 7% | | (125 ng) | | |
| | Phuket | | | | | | | | | |
| | (bicistronic) | | | | | | | | | |
| 15, 17 | TC83- | 66 μg/mL | 98% | 60 | 0.14 | Main: | 100% | HA: 92% | HA: 22 ng | <0.05 |
| | delkozak- | | | | | 86%, | | (125 ng) | NA: 15 ng | EU/mL |
| | HA-SGP- | | | | | LMS: | | NA: 95% | | |
| | NA-80A | | | | | 6% | | (125 ng) | | |
| | Washington | | | | | | | | | |
| | (bicistronic) | | | | | | | | | |
| 16 | 4x | 60 μg/mL | 95% | 66 | 0.19 | Main: | N/A | HA/Wisc: 66% | HA/Wisc: 30 ng | 0.16 |
| | saRNA | | | | | 84%, | | (125 ng) | HA/Cam: 65 ng | EU/mL |
| | bicistronics | | | | | LMS: | | HA/Cam: 55% | HA/Phu: 13 ng | |
| | premixed & | | | | | 5% | | (125 ng) | HA/Phu: 9 ng | |
| | coformulate | | | | | | | HA/Phu: 74% | NA/Wisc: 27 ng | |
| | | | | | | | | (125 ng) | NA/Cam: 69 ng | |
| | | | | | | | | HA/Wash: 80% | NA/Phu: 6 ng | |
| | | | | | | | | (125 ng) | NA/Wash: 8 ng | |
| | | | | | | | | NA/Wisc: 71% | | |
| | | | | | | | | (125 ng) | | |
| | | | | | | | | NA/Cam: 53% | | |
| | | | | | | | | (125 ng) | | |
| | | | | | | | | NA/Phu: 87% | | |
| | | | | | | | | (125 ng) | | |
| | | | | | | | | NA/Wash: 82% | | |
| | | | | | | | | (125 ng) | | |
| 18 | QuadMod | 111 μg/mL | 94% | 71 | 0.16 | Main: | HA/Wisc: | HA/Wisconsin | HA/Wisconsin | 0.5 |
| | Tox | | | | | 92%, | 89% | % Positive | EC50: | EU/mL |
| | material | | | | | LMS: | HA/Cam: | Cells: 90% | 41 ng/well | |
| | | | | | | INMT | 90% | (125 ng/well) | HA/Cambodia | |
| | | | | | | 3% | HA/Phu: | HA/Cambodia | EC50: | |
| | | | | | | | 90% | % Positive | 7 ng/well | |
| | | | | | | | HA/Wash: | Cells: 95% | HA/Phuket | |
| | | | | | | | 88% | (31 ng/well) | EC50: | |
| | | | | | | | | HA/Phuket | 17 ng/well | |
| | | | | | | | | % Positive | HA/Wash | |
| | | | | | | | | Cells: 77% | EC50: | |
| | | | | | | | | (31 ng/well) | 11 ng/well | |
| | | | | | | | | HA/Washington | | |
| | | | | | | | | % Positive | | |
| | | | | | | | | Cells: 86% | | |
| | | | | | | | | (31 ng/well) | | |

Intramuscular injection of mice with one dose of an octavalent saRNA-LNP (i.e., “4x saRNA bicistronics”) vaccine encoding the HA and NA antigens from 4 influenza virus strains elicited robust functional anti-HA (FIG. 14), anti-NA (FIG. 15), and virus neutralizing antibodies (FIG. 16). In general, HAI antibody levels elicited toward IAV strains were higher than those elicited toward IBV strains after a single dose (FIG. 14). Octavalent saRNA-LNP vaccines elicited comparable or slightly higher HAI and neutralizing titers than the quadrivalent modRNA vaccine or FluAd (at either the 12 ug or 2.4 ug dose). Neuraminidase inhibiting (NAI) antibody levels elicited by octavalent saRNA-LNP vaccines at Day 21 were trended slightly lower than single antigen controls, but trended higher than NAI titers elicited by FluAd (at either the 12 ug or 2.4 ug dose) (FIG. 15). In general, virus neutralizing titers elicited after one dose of octavalent saRNA vaccine was comparable or superior to a single dose of the quadrivalent modRNA vaccine or FluAd (at either the 12 ug or 2.4 ug dose) (FIG. 16).

All groups received a second dose of vaccine at Day 28, and blood was collected 14 days later on Day 42. Following the 2 nd dose, functional anti-HA (FIG. 17), anti-NA (FIG. 18), and virus neutralizing antibodies (FIG. 19) were boosted in all vaccine groups. HAI and virus neutralizing titers elicited toward A/Cambodia/e0926360/2020 were the least affected by the multivalent formulation, where octavalent and single antigen vaccines elicited similar titers to this virus (FIG. 17, FIG. 19). After two doses of vaccine, NAI titers elicited by octavalent saRNA-LNP vaccines were comparable or superior to titers elicited by either a 12 ug or 2.4 ug dose of FluAd (FIG. 18). With the exception of virus neutralizing titers elicited to A/Cambodia/e0926360/2020, octavalent saRNA-LNP vaccines elicited comparable or superior neutralizing titers than a quadrivalent HA-encoding modRNA-LNP vaccine or FluAd (12 ug or 2.4 ug doses) after two doses of vaccine (FIG. 19).

To evaluate whether the octavalent vaccine was better prepared by first pre-mixing all saRNA constructs followed by formulation with LNPs (pre-mix) or by mixing saRNA-LNP vaccines post-formulation (post-mix), the immunogenicity of each process was compared. Pre-mix and post-mix formulations were evaluated for octavalent vaccines composed of 8 monocistronic HA or NA saRNAs, as well as for the octavalent vaccine composed of 4 bicistronic saRNAs (HA-SGP-NA). Both preparation methods elicited similar levels of functional anti-HA (FIG. 14, FIG. 17), anti-NA (FIG. 15, FIG. 18), and virus neutralizing antibodies (FIG. 16, FIG. 19) after either one dose (FIG. 14, FIG. 15, FIG. 16) or two doses of vaccine (FIG. 17, FIG. 18, FIG. 19). These data suggest that multivalent saRNA vaccines that are co-formulated and those pooled following formulation have comparable immunogenicity.

The immunogenicity of an octavalent saRNA-LNP vaccine composed of either eight monocistronic saRNA constructs or four bicistronic (HA-SGP-NA) saRNA constructs was also evaluated in this study. No difference in HAI titers against IBV strains was observed at this timepoint for monocistronic or bicistronic octavalent vaccines. By day 42 (2 weeks post dose 2), HAI titers to both IAV and IBV strains were comparable in mice administered octavalent vaccines of either monocistronic or bicistronic saRNA constructs (FIG. 17). Three weeks post dose 1, neutralizing antibody levels elicited by monocistronic or bicistronic octavalent vaccines were overall comparable (FIG. 16), with the exception of titers elicited toward A/Cambodia/e0926360/2020, in which the monocistronic octavalent vaccine formulation elicited ˜10-fold higher titers than the bicistronic octavalent vaccine. After a second dose, neutralizing antibody levels to all 4 viruses were comparable in mice that received either the monocistronic or bicistronic octavalent saRNA vaccine (FIG. 19).

The goal of this study was to assess the immunogenicity of octavalent saRNA-LNP vaccines encoding the HA and NA antigens from four influenza virus strains in a mouse model. Functional anti-HA antibodies, measured by HAI assay, functional anti-NA antibodies, measured by NAI assay, and virus neutralizing antibodies, measured by a 1-Day MNT, were all elicited following two doses of the octavalent vaccine formulations. When comparing octavalent vaccines composed of either 8 monocistronic or 4 bicistronic saRNA constructs, the monocistronic octavalent vaccine was modestly superior after a single dose, but after two doses of vaccine the monocistronic and bicistronic octavalent vaccines had comparable immunogenicity. Similar immunogenicity was observed between octavalent saRNA vaccines that were co-formulated (pre-mix) and those that were pooled following formulation (post-mix). Compared to a quadrivalent HA-encoding modRNA-LNP vaccine and FluAd, octavalent saRNA vaccines in general were able to elicit comparable or superior immunogenicity than these comparators.

FIG. 14 Mice were vaccinated intramuscularly at day 0 with either saRNA(s) encoding HA or both HA/NA formulated in LNPs, a HA-encoding quadrivalent modRNA-LNP vaccine, or the licensed comparator, FluAd. Sera were collected 3 weeks after vaccination (Day 21, 3 wks PD1). Functional anti-HA antibody titers against A/Wisconsin/588/2019 (top left), A/Cambodia/e0926360/2020 (top right), B/Phuket/3073/2013 (bottom left), or B/Washington/02/2019 (bottom right) were measured in serum collected from mice (n=5) by the hemagglutination inhibition (HAI) assay. Geometric mean titers (GMT) at each time point are displayed at the top of each graph and indicated by horizontal lines. Negative titers are plotted at the assay's LOD, indicated by the dotted line.

FIG. 15 Mice were vaccinated intramuscularly at day 0 with either saRNA(s) encoding HA or both HA/NA formulated in LNPs or the licensed comparator, FluAd. Sera were collected 3 weeks after vaccination (Day 21, 3 wks PD1). Functional anti-NA antibody titers against A/Wisconsin/588/2019 (top left), B/Phuket/3073/2013 (bottom left), or B/Washington/02/2019 (bottom right) were measured in serum collected from mice (n=5) by an enzyme linked lectin assay (ELLA). NAI titers were not determined against A/Cambodia/e0926360/2020 due to technical difficulties with the virus. Geometric mean titers (GMT) at each time point are displayed at the top of each graph and indicated by horizontal lines. Negative titers are plotted at the assay's LOD, indicated by the dotted line.

FIG. 16 Mice were vaccinated intramuscularly at day 0 with either saRNA(s) encoding HA or both HA/NA formulated in LNPs, a HA-encoding quadrivalent modRNA-LNP vaccine, or the licensed comparator, FluAd. Sera were collected 3 weeks after vaccination (Day 21, 3 wks PD1). Virus neutralizing antibody titers against A/Wisconsin/588/2019 (top left), A/Cambodia/e0926360/2020 (top right), B/Phuket/3073/2013 (bottom left), or B/Washington/02/2019 (bottom right) were measured in serum collected from mice (n=5) by a 1-Day microneutralization test (MNT). Geometric mean titers (GMT) at each time point are displayed at the top of each graph and indicated by horizontal lines. Negative titers are plotted at the assay's LOD, indicated by the dotted line.

FIG. 17 Mice were vaccinated at day 0 and day 28 intramuscularly with either saRNA(s) encoding HA or both HA/NA formulated in LNPs, a HA-encoding quadrivalent modRNA-LNP vaccine, or the licensed comparator, FluAd. Sera were collected 2 weeks after the second vaccination (Day 42, 2 wks PD2). Functional anti-HA antibody titers against A/Wisconsin/588/2019 (top left), A/Cambodia/e0926360/2020 (top right), B/Phuket/3073/2013 (bottom left), or B/Washington/02/2019 (bottom right) were measured in serum collected from mice (n=5) by the hemagglutination inhibition (HAI) assay. Geometric mean titers (GMT) at each time point are displayed at the top of each graph and indicated by horizontal lines. Negative titers are plotted at the assay's LOD, indicated by the dotted line.

FIG. 18 Mice were vaccinated at day 0 and day 28 intramuscularly with either saRNA(s) encoding HA or both HA/NA formulated in LNPs or the licensed comparator, FluAd. Sera were collected 2 weeks after the second vaccination (Day 42, 2 wks PD2). Functional anti-NA antibody titers against A/Wisconsin/588/2019 (top left), B/Phuket/3073/2013 (bottom left), or B/Washington/02/2019 (bottom right) were measured in serum collected from mice (n=5) by an enzyme-linked lectin assay (ELLA). NAI titers were not determined against A/Cambodia/e0926360/2020 due to technical difficulties with the virus. 50% inhibitory titers are plotted, and the geometric mean titers (GMT) at each time point are displayed at the top of each graph and indicated by horizontal lines. Negative titers are plotted at the assay's LOD, indicated by the dotted line.

FIG. 19 Mice were vaccinated intramuscularly at day 0 and day 28 with either saRNA(s) encoding HA or both HA/NA formulated in LNPs, a HA-encoding quadrivalent modRNA-LNP vaccine, or the licensed comparator, FluAd. Sera were collected 2 weeks after the second vaccination (Day 42, 2 wks PD2). Virus neutralizing antibody titers against A/Wisconsin/588/2019 (top left), A/Cambodia/e0926360/2020 (top right), B/Phuket/3073/2013 (bottom left), or B/Washington/02/2019 (bottom right) were measured in serum collected from mice (n=5) by a 1-Day microneutralization test (MNT). 50% neutralization titers are plotted, and the geometric mean titers (GMT) at each time point are displayed at the top of each graph and indicated by horizontal lines. Negative titers are plotted at the assay's LOD, indicated by the dotted line.

### EXEMPLARY EMBODIMENTS

1. A composition comprising a self-amplifying RNA molecule comprising: a 5′ Cap; a 5′ untranslated region; a coding region for a nonstructural protein derived from an alphavirus; a subgenomic promoter derived from an alphavirus; an open reading frame encoding a gene of interest; a 3′ untranslated region; and a 3′ poly A sequence; wherein at least 5% of a total population of a particular nucleotide in the molecule has been replaced with one or more modified or unnatural nucleotides.

2. The composition of clause 1, wherein the 5′ Cap is represented by Formula I:

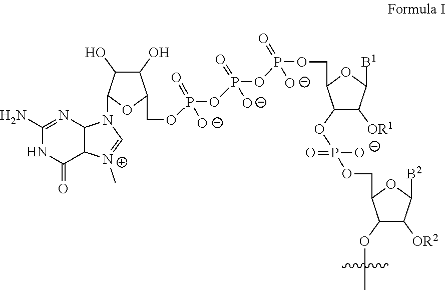

where R¹ and R² are each independently H or Me; and
B¹ and B² are each independently guanine, adenine, or uracil.

3. The composition of clause 1 or 2, wherein B¹ and B² are naturally-occurring bases.

4. The composition of any of clauses 1 to 3, wherein R¹ is methyl and R² is hydrogen.

5. The composition of any of clauses 1 to 4, wherein B¹ is guanine.

6. The composition of any of clauses 1 to 5, wherein B¹ is adenine.

7. The composition of any of clauses 1 to 5, wherein B² is adenine.

8. The composition of any of clauses 1 to 7, wherein B² is uracil.

9. The composition of any of clauses 1 to 8 wherein the nucleotide immediately downstream (5′ to 3′) of the 5′ Cap comprises guanine.

10. The composition of any of clauses 1 to 9, wherein B¹ is adenine and B² is uracil.

11. The composition of any of clauses 1 to 10, wherein B¹ is adenine, B² is uracil, R¹ is methyl, and R² is hydrogen.

12. The composition of any of clauses 1 to 11, wherein the nucleotide immediately downstream (5′ to 3′) of the 5′ Cap comprises guanine, B¹ is adenine, B² is uracil, R¹ is methyl, and R² is hydrogen.

13. The composition of any of clauses 1 to 12, wherein at least 10% of a total population of a particular nucleotide in the molecule has been replaced with one or more modified or unnatural nucleotides.

14. The composition of any of clauses 1 to 13, wherein at least 25% of a total population of a particular nucleotide in the molecule has been replaced with one or more modified or unnatural nucleotides.

15. The composition of any of clauses 1 to 14, wherein at least 50% of a total population of a particular nucleotide in the molecule has been replaced with one or more modified or unnatural nucleotides.

16. The composition of any of clauses 1 to 15, wherein at least 75% of a total population of a particular nucleotide in the molecule has been replaced with one or more modified or unnatural nucleotides.

17. The composition of any of clauses 1 to 16, wherein essentially all of a particular nucleotide population in the molecule has been replaced with one or more modified or unnatural nucleotides.

18. The composition of any of clauses 1 to 17, wherein the one or more modified or unnatural replacement nucleotides comprise two modified or unnatural nucleotides provided in a ratio ranging from 1:99 to 99:1, or any derivable range therein.

19. The composition of any of clauses 1 to 18, wherein at least 10% of a total population of a first particular nucleotide in the molecule has been replaced with one or more modified or unnatural nucleotides, and at least 10% of a total population of a second particular nucleotide in the molecule has been replaced with one or more modified or unnatural nucleotides.

20. The composition of any of clauses 1 to 19, wherein at least 10% of a total population of a first particular nucleotide in the molecule has been replaced with one or more modified or unnatural nucleotides, and at least 25% of a total population of a second particular nucleotide in the molecule has been replaced with one or more modified or unnatural nucleotides.

21. The composition of any of clauses 1 to 20, wherein at least 10% of a total population of a first particular nucleotide in the molecule has been replaced with one or more modified or unnatural nucleotides, and at least 50% of a total population of a second particular nucleotide in the molecule has been replaced with one or more modified or unnatural nucleotides.

22. The composition of any of clauses 1 to 21, wherein at least 10% of a total population of a first particular nucleotide in the molecule has been replaced with one or more modified or unnatural nucleotides, and at least 75% of a total population of a second particular nucleotide in the molecule has been replaced with one or more modified or unnatural nucleotides.

23. The composition of any of clauses 1 to 22, wherein at least 10% of a total population of a first particular nucleotide in the molecule has been replaced with one or more modified or unnatural nucleotides, and essentially all of a total population of a second particular nucleotide in the molecule has been replaced with one or more modified or unnatural nucleotides.

24. The composition of any of clauses 1 to 23, wherein at least 25% of a total population of a first particular nucleotide in the molecule has been replaced with one or more modified or unnatural nucleotides, and at least 25% of a total population of a second particular nucleotide in the molecule has been replaced with one or more modified or unnatural nucleotides.

25. The composition of any of clauses 1 to 24, wherein at least 25% of a total population of a first particular nucleotide in the molecule has been replaced with one or more modified or unnatural nucleotides, and at least 50% of a total population of a second particular nucleotide in the molecule has been replaced with one or more modified or unnatural nucleotides.

26. The composition of any of clauses 1 to 25, wherein at least 25% of a total population of a first particular nucleotide in the molecule has been replaced with one or more modified or unnatural nucleotides, and at least 75% of a total population of a second particular nucleotide in the molecule has been replaced with one or more modified or unnatural nucleotides.

27. The composition of any of clauses 1 to 26, wherein at least 25% of a total population of a first particular nucleotide in the molecule has been replaced with one or more modified or unnatural nucleotides, and essentially all of a total population of a second particular nucleotide in the molecule has been replaced with one or more modified or unnatural nucleotides.

28. The composition of any of clauses 1 to 27, wherein at least 50% of a total population of a first particular nucleotide in the molecule has been replaced with one or more modified or unnatural nucleotides, and at least 75% of a total population of a second particular nucleotide in the molecule has been replaced with one or more modified or unnatural nucleotides.

29. The composition of any of clauses 1 to 28, wherein at least 50% of a total population of a first particular nucleotide in the molecule has been replaced with one or more modified or unnatural nucleotides, and essentially all of a total population of a second particular nucleotide in the molecule has been replaced with one or more modified or unnatural nucleotides.

30. The composition of any of clauses 1 to 29, wherein at least 75% of a total population of a first particular nucleotide in the molecule has been replaced with one or more modified or unnatural nucleotides, and essentially all of a total population of a second particular nucleotide in the molecule has been replaced with one or more modified or unnatural nucleotides.

31. The composition of any of clauses 1 to 30, wherein the modified or unnatural nucleotides are selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, dihydropseudouridine, 5-methoxyuridine and 2′-O-methyl uridine.

32. The composition of any of clauses 1 to 31, wherein the modified or unnatural nucleotides are selected from the group consisting of 5-methyluridine, N1-methylpseudouridine, 5-methoxyuridine, and 5-methylcytosine.

33. The composition of any of clauses 1 to 32, wherein at least 25% of a total population of uridine nucleotides in the molecule has been replaced with N1-methylpseudouridine.

34. The composition of any of clauses 1 to 33, wherein at least 50% of a total population of uridine nucleotides in the molecule has been replaced with N1-methylpseudouridine.

35. The composition of any of clauses 1 to 34, wherein at least 75% of a total population of uridine nucleotides in the molecule has been replaced with N1-methylpseudouridine.

36. The composition of any of clauses 1 to 35, wherein essentially all uridine nucleotides in the molecule have been replaced with N1-methylpseudouridine.

37. The composition of any of clauses 1 to 36, wherein at least 50% of a total population of uridine nucleotides in the molecule has been replaced with 5-methoxyuridine.

38. The composition of any of clauses 1 to 37, wherein essentially all uridine nucleotides in the molecule have been replaced with 5-methoxyuridine.

39. The composition of any of clauses 1 to 38, wherein at least 50% of a total population of uridine nucleotides in the molecule has been replaced with 5-methyluridine.

40. The composition of any of clauses 1 to 39, wherein essentially all uridine nucleotides in the molecule have been replaced with 5-methyluridine.

41. The composition of any of clauses 1 to 40, wherein at least 50% of a total population of cytosine nucleotides in the molecule has been replaced with 5-methylcytosine.

42. The composition of any of clauses 1 to 41, wherein essentially all cytosine nucleotides in the molecule have been replaced with 5-methylcytosine.

43. The composition of any of clauses 1 to 42, wherein at least 50% of a total population of uridine nucleotides in the molecule has been replaced with 2-thiouridine.

44. The composition of any of clauses 1 to 43, wherein essentially all uridine nucleotides in the molecule have been replaced with 2-thiouridine.

45. The composition of any of clauses 1 to 44, wherein at least 50% of a total population of uridine nucleotides in the molecule has been replaced with N1-methylpseudouridine and essentially all cytosine nucleotides in the molecule have been replaced with 5-methylcytosine.

46. The composition of any of clauses 1 to 45, wherein at least 50% of a total population of uridine nucleotides in the molecule has been replaced with 5-methoxyuridine and essentially all cytosine nucleotides in the molecule have been replaced with 5-methylcytosine.

47. The composition of any of clauses 1 to 46, wherein at least 50% of a total population of uridine nucleotides in the molecule has been replaced with 5-methyluridine and essentially all cytosine nucleotides in the molecule have been replaced with 5-methylcytosine.

48. The composition of any of clauses 1 to 47, wherein essentially all uridine nucleotides in the molecule have been replaced with about 50% 5-methoxyuridine and about 50% N1-methylpseudouridine.

49. The composition of any of clauses 1 to 48, wherein essentially all uridine nucleotides in the molecule have been replaced with about 75% 5-methoxyuridine and about 25% N1-methylpseudouridine.

50. The composition of any of clauses 1 to 49, wherein essentially all uridine nucleotides in the molecule have been replaced with about 25% 5-methoxyuridine and about 75% N1-methylpseudouridine.

51. The composition of any of clauses 1 to 50, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with N1-methylpseudouridine, and essentially all cytosine nucleotides in the molecule have been replaced with 5-methylcytosine.

52. The composition of any of clauses 1 to 51, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methoxyuridine, and essentially all cytosine nucleotides in the molecule have been replaced with 5-methylcytosine.

53. The composition of any of clauses 1 to 52, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the molecule has been replaced with 5-methyluridine, and essentially all cytosine nucleotides in the molecule have been replaced with 5-methylcytosine.

54. The composition of any of clauses 1 to 53, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 50% 5-methoxyuridine and about 50% N1-methylpseudouridine.

55. The composition of any of clauses 1 to 54, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 75% 5-methoxyuridine and about 25% N1-methylpseudouridine.

56. The composition of any of clauses 1 to 55, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the molecule have been replaced with about 25% 5-methoxyuridine and about 75% N1-methylpseudouridine.

57. The composition of any of clauses 1 to 56, wherein the subgenomic promoter is operably linked to the open reading frame.

58. The composition of any of clauses 1 to 57, wherein the subgenomic promoter comprises a cis-acting regulatory element.

59. The composition of any of clauses 1 to 58, wherein the cis-acting regulatory element is immediately downstream of B 2.

60. The composition of any of clauses 1 to 59, wherein the cis-acting regulatory element is an AU-rich element.

61. The composition of any of clauses 1 to 60, wherein the nonstructural protein comprises alphavirus nonstructural protein nsP1.

62. The composition of any of clauses 1 to 61, wherein the nonstructural protein comprises alphavirus nonstructural protein nsP2.

63. The composition of any of clauses 1 to 62, wherein the nonstructural protein comprises alphavirus nonstructural protein nsP3.

64. The composition of any of clauses 1 to 63, wherein the nonstructural protein comprises alphavirus nonstructural protein nsP4.

65. The composition of any of clauses 1 to 64, wherein the alphavirus is Venezuelan equine encephalitis virus.

66. The composition of any of clauses 1 to 65, wherein the alphavirus is Semliki Forest virus.

67. The composition of any of clauses 1 to 66, further comprising a pharmaceutically acceptable carrier.

68. The composition of any of clauses 1 to 67, further comprising a cationic lipid.

69. The composition of any of clauses 1 to 68, wherein the RNA molecule is encapsulated in, bound to, or adsorbed on a cationic lipid.

70. The composition of any of clauses 1 to 69, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

71. The composition of any of clauses 1 to 70, wherein the RNA molecule is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

72. A method of inducing an immune response in a subject, comprising administering to the subject in need thereof an effective amount of the composition of any of clauses 1 to 71.

73. A method of vaccinating a subject, comprising administering to the subject in need thereof an effective amount of the composition of any of clauses 1 to 71.

74. A method for treating or preventing an infectious disease, comprising administering to the subject in need thereof an effective amount of the composition of any of clauses 1 to 71.

75. The method of any of clauses 1 to 74, wherein the composition elicits an immune response comprising an antibody response.

76. The method according of any of clauses 1 to 75, wherein the composition elicits an immune response comprising a T cell response.

77. A composition comprising a self-amplifying RNA molecule comprising:
a 5′ Cap represented by Formula I,

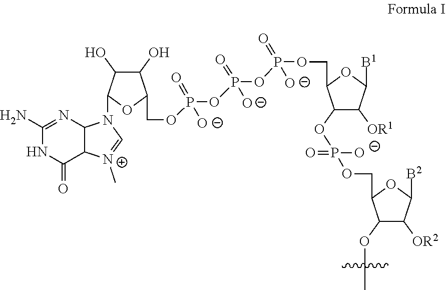

where R¹ and R² are each independently H or Me; and B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and
a 3′ poly A sequence.

78. The composition of clause 77, wherein B¹ and B² are naturally-occurring bases.

79. The composition of clause 77 or 78, wherein R¹ is methyl and R² is hydrogen.

80. The composition of any of clauses 77 to 79, wherein B¹ is guanine.

81. The composition of any of clauses 77 to 80, wherein B¹ is adenine.

82. The composition of any of clauses 77 to 81, wherein B² is adenine.

83. The composition of any of clauses 77 to 82, wherein B² is uracil.

84. The composition of any of clauses 77 to 83, wherein the nucleotide immediately downstream (5′ to 3′) of the 5′ Cap comprises guanine.

85. The composition of any of clauses 77 to 84, wherein B¹ is adenine and B² is uracil.

86. The composition of any of clauses 77 to 85, wherein B¹ is adenine, B² is uracil, R¹ is methyl, and R² is hydrogen.

87. The composition of any of clauses 77 to 86, wherein the nucleotide immediately downstream (5′ to 3′) of the 5′ Cap comprises guanine, B¹ is adenine, B² is uracil, R¹ is methyl, and R² is hydrogen.

88. The composition of any of clauses 77 to 87, wherein the subgenomic promoter is operably linked to the open reading frame.

89. The composition of any of clauses 77 to 88, wherein the subgenomic promoter comprises a cis-acting regulatory element.

90. The composition of any of clauses 77 to 89, wherein the cis-acting regulatory element is immediately downstream of B².

91. The composition of any of clauses 77 to 90, wherein the cis-acting regulatory element is an AU-rich element.

92. The composition of any of clauses 77 to 91, wherein the nonstructural protein comprises alphavirus nonstructural protein nsP1.

93. The composition of any of clauses 77 to 92, wherein the nonstructural protein comprises alphavirus nonstructural protein nsP2.

94. The composition of any of clauses 77 to 93, wherein the nonstructural protein comprises alphavirus nonstructural protein nsP3.

95. The composition of any of clauses 77 to 94, wherein the nonstructural protein comprises alphavirus nonstructural protein nsP4.

96. The composition of any of clauses 77 to 95, wherein the alphavirus is Venezuelan equine encephalitis virus.

97. The composition of any of clauses 77 to 96, wherein the alphavirus is Semliki Forest virus.

98. The composition of any of clauses 77 to 97, further comprising a pharmaceutically acceptable carrier.

99. The composition of any of clauses 77 to 98, further comprising a cationic lipid.

100. The composition of any of clauses 77 to 99, wherein the RNA molecule is encapsulated in, bound to, or adsorbed on a cationic lipid.

101. The composition of any of clauses 77 to 100, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

102. The composition of any of clauses 77 to 101, wherein the RNA molecule is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion and combinations thereof.

103. A method of inducing an immune response in a subject, comprising administering to the subject in need thereof an effective amount of the composition of any of clauses 77 to 102.

104. A method of vaccinating a subject, comprising administering to the subject in need thereof an effective amount of the composition of any of clauses 77 to 103.

105. A method for treating or preventing an infectious disease, comprising administering to the subject in need thereof an effective amount of the composition of any of clauses 77 to 104.

106. The method of any of clauses 77 to 105, wherein the composition elicits an immune response comprising an antibody response.

107. The method according of any of clauses 77 to 106, wherein the composition elicits an immune response comprising a T cell response.

108. A composition comprising (i) a first RNA molecule comprising a modified nucleotide; and (ii) a second RNA molecule comprising a 5′ Cap, a 5′ untranslated region, a coding region for a nonstructural protein derived from an alphavirus, a subgenomic promoter derived from an alphavirus, an open reading frame encoding a gene of interest, a 3′ untranslated region, and a 3′ poly A sequence, wherein at least 5% of a total population of a particular nucleotide in the molecule has been replaced with one or more modified or unnatural nucleotides.

109. The composition of clause 108, wherein the composition reduces cytotoxicity, as compared to an identical composition in the absence of the first RNA molecule.

110. The composition of clause 108 or 109, wherein the second RNA molecule in the presence of the first RNA molecule elicits less cytotoxicity as compared to the cytotoxicity elicited by the second RNA molecule in the absence of the first RNA molecule.

111. The composition of any of clauses 108 to 110, wherein the second RNA molecule in the presence of the first RNA molecule expresses the gene of interest in an amount that is greater than the amount of expression of the gene of interest in the absence of the first RNA molecule.

112. The composition of any of clauses 108 to 111, wherein the composition comprises an amount of the first RNA molecule that is greater than the amount of the second RNA molecule.

113. The composition of any of clauses 108 to 112, wherein the composition comprises an amount of the first RNA molecule that is at least about 2 times greater than the amount of the second RNA molecule.

114. The composition of any of clauses 108 to 113, wherein the first RNA molecule is capable of evading an innate immune response of a cell into which the first RNA molecule is introduced.

115. The composition of any of clauses 108 to 114, wherein at least 10% of a total population of a particular nucleotide in the second RNA molecule has been replaced with one or more modified or unnatural nucleotides.

116. The composition of any of clauses 108 to 115, wherein at least 25% of a total population of a particular nucleotide in the second RNA molecule has been replaced with one or more modified or unnatural nucleotides.

117. The composition of any of clauses 108 to 116, wherein at least 50% of a total population of a particular nucleotide in the second RNA molecule has been replaced with one or more modified or unnatural nucleotides.

118. The composition of any of clauses 108 to 117, wherein at least 75% of a total population of a particular nucleotide in the second RNA molecule has been replaced with one or more modified or unnatural nucleotides.

119. The composition of any of clauses 108 to 118, wherein essentially all of a particular nucleotide population in the second RNA molecule has been replaced with one or more modified or unnatural nucleotides.

120. The composition of any of clauses 108 to 119, wherein at least 10% of a total population of a particular nucleotide in the first RNA molecule has been replaced with one or more modified or unnatural nucleotides.

121. The composition of any of clauses 108 to 120, wherein at least 25% of a total population of a particular nucleotide in the first RNA molecule has been replaced with one or more modified or unnatural nucleotides.

122. The composition of any of clauses 108 to 121, wherein at least 50% of a total population of a particular nucleotide in the first RNA molecule has been replaced with one or more modified or unnatural nucleotides.

123. The composition of any of clauses 108 to 122, wherein at least 75% of a total population of a particular nucleotide in the first RNA molecule has been replaced with one or more modified or unnatural nucleotides.

124. The composition of any of clauses 108 to 123, wherein essentially all of a particular nucleotide population in the first RNA molecule has been replaced with one or more modified or unnatural nucleotides.

125. The composition of any of clauses 108 to 124, wherein the one or more modified or unnatural replacement nucleotides comprise two modified or unnatural nucleotides provided in a ratio ranging from 1:99 to 99:1, or any derivable range therein.

126. The composition of any of clauses 108 to 125, wherein at least 10% of a total population of a first particular nucleotide in the second RNA molecule has been replaced with one or more modified or unnatural nucleotides, and at least 10% of a total population of a second particular nucleotide in the second RNA molecule has been replaced with one or more modified or unnatural nucleotides.

127. The composition of any of clauses 108 to 126, wherein at least 10% of a total population of a first particular nucleotide in the second RNA molecule has been replaced with one or more modified or unnatural nucleotides, and at least 25% of a total population of a second particular nucleotide in the second RNA molecule has been replaced with one or more modified or unnatural nucleotides.

128. The composition of any of clauses 108 to 127, wherein at least 10% of a total population of a first particular nucleotide in the second RNA molecule has been replaced with one or more modified or unnatural nucleotides, and at least 50% of a total population of a second particular nucleotide in the second RNA molecule has been replaced with one or more modified or unnatural nucleotides.

129. The composition of any of clauses 108 to 128, wherein at least 10% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

130. The composition of any of clauses 108 to 129, wherein at least 10% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

131. The composition of any of clauses 108 to 130, wherein at least 25% of a total population of a first particular nucleotide in the second RNA molecule has been replaced with one or more modified or unnatural nucleotides, and at least 25% of a total population of a second particular nucleotide in the second RNA molecule has been replaced with one or more modified or unnatural nucleotides.

132. The composition of any of clauses 108 to 131, wherein at least 25% of a total population of a first particular nucleotide in the second RNA molecule has been replaced with one or more modified or unnatural nucleotides, and at least 50% of a total population of a second particular nucleotide in the second RNA molecule has been replaced with one or more modified or unnatural nucleotides.

133. The composition of any of clauses 108 to 132, wherein at least 25% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

134. The composition of any of clauses 108 to 133, wherein at least 25% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

135. The composition of any of clauses 108 to 134, wherein at least 50% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

136. The composition of any of clauses 108 to 135, wherein at least 50% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

137. The composition of any of clauses 108 to 136, wherein at least 75% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

138. The composition of any of clauses 108 to 137, wherein the modified nucleotide comprises any one nucleotide selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

139. The composition of any of clauses 108 to 138, wherein the modified or unnatural nucleotides are selected from the group consisting of 5-methyluridine, N1-methylpseudouridine, and 5-methylcytosine.

140. The composition of any of clauses 108 to 139, wherein at least 25% of a total population of uridine nucleotides in the first RNA molecule has been replaced with N1-methylpseudouridine.

141. The composition of any of clauses 108 to 140, wherein at least 50% of a total population of uridine nucleotides in the first RNA molecule has been replaced with N1-methylpseudouridine.

142. The composition of any of clauses 108 to 141, wherein at least 75% of a total population of uridine nucleotides in the first RNA molecule has been replaced with N1-methylpseudouridine.

143. The composition of any of clauses 108 to 142, wherein essentially all uridine nucleotides in the first RNA molecule have been replaced with N1-methylpseudouridine.

144. The composition of any of clauses 108 to 143, wherein at least 50% of a total population of uridine nucleotides in the first RNA molecule has been replaced with 5-methoxyuridine.

145. The composition of any of clauses 108 to 144, wherein essentially all uridine nucleotides in the first RNA molecule have been replaced with 5-methoxyuridine.

146. The composition of any of clauses 108 to 145, wherein at least 50% of a total population of uridine nucleotides in the first RNA molecule has been replaced with 5-methyluridine.

147. The composition of any of clauses 108 to 146, wherein essentially all uridine nucleotides in the first RNA molecule have been replaced with 5-methyluridine.

148. The composition of any of clauses 108 to 147, wherein at least 50% of a total population of cytosine nucleotides in the first RNA molecule has been replaced with 5-methylcytosine.

149. The composition of any of clauses 108 to 148, wherein essentially all cytosine nucleotides in the first RNA molecule have been replaced with 5-methylcytosine.

150. The composition of any of clauses 108 to 149, wherein at least 50% of a total population of uridine nucleotides in the first RNA molecule has been replaced with 2-thiouridine.

151. The composition of any of clauses 108 to 150, wherein essentially all uridine nucleotides in the first RNA molecule have been replaced with 2-thiouridine.

152. The composition of any of clauses 108 to 151, wherein at least 25% of a total population of uridine nucleotides in the second RNA molecule has been replaced with N1-methylpseudouridine.

153. The composition of any of clauses 108 to 152, wherein at least 50% of a total population of uridine nucleotides in the second RNA molecule has been replaced with N1-methylpseudouridine.

154. The composition of any of clauses 108 to 153, wherein at least 75% of a total population of uridine nucleotides in the second RNA molecule has been replaced with N1-methylpseudouridine.

155. The composition of any of clauses 108 to 154, wherein essentially all uridine nucleotides in the second RNA molecule have been replaced with N1-methylpseudouridine.

156. The composition of any of clauses 108 to 155, wherein at least 50% of a total population of uridine nucleotides in the second RNA molecule has been replaced with 5-methoxyuridine.

157. The composition of any of clauses 108 to 156, wherein essentially all uridine nucleotides in the second RNA molecule have been replaced with 5-methoxyuridine.

158. The composition of any of clauses 108 to 157, wherein at least 50% of a total population of uridine nucleotides in the second RNA molecule has been replaced with 5-methyluridine.

159. The composition of any of clauses 108 to 158, wherein essentially all uridine nucleotides in the second RNA molecule have been replaced with 5-methyluridine.

160. The composition of any of clauses 108 to 159, wherein at least 50% of a total population of cytosine nucleotides in the second RNA molecule has been replaced with 5-methylcytosine.

161. The composition of any of clauses 108 to 160, wherein essentially all cytosine nucleotides in the second RNA molecule have been replaced with 5-methylcytosine.

162. The composition of any of clauses 108 to 161, wherein at least 50% of a total population of uridine nucleotides in the second RNA molecule has been replaced with 2-thiouridine.

163. The composition of any of clauses 108 to 162, wherein essentially all uridine nucleotides in the second RNA molecule have been replaced with 2-thiouridine.

164. The composition of any of clauses 108 to 163, wherein at least 50% of a total population of uridine nucleotides in the second RNA molecule has been replaced with N1-methylpseudouridine and essentially all cytosine nucleotides in the second RNA molecule have been replaced with 5-methylcytosine.

165. The composition of any of clauses 108 to 164, wherein at least 50% of a total population of uridine nucleotides in the second RNA molecule has been replaced with 5-methoxyuridine and essentially all cytosine nucleotides in the second RNA molecule have been replaced with 5-methylcytosine.

166. The composition of any of clauses 108 to 165, wherein at least 50% of a total population of uridine nucleotides in the second RNA molecule has been replaced with 5-methyluridine and essentially all cytosine nucleotides in the second RNA molecule have been replaced with 5-methylcytosine.

167. The composition of any of clauses 108 to 166, wherein essentially all uridine nucleotides in the second RNA molecule have been replaced with about 50% 5-methoxyuridine and about 50% N1-methylpseudouridine.

168. The composition of any of clauses 108 to 167, wherein essentially all uridine nucleotides in the second RNA molecule have been replaced with about 75% 5-methoxyuridine and about 25% N1-methylpseudouridine.

169. The composition of any of clauses 108 to 168, wherein essentially all uridine nucleotides in the second RNA molecule have been replaced with about 25% 5-methoxyuridine and about 75% N1-methylpseudouridine.

170. The composition of any of clauses 108 to 169, wherein essentially all uridine nucleotides in the first RNA molecule have been replaced with N1-methylpseudouridine and at least 50% of a total population of uridine nucleotides in the second RNA molecule has been replaced with N1-methylpseudouridine.

171. The composition of any of clauses 108 to 170, wherein essentially all uridine nucleotides in the first RNA molecule have been replaced with N1-methylpseudouridine and essentially all uridine nucleotides in the second RNA molecule have been replaced with N1-methylpseudouridine.

172. The composition of any of clauses 108 to 171, wherein essentially all uridine nucleotides in the first RNA molecule have been replaced with N1-methylpseudouridine and at least 50% of a total population of uridine nucleotides in the second RNA molecule has been replaced with 5-methoxyuridine.

173. The composition of any of clauses 108 to 172, wherein essentially all uridine nucleotides in the first RNA molecule have been replaced with N1-methylpseudouridine, at least 50% of a total population of uridine nucleotides in the second RNA molecule has been replaced with 5-methyluridine, and essentially all cytosine nucleotides in the second RNA molecule have been replaced with 5-methylcytosine.

174. The composition of any of clauses 108 to 173, wherein essentially all uridine nucleotides in the first RNA molecule have been replaced with N1-methylpseudouridine and essentially all uridine nucleotides in the second RNA molecule have been replaced with about 50% 5-methoxyuridine and about 50% N1-methylpseudouridine.

175. The composition of any of clauses 108 to 174, wherein the first RNA molecule does not comprise a subgenomic promoter.

176. The composition of any of clauses 108 to 175, wherein the first RNA molecule is not a self-amplifying RNA molecule.

177. The composition of any of clauses 108 to 176, wherein the first RNA molecule further comprises a 5′ cap moiety.

178. The composition of any of clauses 108 to 177, wherein the first RNA molecule further comprises a 5′ untranslated region.

179. The composition of any of clauses 108 to 178, wherein the first RNA molecule further comprises a 3′ untranslated region.

180. The composition of any of clauses 108 to 179, wherein the first RNA molecule further comprises a 3′ poly A sequence.

181. The composition of any of clauses 108 to 180, wherein the first RNA molecule further comprises an open reading frame.

182. The composition of any of clauses 108 to 181, wherein the first RNA molecule does not comprise any one of a 5′ cap moiety, untranslated region, and poly A sequence.

183. The composition of any of clauses 108 to 182, wherein the first RNA molecule comprises a 5′ untranslated region and a 3′ untranslated region.

184. The composition of any of clauses 108 to 183, wherein the first RNA molecule comprises a cap moiety, a 5′ untranslated region (5′ UTR), a modified nucleotide, an open reading frame, a 3′ untranslated region (3′ UTR), a 3′ poly A sequence.

185. The composition of any of clauses 108 to 184, wherein the first RNA molecule does not comprise an open reading frame encoding an antigen.

186. The composition of any of clauses 108 to 185, wherein the first RNA molecule comprises a noncoding RNA region.

187. The composition of any of clauses 108 to 186, wherein the first RNA molecule comprises a coding RNA region.

188. The composition of any of clauses 108 to 187, wherein the 5′-cap moiety of any one of the first and second RNA molecules is a natural 5′-cap.

189. The composition of any of clauses 108 to 188, wherein the 5′-cap moiety of any one of the first and second RNA molecules is a 5′-cap analog.

190. The composition of any of clauses 108 to 189, wherein the nonstructural protein comprises alphavirus nonstructural protein nsP1.

191. The composition of any of clauses 108 to 190, wherein the nonstructural protein comprises alphavirus nonstructural protein nsP2.

192. The composition of any of clauses 108 to 191, wherein the nonstructural protein comprises alphavirus nonstructural protein nsP3.

193. The composition of any of clauses 108 to 192, wherein the nonstructural protein comprises alphavirus nonstructural protein nsP4.

194. The composition of any of clauses 108 to 193, wherein the nonstructural protein comprises alphavirus nonstructural protein nsP1, nsP2, and nsP3.

195. The composition of any of clauses 108 to 194, wherein the first RNA molecule does not comprise the alphavirus nonstructural protein 4 (nsP4).

196. The composition of any of clauses 108 to 195, wherein the second RNA molecule does not comprise the alphavirus nonstructural protein 4 (nsP4).

197. The composition of any of clauses 108 to 196, wherein the first RNA molecule and the second RNA molecule comprise one or more modified nucleotides.

198. The composition of any of clauses 108 to 197, wherein the subgenomic promoter is operably linked to the open reading frame.

199. The composition of any of clauses 108 to 198, wherein the subgenomic promoter comprises a cis-acting regulatory element.

200. The composition of any of clauses 108 to 199, wherein the cis-acting regulatory element is immediately downstream of B 2.

201. The composition of any of clauses 108 to 200, wherein the cis-acting regulatory element is an AU-rich element.

202. The composition of any of clauses 108 to 201 wherein the second RNA molecule further comprises: (1) an alphavirus 5′ replication recognition sequence, and (2) an alphavirus 3′ replication recognition sequence.

203. The composition of any of clauses 108 to 202 wherein the second RNA molecule encodes at least one antigen.

204. The composition of any of clauses 108 to 203 wherein the second RNA molecule comprises at least 7000 nucleotides.

205. The composition of any of clauses 108 to 204 wherein the second RNA molecule comprises at least 8000 nucleotides.

206. The composition of any of clauses 108 to 205 wherein at least 80% of the total second RNA molecules are full length.

207. The composition of any of clauses 108 to 206 wherein the alphavirus is Venezuelan equine encephalitis virus.

208. The composition of any of clauses 108 to 207 wherein the alphavirus is Semliki Forest virus.

209. The composition of any of clauses 108 to 208, further comprising a pharmaceutically acceptable carrier.

210. The composition of any of clauses 108 to 209, further comprising a cationic lipid.

211. The composition of any of clauses 108 to 210, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

212. The composition of any of clauses 108 to 211, wherein the first and the second RNA molecules are encapsulated in, bound to, or adsorbed on a cationic lipid.

213. The composition of any of clauses 108 to 212, wherein the first and the second RNA molecules are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

214. The composition of any of clauses 108 to 213, wherein the first and the second RNA molecules are purified.

215. A method of expressing a polypeptide in a mammalian cell, comprising administering to the mammalian cell a composition comprising (i) a first RNA molecule according to any one of clauses 108 to 214, and (ii) a second RNA molecule according to any one of clauses 108 to 214, wherein the method expresses the polypeptide of interest in an amount that is, when measured under identical conditions, greater than a method that comprises administering to the mammalian cell a composition comprising the second RNA molecule, in the absence of the first RNA molecule.

216. A method of inducing an immune response in a subject, comprising administering to the subject in need thereof an effective amount of a composition according to any one of clauses 108 to 214.

217. A method of vaccinating a subject, comprising administering to the subject in need thereof an effective amount of a composition according to any one of clauses 108 to 214.

218. A method for treating or preventing an infectious disease, comprising administering to the subject in need thereof an effective amount of a composition according to any one of clauses 108 to 214.

219. The method according to any one of clauses 215 to 218, wherein the composition elicits an immune response comprising an antibody response.

220. The method according to any one of clauses 215 to 219, wherein the composition elicits an immune response comprising a T cell response.

221. A composition comprising (i) a first RNA molecule comprising a modified nucleotide; and (ii) a second RNA molecule comprising a 5′ Cap represented by Formula I,

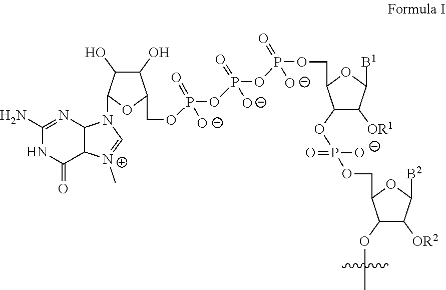

where R¹ and R² are each independently H or Me, and B¹ and B² are each independently guanine, adenine, or uracil, a 5′ untranslated region, a coding region for a nonstructural protein derived from an alphavirus, a subgenomic promoter derived from an alphavirus, an open reading frame encoding a gene of interest, a 3′ untranslated region, and a 3′ poly A sequence.

222. The composition of clause 221, wherein B¹ and B² are naturally-occurring bases.

223. The composition of clause 221 or 222, wherein R¹ is methyl and R² is hydrogen.

224. The composition of any of clauses 221 to 223, wherein B¹ is guanine.

225. The composition of any of clauses 221 to 224, wherein B¹ is adenine.

226. The composition of any of clauses 221 to 225, wherein B² is adenine.

227. The composition of any of clauses 221 to 226, wherein B² is uracil.

228. The composition of any of clauses 221 to 227, wherein the nucleotide immediately downstream (5′ to 3′) of the 5′ Cap comprises guanine.

229. The composition of any of clauses 221 to 228, wherein B¹ is adenine and B² is uracil.

230. The composition of any of clauses 221 to 229, wherein B¹ is adenine, B² is uracil, R¹ is methyl, and R² is hydrogen.

231. The composition of any of clauses 221 to 230, wherein the nucleotide immediately downstream (5′ to 3′) of the 5′ Cap comprises guanine, B¹ is adenine, B² is uracil, R¹ is methyl, and R² is hydrogen.

232. The composition of any of clauses 221 to 231, wherein the composition reduces cytotoxicity, as compared to an identical composition in the absence of the first RNA molecule.

233. The composition of any of clauses 221 to 232, wherein the second RNA molecule in the presence of the first RNA molecule elicits less cytotoxicity as compared to the cytotoxicity elicited by the second RNA molecule in the absence of the first RNA molecule.

234. The composition of any of clauses 221 to 233, wherein the second RNA molecule in the presence of the first RNA molecule expresses the gene of interest in an amount that is greater than the amount of expression of the gene of interest in the absence of the first RNA molecule.

235. The composition of any of clauses 221 to 234, wherein the composition comprises an amount of the first RNA molecule that is greater than the amount of the second RNA molecule.

236. The composition of any of clauses 221 to 235, wherein the composition comprises an amount of the first RNA molecule that is at least about 2 times greater than the amount of the second RNA molecule.

237. The composition of any of clauses 221 to 236, wherein the first RNA molecule is capable of evading an innate immune response of a cell into which the first RNA molecule is introduced.

238. The composition of any of clauses 221 to 237, wherein the modified nucleotide comprises any one nucleotide selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, dihydropseudouridine, 5-methoxyuridine and 2′-O-methyl uridine.

239. The composition of any of clauses 221 to 238, wherein the modified or unnatural nucleotides are selected from the group consisting of 5-methyluridine, N1-methylpseudouridine, and 5-methylcytosine.

240. The composition of any of clauses 221 to 239, wherein at least 10% of a total population of a particular nucleotide in the first RNA molecule has been replaced with one or more modified or unnatural nucleotides.

241. The composition of any of clauses 221 to 240, wherein at least 25% of a total population of a particular nucleotide in the first RNA molecule has been replaced with one or more modified or unnatural nucleotides.

242. The composition of any of clauses 221 to 241, wherein at least 50% of a total population of a particular nucleotide in the first RNA molecule has been replaced with one or more modified or unnatural nucleotides.

243. The composition of any of clauses 221 to 242, wherein at least 75% of a total population of a particular nucleotide in the first RNA molecule has been replaced with one or more modified or unnatural nucleotides.

244. The composition of any of clauses 221 to 243, wherein essentially all of a particular nucleotide population in the first RNA molecule has been replaced with one or more modified or unnatural nucleotides.

245. The composition of any of clauses 221 to 244, wherein the first RNA molecule does not comprise a subgenomic promoter.

246. The composition of any of clauses 221 to 245, wherein the first RNA molecule is not a self-amplifying RNA molecule.

247. The composition of any of clauses 221 to 246, wherein the first RNA molecule further comprises a 5′ cap moiety.

248. The composition of any of clauses 221 to 247, wherein the first RNA molecule further comprises a 5′ untranslated region.

249. The composition of any of clauses 221 to 248, wherein the first RNA molecule further comprises a 3′ untranslated region.

250. The composition of any of clauses 221 to 249, wherein the first RNA molecule further comprises a 3′ poly A sequence.

251. The composition of any of clauses 221 to 250, wherein the first RNA molecule further comprises an open reading frame.

252. The composition of any of clauses 221 to 251, wherein the first RNA molecule does not comprise any one of a 5′ cap moiety, untranslated region, and poly A sequence.

253. The composition of any of clauses 221 to 252, wherein the first RNA molecule comprises a untranslated region and a 3′ untranslated region.

254. The composition of any of clauses 221 to 253, wherein the first RNA molecule comprises a cap moiety, a 5′ untranslated region (5′ UTR), a modified nucleotide, an open reading frame, a 3′ untranslated region (3′ UTR), a 3′ poly A sequence.

255. The composition of any of clauses 221 to 254, wherein the first RNA molecule does not comprise an open reading frame encoding an antigen.

256. The composition of any of clauses 221 to 255, wherein the first RNA molecule comprises a noncoding RNA region.

257. The composition of any of clauses 221 to 256, wherein the first RNA molecule comprises a coding RNA region.

258. The composition of any of clauses 221 to 257, wherein the 5′-cap moiety of the first RNA molecule is a natural 5′-cap.

259. The composition of any of clauses 221 to 258, wherein the 5′-cap moiety of the first RNA molecule is a 5′-cap analog.

260. The composition of any of clauses 221 to 259, wherein the nonstructural protein comprises alphavirus nonstructural protein nsP1.

261. The composition of any of clauses 221 to 260, wherein the nonstructural protein comprises alphavirus nonstructural protein nsP2.

262. The composition of any of clauses 221 to 261, wherein the nonstructural protein comprises alphavirus nonstructural protein nsP3.

263. The composition of any of clauses 221 to 262, wherein the nonstructural protein comprises alphavirus nonstructural protein nsP4.

264. The composition of any of clauses 221 to 263, wherein the nonstructural protein comprises alphavirus nonstructural protein nsP1, nsP2, and nsP3.

265. The composition of any of clauses 221 to 264, wherein the first RNA molecule does not comprise the alphavirus nonstructural protein 4 (nsP4).

266. The composition of any of clauses 221 to 265, wherein the second RNA molecule does not comprise the alphavirus nonstructural protein 4 (nsP4).

267. The composition of any of clauses 221 to 266 wherein the second RNA molecule does not comprise a modified nucleotide.

268. The composition of any of clauses 221 to 267, wherein the first RNA molecule and the second RNA molecule comprise one or more modified nucleotides.

269. The composition of any of clauses 221 to 268, wherein the subgenomic promoter is operably linked to the open reading frame.

270. The composition of any of clauses 221 to 269, wherein the subgenomic promoter comprises a cis-acting regulatory element.

271. The composition of any of clauses 221 to 270, wherein the cis-acting regulatory element is immediately downstream of B 2.

272. The composition of any of clauses 221 to 271, wherein the cis-acting regulatory element is an AU-rich element.

273. The composition of any of clauses 221 to 272, wherein the second RNA molecule further comprises: (1) an alphavirus 5′ replication recognition sequence, and (2) an alphavirus 3′ replication recognition sequence.

274. The composition of any of clauses 221 to 273, wherein the second RNA molecule encodes at least one antigen.

275. The composition of any of clauses 221 to 274, wherein the second RNA molecule comprises at least 7000 nucleotides.

276. The composition of any of clauses 221 to 275, wherein the second RNA molecule comprises at least 8000 nucleotides.

277. The composition of any of clauses 221 to 276, wherein at least 80% of the total second RNA molecules are full length.

278. The composition of any of clauses 221 to 277, wherein the alphavirus is Venezuelan equine encephalitis virus.

279. The composition of any of clauses 221 to 278, wherein the alphavirus is Semliki Forest virus.

280. The composition of any of clauses 221 to 279, further comprising a pharmaceutically acceptable carrier.

281. The composition of any of clauses 221 to 280, further comprising a cationic lipid.

282. The composition of any of clauses 221 to 281, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

283. The composition of any of clauses 221 to 282, wherein the first and the second RNA molecules are encapsulated in, bound to, or adsorbed on a cationic lipid.

284. The composition of any of clauses 221 to 283, wherein the first and the second RNA molecules are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

285. The composition of any of clauses 221 to 284, wherein the first and the second RNA molecules are purified.

286. A method of expressing a polypeptide in a mammalian cell, comprising administering to the mammalian cell a composition comprising (i) a first RNA molecule according to any one of clauses 221 to 285, and (ii) a second RNA molecule according to any one of clauses 221 to 285, wherein the method expresses the polypeptide of interest in an amount that is, when measured under identical conditions, greater than a method that comprises administering to the mammalian cell a composition comprising the second RNA molecule, in the absence of the first RNA molecule.

287. A method of inducing an immune response in a subject, comprising administering to the subject in need thereof an effective amount of a composition according to any one of clauses 221 to 285.

288. A method of vaccinating a subject, comprising administering to the subject in need thereof an effective amount of a composition according to any one of clauses 221 to 285.

289. A method for treating or preventing an infectious disease, comprising administering to the subject in need thereof an effective amount of a composition according to any one of clauses 221 to 285.

290. The method according to any one of clauses 286 to 289, wherein the composition elicits an immune response comprising an antibody response.

291. The method according to any one of clauses 286 to 290, wherein the composition elicits an immune response comprising a T cell response.

292. A composition comprising (i) a first RNA molecule comprising a modified nucleotide; and (ii) a second RNA molecule comprising a 5′ Cap represented by Formula I,

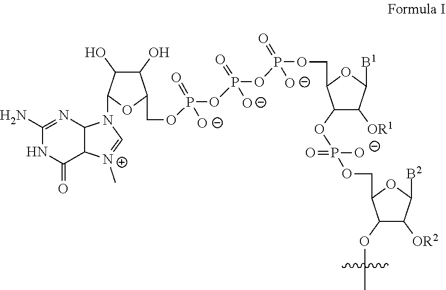

where R¹ and R² are each independently H or Me, and, B¹ and B² are each independently guanine, adenine, or uracil, a 5′ untranslated region, a coding region for a nonstructural protein derived from an alphavirus, a subgenomic promoter derived from an alphavirus, an open reading frame encoding a gene of interest, a 3′ untranslated region, and a 3′ poly A sequence, wherein at least 5% of a total population of a particular nucleotide in the second RNA molecule has been replaced with one or more modified or unnatural nucleotides.

293. The composition of clause 292, wherein B¹ and B² are naturally-occurring bases.

294. The composition of clause 292 or 293, wherein R¹ is methyl and R² is hydrogen.

295. The composition of any of clauses 292 to 294, wherein B¹ is guanine.

296. The composition of any of clauses 292 to 295, wherein B¹ is adenine.

297. The composition of any of clauses 292 to 296, wherein B² is adenine.

298. The composition of any of clauses 292 to 298, wherein B² is uracil.

299. The composition of any of clauses 292 to 298, wherein the nucleotide immediately downstream (5′ to 3′) of the 5′ Cap comprises guanine.

300. The composition of any of clauses 292 to 299, wherein B¹ is adenine and B² is uracil.

301. The composition of any of clauses 292 to 300, wherein B¹ is adenine, B² is uracil, R¹ is methyl, and R² is hydrogen.

302. The composition of any of clauses 292 to 301, wherein the nucleotide immediately downstream (5′ to 3′) of the 5′ Cap comprises guanine, B¹ is adenine, B² is uracil, R¹ is methyl, and R² is hydrogen.

303. The composition of any of clauses 292 to 302, wherein the composition reduces cytotoxicity, as compared to an identical composition in the absence of the first RNA molecule.

304. The composition of any of clauses 292 to 303, wherein the second RNA molecule in the presence of the first RNA molecule elicits less cytotoxicity as compared to the cytotoxicity elicited by the second RNA molecule in the absence of the first RNA molecule.

305. The composition of any of clauses 292 to 304, wherein the second RNA molecule in the presence of the first RNA molecule expresses the gene of interest in an amount that is greater than the amount of expression of the gene of interest in the absence of the first RNA molecule.

306. The composition of any of clauses 292 to 305, wherein the composition comprises an amount of the first RNA molecule that is greater than the amount of the second RNA molecule.

307. The composition of any of clauses 292 to 306, wherein the composition comprises an amount of the first RNA molecule that is at least about 2 times greater than the amount of the second RNA molecule.

308. The composition of any of clauses 292 to 307, wherein the first RNA molecule is capable of evading an innate immune response of a cell into which the first RNA molecule is introduced.

309. The composition of any of clauses 292 to 308, wherein at least 10% of a total population of a particular nucleotide in the first RNA molecule has been replaced with one or more modified or unnatural nucleotides.

310. The composition of any of clauses 292 to 309, wherein at least 25% of a total population of a particular nucleotide in the first RNA molecule has been replaced with one or more modified or unnatural nucleotides.

311. The composition of any of clauses 292 to 310, wherein at least 50% of a total population of a particular nucleotide in the first RNA molecule has been replaced with one or more modified or unnatural nucleotides.

312. The composition of any of clauses 292 to 311, wherein at least 75% of a total population of a particular nucleotide in the first RNA molecule has been replaced with one or more modified or unnatural nucleotides.

313. The composition of any of clauses 292 to 312, wherein essentially all of a particular nucleotide population in the first RNA molecule has been replaced with one or more modified or unnatural nucleotides.

314. The composition of any of clauses 292 to 313, wherein at least 10% of a total population of a particular nucleotide in the second RNA molecule has been replaced with one or more modified or unnatural nucleotides.

315. The composition of any of clauses 292 to 314, wherein at least 25% of a total population of a particular nucleotide in the second RNA molecule has been replaced with one or more modified or unnatural nucleotides.

316. The composition of any of clauses 292 to 315, wherein at least 50% of a total population of a particular nucleotide in the second RNA molecule has been replaced with one or more modified or unnatural nucleotides.

317. The composition of any of clauses 292 to 316, wherein at least 75% of a total population of a particular nucleotide in the second RNA molecule has been replaced with one or more modified or unnatural nucleotides.

318. The composition of any of clauses 292 to 317, wherein essentially all of a particular nucleotide population in the second RNA molecule has been replaced with one or more modified or unnatural nucleotides.

319. The composition of any of clauses 292 to 318, wherein the one or more modified or unnatural replacement nucleotides comprise two modified or unnatural nucleotides provided in a ratio ranging from 1:99 to 99:1, or any derivable range therein.

320. The composition of any of clauses 292 to 319, wherein at least 10% of a total population of a first particular nucleotide in the second RNA molecule has been replaced with one or more modified or unnatural nucleotides, and at least 10% of a total population of a second particular nucleotide in the second RNA molecule has been replaced with one or more modified or unnatural nucleotides.

321. The composition of any of clauses 292 to 320, wherein at least 10% of a total population of a first particular nucleotide in the second RNA molecule has been replaced with one or more modified or unnatural nucleotides, and at least 25% of a total population of a second particular nucleotide in the second RNA molecule has been replaced with one or more modified or unnatural nucleotides.

322. The composition of any of clauses 292 to 321, wherein at least 10% of a total population of a first particular nucleotide in the second RNA molecule has been replaced with one or more modified or unnatural nucleotides, and at least 50% of a total population of a second particular nucleotide in the second RNA molecule has been replaced with one or more modified or unnatural nucleotides.

323. The composition of any of clauses 292 to 322, wherein at least 10% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

324. The composition of any of clauses 292 to 323, wherein at least 10% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

325. The composition of any of clauses 292 to 324, wherein at least 25% of a total population of a first particular nucleotide in the second RNA molecule has been replaced with one or more modified or unnatural nucleotides, and at least 25% of a total population of a second particular nucleotide in the second RNA molecule has been replaced with one or more modified or unnatural nucleotides.

326. The composition of any of clauses 292 to 325, wherein at least 25% of a total population of a first particular nucleotide in the second RNA molecule has been replaced with one or more modified or unnatural nucleotides, and at least 50% of a total population of a second particular nucleotide in the second RNA molecule has been replaced with one or more modified or unnatural nucleotides.

327. The composition of any of clauses 292 to 326, wherein at least 25% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

328. The composition of any of clauses 292 to 327, wherein at least 25% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

329. The composition of any of clauses 292 to 328, wherein at least 50% of a total population of a first particular nucleotide in the second RNA molecule has been replaced with one or more modified or unnatural nucleotides, and at least 75% of a total population of a second particular nucleotide in the second RNA molecule has been replaced with one or more modified or unnatural nucleotides.

330. The composition of any of clauses 292 to 329, wherein at least 50% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

331. The composition of any of clauses 292 to 330, wherein at least 75% of a total population of a first particular nucleotide in the second RNA molecule has been replaced with one or more modified or unnatural nucleotides, and essentially all of a total population of a second particular nucleotide in the second RNA molecule has been replaced with one or more modified or unnatural nucleotides.

332. The composition of any of clauses 292 to 331, wherein the modified or unnatural nucleotide comprises any one nucleotide selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

333. The composition of any of clauses 292 to 332, wherein the modified or unnatural nucleotides are selected from the group consisting of 5-methyluridine, N1-methylpseudouridine, and 5-methylcytosine.

334. The composition of any of clauses 292 to 333, wherein at least 25% of a total population of uridine nucleotides in the first RNA molecule has been replaced with N1-methylpseudouridine.

335. The composition of any of clauses 292 to 334, wherein at least 50% of a total population of uridine nucleotides in the first RNA molecule has been replaced with N1-methylpseudouridine.

336. The composition of any of clauses 292 to 335, wherein at least 75% of a total population of uridine nucleotides in the first RNA molecule has been replaced with N1-methylpseudouridine.

337. The composition of any of clauses 292 to 336, wherein essentially all uridine nucleotides in the first RNA molecule have been replaced with N1-methylpseudouridine.

338. The composition of any of clauses 292 to 337, wherein at least 50% of a total population of uridine nucleotides in the first RNA molecule has been replaced with 5-methoxyuridine.

339. The composition of any of clauses 292 to 338, wherein essentially all uridine nucleotides in the first RNA molecule have been replaced with 5-methoxyuridine.

340. The composition of any of clauses 292 to 339, wherein at least 50% of a total population of uridine nucleotides in the first RNA molecule has been replaced with 5-methyluridine.

341. The composition of any of clauses 292 to 340, wherein essentially all uridine nucleotides in the first RNA molecule have been replaced with 5-methyluridine.

342. The composition of any of clauses 292 to 341, wherein at least 50% of a total population of cytosine nucleotides in the first RNA molecule has been replaced with 5-methylcytosine.

343. The composition of any of clauses 292 to 342, wherein essentially all cytosine nucleotides in the first RNA molecule have been replaced with 5-methylcytosine.

344. The composition of any of clauses 292 to 343, wherein at least 50% of a total population of uridine nucleotides in the first RNA molecule has been replaced with 2-thiouridine.

345. The composition of any of clauses 292 to 344, wherein essentially all uridine nucleotides in the first RNA molecule have been replaced with 2-thiouridine.

346. The composition of any of clauses 292 to 345, wherein at least 25% of a total population of uridine nucleotides in the second RNA molecule has been replaced with N1-methylpseudouridine.

347. The composition of any of clauses 292 to 346, wherein at least 50% of a total population of uridine nucleotides in the second RNA molecule has been replaced with N1-methylpseudouridine.

348. The composition of any of clauses 292 to 347, wherein at least 75% of a total population of uridine nucleotides in the second RNA molecule has been replaced with N1-methylpseudouridine.

349. The composition of any of clauses 292 to 348, wherein essentially all uridine nucleotides in the second RNA molecule have been replaced with N1-methylpseudouridine.

350. The composition of any of clauses 292 to 349, wherein at least 50% of a total population of uridine nucleotides in the second RNA molecule has been replaced with 5-methoxyuridine.

351. The composition of any of clauses 292 to 350, wherein essentially all uridine nucleotides in the second RNA molecule have been replaced with 5-methoxyuridine.

352. The composition of any of clauses 292 to 351, wherein at least 50% of a total population of uridine nucleotides in the second RNA molecule has been replaced with 5-methyluridine.

353. The composition of any of clauses 292 to 352, wherein essentially all uridine nucleotides in the second RNA molecule have been replaced with 5-methyluridine.

354. The composition of any of clauses 292 to 353, wherein at least 50% of a total population of cytosine nucleotides in the second RNA molecule has been replaced with 5-methylcytosine.

355. The composition of any of clauses 292 to 354, wherein essentially all cytosine nucleotides in the second RNA molecule have been replaced with 5-methylcytosine.

356. The composition of any of clauses 292 to 355, wherein at least 50% of a total population of uridine nucleotides in the second RNA molecule has been replaced with 2-thiouridine.

357. The composition of any of clauses 292 to 356, wherein essentially all uridine nucleotides in the second RNA molecule have been replaced with 2-thiouridine.

358. The composition of any of clauses 292 to 357, wherein at least 50% of a total population of uridine nucleotides in the second RNA molecule has been replaced with N1-methylpseudouridine and essentially all cytosine nucleotides in the second RNA molecule have been replaced with 5-methylcytosine.

359. The composition of any of clauses 292 to 358, wherein at least 50% of a total population of uridine nucleotides in the second RNA molecule has been replaced with 5-methoxyuridine and essentially all cytosine nucleotides in the second RNA molecule have been replaced with 5-methylcytosine.

360. The composition of any of clauses 292 to 359, wherein at least 50% of a total population of uridine nucleotides in the second RNA molecule has been replaced with 5-methyluridine and essentially all cytosine nucleotides in the second RNA molecule have been replaced with 5-methylcytosine.

361. The composition of any of clauses 292 to 360, wherein essentially all uridine nucleotides in the second RNA molecule have been replaced with about 50% 5-methoxyuridine and about 50% N1-methylpseudouridine.

362. The composition of any of clauses 292 to 361, wherein essentially all uridine nucleotides in the second RNA molecule have been replaced with about 75% 5-methoxyuridine and about 25% N1-methylpseudouridine.

363. The composition of any of clauses 292 to 362, wherein essentially all uridine nucleotides in the second RNA molecule have been replaced with about 25% 5-methoxyuridine and about 75% N1-methylpseudouridine.

364. The composition of any of clauses 292 to 363, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the second RNA molecule has been replaced with N1-methylpseudouridine, and essentially all cytosine nucleotides in the second RNA molecule have been replaced with 5-methylcytosine.

365. The composition of any of clauses 292 to 364, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the second RNA molecule has been replaced with 5-methoxyuridine, and essentially all cytosine nucleotides in the second RNA molecule have been replaced with 5-methylcytosine.

366. The composition of any of clauses 292 to 365, wherein the nucleotide immediately downstream (5′ to 3′) of the 5′ Cap comprises guanine, B¹ is adenine, B² is uracil, R¹ is methyl, and R² is hydrogen, at least 50% of a total population of uridine nucleotides in the second RNA molecule has been replaced with 5-methyluridine, and essentially all cytosine nucleotides in the second RNA molecule have been replaced with 5-methylcytosine.

367. The composition of any of clauses 292 to 366, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the second RNA molecule have been replaced with about 50% 5-methoxyuridine and about 50% N1-methylpseudouridine.

368. The composition of any of clauses 292 to 367, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the second RNA molecule have been replaced with about 75% 5-methoxyuridine and about 25% N1-methylpseudouridine.

369. The composition of any of clauses 292 to 368, wherein the nucleotide immediately downstream (5′ to 3′) of the 5′ Cap comprises guanine, B¹ is adenine, B² is uracil, R¹ is methyl, and R² is hydrogen, essentially all uridine nucleotides in the second RNA molecule have been replaced with about 25% 5-methoxyuridine and about 75% N1-methylpseudouridine.

370. The composition of any of clauses 292 to 369, wherein the first RNA molecule does not comprise a subgenomic promoter.

371. The composition of any of clauses 292 to 370, wherein the first RNA molecule is not a self-amplifying RNA molecule.

372. The composition of any of clauses 292 to 371, wherein the first RNA molecule further comprises a 5′ cap moiety.

373. The composition of any of clauses 292 to 372, wherein the first RNA molecule further comprises a 5′ untranslated region.

374. The composition of any of clauses 292 to 373, wherein the first RNA molecule further comprises a 3′ untranslated region.

375. The composition of any of clauses 292 to 374, wherein the first RNA molecule further comprises a 3′ poly A sequence.

376. The composition of any of clauses 292 to 375, wherein the first RNA molecule further comprises an open reading frame.

377. The composition of any of clauses 292 to 376, wherein the first RNA molecule does not comprise any one of a 5′ cap moiety, untranslated region, and poly A sequence.

378. The composition of any of clauses 292 to 377, wherein the first RNA molecule comprises a untranslated region and a 3′ untranslated region.

379. The composition of any of clauses 292 to 378, wherein the first RNA molecule comprises a cap moiety, a 5′ untranslated region (5′ UTR), a modified nucleotide, an open reading frame, a 3′ untranslated region (3′ UTR), a 3′ poly A sequence.

380. The composition of any of clauses 292 to 379, wherein the first RNA molecule does not comprise an open reading frame encoding an antigen.

381. The composition of any of clauses 292 to 380, wherein the first RNA molecule comprises a noncoding RNA region.

382. The composition of any of clauses 292 to 381, wherein the first RNA molecule comprises a coding RNA region.

383. The composition of any of clauses 292 to 382, wherein the 5′-cap moiety of the first RNA molecule is a natural 5′-cap.

384. The composition of any of clauses 292 to 383, wherein the 5′-cap moiety of the first RNA molecule is a 5′-cap analog.

385. The composition of any of clauses 292 to 384, wherein the nonstructural protein comprises alphavirus nonstructural protein nsP1.

386. The composition of any of clauses 292 to 385, wherein the nonstructural protein comprises alphavirus nonstructural protein nsP2.

387. The composition of any of clauses 292 to 386, wherein the nonstructural protein comprises alphavirus nonstructural protein nsP3.

388. The composition of any of clauses 292 to 387, wherein the nonstructural protein comprises alphavirus nonstructural protein nsP4.

389. The composition of any of clauses 292 to 388, wherein the nonstructural protein comprises alphavirus nonstructural protein nsP1, nsP2, and nsP3.

390. The composition of any of clauses 292 to 389, wherein the first RNA molecule does not comprise the alphavirus nonstructural protein 4 (nsP4).

391. The composition of any of clauses 292 to 390, wherein the second RNA molecule does not comprise the alphavirus nonstructural protein 4 (nsP4).

392. The composition of any of clauses 292 to 391, wherein the first RNA molecule comprises one or more modified nucleotides.

393. The composition of any of clauses 292 to 392, wherein the subgenomic promoter is operably linked to the open reading frame.

394. The composition of any of clauses 292 to 393, wherein the subgenomic promoter comprises a cis-acting regulatory element.

395. The composition of any of clauses 292 to 394, wherein the cis-acting regulatory element is immediately downstream of B 2.

396. The composition of any of clauses 292 to 395, wherein the cis-acting regulatory element is an AU-rich element.

397. The composition of any of clauses 292 to 396, wherein the second RNA molecule further comprises: (1) an alphavirus 5′ replication recognition sequence, and (2) an alphavirus 3′ replication recognition sequence.

398. The composition of any of clauses 292 to 397, wherein the second RNA molecule encodes at least one antigen.

399. The composition of any of clauses 292 to 398, wherein the second RNA molecule comprises at least 7000 nucleotides.

400. The composition of any of clauses 292 to 399, wherein the second RNA molecule comprises at least 8000 nucleotides.

401. The composition of any of clauses 292 to 400, wherein at least 80% of the total second RNA molecules are full length.

402. The composition of any of clauses 292 to 401, wherein the alphavirus is Venezuelan equine encephalitis virus.

403. The composition of any of clauses 292 to 402, wherein the alphavirus is Semliki Forest virus.

404. The composition of any of clauses 292 to 403, further comprising a pharmaceutically acceptable carrier.

405. The composition of any of clauses 292 to 404, further comprising a cationic lipid.

406. The composition of any of clauses 292 to 405, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

407. The composition of any of clauses 292 to 406, wherein the first and the second RNA molecules are encapsulated in, bound to, or adsorbed on a cationic lipid.

408. The composition of any of clauses 292 to 407, wherein the first and the second RNA molecules are encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

409. The composition of any of clauses 292 to 408, wherein the first and the second RNA molecules are purified.

410. A method of expressing a polypeptide in a mammalian cell, comprising administering to the mammalian cell a composition comprising (i) a first RNA molecule according to any one of clauses 292 to 409, and (ii) a second RNA molecule according to any one of clauses 292 to 409, wherein the method expresses the polypeptide of interest in an amount that is, when measured under identical conditions, greater than a method that comprises administering to the mammalian cell a composition comprising the second RNA molecule, in the absence of the first RNA molecule.

411. A method of inducing an immune response in a subject, comprising administering to the subject in need thereof an effective amount of a composition according to any one of clauses 292 to 409.

412. A method of vaccinating a subject, comprising administering to the subject in need thereof an effective amount of a composition according to any one of clauses 292 to 409.

413. A method for treating or preventing an infectious disease, comprising administering to the subject in need thereof an effective amount of a composition according to any one of clauses 292 to 397.

414. The method according to any one of clauses 292 to 413, wherein the composition elicits an immune response comprising an antibody response.

415. The method according to any one of clauses 292 to 414, wherein the composition elicits an immune response comprising a T cell response.

416. A composition comprising a self-amplifying RNA (saRNA) comprising: a 5′ Cap; a 5′ untranslated region (5′ UTR); a coding region for a nonstructural protein derived from an alphavirus; a first subgenomic promoter derived from an alphavirus; a first open reading frame encoding a first gene of interest derived from influenza virus hemagglutinin (HA); a second subgenomic promoter derived from an alphavirus; a second open reading frame encoding a second gene of interest derived from influenza virus; a 3′ untranslated region (3′ UTR); and a 3′ poly A sequence.

417. The composition according to clause 416, wherein the 5′ UTR comprises the sequence:

(SEQ ID NO: 8)
AUAGGCGGC GCAUGAGAGA AGCCCAGACC AAUUACCUAC CCAAA.

418. The composition according to any one of clauses 416-417, wherein the first subgenomic promoter is derived from Venezuelan Equine Encephalitis Virus (VEEV).

419. The composition according to clause 418, wherein the promoter comprises the sequence:

(SEQ ID NO: 9)
GGGCCCCUA UAACUCUCUA CGGCUAACCU GAAUGGACUA
CGACAUAGUC UAGUCCGCCA AG.

420. The composition according to any one of clauses 416-420, wherein the 3′ UTR comprises

(SEQ ID NO: 10)
AUAC AGCAGCAAUU GGCAAGCUGC UUACAUAGAA CUCGCGGCGA
UUGGCAUGCC GCCUUAAAAU UUUUAUUUUA UUUUUCUUUU
CUUUUCCGAA UCGGAUUUUG UUUUUAAUAU UUC.

421. The composition according to any one of clauses 416-420, wherein the second subgenomic promoter is derived from Venezuelan Equine Encephalitis Virus (VEEV) and comprises the sequence:

(SEQ ID NO: 9)
GGGCCCCUA UAACUCUCUA CGGCUAACCU GAAUGGACUA
CGACAUAGUC UAGUCCGCCA AG.

422. The composition according to any one of clauses 416-421, wherein the second gene of interest is derived from influenza virus neuraminidase (NA).

423. The composition according to any one of clauses 416-422, wherein the saRNA comprises a Kozak sequence,

(SEQ ID NO: 11)
AUAUCGCA CC.

424. A composition comprising a self-amplifying RNA (saRNA) comprising: a 5′ Cap; a 5′ untranslated region (5′ UTR); a coding region for a nonstructural protein derived from an alphavirus; a subgenomic promoter derived from an alphavirus; an open reading frame encoding a gene of interest derived from influenza virus; a 3′ untranslated region 3′ UTR); and a 3′ poly A sequence; wherein at least 5% of a total population of a particular nucleotide in the molecule has been replaced with one or more modified or unnatural nucleotides.

425. The composition according to clause 424, wherein the 5′ UTR comprises the sequence:

(SEQ ID NO: 8)
AUAGGCGGC GCAUGAGAGA AGCCCAGACC AAUUACCUAC CCAAA.

426. The composition according to any one of clauses 424-425, wherein the first subgenomic promoter is derived from Venezuelan Equine Encephalitis Virus (VEEV).

427. The composition according to clause 426, wherein the promoter comprises the sequence:

(SEQ ID NO: 9)
GGGCCCCUA UAACUCUCUA CGGCUAACCU GAAUGGACUA
CGACAUAGUC UAGUCCGCCA AG.

428. The composition according to any one of clauses 424-427, wherein the 3′ UTR comprises

(SEQ ID NO: 10)
AUAC AGCAGCAAUU GGCAAGCUGC UUACAUAGAA CUCGCGGCGA
UUGGCAUGCC GCCUUAAAAU UUUUAUUUUA UUUUUCUUUU
CUUUUCCGAA UCGGAUUUUG UUUUUAAUAU UUC.

429. The composition according to any one of clauses 424-428, wherein the saRNA comprises a Kozak sequence,

(SEQ ID NO: 11)
AUAUCGCA CC.

430. A composition comprising a self-amplifying RNA (saRNA) comprising: a 5′ Cap; a 5′ untranslated region (5′ UTR); a coding region for a nonstructural protein derived from an alphavirus; a first subgenomic promoter derived from an alphavirus; a first open reading frame encoding a first gene of interest derived from influenza virus hemagglutinin (HA); a second subgenomic promoter derived from an alphavirus; a second open reading frame encoding a second gene of interest derived from influenza virus; a 3′ untranslated region (3′ UTR); and a 3′ poly A sequence.

431. The composition of clause 430, wherein the 5′ cap is represented by Formula II:

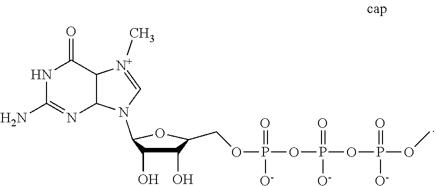

432. The composition of any one of clauses 430-431, wherein the nucleotides are naturally-occurring.

433. The composition of any one of clauses 430-431, wherein at least 10% of the total nucleotides in the saRNA has been replaced with modified or unnatural nucleotides.

434. The composition of any of clauses 430-433, wherein the modified or unnatural nucleotides are selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

435. The composition of any of clauses 430-434, wherein the nucleotide immediately downstream (5′ to 3′) of the 5′ Cap comprises guanine.

436. The composition of any of clauses 430-435, wherein the subgenomic promoter is operably linked to the open reading frame.

437. The composition of any of clauses 430-436, wherein the subgenomic promoter comprises a cis-acting regulatory element.

438. The composition of any of clauses 430-437, wherein the nonstructural protein comprises alphavirus nonstructural protein nsP1 and nsP2.

439. The composition of any of clauses 430 to 438, wherein the nonstructural protein comprises alphavirus nonstructural protein nsP3.

440. The composition of any of clauses 430 to 439, wherein the nonstructural protein comprises alphavirus nonstructural protein nsP4.

441. The composition of any of clauses 430 to 440, wherein the alphavirus is Venezuelan equine encephalitis virus.

442. The composition of any of clauses 430 to 441, wherein the alphavirus is Semliki Forest virus.

443. The composition of any of clauses 430 to 442, further comprising a pharmaceutically acceptable carrier.

444. The composition of any of clauses 430 to 443, further comprising a cationic lipid.

445. The composition of any of clauses 430 to 444, wherein the saRNA molecule is encapsulated in, bound to, or adsorbed on a cationic lipid.

446. The composition of any of clauses 430 to 445, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

447. The composition of any of clauses 430 to 446, wherein the RNA molecule is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

448. The composition of any of clauses 430 to 447, wherein at least 50% of the total saRNA molecules is full length.

449. The composition of any of clauses 430 to 448, wherein at least 80% of the total saRNA molecules is full length.

450. The composition of any of clauses 430 to 449, wherein the saRNA is complexed or associated with lipid nanoparticles (LNP) and wherein the LNP comprises: (i) at least one cationic lipid according to

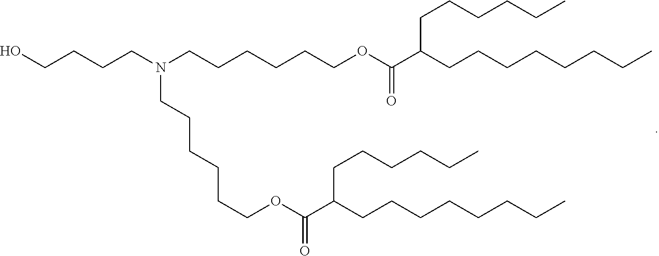 [SMILES: CCCCCCCCC(CCCCCC)C(=O)OCCCCCCN(CCCCO)CCCCCCOC(=O)C(CCCCCC)CCCCCCCC]

451. The composition of any of clauses 430 to 450, wherein the saRNA is complexed or associated with lipid nanoparticles (LNP) and wherein the LNP comprises: (ii) at least one neutral lipid, comprising 1,2-distearoyl-sn-glycero-3-phosphocholine (DSPC).

452. The composition of any of clauses 430 to 451, wherein the saRNA is complexed or associated with lipid nanoparticles (LNP) and wherein the LNP comprises: (iii) at least one steroid, comprising cholesterol.

453. The composition of any of clauses 430 to 452, wherein the saRNA is complexed or associated with lipid nanoparticles (LNP) and wherein the LNP comprises: (iv) at least one

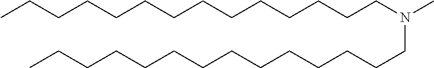 [SMILES: CCCCCCCCCCCCCCN(C)CCCCCCCCCCCCCC]

PEG-lipid according to

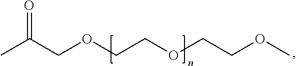

wherein n has a mean value ranging from 30 to 60.

454. The composition of any of clauses 430 to 453, wherein the saRNA is complexed or associated with lipid nanoparticles (LNP) and wherein the LNP comprises: (i) at least one cationic lipid according to

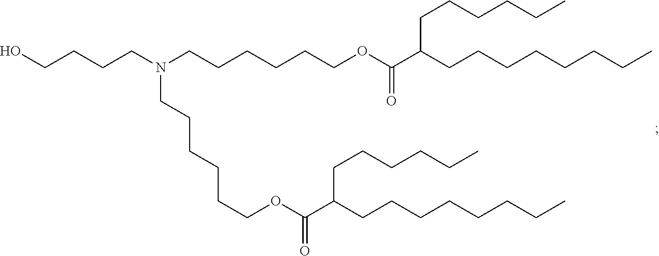 [SMILES: CCCCCCCCC(CCCCCC)C(=O)OCCCCCCN(CCCCO)CCCCCCOC(=O)C(CCCCCC)CCCCCCCC]

(ii) at least one neutral lipid, comprising 1,2-distearoyl-sn-glycero-3-phosphocholine (DSPC); (iii) at least one steroid, comprising cholesterol; and (iv) at least one PEG-lipid according to

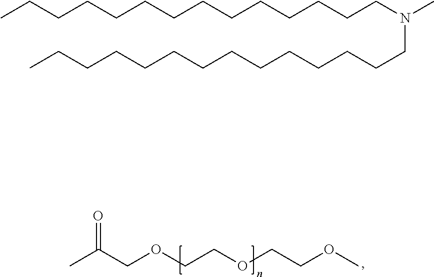

wherein n has a mean value ranging from 30 to 60.

455. The composition of any of clauses 430 to 454, wherein (i) to (iv) are in a molar ratio of about 20-60% cationic lipid, 5-25% neutral lipid, 25-55% sterol, and 0.5-15% PEG-lipid.

456. The composition of any of clauses 430 to 455, wherein the saRNA comprises at least one poly(A) sequence comprising 30 to 200 adenosine nucleotides.

457. The composition of any of clauses 430 to 456, wherein the at least open reading frame of the saRNA comprises a codon optimized sequence.

458. The composition of any of clauses 430 to 457, wherein the 5′ UTR comprises the sequence:

AUAGGCGGC GCAUGAGAGA AGCCCAGACC AAUUACCUAC CCAAA.

459. The composition of any of clauses 430 to 458, wherein the 3′ UTR comprises the sequence

(SEQ ID NO: 10)
AUAC AGCAGCAAUU GGCAAGCUGC UUACAUAGAA CUCGCGGCGA
UUGGCAUGCC GCCUUAAAAU UUUUAUUUUA UUUUUCUUUU
CUUUUCCGAA UCGGAUUUUG UUUUUAAUAU UUC.

460. The composition of any of clauses 430 to 459, wherein the saRNA does not comprise a modified nucleotide substitution.

461. The composition of any of clauses 430 to 460, wherein the saRNA does not comprise a 1-methylpseudouridine substitution.

462. A composition comprising a self-amplifying RNA (saRNA) comprising: a 5′ Cap; a 5′ untranslated region (5′ UTR); a coding region for a nonstructural protein derived from an alphavirus; a subgenomic promoter derived from an alphavirus; an open reading frame encoding a gene of interest derived from influenza virus; a 3′ untranslated region 3′ UTR); and a 3′ poly A sequence; wherein at least 5% of a total population of a particular nucleotide in the molecule has been replaced with one or more modified or unnatural nucleotides.

463. The composition of clause 462, wherein the 5′ Cap is represented by Formula II:

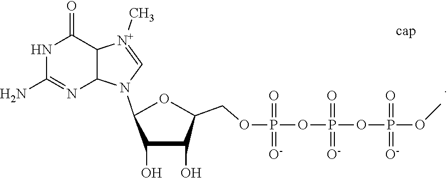

464. The composition of any of clauses 462 to 463, wherein the nucleotide immediately downstream (5′ to 3′) of the 5′ Cap comprises guanine.

465. The composition of any of clauses 462 to 464, wherein at least 10% of a total population of a particular nucleotide in the molecule has been replaced with one or more modified or unnatural nucleotides.

466. The composition of any of clauses 462 to 465, wherein at least 25% of a total population of a particular nucleotide in the molecule has been replaced with one or more modified or unnatural nucleotides.

467. The composition of any of clauses 462 to 466, wherein at least 50% of a total population of a particular nucleotide in the molecule has been replaced with one or more modified or unnatural nucleotides.

468. The composition of any of clauses 462 to 467, wherein at least 75% of a total population of a particular nucleotide in the molecule has been replaced with one or more modified or unnatural nucleotides.

469. The composition of any of clauses 462 to 468, wherein essentially all of a particular nucleotide population in the molecule has been replaced with one or more modified or unnatural nucleotides.

470. The composition of any of clauses 462 to 469, wherein the one or more modified or unnatural replacement nucleotides comprise two modified or unnatural nucleotides provided in a ratio ranging from 1:99 to 99:1, or any derivable range therein.

471. The composition of any of clauses 462 to 470, wherein the modified or unnatural nucleotides are selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

472. The composition of any of clauses 462 to 471, wherein the modified or unnatural nucleotides are selected from the group consisting of 5-methyluridine, N1-methylpseudouridine, 5-methoxyuridine, and 5-methylcytosine.

473. The composition of any of clauses 462 to 472, wherein at least 25% of a total population of uridine nucleotides in the molecule has been replaced with N1-methylpseudouridine.

474. The composition of any of clauses 462 to 473, wherein at least 50% of a total population of uridine nucleotides in the molecule has been replaced with N1-methylpseudouridine.

475. The composition of any of clauses 462 to 474, wherein at least 75% of a total population of uridine nucleotides in the molecule has been replaced with N1-methylpseudouridine.

476. The composition of any of clauses 462 to 475, wherein essentially all uridine nucleotides in the molecule have been replaced with N1-methylpseudouridine.

477. The composition of any of clauses 462 to 476, wherein at least 50% of a total population of uridine nucleotides in the molecule has been replaced with 5-methoxyuridine.

478. The composition of any of clauses 462 to 477, wherein essentially all uridine nucleotides in the molecule have been replaced with 5-methoxyuridine.

479. The composition of any of clauses 462 to 478, wherein at least 50% of a total population of uridine nucleotides in the molecule has been replaced with 5-methyluridine.

480. The composition of any of clauses 462 to 479, wherein essentially all uridine nucleotides in the molecule have been replaced with 5-methyluridine.

481. The composition of any of clauses 462 to 480, wherein at least 50% of a total population of cytosine nucleotides in the molecule has been replaced with 5-methylcytosine.

482. The composition of any of clauses 462 to 481, wherein essentially all cytosine nucleotides in the molecule have been replaced with 5-methylcytosine.

483. The composition of any of clauses 462 to 482, wherein at least 50% of a total population of uridine nucleotides in the molecule has been replaced with 2-thiouridine.

484. The composition of any of clauses 462 to 483, wherein essentially all uridine nucleotides in the molecule have been replaced with 2-thiouridine.

485. The composition of any of clauses 462 to 484, wherein at least 50% of a total population of uridine nucleotides in the molecule has been replaced with N1-methylpseudouridine and essentially all cytosine nucleotides in the molecule have been replaced with 5-methylcytosine.

486. The composition of any of clauses 462 to 485, wherein at least 50% of a total population of uridine nucleotides in the molecule has been replaced with 5-methoxyuridine and essentially all cytosine nucleotides in the molecule have been replaced with 5-methylcytosine.

487. The composition of any of clauses 462 to 486, wherein at least 50% of a total population of uridine nucleotides in the molecule has been replaced with 5-methyluridine and essentially all cytosine nucleotides in the molecule have been replaced with 5-methylcytosine.

488. The composition of any of clauses 462 to 487, wherein essentially all uridine nucleotides in the molecule have been replaced with about 50% 5-methoxyuridine and about 50% N1-methylpseudouridine.

489. The composition of any of clauses 462 to 488, wherein essentially all uridine nucleotides in the molecule have been replaced with about 75% 5-methoxyuridine and about 25% N1-methylpseudouridine.

490. The composition of any of clauses 462 to 489, wherein essentially all uridine nucleotides in the molecule have been replaced with about 25% 5-methoxyuridine and about 75% N1-methylpseudouridine.

491. The composition of any of clauses 462 to 490, wherein the subgenomic promoter is operably linked to the open reading frame.

492. The composition of any of clauses 462 to 491, wherein the subgenomic promoter comprises a cis-acting regulatory element.

493. The composition of any of clauses 462 to 492, wherein the nonstructural protein comprises alphavirus nonstructural protein nsP1 and nsP2.

494. The composition of any of clauses 462 to 493, wherein the nonstructural protein comprises alphavirus nonstructural protein nsP3.

495. The composition of any of clauses 462 to 494, wherein the nonstructural protein comprises alphavirus nonstructural protein nsP4.

496. The composition of any of clauses 462 to 495, wherein the alphavirus is Venezuelan equine encephalitis virus.

497. The composition of any of clauses 462 to 496, wherein the alphavirus is Semliki Forest virus.

498. The composition of any of clauses 462 to 497, further comprising a pharmaceutically acceptable carrier.

499. The composition of any of clauses 462 to 498, further comprising a cationic lipid.

500. The composition of any of clauses 462 to 499, wherein the saRNA molecule is encapsulated in, bound to, or adsorbed on a cationic lipid.

501. The composition of any of clauses 462 to 500, further comprising a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

502. The composition of any of clauses 462 to 501, wherein the saRNA molecule is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

503. The composition of any of clauses 462 to 502, wherein at least 50% of the total saRNA molecules is full length.

504. The composition of any of clauses 462 to 503, wherein at least 80% of the total saRNA molecules is full length.

505. The composition of any of clauses 462 to 504, wherein the saRNA is complexed or associated with lipid nanoparticles (LNP) and wherein the LNP comprises: (i) at least one cationic lipid according to

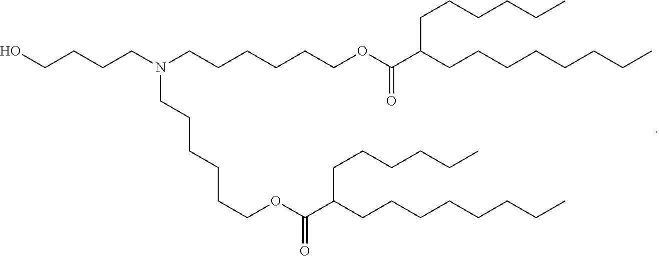 [SMILES: CCCCCCCCC(CCCCCC)C(=O)OCCCCCCN(CCCCO)CCCCCCOC(=O)C(CCCCCC)CCCCCCCC]

506. The composition of any of clauses 462 to 505, wherein the saRNA is complexed or associated with lipid nanoparticles (LNP) and wherein the LNP comprises: (ii) at least one neutral lipid, comprising 1,2-distearoyl-sn-glycero-3-phosphocholine (DSPC).

507. The composition of any of clauses 462 to 506, wherein the saRNA is complexed or associated with lipid nanoparticles (LNP) and wherein the LNP comprises: (iii) at least one steroid, comprising cholesterol.

508. The composition of any of clauses 462 to 507, wherein the saRNA is complexed or associated with lipid nanoparticles (LNP) and wherein the LNP comprises: (iv) at least one

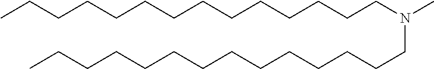 [SMILES: CCCCCCCCCCCCCCN(C)CCCCCCCCCCCCCC]

PEG-lipid according to ranging from 30 to 60.

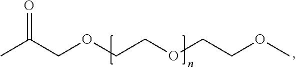

wherein n has a mean value 509. The composition of any of clauses 462 to 508, wherein the saRNA is complexed or associated with lipid nanoparticles (LNP) and wherein the LNP comprises: (i) at least one

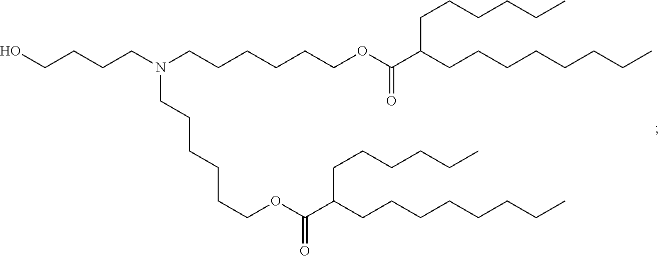 [SMILES: CCCCCCCCC(CCCCCC)C(=O)OCCCCCCN(CCCCO)CCCCCCOC(=O)C(CCCCCC)CCCCCCCC]

cationic lipid according tc (ii) at least one neutral lipid, comprising 1,2-distearoyl-sn-glycero-3-phosphocholine (DSPC); (iii) at least one steroid, comprising cholesterol; and (iv) at least one PEG-lipid according to

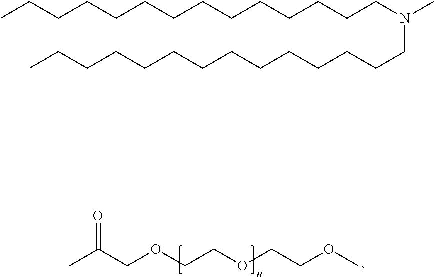

wherein n has a mean value ranging from 30 to 60.

510. The composition of any of clauses 462 to 509, wherein (i) to (iv) are in a molar ratio of about 20-60% cationic lipid, 5-25% neutral lipid, 25-55% sterol, and 0.5-15% PEG-lipid.

511. The composition of any of clauses 462 to 510, wherein the saRNA comprises at least one poly(A) sequence comprising 30 to 200 adenosine nucleotides.

512. The composition of any of clauses 462 to 82, wherein the at least open reading frame of the saRNA comprises a codon optimized sequence.

513. The composition of any of clauses 462 to 511, wherein the 5′ UTR comprises the sequence SEQ ID NO: :

(SEQ ID NO: 9)
GGGCCCCUA UAACUCUCUA CGGCUAACCU GAAUGGACUA
CGACAUAGUC UAGUCCGCCA AG.

514. The composition of any of clauses 462 to 513, wherein the 3′ UTR comprises the sequence SEQ ID NO:

(SEQ ID NO: 10)
AUAC AGCAGCAAUU GGCAAGCUGC UUACAUAGAA CUCGCGGCGA
UUGGCAUGCC GCCUUAAAAU UUUUAUUUUA UUUUUCUUUU
CUUUUCCGAA UCGGAUUUUG UUUUUAAUAU UUC.

515. The composition of any of clauses 462 to 514, wherein the saRNA does not comprise a modified nucleotide substitution.

516. The composition of any of clauses 462 to 515, wherein the saRNA does not comprise a 1-methylpseudouridine substitution.

517. The composition of any of clauses 462 to 516, Wherein the saRNA does not comprise a Kozak sequence.

518. A method of inducing an immune response in a subject, comprising administering to the subject in need thereof an effective amount of the composition of any of clauses 430 to 517.

519. A method of vaccinating a subject, comprising administering to the subject in need thereof an effective amount of the composition of any of clauses 430 to 517.

520. A method for treating or preventing an infectious disease, comprising administering to the subject in need thereof an effective amount of the composition of any of clauses 430 to 517.

521. The method of any of clauses 430 to 520, wherein the composition elicits an immune response comprising an antibody response.

522. The method of any of clauses 430 to 521, wherein the composition elicits an immune response comprising a T cell response.

523. A method of inducing an immune response in a subject, comprising administering to the subject in need thereof an effective amount of the composition of any of clauses 430 to 517.

524. A method of vaccinating a subject, comprising administering to the subject in need thereof an effective amount of the composition of any of clauses 430 to 517.

525. A method for treating or preventing an infectious disease, comprising administering to the subject in need thereof an effective amount of the composition of any of clauses 430 to 517.

526. The method of any of clauses 518-525, wherein the composition elicits an immune response comprising an antibody response.

527. The method according of any of clauses 518-526, wherein the composition elicits an immune response comprising a T cell response.

528. A composition according to any one of clauses 430-517 wherein the composition comprises a plurality of the saRNA encapsulated in a lipid nanoparticle.

529. The composition according to any one of clauses 430-517 wherein the composition comprises four of the saRNA encapsulated in a lipid nanoparticle.

1. A composition comprising a self-amplifying RNA (saRNA) comprising: a 5′ Cap; a untranslated region (5′ UTR); a coding region for a nonstructural protein derived from an alphavirus; a first subgenomic promoter derived from an alphavirus; a first open reading frame encoding a first gene of interest derived from influenza virus hemagglutinin (HA); a second subgenomic promoter derived from an alphavirus; a second open reading frame encoding a second gene of interest derived from influenza virus; a 3′ untranslated region (3′ UTR); and a 3′ poly A sequence.

2. The composition according to claim 1, wherein the 5′ UTR sequence has at least 70%, sequence identity to SEQ ID NO: 12; wherein the coding region for a nonstructural protein comprises a polynucleotide sequence having at least 70% sequence identity to SEQ ID NO: 13, a polynucleotide sequence having at least 70% sequence identity to SEQ ID NO: 14, a polynucleotide sequence having at least 70% sequence identity to SEQ ID NO: 15, and a polynucleotide sequence having at least 70% sequence identity to SEQ ID NO: 16; wherein the first subgenomic promoter has a polynucleotide sequence having at least 70% sequence identity to SEQ ID NO: 17; wherein the polynucleotide sequence encoding the HA has at least 70% sequence identity to SEQ ID NO: 18; wherein the second subgenomic promoter comprises a polynucleotide sequence having at least 70% sequence identity to SEQ ID NO: 19; wherein the 3′ UTR comprises a polynucleotide sequence having at least 70% sequence identity to SEQ ID NO: 21; and the poly A tail comprises at least 20 consecutive adenines.

3. The composition according to claim 1, wherein the second gene of interest derived from influenza virus encodes any one polypeptide selected from HA, NA, NP, M1, M2, NS1 and NS2.

4. The composition according to claim 1, wherein the 5′ cap is represented by Formula II:

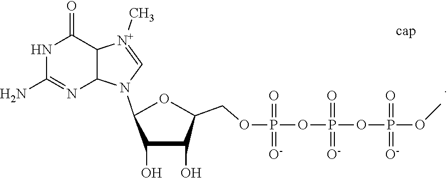

5. The composition according to claim 1, wherein at least 10% of the total nucleotides in the saRNA has been replaced with modified or unnatural nucleotides, selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

6. The composition according to claim 1, wherein the nucleotide immediately downstream (5′ to 3′) of the 5′ Cap comprises guanine.

7. The composition according to claim 1, wherein at least 50% of the total saRNA molecules is full length.

8. The composition according to claim 1, wherein at least 80% of the total saRNA molecules is full length.

9. The composition according to claim 1, wherein the saRNA is complexed or associated with a lipid nanoparticle (LNP), which comprises an ionizable lipid, a neutral lipid, a steroid, and a polymer-conjugated lipid.

10. The composition according to claim 1, wherein the saRNA is complexed or associated with a lipid nanoparticle (LNP), which comprises (i) at least one cationic lipid according to 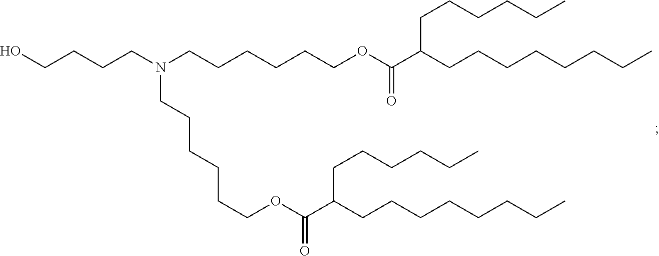 [SMILES: CCCCCCCCC(CCCCCC)C(=O)OCCCCCCN(CCCCO)CCCCCCOC(=O)C(CCCCCC)CCCCCCCC]
(ii) at least one neutral lipid, comprising 1,2-distearoyl-sn-glycero-3-phosphocholine (DSPC); (iii) at least one steroid, comprising cholesterol; and (iv) at least one PEG-lipid according to 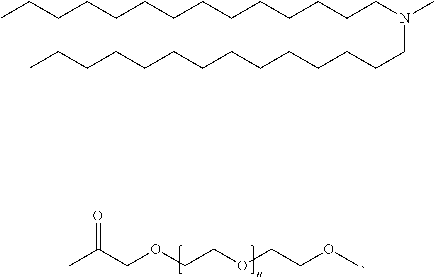
wherein n has a mean value ranging from 30 to 60.

11. The composition according to claim 1, wherein (i) to (iv) are in a molar ratio of about 20-60% cationic lipid, 5-25% neutral lipid, 25-55% sterol, and 0.5-15% PEG-lipid.

12. The composition according to claim 1, wherein the saRNA comprises at least one poly(A) sequence comprising 30 to 200 adenosine nucleotides.

13. A composition comprising a self-amplifying RNA (saRNA) comprising: a 5′ Cap; a untranslated region (5′ UTR); a coding region for a nonstructural protein derived from an alphavirus; a subgenomic promoter derived from an alphavirus; an open reading frame encoding a gene of interest derived from influenza virus; a 3′ untranslated region (3′ UTR); and a 3′ poly A sequence; wherein at least 25% of a total population of a particular nucleotide in the saRNA has been replaced with one or more modified or unnatural nucleotides selected from the group consisting of 5-methyluridine, N1-methylpseudouridine, and 5-methylcytosine.

14. The composition according to claim 13, wherein the 5′ Cap is represented by Formula II:

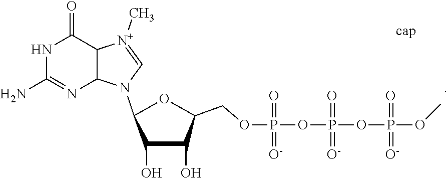

15. The composition according to claim 13, wherein the nucleotide immediately downstream (5′ to 3′) of the 5′ Cap comprises guanine.

16. The composition according to claim 13, wherein the saRNA molecule is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

17. The composition according to claim 13, wherein at least 50% of the total saRNA molecules is full length.

18. The composition according to claim 13, wherein at least 80% of the total saRNA molecules is full length.

19. A method for inducing an immune response in a subject, comprising a composition, which comprises a self-amplifying RNA (saRNA) comprising: a 5′ Cap; a 5′ untranslated region (5′ UTR); a coding region for a nonstructural protein derived from an alphavirus; a first subgenomic promoter derived from an alphavirus; a first open reading frame encoding a first gene of interest derived from influenza virus hemagglutinin (HA); a second subgenomic promoter derived from an alphavirus; a second open reading frame encoding a second gene of interest derived from influenza virus; a 3′ untranslated region (3′ UTR); and a 3′ poly A sequence.

20. The method according to claim 19, wherein the composition elicits an immune response comprising a T cell response.

21. The composition according to claim 1, wherein the composition comprises a plurality of the saRNA, encapsulated in a lipid nanoparticle.

22. The composition according to claim 21, wherein the composition comprises four of the saRNA, encapsulated in a lipid nanoparticle.
